(19) United States
(12) Patent Application Publication
(10) Pub. No.: US 2022/0169645 A1
(43) Pub. Date: Jun. 2, 2022

(54) COMPOUNDS AS BRUTON TYROSINE KINASE INHIBITORS, PREPARATION METHODS AND MEDICAL APPLICATIONS THEREOF

(71) Applicant: Eternity Bioscience Inc., Cranbury, NJ (US)

(72) Inventors: Puhui Li, Plainsboro, NJ (US); Yinfa Yan, Bedminster, NJ (US); Jian Liu, Edison, NJ (US); Dong Liu, Basking Ridge, NJ (US); Chunying Song, Newtown, PA (US); Suxing Liu, Edison, NJ (US)

(21) Appl. No.: 17/540,763

(22) Filed: Dec. 2, 2021

Related U.S. Application Data
(60) Provisional application No. 63/163,091, filed on Mar. 19, 2021.
(60) Provisional application No. 63/120,455, filed on Dec. 2, 2020.

(51) Int. Cl. C07D 471/04; C07D 231/38; C07D 405/04; C07D 409/04; C07D 401/04

(52) U.S. Cl. CPC C07D 471/04; C07D 231/38; C07D 401/04; C07D 409/04; C07D 405/04

(57) ABSTRACT

This application discloses Bruton Tyrosine Kinase (BTK) inhibitors of the general formula (IM) and analogs thereof, pharmaceutical compositions containing these compounds, methods of preparing them, and use of these compounds as therapeutic agents for the treatment of various disorders related to the excessive BTK activity, including cancers, immune disorders, cardiovascular diseases, viral infections, inflammation, metabolism/endocrine function disorders, and neurological disorders.

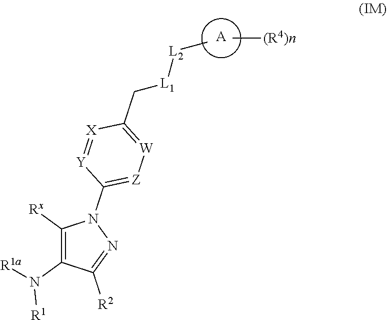

### CROSS-REFERENCE TO RELATED APPLICATIONS

This application claims priority under 35 U.S.C. § 119(e) to U.S. Provisional Patent Application No. 63/120,455, filed on Dec. 2, 2020, and Provisional Patent Application No. 63/163,091, filed on Mar. 19, 2021, the disclosures of both of which are incorporated herein by reference in their entirety.

### FIELD OF THE DISCLOSURE

The present application relates to inhibitors of Bruton Tyrosine Kinase (BTK), including wild type and mutant BTK, useful for the treatment of BTK-associated diseases or disorders, such as cancers, immune disorders, cardiovascular diseases, viral infections, inflammation, metabolism/endocrine function disorders, and neurological disorders.

### BACKGROUND OF THE DISCLOSURE

Bruton tyrosine kinase (BTK) is a TEC family cytoplasmic nonreceptor tyrosine kinase. Its protein structure contains an N-terminal pleckstrin homology (PH) domain, a TEC homology (TH) domain, SRC homology (SH) domains SH2 and SH3, and a kinase domain with enzymatic activity (Hendriks R W, et al., Nat. Rev. Cancer, 2014, 14: 219-232). Its PH domain recruits BTK to the cell membrane by interacting with phosphatidylinositol-3,4,5-triphosphate (PIP3), which is generated by phosphatidylinositol-3 kinase (PI3K). Transmembrane proteins such as B-cell receptor (BCR) complex promote phosphorylation of BTK at Y551 by SYK or SRC family kinases, leading to BTK kinase activation, and subsequently to its Y223 autophosphorylation in the SH3 domain (Rawlings, D J et al., Science, 1996, 271: 822-825). BTK is expressed in B lymphocytes and is essential at various stages of B lymphocyte development (Burger, J A et al., Nat. Rev. Cancer, 2018, 18: 148-167). BTK was initially shown to be mutated in the human primary immunodeficiency X-linked agammaglobulinemia (XLA). XLA patients are characterized with low numbers of B cells and almost absence of antibodies in their circulation (Vetrie D, et al., Nature, 1993, 361: 226-233; Tsukada S et al., Cell, 1993, 72: 279-290). BTK is also expressed in some types of myeloid cells such as macrophages, neutrophils and mast cells. In these innate immune cells, BTK is indicated in toll-like receptors (TLRs), Fc receptors (FCRs) and chemokine receptors mediated signaling (Crofford et al., Expert Rev. Clin. Immunol., 2016, 12: 763-773). Activation of BTK stimulates several downstream signaling pathways, such as NFκB and MAP (mitogen-activated protein) kinase pathways. Aberrant expression and/or activation of BTK has been found in various B cell malignancies and is critical for cancer cell survival and autoimmunity disorders.

BTK inhibitors have been developed with the goal to treat cancers and autoimmune diseases, such as chronic lymphocytic leukemia (CLL) and rheumatoid arthritis (RA) or lupus. Several covalent BTK inhibitors have been in clinical use for B-cell malignancies. However, these inhibitors target a cysteine residue C481 in the BTK kinase domain for a covalent binding with the side chain thiol. With the treatment of clinical covalent BTK inhibitors in cancer patients, resistances have emerged. Mutations within BTK protein, such as C481S, C481Y, C481R, and C481F, have been reported in the relapsed cancers and shown to cause loss of the drug covalent binding site (Liu L, et al., Future Med. Chem., 2018, 10: 343-356).

Relapse of cancers, such as CLL or mantle cell lymphoma (MCL), after covalent BTK inhibitor therapies is an issue of increasing clinical significance (Wayach J A, et al., J. Clin. Oncol., 2017, 35: 1437-1443).

### SUMMARY OF THE DISCLOSURE

To circumvent the limitation of the covalent binding Bruton Tyrosine Kinase (BTK) inhibitors, the present disclosure provides compounds and methods useful in the suppression of BTK, and use of these compounds to treat disorders related to hyperactive BTK, including cancers, immune disorders, cardiovascular diseases, viral infections, inflammation, metabolism/endocrine function disorders, and neurological disorders.

The present disclosure, in one aspect, provides a compound of formula (IM) having the following structure:

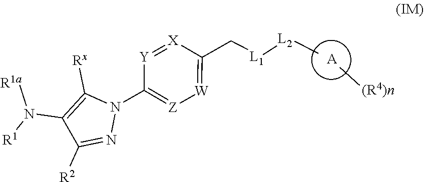

or a pharmaceutically acceptable salt, solvate, or prodrug thereof, wherein:

R^{x} is selected from the group consisting of C(O)NR^{A}R^{B}, alkyl, haloalkyl and hydroxyalkyl; wherein the alkyl is optionally substituted with one or more, sometimes preferably one to three, groups independently selected from the group consisting of alkoxy and haloalkoxy;

R¹ and R^{1a} are identical or different, and each is independently selected from the group consisting of hydrogen, alkyl, haloalkyl, —C(O)R⁵, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;

R² is selected from the group consisting of alkyl, haloalkyl, hydroxyalkyl, —COR⁵, cyano, —OR⁵, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;

or R¹ and R² together with the nitrogen and the carbon to which they are attached form a heterocycle; wherein the heterocycle is optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl and hydroxyalkyl;

X, Y, Z and W are identical or different, and each is independently CR³ or N, provided that X, Y, Z and W are not all N at the same time;

R³ at each occurrence is identical or different, and each is selected from the group consisting of hydrogen, halogen, alkyl, haloalkyl, alkoxy, haloalkoxy, cyano, amino, hydroxyl and hydroxyalkyl;

L₁ and L₂ are different, and each is independently —NR^{7a} or C(O);

R^{7a} is selected from the group consisting of hydrogen, alkyl, haloalkyl and hydroxyalkyl;

ring A is aryl or heteroaryl;

R⁴ at each occurrence is identical or different, and each is selected from the group consisting of hydrogen, halogen, alkyl, haloalkyl, cyano, —OR^{6a}, —NR^{7b}R^{8b} and hydroxyalkyl;

R^{A}, R^{B}, R⁵, R⁶, R^{6a}, R⁷, R^{7b}, R⁸ and R^{8b} are identical or different, and each is independently selected from the group consisting of hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and

n is 0, 1, 2, 3, 4, 5 or 6.

In another aspect, the present disclosure provides a pharmaceutical composition, comprising a compound of formula (IM), or a pharmaceutically acceptable salt, solvate, or prodrug thereof, and one or more pharmaceutically acceptable carriers, diluents and/or other excipients.

In another aspect, the present disclosure provides a method of treatment of a condition which is modulated by BTK, wherein the method comprises a step of administering to a subject in need thereof a therapeutically effective amount of the compound of formula (IM), or a pharmaceutically acceptable salt, solvate, or prodrug thereof, or the pharmaceutical composition thereof.

The condition modulated by BTK is selected from the group consisting of cancers, immune disorders, cardiovascular diseases, viral infections, inflammation, metabolism/endocrine function disorders, and neurological disorders; preferably, wherein the condition modulated by BTK is selected from the group consisting of B-cell malignancy, B-cell lymphoma, diffuse large B cell lymphoma, chronic lymphocyte leukemia, non-Hodgkin lymphoma (for example ABC-DLBCL), mantle cell lymphoma, follicular lymphoma, hairy cell leukemia, B-cell non-Hodgi lymphoma, Waldenstrom's macroglobulinemia, multiple myeloma, bone cancer, bone metastasis, arthritis, multiple sclerosis, osteoporosis, irritable bowel syndrome, inflammatory bowel disease, Crohn's disease, Sjogren's syndrome and lupus.

Other aspects and advantages of the present disclosure will be better appreciated in view of the following detailed description, examples, and claims.

### DETAILED DESCRIPTION OF THE DISCLOSURE

In one aspect, the present disclosure provides a compound of formula (IM):

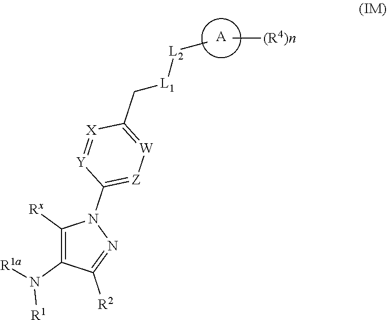

or a pharmaceutically acceptable salt, solvate, or prodrug thereof, wherein:

R^{x} is selected from the group consisting of C(O)NR^{A}R^{B}, alkyl, haloalkyl and hydroxyalkyl; wherein the alkyl is optionally substituted with one or more, sometimes preferably one to three, groups independently selected from the group consisting of alkoxy and haloalkoxy;

R¹ and R^{1a} are identical or different, and each is independently selected from the group consisting of hydrogen, alkyl, haloalkyl, —C(O)R⁵, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;

R² is selected from the group consisting of alkyl, haloalkyl, hydroxyalkyl, —COR⁵, cyano, —OR⁵, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;

or R¹ and R² together with the nitrogen and the carbon to which they are attached form a heterocycle; wherein the heterocycle is optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl and hydroxyalkyl;

X, Y, Z and W are identical or different, and each is independently CR³ or N, provided that X, Y, Z and W are not all N at the same time;

R³ at each occurrence is identical or different, and each is selected from the group consisting of hydrogen, halogen, alkyl, haloalkyl, alkoxy, haloalkoxy, cyano, amino, hydroxyl and hydroxyalkyl;

L₁ and L₂ are different, and each is independently —NR^{7a} or C(O);

R^{7a} is selected from the group consisting of hydrogen, alkyl, haloalkyl and hydroxyalkyl;

ring A is aryl or heteroaryl;

R⁴ at each occurrence is identical or different, and each is selected from the group consisting of hydrogen, halogen, alkyl, haloalkyl, cyano, —OR^{6a}, —NR^{7b}R^{8b} and hydroxyalkyl;

R^{A}, R^{B}, R⁵, R⁶, R^{6a}, R⁷, R^{7b}, R⁸ and R^{8b} are identical or different, and each is independently selected from the group consisting of hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and

n is 0, 1, 2, 3, 4, 5 or 6.

In one embodiment, the disclosure provides a compound of formula (IM), or a pharmaceutically acceptable salt, solvate, or prodrug thereof, wherein R^{1a} is hydrogen.

In one embodiment of the disclosure, the compound of formula (IM) is selected from a compound of formula (I):

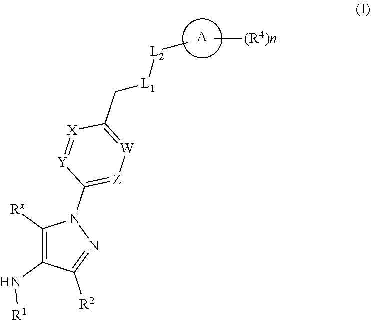

or a pharmaceutically acceptable salt, solvate, or prodrug thereof, wherein:

R^{x} is selected from the group consisting of C(O)NR^{A}R^{B}, alkyl, haloalkyl and hydroxyalkyl; wherein the alkyl is optionally substituted with one or more, sometimes preferably one to three, groups independently selected from the group consisting of alkoxy and haloalkoxy;

R¹ is selected from the group consisting of hydrogen, alkyl, haloalkyl, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;

R² is selected from the group consisting of alkyl, haloalkyl, hydroxyalkyl, —COR⁵, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;

or R¹ and R² together with the nitrogen and the carbon to which they are attached form a heterocycle; wherein the heterocycle is optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl and hydroxyalkyl;

X, Y, Z and W are identical or different, and each is independently CR³ or N, provided that X, Y, Z and W are not all N at the same time;

R³ at each occurrence is identical or different, and each is selected from the group consisting of hydrogen, halogen, alkyl, haloalkyl, alkoxy, haloalkoxy, cyano, amino, hydroxyl and hydroxyalkyl;

L₁ and L₂ are different, and each is independently —NR^{7a} or C(O);

R^{7a} is selected from the group consisting of hydrogen, alkyl, haloalkyl and hydroxyalkyl;

ring A is aryl or heteroaryl;

R⁴ at each occurrence is identical or different, and each is selected from the group consisting of hydrogen, halogen, alkyl, haloalkyl, cyano, —OR^{6a}, —NR^{7b}R^{8b} and hydroxyalkyl;

R^{A}, R^{B}, R⁵, R⁶, R^{6a}, R⁷, R^{7b}, R⁸ and R^{8b} are identical or different, and each is independently selected from the group consisting of hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more, sometimes preferably one to five, and sometimes more preferably one to three, groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and

n is 0, 1, 2, 3, 4, 5 or 6.

In one embodiment, the disclosure provides a compound of formula (IM) or formula (I), or a pharmaceutically acceptable salt, solvate, or prodrug thereof, wherein R^{x} is C(O)NR^{A}R^{B}, R^{A} and R^{B} are as defined in formula (IM).

In one embodiment, the disclosure provides a compound of formula (IM) or formula (I), or a pharmaceutically acceptable salt, solvate, or prodrug thereof, wherein L₁ is NR^{7a}, L₂ is C(O), and R^{7a} is as defined in formula (IM).

In one embodiment, the disclosure provides a compound of formula (IM) or formula (I), or a pharmaceutically acceptable salt, solvate, or prodrug thereof, wherein L₁ is NR^{7a}, L₂ is C(O), and R^{7a} is selected from the group consisting of hydrogen, C₁₋₆ alkyl, C₁₋₆ haloalkyl and C₁₋₆ hydroxyalkyl; preferably R^{7a} is hydrogen.

In one embodiment of the disclosure, the compound of formula (IM) or formula (I) is selected from a compound of formula (II):

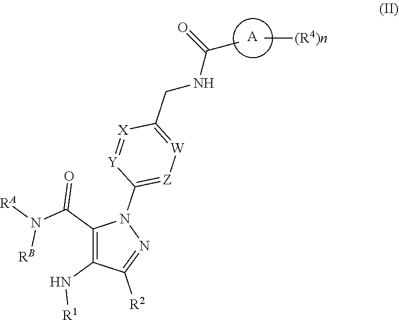

or a pharmaceutically acceptable salt, solvate, or prodrug thereof, wherein:

R^{A}, R^{B}, W, X, Y, Z, R¹, R², R⁴, ring A and n are as defined in formula (IM).

In one embodiment, the disclosure provides a compound of formula (IM), formula (I), formula (II), or a pharmaceutically acceptable salt, solvate, or prodrug thereof, wherein ring A is selected from the group consisting of 6-10 membered aryl or 5-10 membered heteroaryl; preferably ring A is phenyl or

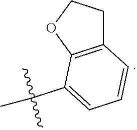 [SMILES: *c1cccc2c1OCC2]

In one embodiment, the disclosure provides a compound of formula (I), formula (II), or a pharmaceutically acceptable salt, solvate, or prodrug thereof, wherein ring A is selected from the group consisting of 6-10 membered aryl or 5-10 membered heteroaryl; preferably ring A is phenyl.

In one embodiment, the disclosure provides a compound of formula (IM), formula (I), formula (II), or a pharmaceutically acceptable salt, solvate, or prodrug thereof, wherein R^{A} and R^{B} are identical or different, and each is independently selected from the group consisting of hydrogen, C₁₋₆ alkyl, C₁₋₆ haloalkyl, C₁₋₆ hydroxyalkyl, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, C₁₋₆ alkoxy, C₁₋₆ haloalkoxy, cyano, amino, hydroxyl, oxo, C₁₋₆ hydroxyalkyl, 3-8 membered cycloalkyl, 3-12 membered heterocyclyl, 6-10 membered aryl and 5-10 membered heteroaryl; preferably R^{A} and R^{B} are identical, and each is hydrogen.

In one embodiment of the disclosure, the compound of formula (IM), formula (I) or formula (II), is selected from a compound of formula (III):

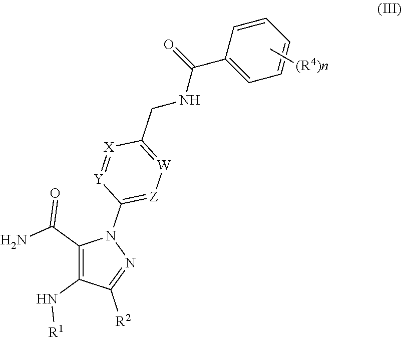

or a pharmaceutically acceptable salt, solvate, or prodrug thereof, wherein:

W, X, Y, Z, R¹, R², R⁴ and n are as defined in formula (IM).

In one embodiment, the disclosure provides a compound of formula (IM), formula (I), formula (II), formula (III), or a pharmaceutically acceptable salt, solvate, or prodrug thereof, wherein X, Y, Z and W are identical or different, and each is independently CR³, R³ is as defined in formula (IM).

In one embodiment of the disclosure, the compound of formula (IM), formula (I), formula (II), formula (III), is a compound of formula (IV):

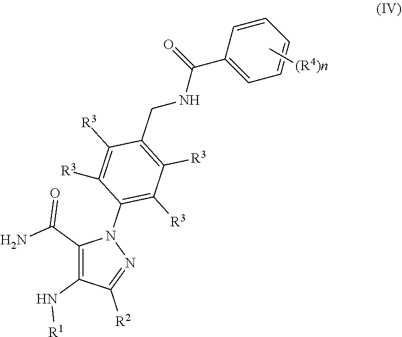

or a pharmaceutically acceptable salt, solvate, or prodrug thereof, wherein:

R¹, R², R³, R⁴ and n are as defined in formula (IM).

In one embodiment, the disclosure provides a compound of formula (IM), formula (I), formula (II), formula (III), formula (IV), or a pharmaceutically acceptable salt, solvate, or prodrug thereof, wherein R¹ is selected from the group consisting of hydrogen, C₁₋₆ alkyl, C₁₋₆ haloalkyl, —C(O)R⁵, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, C₁₋₆ alkoxy, C₁₋₆ haloalkoxy, cyano, amino, hydroxyl, C₁₋₆ hydroxyalkyl, 3-8 membered cycloalkyl, 3-12 membered heterocyclyl, 6-10 membered aryl and 5-10 membered heteroaryl; preferably R¹ is selected from the group consisting of hydrogen, C₁₋₆ alkyl, C₁₋₆ haloalkyl and —C(O)R⁵; wherein the C₁₋₆ alkyl is each optionally substituted with one or more, sometimes preferably one to five, sometimes more preferably one to three, groups independently selected from the group consisting of halogen, C₁₋₆ alkoxy, C₁₋₆ haloalkoxy, cyano, amino, hydroxyl, C₁₋₆ hydroxyalkyl, 3-8 membered cycloalkyl, 3-12 membered heterocyclyl, 6-10 membered aryl and 5-10 membered heteroaryl, and R⁵ is as defined in formula (IM); more preferably R¹ is selected from the group consisting of hydrogen, C₁₋₆ alkyl and —C(O)R⁵, and R⁵ is C₁₋₆ alkyl.

In one embodiment, the disclosure provides a compound of formula (I), formula (II), formula (III), formula (IV), or a pharmaceutically acceptable salt, solvate, or prodrug thereof, wherein R¹ is selected from the group consisting of hydrogen, C₁₋₆ alkyl, C₁₋₆ haloalkyl, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, C₁₋₆ alkoxy, C₁₋₆ haloalkoxy, cyano, amino, hydroxyl, C₁₋₆ hydroxyalkyl, 3-8 membered cycloalkyl, 3-12 membered heterocyclyl, 6-10 membered aryl and 5-10 membered heteroaryl; preferably R¹ is selected from the group consisting of hydrogen, C₁₋₆ alkyl and C₁₋₆ haloalkyl; wherein the C₁₋₆ alkyl is each optionally substituted with one or more, sometimes preferably one to five, sometimes more preferably one to three, groups independently selected from the group consisting of C₁₋₆ alkoxy, C₁₋₆ haloalkoxy, cyano, amino, hydroxyl, C₁₋₆ hydroxyalkyl, 3-8 membered cycloalkyl, 3-12 membered heterocyclyl, 6-10 membered aryl and 5-10 membered heteroaryl; more preferably R¹ is hydrogen.

In one embodiment, the disclosure provides a compound of formula (IM), formula (I), formula (II), formula (III), formula (IV), or a pharmaceutically acceptable salt, solvate, or prodrug thereof, wherein R² is selected from the group consisting of C₁₋₆ alkyl, C₁₋₆ haloalkyl, C₁₋₆ hydroxyalkyl, cyano, —OR⁵, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl, and R⁵ is C₁₋₆ alkyl; preferably R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; more preferably R² is selected from the group consisting of trifluoromethyl, isopropyl, 1,1,1-trifluoropropan-2-yl, cyano, cyclopentyl, cyclohexyl, piperidinyl, morpholinyl, thienyl, tetrahydrofuranyl and tetrahydropyranyl.

In one embodiment, the disclosure provides a compound of formula (I), formula (II), formula (III), formula (IV), or a pharmaceutically acceptable salt, solvate, or prodrug thereof, wherein R² is selected from the group consisting of C₁₋₆ alkyl, C₁₋₆ haloalkyl, C₁₋₆ hydroxyalkyl, 3-8 membered cycloalkyl and 3-12 membered heterocyclyl; wherein the C₁₋₆ alkyl, 3-8 membered cycloalkyl and 3-12 membered heterocyclyl are each optionally substituted with one or more, sometimes preferably one to five, sometimes more preferably one to three, groups independently selected from the group consisting of halogen, C₁₋₆ alkyl, C₁₋₆ haloalkyl, C₁₋₆ alkoxy, C₁₋₆ haloalkoxy, oxo and C₁₋₆ hydroxyalkyl; preferably R² is selected from the group consisting of trifluoromethyl, isopropyl, 1,1,1-trifluoropropan-2-yl, cyclopentyl, tetrahydrofuranyl and tetrahydropyranyl.

In one embodiment, the disclosure provides a compound of formula (IM), formula (I), formula (II), formula (III), formula (IV), or a pharmaceutically acceptable salt, solvate, or prodrug thereof, wherein R¹ and R² together with the nitrogen and the carbon to which they are attached form a 3-12 membered heterocycle; wherein the 3-12 membered heterocycle is optionally substituted with one or more, sometimes preferably one to five, sometimes more preferably one to three, groups selected from the group consisting of halogen, C₁₋₆ alkyl, C₁₋₆ haloalkyl, C₁₋₆ alkoxy, C₁₋₆ haloalkoxy, cyano, amino, hydroxyl and C₁₋₆ hydroxyalkyl; preferably R¹ and R² together with the nitrogen and the carbon to which they are attached form a 3-6 membered heterocycle; wherein the 3-6 membered heterocycle is optionally substituted with one or more, sometimes preferably one to five, sometimes more preferably one to three, groups selected from the group consisting of halogen, C₁₋₆ alkyl, C₁₋₆ haloalkyl, C₁₋₆ alkoxy, C₁₋₆ haloalkoxy and C₁₋₆ hydroxyalkyl; more preferably R¹ and R² together with the nitrogen and the carbon to which they are attached form a 3-6 membered heterocycle; wherein the 3-6 membered heterocycle is optionally substituted with C₁₋₆ alkyl; most preferably R¹ and R² together with the nitrogen and the carbon to which they are attached form piperidine, wherein the piperidine is optionally substituted with methyl.

In one embodiment, the disclosure provides a compound of formula (IM), formula (I), formula (II), formula (III), formula (IV), or a pharmaceutically acceptable salt, solvate, or prodrug thereof, wherein R⁴ at each occurrence is identical or different, and each is selected from the group consisting of hydrogen, halogen, C₁₋₆ alkyl, C₁₋₆ haloalkyl, cyano, —OR^{6a}, —NR^{7b}R^{8b} and C₁₋₆ hydroxyalkyl; R^{6a} is selected from the group consisting of hydrogen, C₁₋₆ alkyl, C₁₋₆ haloalkyl, C₁₋₆ hydroxyalkyl, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more, sometimes preferably one to five, sometimes more preferably one to three, groups independently selected from the group consisting of halogen, C₁₋₆ alkyl, C₁₋₆ haloalkyl, C₁₋₆ alkoxy, C₁₋₆ haloalkoxy, cyano, amino, hydroxyl, oxo, hydroxyalkyl, 3-8 membered cycloalkyl, 3-12 membered heterocyclyl, 6-10 membered aryl and 5-10 membered heteroaryl.

In one embodiment, the disclosure provides a compound of formula (IM), formula (I), formula (II), formula (III), formula (IV), or a pharmaceutically acceptable salt, solvate, or prodrug thereof, wherein R⁴ at each occurrence is identical or different, and each is selected from the group consisting of hydrogen, halogen, C₁₋₆ alkyl, C₁₋₆ haloalkyl and —OR^{6a}; R^{6a} is selected from the group consisting of hydrogen, C₁₋₆ alkyl, C₁₋₆ haloalkyl, 3-8 membered cycloalkyl and 3-12 membered heterocyclyl; preferably R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; more preferably R⁴ at each occurrence is different, and each is selected from the group consisting of hydrogen, fluoro, methoxy, ethoxy and cyclopropoxy.

In one embodiment, the disclosure provides a compound of formula (I), formula (II), formula (III), formula (IV), or a pharmaceutically acceptable salt, solvate, or prodrug thereof, wherein R⁴ at each occurrence is identical or different, and each is selected from the group consisting of hydrogen, halogen, C₁₋₆ alkyl, C₁₋₆ haloalkyl and —OR^{6a}; R^{6a} is 3-8 membered cycloalkyl; preferably R⁴ at each occurrence is different, and each is selected from the group consisting of hydrogen, fluoro, methoxy, ethoxy and cyclopropoxy.

In one embodiment, the disclosure provides a compound of formula (IM), formula (I), formula (II), formula (III), formula (IV), or a pharmaceutically acceptable salt, solvate, or prodrug thereof, wherein R³ at each occurrence is selected from the group consisting of hydrogen, halogen, C₁₋₆ alkyl, C₁₋₆ haloalkyl, C₁₋₆ alkoxy and C₁₋₆ haloalkoxy; preferably R³ at each occurrence is selected from the group consisting of hydrogen, fluoro, ethoxy and methoxy.

In one embodiment, the disclosure provides a compound of formula (IM), formula (I), formula (II), formula (III), formula (IV), or a pharmaceutically acceptable salt, solvate, or prodrug thereof, wherein n is 0, 1 or 2.

In one embodiment, the disclosure provides a compound of formula (IM), formula (I) or formula (II), or a pharmaceutically acceptable salt, solvate, or prodrug thereof, wherein

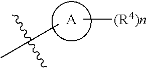

is selected from the group consisting of

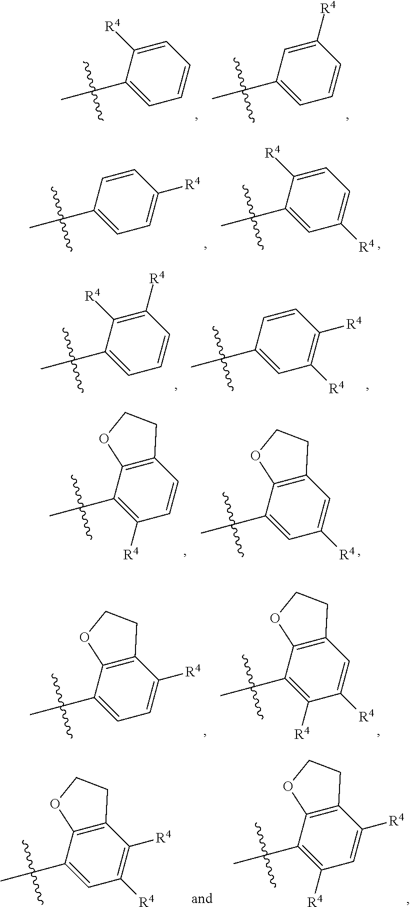

wherein R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; preferably

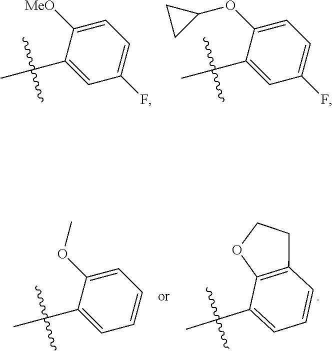

In one embodiment, the disclosure provides a compound of formula (I) or formula (II), or a pharmaceutically acceptable salt, solvate, or prodrug thereof, wherein

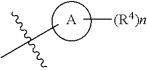

is

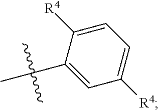

preferably

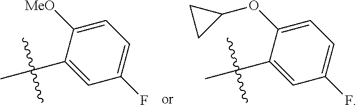 [SMILES: *c1cc(F)ccc1OC ; "or" ; *c1cc(F)ccc1OC1CC1]

In one embodiment, the disclosure provides a compound of formula (III) or formula (IV), or a pharmaceutically acceptable salt, solvate, or prodrug thereof, wherein

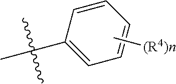

is

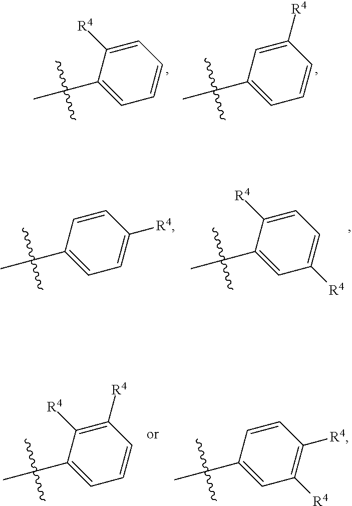

wherein R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; preferably

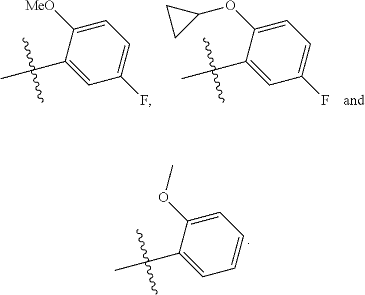

In one embodiment, the disclosure provides a compound of formula (III) or formula (IV), or a pharmaceutically acceptable salt, solvate, or prodrug thereof, wherein

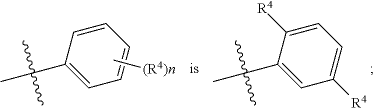

preferably

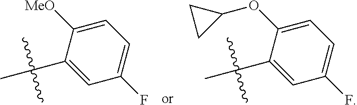 [SMILES: *c1cc(F)ccc1OC ; "or" ; *c1cc(F)ccc1OC1CC1]

In one embodiment, the disclosure provides a compound of formula (IM), formula (I), formula (II) or formula (III), or a pharmaceutically acceptable salt, solvate, or prodrug thereof, wherein

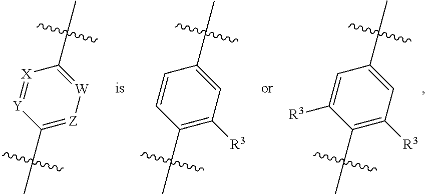

wherein R³ at each occurrence is selected from the group consisting of hydrogen, halogen, C₁₋₆ alkyl, C₁₋₆ haloalkyl, C₁₋₆ alkoxy and C₁₋₆ haloalkoxy; preferably

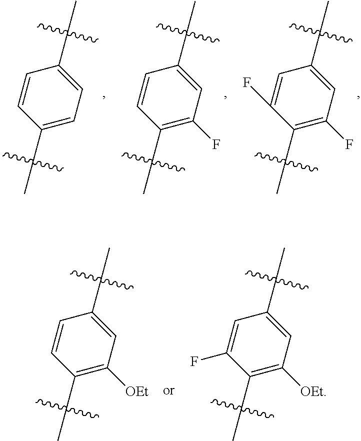

In one embodiment, the disclosure provides a compound of formula (I), formula (II) or formula (III), or a pharmaceutically acceptable salt, solvate, or prodrug thereof, wherein

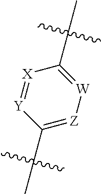

is selected from the group consisting of

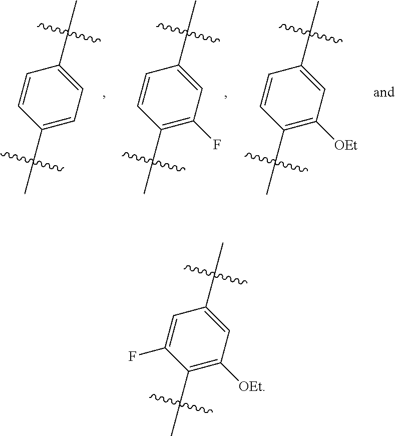 [SMILES: *c1ccc(*)cc1 ; *c1ccc(*)c(F)c1 ; *c1ccc(*)c(OCC)c1 ; *c1cc(F)c(*)c(OCC)c1]

In one embodiment, the disclosure provides a compound of formula (IV) or a pharmaceutically acceptable salt, solvate, or prodrug thereof, wherein

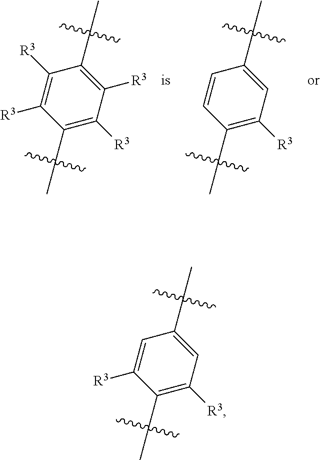

wherein R³ at each occurrence is selected from the group consisting of hydrogen, halogen, C₁₋₆ alkyl, C₁₋₆ haloalkyl, C₁₋₆ alkoxy and C₁₋₆ haloalkoxy; preferably

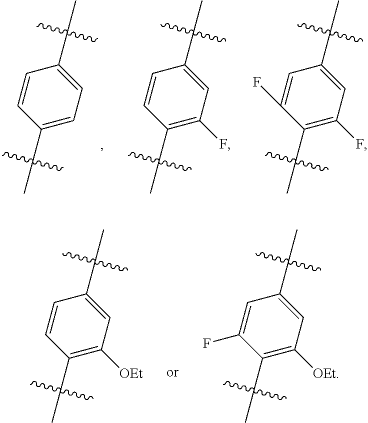

In one embodiment, the disclosure provides a compound of formula (IV), or a pharmaceutically acceptable salt, solvate, or prodrug thereof, wherein

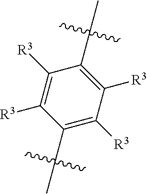

is selected from the group consisting of

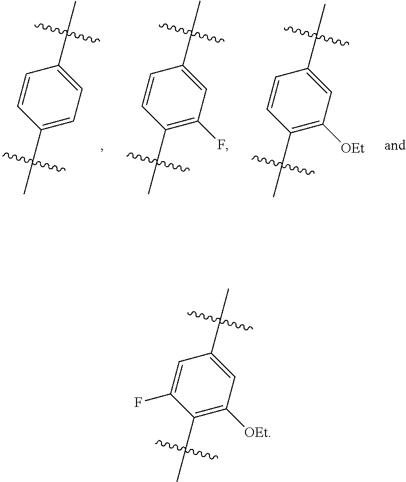 [SMILES: *c1ccc(*)cc1 ; "," ; *c1ccc(*)c(F)c1 ; *c1ccc(*)c(OCC)c1 ; "and" ; *c1cc(F)c(*)c(OCC)c1]

In one embodiment, the disclosure provides a compound of formula (IM), or a pharmaceutically acceptable salt, solvate, or prodrug thereof, wherein R^{x} is C(O)NR^{A}R^{B}, R^{A} and R^{B} are identical, and each is hydrogen; R^{1a} is hydrogen; R¹ is selected from the group consisting of hydrogen, C₁₋₆ alkyl and —C(O)R⁵; R² is selected from the group consisting of C₁₋₆ alkyl, C₁₋₆ haloalkyl, C₁₋₆ hydroxyalkyl, cyano, —OR⁵, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one to three groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; or R¹ and R² together with the nitrogen and the carbon to which they are attached form a 3-12 membered heterocycle; wherein the 3-12 membered heterocycle is optionally substituted with one to three groups selected from the group consisting of halogen, C₁₋₆ alkyl, C₁₋₆ haloalkyl, C₁₋₆ alkoxy, C₁₋₆ haloalkoxy, cyano, amino, hydroxyl and C₁₋₆ hydroxyalkyl; L₁ is NR^{7a}, L₂ is C(O), and R^{7a} is hydrogen; X, Y, Z and W are identical or different, and each is independently CR³; R³ at each occurrence is identical or different, and each is selected from the group consisting of hydrogen, halogen, C₁₋₆ alkyl, C₁₋₆ haloalkyl, C₁₋₆ alkoxy and C₁₋₆ haloalkoxy; ring A is selected from the group consisting of 6-10 membered aryl or 5-10 membered heteroaryl; R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; R⁵ is C₁₋₆ alkyl; n is 0, 1 or 2.

In one embodiment, the disclosure provides a compound of formula (I), or a pharmaceutically acceptable salt, solvate, or prodrug thereof, wherein R^{x} is C(O)NR^{A}R^{B}, R^{A} and R^{B} are identical, and each is hydrogen; R¹ is selected from the group consisting of hydrogen, C₁₋₆ alkyl and —C(O)R⁵; R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one to three groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; or R¹ and R² together with the nitrogen and the carbon to which they are attached form a 3-6 membered heterocycle; wherein the 3-6 membered heterocycle is optionally substituted with one to three groups selected from the group consisting of halogen, C₁₋₆ alkyl, C₁₋₆ haloalkyl, C₁₋₆ alkoxy, C₁₋₆ haloalkoxy and C₁₋₆ hydroxyalkyl; L₁ is NR^{7a}, L₂ is C(O), and R^{7a} is hydrogen; X, Y, Z and W are identical or different, and each is independently CR³; R³ at each occurrence is identical or different, and each is selected from the group consisting of hydrogen, halogen, C₁₋₆ alkyl, C₁₋₆ haloalkyl, C₁₋₆ alkoxy and C₁₋₆ haloalkoxy; ring A is 6-10 membered aryl; R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; R⁵ is C₁₋₆ alkyl; n is 0, 1 or 2.

In one embodiment, the disclosure provides a compound of formula (II), or a pharmaceutically acceptable salt, solvate, or prodrug thereof, wherein R^{A} and R^{B} are identical, and each is hydrogen; R¹ is selected from the group consisting of hydrogen, C₁₋₆ alkyl and —C(O)R⁵; R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one to three groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; or R¹ and R² together with the nitrogen and the carbon to which they are attached form a 3-6 membered heterocycle; wherein the 3-6 membered heterocycle is optionally substituted with one to three groups selected from the group consisting of halogen, C₁₋₆ alkyl, C₁₋₆ haloalkyl, C₁₋₆ alkoxy, C₁₋₆ haloalkoxy and C₁₋₆ hydroxyalkyl; X, Y, Z and W are identical or different, and each is independently CR³; R³ at each occurrence is identical or different, and each is selected from the group consisting of hydrogen, halogen, C₁₋₆ alkyl, C₁₋₆ haloalkyl, C₁₋₆ alkoxy and C₁₋₆ haloalkoxy; ring A is phenyl or

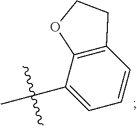 [SMILES: *c1cccc2c1OCC2]

R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; R⁵ is C₁₋₆ alkyl; n is 0, 1 or 2.

In one embodiment, the disclosure provides a compound of formula (III), or a pharmaceutically acceptable salt, solvate, or prodrug thereof, wherein R¹ is selected from the group consisting of hydrogen, C₁₋₆ alkyl and —C(O)R⁵; R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one to three groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; or R¹ and R² together with the nitrogen and the carbon to which they are attached form a 3-6 membered heterocycle; wherein the 3-6 membered heterocycle is optionally substituted with C₁₋₆ alkyl; X, Y, Z and W are identical or different, and each is independently CR³; R³ at each occurrence is identical or different, and each is selected from the group consisting of hydrogen, halogen, C₁₋₆ alkyl, C₁₋₆ haloalkyl, C₁₋₆ alkoxy and C₁₋₆ haloalkoxy; R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; R⁵ is C₁₋₆ alkyl; n is 0, 1 or 2.

In one embodiment, the disclosure provides a compound of formula (IV), or a pharmaceutically acceptable salt, solvate, or prodrug thereof, wherein R¹ is selected from the group consisting of hydrogen, C₁₋₆ alkyl and —C(O)R⁵; R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one to three groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; or R¹ and R² together with the nitrogen and the carbon to which they are attached form a 3-6 membered heterocycle; wherein the 3-6 membered heterocycle is optionally substituted with C₁₋₆ alkyl;

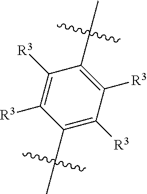

is

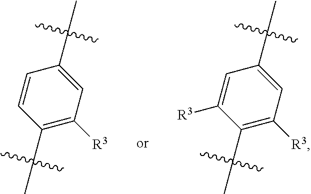

wherein R³ at each occurrence is independently selected from the group consisting of hydrogen, halogen, C₁₋₆ alkyl, C₁₋₆ haloalkyl, C₁₋₆ alkoxy and C₁₋₆ haloalkoxy;

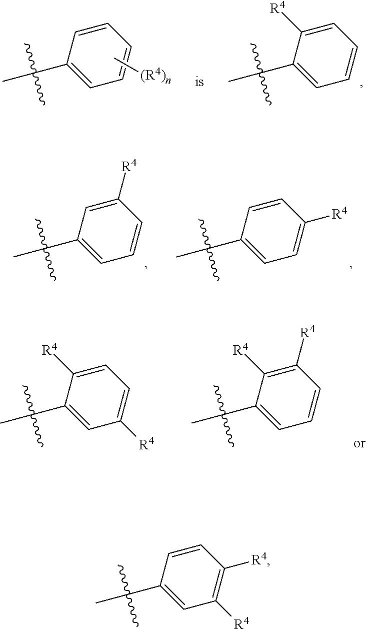

wherein R⁴ at each occurrence is identical or different, and each is selected from the group consisting of hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; R⁵ is C₁₋₆ alkyl.

As a person of ordinary skill in the art would understand, any and all plausible combinations of the embodiments disclosed herein, in particular, with regard to the definitions of any substituents, e.g., L₁, L₂, R^{x}, R^{1a}, W, X, Y, Z, R¹, R², R⁴, ring A and n, or the like, in the compounds of formulae (IM), (I), (II), (III), (IV), (IIA), (IIB), or the like, are all encompassed by the present disclosure.

For example, in one embodiment, the present disclosure provides a compound of formula (I), wherein:

L₁ is NH;

L₂ is C(O);

W, X, Y, and Z are each CR³;

A is C₆₋₁₀ aryl or 5- to 10-membered heteroaryl;

R¹ is selected from the group consisting of hydrogen, C₁₋₆ alkyl and —C(O)R⁵, and R⁵ is C₁₋₆ alkyl;

R² is selected from the group consisting of C₁₋₆ alkyl, C₁₋₆ haloalkyl, C₁₋₆ hydroxyalkyl, cyano, —OR⁵, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl, and R⁵ is C₁₋₆ alkyl;

R³ at each occurrence is independently selected from the group consisting of hydrogen, halogen, C₁₋₆ alkyl, C₁₋₆ haloalkyl, C₁₋₆ alkoxy and C₁₋₆ haloalkoxy; and

R⁴ at each occurrence is identical or different, and each is independently selected from the group consisting of hydrogen, halogen, C₁₋₆ alkyl, C₁₋₆ haloalkyl and —OR^{6a}; wherein R^{6a} is selected from the group consisting of hydrogen, C₁₋₆ alkyl, C₁₋₆ haloalkyl, 3-8 membered cycloalkyl and 3-12 membered heterocyclyl.

In the foregoing embodiment, sometimes preferably, R² is selected from the group consisting of C₁₋₆ alkyl, C₁₋₆ haloalkyl, cyano, 3-6 membered cycloalkyl, 3-6 membered heterocyclyl and 5-6 membered heteroaryl; wherein the C₁₋₆ alkyl, 3-6 membered cycloalkyl, 3-6 membered heterocyclyl and 5-6 membered heteroaryl are each optionally substituted with one to five, sometimes preferably one to three, groups independently selected from halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl; sometimes more preferably, R² is selected from trifluoromethyl, isopropyl, 1,1,1-trifluoropropan-2-yl, cyano, cyclopentyl, cyclohexyl, piperidinyl, morpholinyl, thienyl, tetrahydrofuranyl and tetrahydropyranyl.

In the foregoing embodiments, sometimes preferably, R⁴ at each occurrence is independently selected from hydrogen, halogen and —OR^{6a}; R^{6a} is selected from the group consisting of C₁₋₆ alkyl, C₁₋₆ haloalkyl, 3-6 membered cycloalkyl and 3-6 membered heterocyclyl; sometimes more preferably, R⁴ at each occurrence is independently selected from hydrogen, fluoro, methoxy, ethoxy and cyclopropyloxy.

In the foregoing embodiments, sometimes preferably, R¹ and R² together with the nitrogen and the carbon to which they are attached form a 5-8 membered heterocycle; wherein the 5-8 membered heterocycle is optionally substituted with one or more groups independently selected from the group consisting of halogen, C₁₋₆ alkyl, C₁₋₆ haloalkyl, C₁₋₆ alkoxy, C₁₋₆ haloalkoxy, cyano, amino, hydroxyl and C₁₋₆ hydroxyalkyl.

TABLE A
Exemplified compounds of the disclosure include, but are not limited to:

| Com- | |
|---|---|
| pound | |
| No. | Compound structure and name |
| A1 | 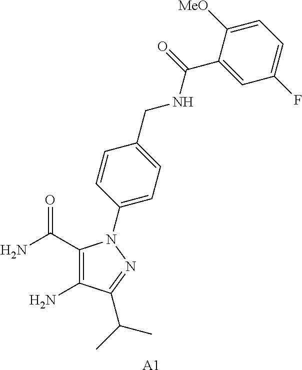 |
| | 4-amino-1-(4-((5-fluoro-2-methoxybenzamido)methyl)phenyl)-3- |
| | isopropyl-1H-pyrazole-5-carboxamide A1 |
| A2 | 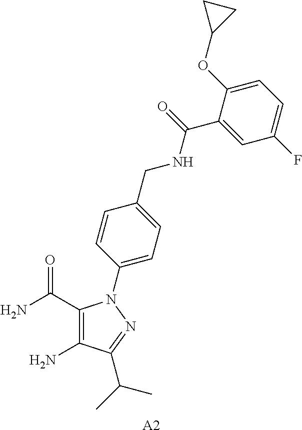 |
| | 4-amino-1-(4-((2-cyclopropoxy-5-fluorobenzamido)methyl)phenyl)- |
| | 3-isopropyl-1H-pyrazole-5-carboxamide A2 |
| A3 | 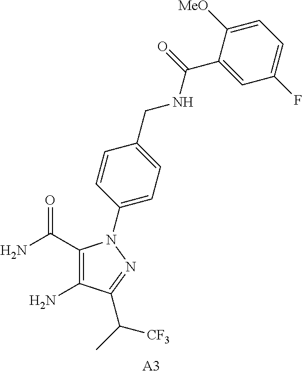 |
| | 4-amino-1-(4-((5-fluoro-2-methoxybenzamido)methyl)phenyl)-3- |
| | (1,1,1-trifluoropropan-2-yl)-1H-pyrazole-5-carboxamide A3 |
| A4 | 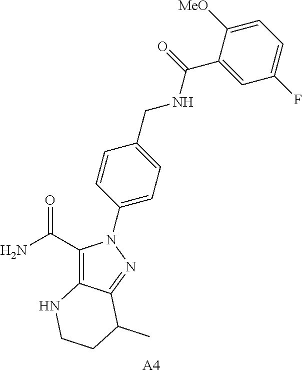 |
| | 2-(4-((5-fluoro-2-methoxybenzamido)methyl)phenyl)-7-methyl- |
| | 4,5,6,7-tetrahydro-2H-pyrazolo[4,3-b]pyridine-3-carboxamide A4 |
| A5 | 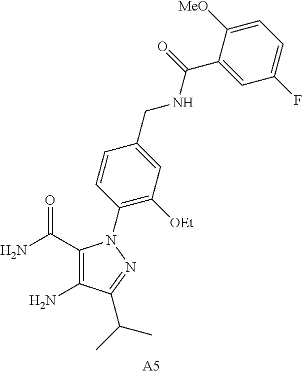 |
| | 4-amino-1-(2-ethoxy-4-((5-fluoro-2- |
| | methoxybenzamido)methyl)phenyl)-3-isopropyl-1H-pyrazole-5- |
| | carboxamide A5 |
| A6 | 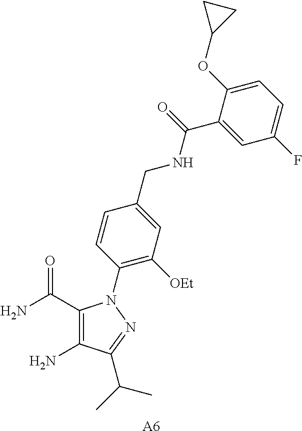 |
| | 4-amino-1-(4-((2-cyclopropoxy-5-fluorobenzamido)methyl)-2- |
| | ethoxyphenyl)-3-isopropyl-1H-pyrazole-5-carboxamide A6 |
| A7 | 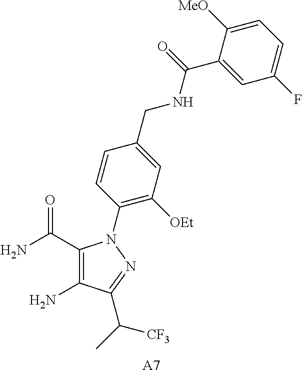 |
| | 4-amino-1-(2-ethoxy-4-((5-fluoro-2- |
| | methoxybenzamido)methyl)phenyl)-3-(1,1,1-trifluoropropan-2-yl)- |
| | 1H-pyrazole-5-carboxamide A7 |
| A8 | 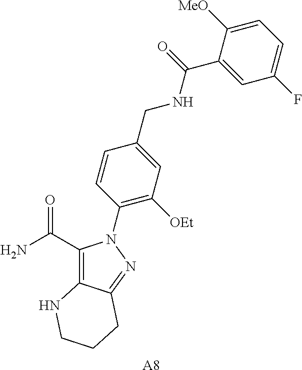 [SMILES: CCOc1cc(CNC(=O)c2cc(F)ccc2OC)ccc1-n1nc2c(c1C(N)=O)NCCC2 ; "A8"] |
| | 2-(2-ethoxy-4-((5-fluoro-2-methoxybenzamido)methyl)phenyl)- |
| | 4,5,6,7-tetrahydro-2H-pyrazolo[4,3-b]pyridine-3-carboxamide A8 |
| A9 | 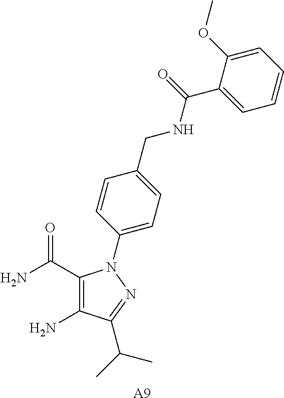 |
| | 4-amino-3-isopropyl-1-(4-((2-methoxybenzamido)methyl)phenyl)- |
| | 1H-pyrazole-5-carboxamide A9 |
| A10 | 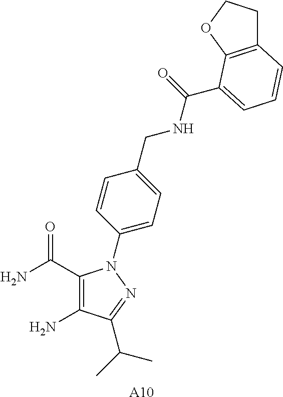 |
| | 4-amino-1-(4-((2,3-dihydrobenzofuran-7- |
| | carboxamido)methyl)phenyl)-3-isopropyl-1H-pyrazole-5- |
| | carboxamide A10 |
| A11 | 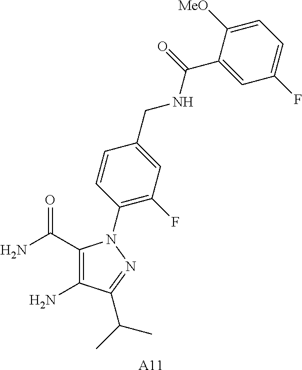 |
| | 4-amino-1-(2-fluoro-4-((5-fluoro-2- |
| | methoxybenzamido)methyl)phenyl)-3-isopropyl-1H-pyrazole-5- |
| | carboxamide A11 |
| A12 | 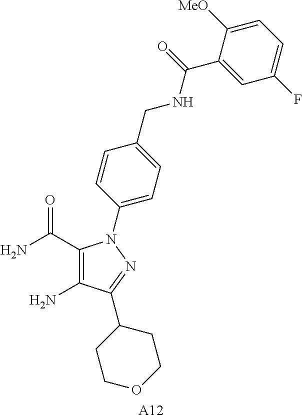 |
| | 4-amino-1-(4-((5-fluoro-2-methoxybenzamido)methyl)phenyl)-3- |
| | (tetrahydro-2H-pyran-4-yl)-1H-pyrazole-5-carboxamide A12 |
| A13 | 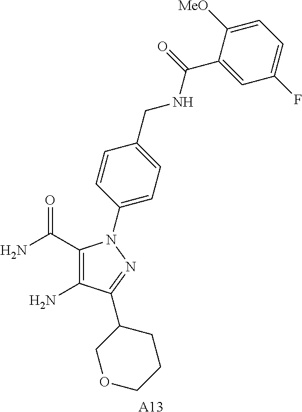 |
| | 4-amino-1-(4-((5-fluoro-2-methoxybenzamido)methyl)phenyl)-3- |
| | (tetrahydro-2H-pyran-3-yl)-1H-pyrazole-5-carboxamide A13 |
| A14 | 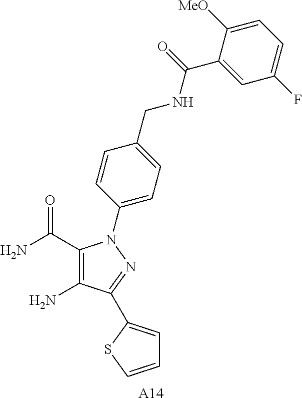 |
| | 4-amino-1-(4-((5-fluoro-2-methoxybenzamido)methyl)phenyl)-3- |
| | (thiophen-2-yl)-1H-pyrazole-5-carboxamide A14 |
| A15 | 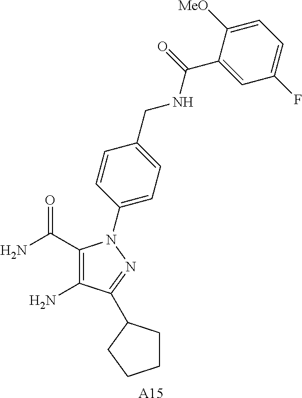 |
| | 4-amino-3-cyclopentyl-1-(4-((5-fluoro-2- |
| | methoxybenzamido)methyl)phenyl)-1H-pyrazole-5-carboxamide |
| | A15 |
| A16 | 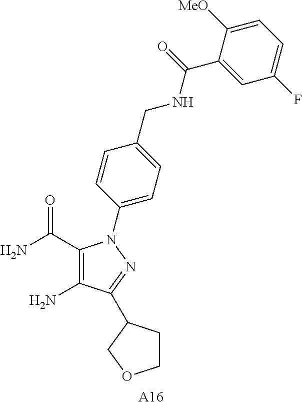 |
| | 4-amino-1-(4-((5-fluoro-2-methoxybenzamido)methyl)phenyl)-3- |
| | (tetrahydrofuran-3-yl)-1H-pyrazole-5-carboxamide A16 |
| A17 | 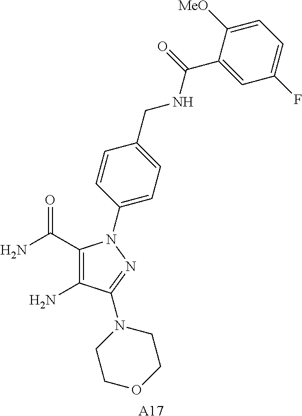 |
| | 4-amino-1-(4-((5-fluoro-2-methoxybenzamido)methyl)phenyl)-3- |
| | morpholino-1H-pyrazole-5-carboxamide A17 |
| A18 | 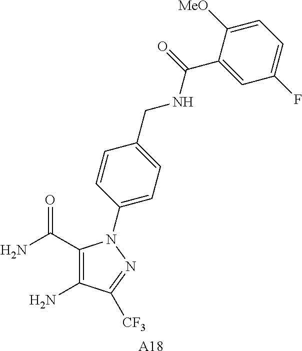 |
| | 4-amino-1-(4-((5-fluoro-2-methoxybenzamido)methyl)phenyl)-3- |
| | (trifluoromethyl)-1H-pyrazole-5-carboxamide A18 |
| A19 | 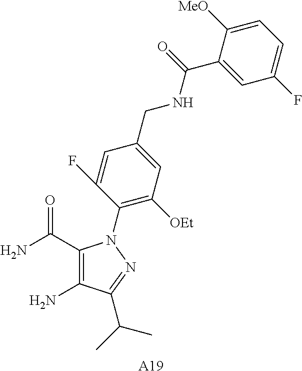 |
| | 4-amino-1-(2-ethoxy-6-fluoro-4-((5-fluoro-2- |
| | methoxybenzamido)methyl)phenyl)-3-isopropyl-1H-pyrazole-5- |
| | carboxamide A19 |
| A20 | 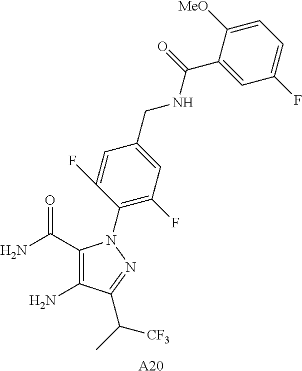 |
| | 4-amino-1-(2,6-difluoro-4-((5-fluoro-2- |
| | methoxybenzamido)methyl)phenyl)-3-(1,1,1-trifluoropropan-2-yl)- |
| | 1H-pyrazole-5-carboxamide A20 |
| A21 | 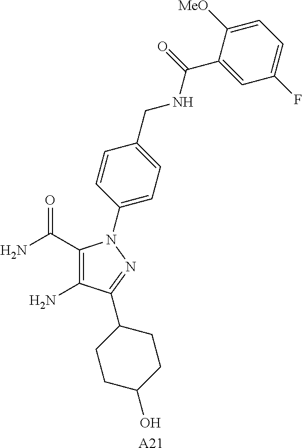 |
| | 4-amino-1-(4-((5-fluoro-2-methoxybenzamido)methyl)phenyl)-3-(4- |
| | hydroxycyclohexyl)-1H-pyrazole-5-carboxamide A21 |
| A22 | 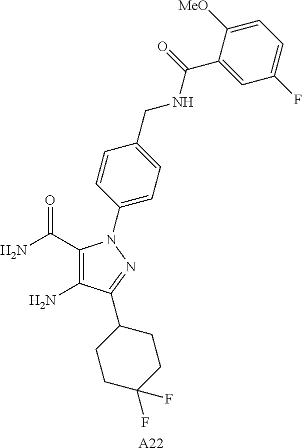 |
| | 4-amino-3-(4,4-difluorocyclohexyl)-1-(4-((5-fluoro-2- |
| | methoxybenzamido)methyl)phenyl)-1H-pyrazole-5-carboxamide |
| | A22 |
| A23A | 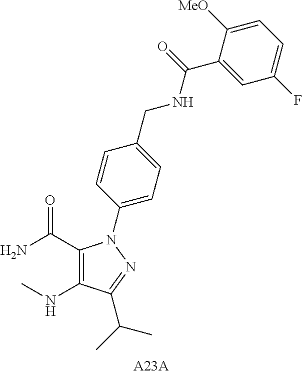 |
| | 1-(4-((5-fluoro-2-methoxybenzamido)methyl)phenyl)-3-isopropyl- |
| | 4-(methylamino)-1H-pyrazole-5-carboxamide A23A |
| A23B | 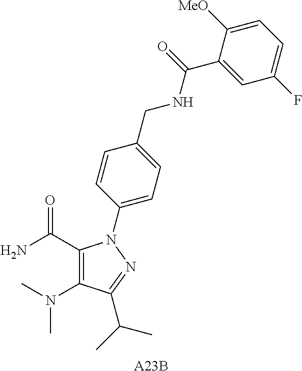 |
| | 4-(dimethylamino)-1-(4-((5-fluoro-2- |
| | methoxybenzamido)methyl)phenyl)-3-isopropyl-1H-pyrazole-5- |
| | carboxamide A23B |
| A24 | 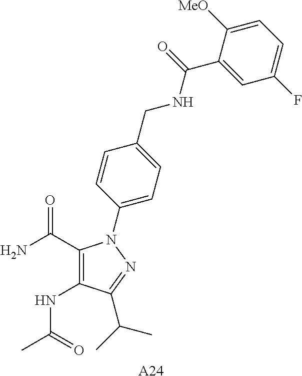 |
| | 4-acetamido-1-(4-((5-fluoro-2-methoxybenzamido)methyl)phenyl)- |
| | 3-isopropyl-1H-pyrazole-5-carboxamide A24 |
| A25 | 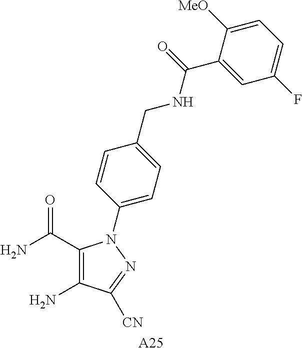 |
| | 4-amino-3-cyano-1-(4-((5-fluoro-2- |
| | methoxybenzamido)methyl)phenyl)-1H-pyrazole-5-carboxamide |
| | A25 |
| A26 | 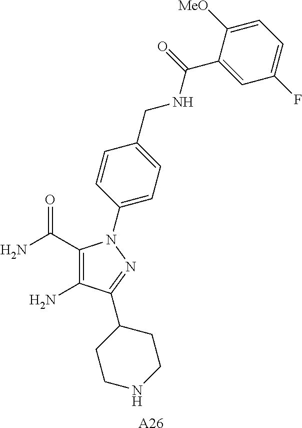 |
| | 4-amino-1-(4-((5-fluoro-2-methoxybenzamido)methyl)phenyl)-3- |
| | (piperidin-4-yl)-1H-pyrazole-5-carboxamide A26 |

In another aspect, this disclosure provides a compound of formula (IIA):

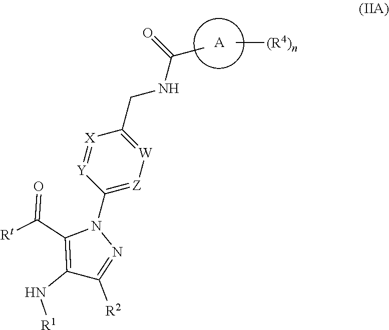

or a salt thereof, wherein:

R^{t} is selected from the group consisting of halogen, hydroxyl and alkoxy; preferably R^{t} is hydroxyl; and

W, X, Y, Z, R¹, R², R⁴, ring A and n are as defined in formula (II).

In another aspect, this disclosure provides a compound of formula (IIIA):

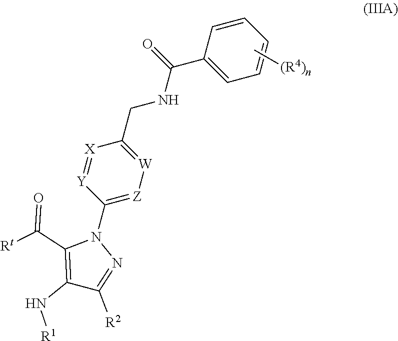

or a salt thereof, wherein:

R^{t} is selected from the group consisting of halogen, hydroxyl and alkoxy; preferably R^{t} is hydroxyl; and

W, X, Y, Z, R¹, R², R⁴ and n are as defined in formula (III).

In another aspect, this disclosure provides a compound of formula (IVA):

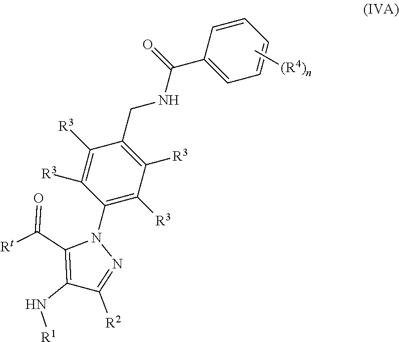

or a salt thereof, wherein:

R^{t} is selected from the group consisting of halogen, hydroxyl and alkoxy; preferably R^{t} is hydroxyl; and

R¹, R², R³, R⁴ and n are as defined in formula (IV).

TABLE B
Exemplified compounds of the disclosure include, but are not limited to:

| Com- | |
|---|---|
| pound | |
| No. | Compound structure and name |
| A3g | 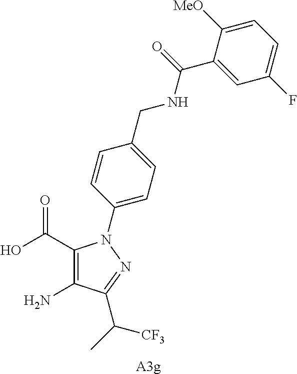 |
| | 4-amino-1-(4-((5-fluoro-2-methoxybenzamido)methyl)phenyl)-3- |
| | (1,1,1-trifluoropropan-2-yl)-1H-pyrazole-5-carboxylic acid A3g |
| A4i | 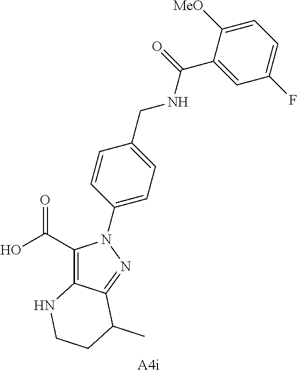 |
| | 2-(4-((5-fluoro-2-methoxybenzamido)methyl)phenyl)-7-methyl- |
| | 4,5,6,7-tetrahydro-2H-pyrazolo[4,3-b]pyridine-3-carboxylic acid |
| | A4i |
| A7g | 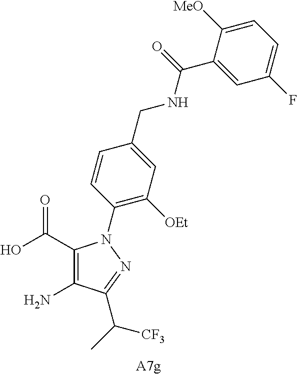 |
| | 4-amino-1-(2-ethoxy-4-((5-fluoro-2- |
| | methoxybenzamido)methyl)phenyl)-3-(1,1,1-trifluoropropan-2-yl)- |
| | 1H-pyrazole-5-carboxylic acid A7g |
| A12h | 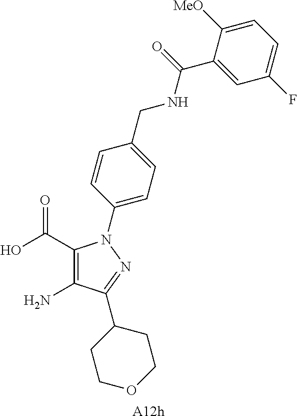 |
| | 4-amino-1-(4-((5-fluoro-2-methoxybenzamido)methyl)phenyl)-3- |
| | (tetrahydro-2H-pyran-4-yl)-1H-pyrazole-5-carboxylic acid A12h |
| A13j | 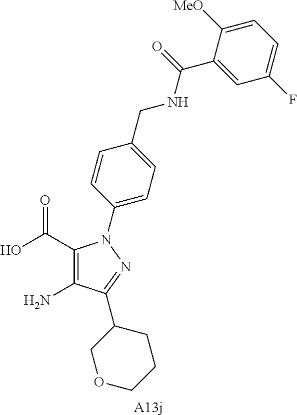 |
| | 4-amino-1-(4-((5-fluoro-2-methoxybenzamido)methyl)phenyl)-3- |
| | (tetrahydro-2H-pyran-3-yl)-1H-pyrazole-5-carboxylic acid A13j |
| A14d | 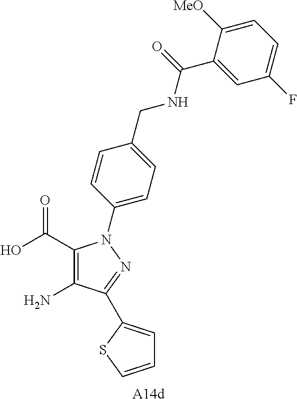 |
| | 4-amino-1-(4-((5-fluoro-2-methoxybenzamido)methyl)phenyl)-3- |
| | (thiophen-2-yl)-1H-pyrazole-5-carboxylic acid A14d |
| A15d | 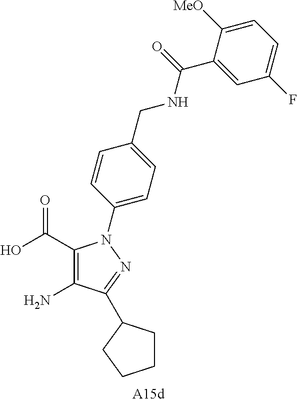 |
| | 4-amino-3-cyclopentyl-1-(4-((5-fluoro-2- |
| | methoxybenzamido)methyl)phenyl)-1H-pyrazole-5-carboxylic acid |
| | A15d |
| A16d | 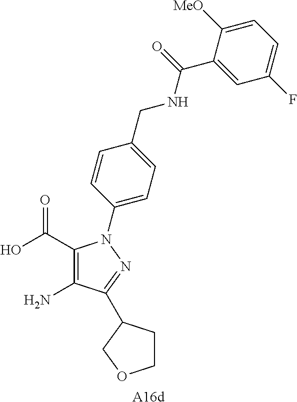 |
| | 4-amino-1-(4-((5-fluoro-2-methoxybenzamido)methyl)phenyl)-3- |
| | (tetrahydrofuran-3-yl)-1H-pyrazole-5-carboxylic acid A16d |
| A17j | 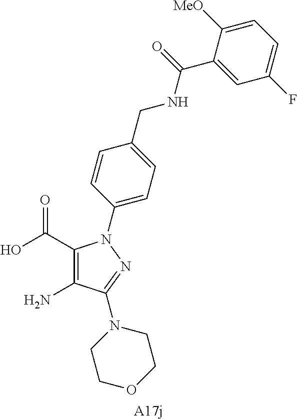 |
| | 4-amino-1-(4-((5-fluoro-2-methoxybenzamido)methyl)phenyl)-3- |
| | morpholino-1H-pyrazole-5-carboxylic acid A17j |
| A20e | 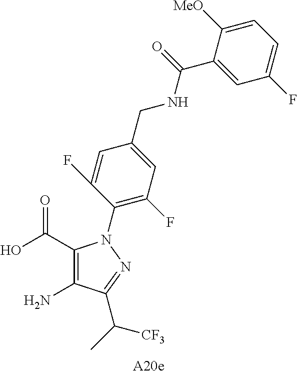 |
| | 4-amino-1-(2,6-difluoro-4-((5-fluoro-2- |
| | methoxybenzamido)methyl)phenyl)-3-(1,1,1-trifluoropropan-2-yl)- |
| | 1H-pyrazole-5-carboxylic acid A20e |
| A21f | 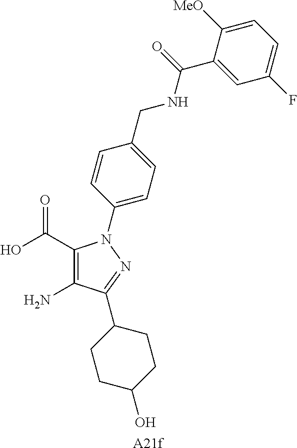 |
| | 4-amino-1-(4-((5-fluoro-2-methoxybenzamido)methyl)phenyl)-3-(4- |
| | hydroxycyclohexyl)-1H-pyrazole-5-carboxylic acid A21f |
| A22d | 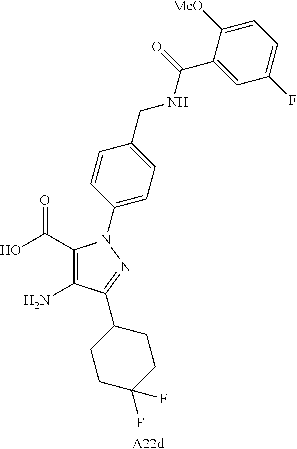 |
| | 4-amino-3-(4,4-difluorocyclohexyl)-1-(4-((5-fluoro-2- |
| | methoxybenzamido)methyl)phenyl)-1H-pyrazole-5-carboxylic acid |
| | A22d |
| A25f | 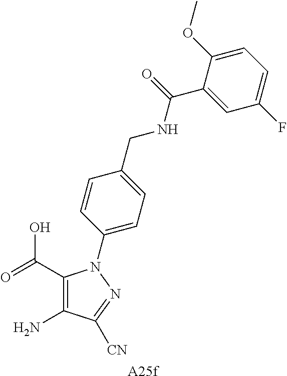 |
| | 4-amino-3-cyano-1-(4-((5-fluoro-2- |
| | methoxybenzamido)methyl)phenyl)-1H-pyrazole-5-carboxylic acid |
| | A25f |

TABLE C
Exemplified compounds of the disclosure include, but are not limited to:

| Com- | |
|---|---|
| pound | |
| No. | Compound structure and name |
| A8i | 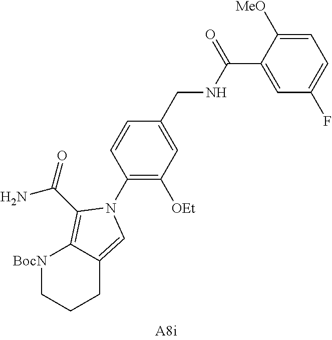 [SMILES: CCOc1cc(CNC(=O)c2cc(F)ccc2OC)ccc1-n1cc2c(c1C(N)=O)N(C(=O)OC(C)(C)C)CCC2] |
| | tert-butyl 3-carbamoyl-2-(2-ethoxy-4-((5-fluoro-2- |
| | methoxybenzamido)methyl)phenyl)-2,5,6,7-tetrahydro-4H- |
| | pyrazolo[4,3-b]pyridine-4-carboxylate A8i |
| A26d | 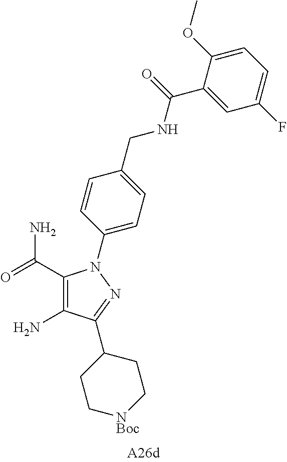 [SMILES: COc1ccc(F)cc1C(=O)NCc1ccc(-n2nc(C3CCN(C(=O)OC(C)(C)C)CC3)c(N)c2C(N)=O)cc1] |
| | tert-butyl 4-(4-amino-5-carbamoyl-1-(4-((5-fluoro-2- |
| | methoxybenzamido)methyl)phenyl)-1H-pyrazol-3-yl)piperidine-1- |
| | carboxylate A26d |

In another aspect, this disclosure provides a compound of formula (IIB):

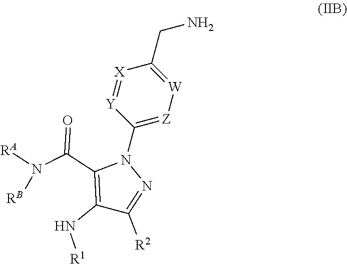

or a salt thereof, wherein:

R^{A}, R^{B}, W, X, Y, Z, R¹ and R² are as defined in formula (II).

In another aspect, this disclosure provides a compound of formula (IIIB):

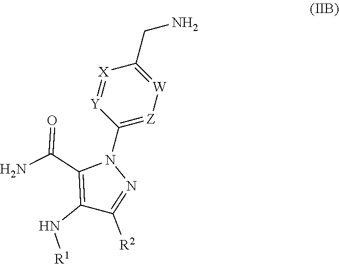

or a salt thereof, wherein:

W, X, Y, Z, R¹ and R² are as defined in formula (III).

In another aspect, this disclosure provides a compound of formula (IVB):

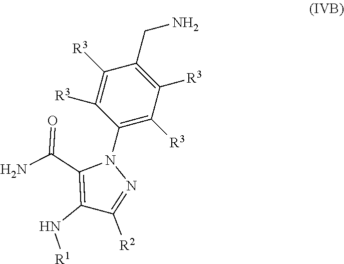

or a salt thereof, wherein:

R¹, R² and R³ are as defined in formula (IV).

TABLE D
Exemplified compounds of the disclosure include, but are not limited to:

| Com- | |
|---|---|
| pound | |
| No. | Compound structure and name |
| Int-1 | 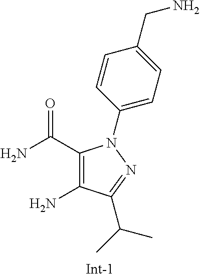 |
| | 4-amino-1-(4-(aminomethyl)phenyl)-3-isopropyl-1H-pyrazole-5- |
| | carboxamide Int-1 |
| Int-2 | 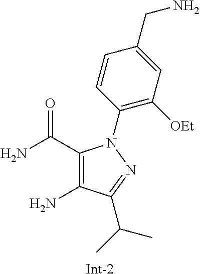 |
| | 4-amino-1-(4-(aminomethyl)-2-ethoxyphenyl)-3-isopropyl-1H- |
| | pyrazole-5-carboxamide Int-2 |
| Int-3 | 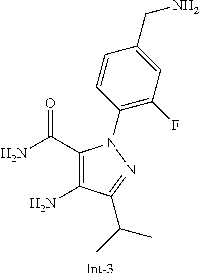 |
| | 4-amino-1-(4-(aminomethyl)-2-fluorophenyl)-3-isopropyl-1H- |
| | pyrazole-5-carboxamide Int-3 |
| Int-4 | 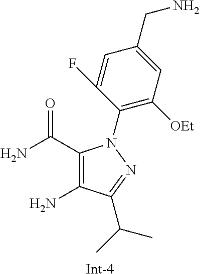 |
| | 4-amino-1-(4-(aminomethyl)-2-ethoxy-6-fluorophenyl)-3-isopropyl- |
| | 1H-pyrazole-5-carboxamide Int-4 |
| A18g | 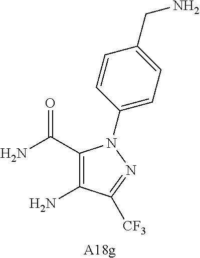 |
| | 4-amino-1-(4-(aminomethyl)phenyl)-3-(trifluoromethyl)-1H- |
| | pyrazole-5-carboxamide A18g |

In another aspect, this disclosure provides a process of preparing the compound of formula (II), or a pharmaceutically acceptable salt, solvate, or prodrug thereof, comprises a step of:

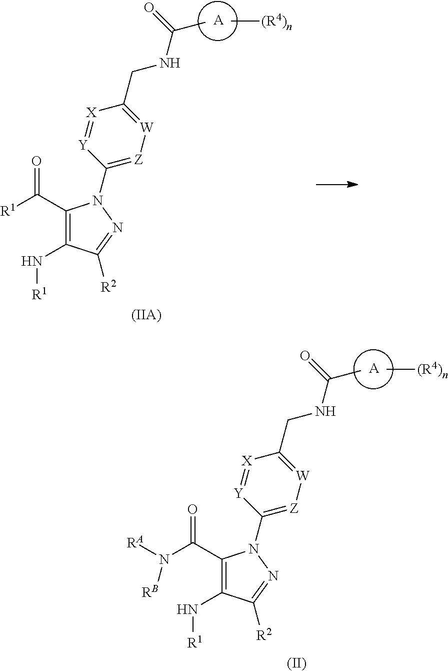

reacting a compound of Formula (IIA) or a salt thereof with ammonia water or NHR^{A}R^{B} or a pharmaceutically acceptable salt thereof (preferably NH₄Cl), to obtain the compound of formula (II) or a pharmaceutically acceptable salt, solvate, or prodrug thereof;

wherein:

R^{t} is selected from the group consisting of halogen, hydroxyl and alkoxy; preferably R^{t} is hydroxyl;

R^{A} and R^{B} are identical, and each is hydrogen; and

W, X, Y, Z, R¹, R², R⁴, ring A and n are as defined in formula (II).

In another aspect, this disclosure provides a process of preparing the compound of formula (III), or a pharmaceutically acceptable salt, solvate, or prodrug thereof, comprises a step of:

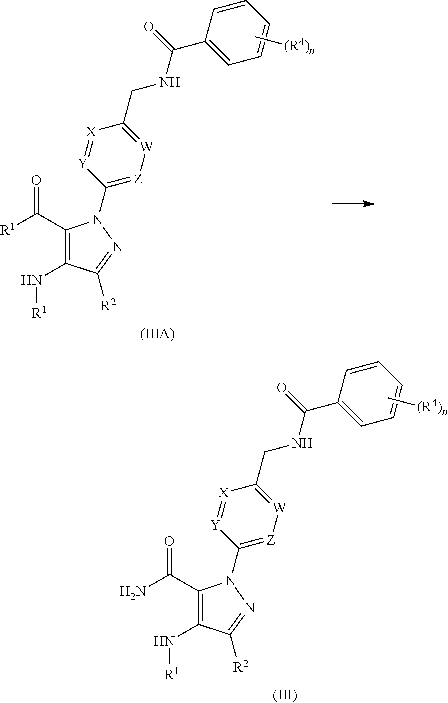

reacting a compound of Formula (IIIA) or a salt thereof with ammonia water or NHR^{A}R^{B} or a pharmaceutically acceptable salt thereof (preferably NH₄Cl), to obtain the compound of formula (III) or a pharmaceutically acceptable salt, solvate, or prodrug thereof;

wherein:

R^{t} is selected from the group consisting of halogen, hydroxyl and alkoxy; preferably R^{t} is hydroxyl;

R^{A} and R^{B} are identical, and each is hydrogen; and

W, X, Y, Z, R¹, R², R⁴ and n are as defined in formula (III).

In another aspect, this disclosure provides a process of preparing the compound of formula (IV), or a pharmaceutically acceptable salt, solvate, or prodrug thereof, comprises a step of:

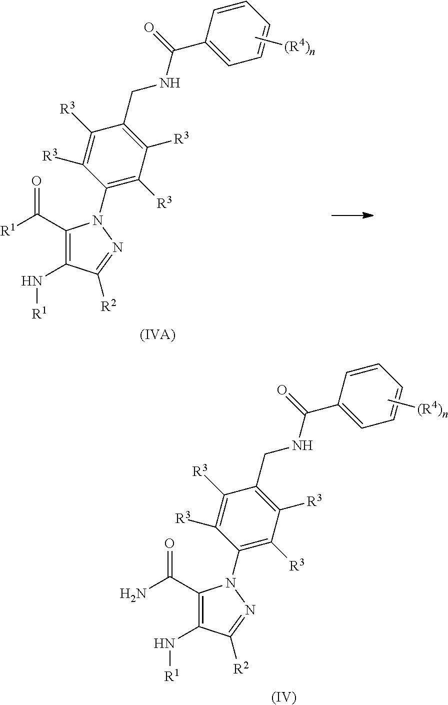

reacting a compound of Formula (IVA) or a salt thereof with ammonia water or NHR^{A}R^{B} or a pharmaceutically acceptable salt thereof (preferably NH₄Cl), to obtain the compound of formula (IV) or a pharmaceutically acceptable salt, solvate, or prodrug thereof, wherein:

R^{t} is selected from the group consisting of halogen, hydroxyl and alkoxy; preferably R^{t} is hydroxyl; and

R¹, R², R³, R⁴ and n are as defined in formula (IV).

In another aspect, this disclosure provides a process of preparing the compound of formula (II), or a pharmaceutically acceptable salt, solvate, or prodrug thereof, comprises a step of:

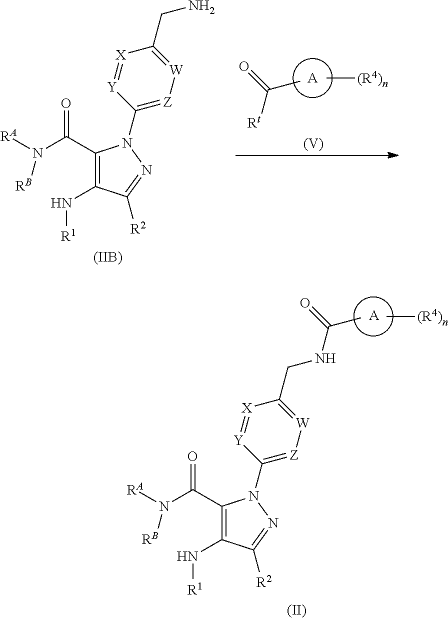

reacting a compound of Formula (IIB) or a salt thereof with a compound of Formula (V) to obtain the compound of formula (II) or a pharmaceutically acceptable salt, solvate, or prodrug thereof;

wherein:

R^{t} is selected from the group consisting of halogen, hydroxyl and alkoxy; preferably R^{t} is hydroxyl; and

R^{A}, R^{B}, W, X, Y, Z, R¹, R², R⁴, ring A and n are as defined in formula (II).

In another aspect, this disclosure provides a process of preparing the compound of formula (III), or a pharmaceutically acceptable salt, solvate, or prodrug thereof, comprises a step of:

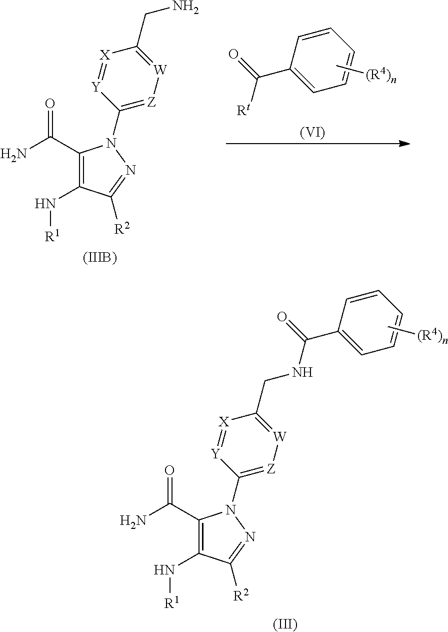

reacting a compound of Formula (IIIB) or a salt thereof with a compound of Formula (VI) to obtain the compound of formula (III) or a pharmaceutically acceptable salt, solvate, or prodrug thereof;

wherein:

R^{t} is selected from the group consisting of halogen, hydroxyl and alkoxy; preferably R^{t} is hydroxyl; and

W, X, Y, Z, R¹, R², R⁴ and n are as defined in formula (III).

In another aspect, this disclosure provides a process of preparing the compound of formula (IV), or a pharmaceutically acceptable salt, solvate, or prodrug thereof, comprises a step of:

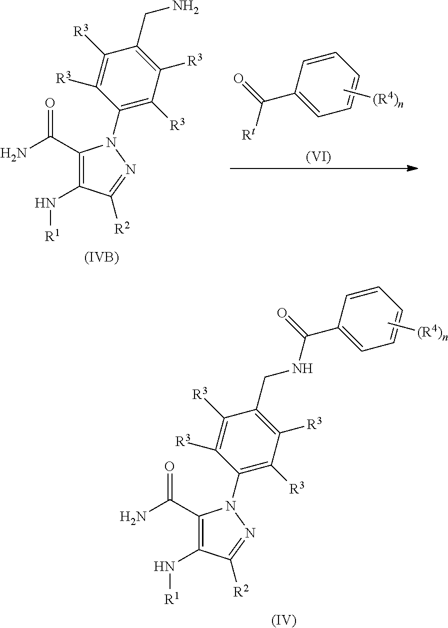

reacting a compound of Formula (IVB) or a salt thereof with a compound of Formula (VI) to obtain the compound of formula (IV) or a pharmaceutically acceptable salt, solvate, or prodrug thereof, wherein:

R^{t} is selected from the group consisting of halogen, hydroxyl and alkoxy; preferably R^{t} is hydroxyl; and

R¹, R², R³, R⁴ and n are as defined in formula (IV).

The present disclosure also provides a pharmaceutical composition, comprising a compound of formula (IM), formula (I), formula (II), formula (III), formula (IV), Table A or a pharmaceutically acceptable salt, solvate, or prodrug thereof, and one or more pharmaceutically acceptable carriers, diluents and/or other excipients.

The present disclosure also provides a method of treatment of a disease or condition which is modulated by BTK, wherein the method comprises a step of administering to a subject in need thereof a therapeutically effective amount of the compound of formula (IM), formula (I), formula (II), formula (III), formula (IV), Table A or a pharmaceutically acceptable salt, solvate, or prodrug thereof, or a pharmaceutical composition thereof.

The present disclosure also provides a method of treatment of a disease or condition which is inhibited by BTK, wherein the method comprises a step of administering to a subject in need thereof a therapeutically effective amount of the compound of formula (IM), formula (I), formula (II), formula (III), Table A, or a pharmaceutically acceptable salt thereof, or a pharmaceutical composition thereof.

In another aspect, the present disclosure also relates to use of a compound of formula (IM), formula (I), formula (II), formula (III), formula (IV), Table A or a pharmaceutically acceptable salt, solvate, or prodrug thereof, or a pharmaceutical composition thereof, in the preparation of a medicament for treating a disease or condition which is modulated by BTK.

In another aspect, the present disclosure also relates to use of a compound of formula (I), formula (I), formula (II), formula (III), formula (IV), Table A or a pharmaceutically acceptable salt, solvate, or prodrug thereof, or a pharmaceutical composition thereof, in the preparation of a medicament for inhibition of BTK.

The present disclosure further relates to the compound of formula (I), formula (I), formula (II), formula (III), formula (IV), Table A or a pharmaceutically acceptable salt, solvate, or prodrug thereof, or a pharmaceutical composition thereof, for use as a medicament.

The present disclosure also relates to the compound of formula (IM), formula (I), formula (II), formula (III), formula (IV), Table A or a pharmaceutically acceptable salt, solvate, or prodrug thereof, or a pharmaceutical composition thereof, for use in treating a disease or condition which is modulated by BTK.

The present disclosure also relates to the compound of formula (IM), formula (I), formula (II), formula (III), formula (IV), Table A or a pharmaceutically acceptable salt, solvate, or prodrug thereof, or a pharmaceutical composition thereof, for use in inhibiting BTK.

In some embodiments, the disease or condition treatable by the modulation/inhibition of BTK may be selected from: cancers, immune disorders, cardiovascular diseases, viral infections, inflammation, metabolism/endocrine function disorders, and neurological disorders; preferably, the condition modulated by BTK is selected from the group consisting of B-cell malignancy, B-cell lymphoma, diffuse large B cell lymphoma, chronic lymphocyte leukemia, non-Hodgkin lymphoma (for example ABC-DLBCL), mantle cell lymphoma, follicular lymphoma, hairy cell leukemia, B-cell non-Hodgi lymphoma, Waldenstrom's macroglobulinemia, multiple myeloma, bone cancer, bone metastasis, arthritis, multiple sclerosis, osteoporosis, irritable bowel syndrome, inflammatory bowel disease, Crohn's disease, Sjogren's syndrome and lupus.

In some embodiments, the disclosure is related to use of a compound of formula (IM), formula (I), formula (II), formula (III), Table A, or a pharmaceutically acceptable salt thereof, or a pharmaceutical composition thereof, in the preparation of a medicament for treating disease or condition, wherein the disease or condition is selected from the group consisting of: B-cell malignancy, B-cell lymphoma, diffuse large B cell lymphoma, chronic lymphocyte leukemia, non-Hodgkin lymphoma (for example ABC-DLBCL), mantle cell lymphoma, follicular lymphoma, hairy cell leukemia, B-cell non-Hodgkin lymphoma, Waldenstrom's macroglobulinemia, multiple myeloma, bone cancer, bone metastasis, follicular lymphoma, chronic lymphocytic lymphoma, B-cell prolymphocyte leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, burkitt lymphoma/leukemia, lymphomatoid granulomatosis, inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, vulvodynia, graft versus host disease, transplantation, transfusion, anaphylaxis, allergy, type I hypersensitivity, allergic conjunctivitis, allergic rhinitis, atopic dermatitis, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, vulvitis, pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, bronchiectasis, fatty liver disease, steatosis (for example nonalcoholic steatohepatitis (NASH), cholestatic liver disease (for example primary biliary cirrhosis (PBC), cirrhosis, alcohol-induced liver fibrosis, biliary duct injury, biliary fibrosis, cholestasis or cholangiopathies, hepatic or liver fibrosis (includes, but is not limited to, hepatic fibrosis associated with alcoholism), viral infection (for example hepatitis, for example hepatitis C, B or D), autoimmune hepatitis, nonalcoholic fatty liver disease (NAFLD), progressive massive fibrosis, exposure to toxins or irritants (for example alcohol, pharmaceutical drugs and environmental toxins), renal fibrosis (for example chronic kidney fibrosis), nephropathies associated with injury/fibrosis (for example chronic nephropathies associated with diabetes (for example diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent, fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease, age-related macular degeneration, diabetic retinopathy, retinopathy of prematurity and neovascular glaucoma.

In some embodiments, the disclosure is related to a method of treatment of a disease or condition selected from: B-cell malignancy, B-cell lymphoma, diffuse large B cell lymphoma, chronic lymphocyte leukemia, non-Hodgkin lymphoma for example ABC-DLBCL, mantle cell lymphoma, follicular lymphoma, hairy cell leukemia B-cell non-Hodgkin lymphoma, Waldenstrom's macroglobulinemia, multiple myeloma, bone cancer, bone metastasis, follicular lymphoma, chronic lymphocytic lymphoma, B-cell prolymphocyte leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, burkitt lymphoma/leukemia, lymphomatoid granulomatosis, inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, vulvodynia, graft versus host disease, transplantation, transfusion, anaphylaxis, allergy, type I hypersensitivity, allergic conjunctivitis, allergic rhinitis, atopic dermatitis, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, vulvitis, pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, bronchiectasis, fatty liver disease, steatosis (e.g., nonalcoholic steatohepatitis (NASH), cholestatic liver disease (e.g., primary biliary cirrhosis (PBC), cirrhosis, alcohol-induced liver fibrosis, biliary duct injury, biliary fibrosis, cholestasis or cholangiopathies. In some embodiments, hepatic or liver fibrosis includes, but is not limited to, hepatic fibrosis associated with alcoholism, viral infection, e.g., hepatitis (e.g., hepatitis C, B or D), autoimmune hepatitis, nonalcoholic fatty liver disease (NAFLD), progressive massive fibrosis, exposure to toxins or irritants (e.g., alcohol, pharmaceutical drugs and environmental toxins), renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent, fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease, age-related macular degeneration, diabetic retinopathy, retinopathy of prematurity and neovascular glaucoma, wherein the method comprises a step of administering to a subject in need thereof a therapeutically effective amount of the compound of formula (IM), formula (I), formula (II), formula (III), formula (IV), Table A or a pharmaceutically acceptable salt, solvate, or prodrug thereof, or a pharmaceutical composition thereof.

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration.

The compounds of this disclosure can also be formulated as sustained release dosage forms.

Common formulations include a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from the group consisting of sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water-soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS.™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound or the type of pharmaceutically acceptable salt, solvate, or prodrug thereof can be verified by traditional therapeutic regimens.

Unless otherwise stated, the terms used in the specification and claims have the meanings described below.

“Alkyl” refers to a saturated aliphatic hydrocarbon group including C₁-C₂ straight chain and branched chain groups. In some embodiments, sometimes preferably, an alkyl group is an alkyl having 1 to 8 carbon atom(s) (such as 1, 2, 3, 4, 5, 6, 7 and 8 carbon atom(s)). Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. In some embodiments, sometimes more preferably an alkyl group is a lower alkyl having 1 to 6 carbon atom(s), and sometimes more preferably 1 to 4 carbon atom(s). Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more, sometimes preferably 1 to 5, and sometimes more preferably 1 to 3, group(s) independently selected from the group consisting of halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₁₂ alkenyl, more preferably C₂₋₈ alkenyl, and sometimes more preferably C₂₋₆ alkenyl, and sometimes even more preferably C₂₋₄ alkenyl. The alkenyl group can be substituted or unsubstituted. When substituted, the substituent group(s) is preferably one or more, sometimes preferably 1 to 5, and sometimes more preferably 1 to 3, group(s) independently selected from the group consisting of halogen, alkoxy, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₁₂ alkynyl, sometimes more preferably C2-s alkynyl, sometimes more preferably C₂₋₆ alkynyl, and sometimes even more preferably C₂₋₄ alkynyl. The alkynyl group can be substituted or unsubstituted. When substituted, the substituent group(s) is preferably one or more, preferably 1 to 5, and sometimes more preferably 1 to 3, group(s) independently selected from the group consisting of alkenyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxo group.

“Alkylene” refers to a saturated linear or branched divalent aliphatic hydrocarbon group, derived by removing two hydrogen atoms from the same carbon atom or two different carbon atoms of the parent alkane. The straight or branched chain group containing 1 to 12 carbon atom(s) (such as 1, 2, 3, 4, 5, 6, 7, 8, 9, 10, 11 and 12 carbon atom(s)), preferably has 1 to 8 carbon atom(s), more preferably 1 to 6 carbon atom(s), and sometimes more preferably 1 to 4 carbon atom(s). Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably 1 to 5, and sometimes more preferably 1 to 3, group(s) independently selected from the group consisting of selected from alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxo group.

“Alkenylene” refers to an alkylene defined as above that has at least two carbon atoms and at least one carbon-carbon double bond, preferably C₂₋₁₂ alkenylene, more preferably C2-s alkenylene, sometimes more preferably C₂₋₆ alkenylene, and sometimes even more preferably C₂₋₄ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably 1 to 5, and sometimes more preferably 1 to 3, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxo group.

“Cycloalkyl” refers to a saturated and/or partially unsaturated monocyclic or polycyclic hydrocarbon group having 3 to 12 carbon atoms (such as 3, 4, 5, 6, 7, 8, 9, 10, 11 and 12 carbon atoms), sometimes more preferably 3 to 8 carbon atoms, and sometimes even more preferably 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more, preferably one to three, double bonds, it can be aryl and heteroaryl. Preferably a spiro cycloalkyl is 6 to 14 membered, and more preferably 7 to 10 membered (such as 7, 8, 9 and 10 membered). According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, 5-membered/6-membered, 6-membered/6-membered mono-spiro cycloalkyl. Representative examples of spiro cycloalkyl include, but are not limited to the following groups:

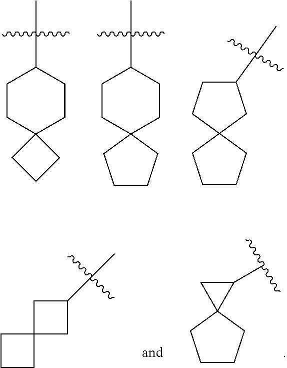

“Fused Cycloalkyl” refers to a polycyclic group, which is a cycloalkyl attached together with one or more, preferably one to five, and sometimes more preferably one to three, group(s) independently selected from cycloalkyl, heterocyclyl, aryl and heteroaryl in a fused manner. Wherein cycloalkyl, heterocyclyl, aryl and heteroaryl are as defined in the present disclosure. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably refers to aryl fused C₅₋₈cycloalkyl, heteroaryl fused C₅₋₈cycloalkyl. 4-membered heterocyclyl fused C₅₋₈ cycloalkyl, 5-membered heterocyclyl fused C₅₋₈ cycloalkyl, C₆ cycloalkyl fused C₅₋₈ cycloalkyl or C₅ cycloalkyl fused C₅₋₈ cycloalkyl, Representative examples of fused cycloalkyls include, but are not limited to, the following groups:

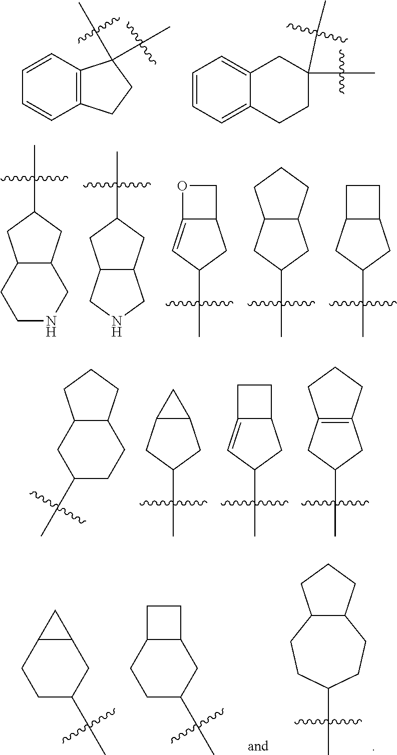

“Bridged Cycloalkyl” refers to a 5 to 20 membered polycyclic hydrocarbon group, wherein every two rings in the system share two disconnected carbon atoms. The rings can have one or more, preferably one to three, double bonds. Preferably, a bridged cycloalkyl is 6 to 14 membered, and more preferably 7 to 10 membered (such as 7, 8, 9 and 10 membered). According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

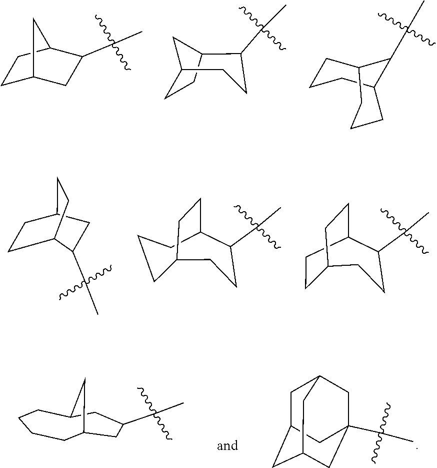

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably 1 to 5, and sometimes more preferably 1 to 3, groups independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more, preferably one to five, and sometimes more preferably one to three, heteroatoms selected from the group consisting of N, O, S, S(O) and S(O)₂ as ring atoms, but excluding —O—O—, —O—S— or —S—S— in the ring, the remaining ring atoms being C. Preferably, heterocyclyl is a 3 to 12 membered (such as 3, 4, 5, 6, 7, 8, 9, 10, 11 and 12 membered) having 1 to 4 heteroatoms (such as 1, 2, 3 and 4 heteroatom(s)); more preferably a 3 to 8 membered (such as 3, 4, 5, 6, 7 and 8 membered) having 1 to 3 heteroatoms (such as 1, 2 and 3 heteroatom(s)); even more preferably a 3 to 6 membered (such as 3, 4, 5 and 6 membered) having 1 to 3 heteroatom(s) (such as 1, 2 and 3 heteroatom(s)); most preferably a 5 to 6 membered having 1 to 3 heteroatom(s) (such as 1, 2 and 3 heteroatom(s)). Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more heteroatoms selected from the group consisting of N, O, S, S(O) and S(O)₂ as ring atoms, the remaining ring atoms being C, wherein one or more rings can contain one or more double bonds. Preferably a spiro heterocyclyl is 6 to 14 membered (such as 6, 7, 8, 9, 10, 11, 12, 13 and 14 membered), and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to monospiro heterocyclyl or di-spiro heterocyclyl, more preferably 4-membered/4-membered, 4-membered/5-membered, 4-membered/6-membered, 5-membered/5-membered, 5-membered/6-membered, 6-membered/6-membered mono-spiro heterocyclyl. Representative examples of spiro heterocyclyl include, but are not limited to the following groups:

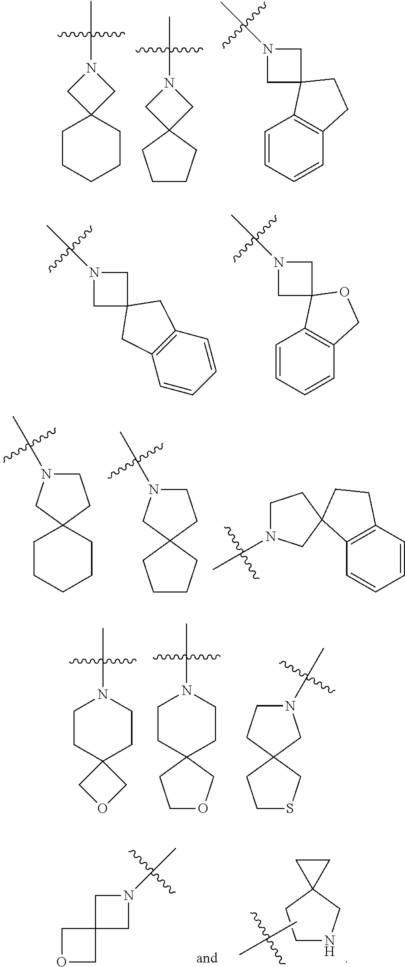

“Fused Heterocyclyl” refers to a polycyclic group, which is a heterocyclyl attached together with one or more, preferably one to three, group(s) selected from cycloalkyl, heterocyclyl, aryl and heteroaryl in a fused manner. Wherein cycloalkyl, heterocyclyl, aryl and heteroaryl are as defined in the present disclosure. According to the number of membered rings, fused heterocyclyl is divided into bicyclic, tricyclic, tetracyclic or polycyclic fused heterocyclyl, and preferably refers to a bicyclic or tricyclic fused cycloalkyl, more preferably refers to aryl fused 5 to 8-member heterocyclyl, heteroaryl fused 5 to 8-member heterocyclyl. C₅₋₈ cycloalkyl fused 4-membered heterocyclyl, C₅₋₈ cycloalkyl fused 5-membered heterocyclyl, C₅₋₈ cycloalkyl fused 6-member heterocyclyl. Representative examples of fused heterocyclyl include, but are not limited to, the following groups:

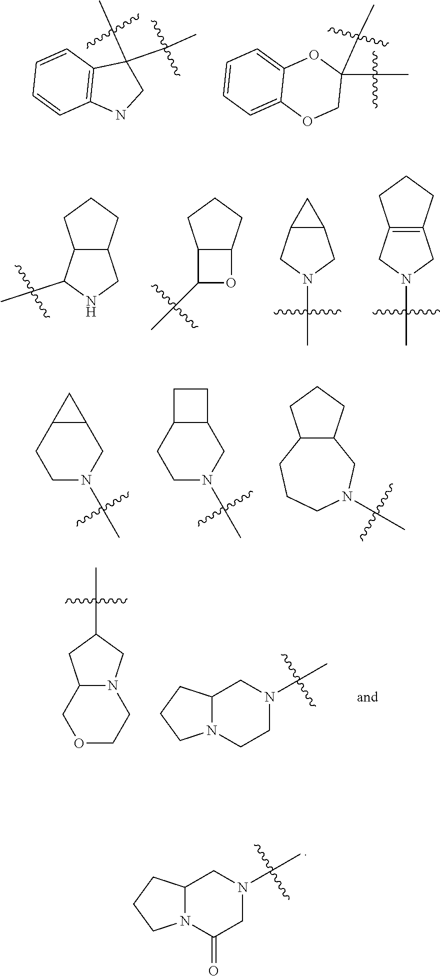

“Bridged Heterocyclyl” refers to a 5 to 14 membered (such as 5, 6, 7, 8, 9, 10, 11, 12, 13 and 14 membered) polycyclic heterocyclic alkyl group, wherein every two rings in the system share two disconnected atoms, the rings can have one or more, preferably one to three, double bonds, and the rings have one or more, preferably one to five, and sometimes more preferably one to three, heteroatoms independently selected from the group consisting of N, O, S, S(O) and S(O)₂ as ring atoms, the remaining ring atoms being C. Preferably a bridged heterocyclyl is 6 to 14 membered (such as 6, 7, 8, 9, 10, 11, 12, 13 and 14 membered), and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

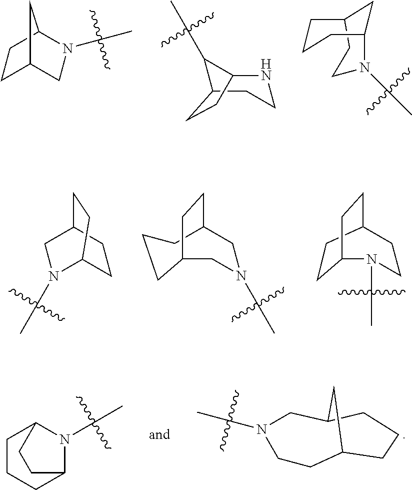

The ring of said heterocyclyl can be fused to the ring of an aryl, heteroaryl or cycloalkyl, wherein the ring bound to the parent structure is heterocyclyl. Representative examples include, but are not limited to the following groups:

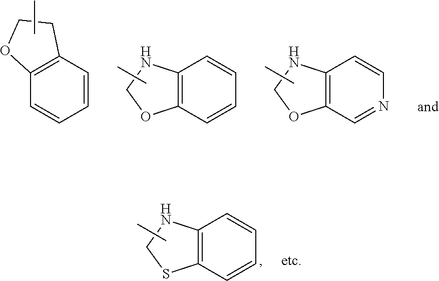

The heterocyclyl is optionally substituted or unsubstituted. When substituted, the substituent group(s) is preferably one or more, sometimes preferably 1 to 5 (such as 1, 2, 3, 4 and 5), and sometimes more preferably 1 to 3, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxy group.

“Aryl” refers to a 6 to 14 membered (such as 6, 7, 8, 9, 10, 11, 12, 13 and 14 membered) all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered (such as 6, 7, 8, 9 and 10 membered), such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following groups:

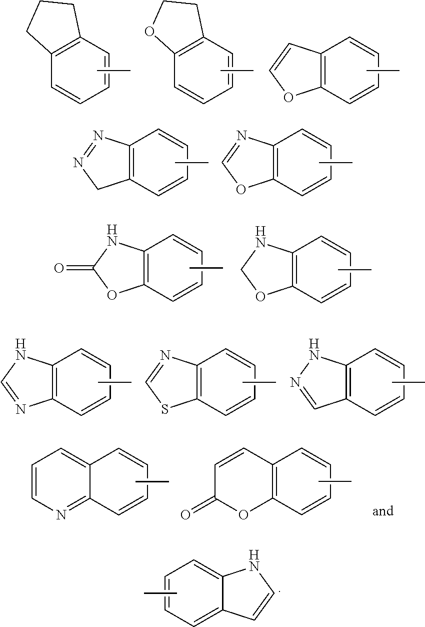

The aryl group can be substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxy group.

“Heteroaryl” refers to an aryl system having 1 to 4 heteroatom(s) (such as 1, 2, 3 and 4 heteroatom(s)) selected from the group consisting of O, S and N as ring atoms and having 5 to 14 annular atoms (such as 5, 6, 7, 8, 9, 10, 11, 12, 13 and 14). Preferably a heteroaryl is 5- to 10-membered (such as 5, 6, 7, 8, 9 and 10 membered), more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. Representative examples include, but are not limited to, the following groups:

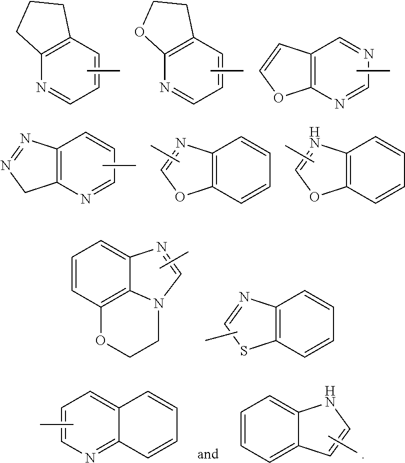

The heteroaryl group can be substituted or unsubstituted. When substituted, the substituent group(s) is preferably one or more, sometimes preferably 1 to 5 (such as 1, 2, 3, 4 and 5), and sometimes more preferably 1 to 3, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxy group.

“Alkoxy” refers to both an —O-(alkyl) group, wherein the alkyl is defined as above. Representative examples include, but are not limited to, methoxy, ethoxy, propoxy, butoxy and the like. The alkoxyl can be substituted or unsubstituted. When substituted, the substituent is preferably one or more, sometimes preferably 1 to 5 (such as 1, 2, 3, 4 and 5), and sometimes more preferably 1 to 3, group(s) independently selected from the group consisting of alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocyclyloxy, cycloalkylthio, heterocylic alkylthio and oxy group.

“Amino protecting group” refers to a derivative of the groups commonly employed to block or protect an amino group while reactions are carried out on other functional groups on the compound. Examples of such protecting groups include carbamates, amides, alkyl and aryl groups, and imines, as well as many N-heteroatom derivatives which can be removed to regenerate the desired amine group. Non-limiting examples include (trimethylsilyl)ethoxymethyl (SEM), tetrahydropyranyl, tert-butoxycarbonyl (Boc), benzyloxycarbonyl (Cbz), (9-Fluorenylmethyloxycarbonyl) (Fmoc), acetyl, benzyl, allyl Group and p-methoxybenzyl (Pmb), etc.

“Hydroxyl protecting group” refers to a derivative of the hydroxy group commonly employed to block or protect the hydroxy group while reactions are carried out on other functional groups on the compound. Examples of such protecting groups include triethylsilyl, triisopropylsilyl, tert-butyldimethylsilyl Group (TBS), tert-butyldiphenylsilyl, etc.; or C₁₋₁₀ alkyl or substituted alkyl, preferably alkoxy or aryl substituted alkyl, more preferably C₁₋₆ alkoxy substituted C₁₋₆ alkyl or phenyl substituted C₁₋₆ alkyl, most preferably C₁₋₄ alkoxy substituted C₁₋₄ alkyl, for example: methyl, tert-butyl, allyl, benzyl, methoxy Methyl (MOM), ethoxyethyl, 2-tetrahydropyranyl (THP), etc.; or (C₁₋₁₀ alkyl or aryl) acyl, such as formyl, acetyl, benzoyl, P-nitrobenzoyl, etc.; (C₁₋₆ alkyl or C₆₋₁₀ aryl)sulfonyl; or (C₁₋₆ alkoxy or C₆₋₁₀ aryloxy) carbonyl.

“Bond” refers to a covalent bond using a sign of “—”.

“deuterated alkyl” refers to an alkyl group substituted by a or more deuterium atom, wherein alkyl is as defined above.

“Hydroxyalkyl” refers to an alkyl group substituted by one or more hydroxy group(s), wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O)OH group.

“Alkoxycarbonyl” refers to a —C(O)O(alkyl) or —C(O)O(cycloalkyl) group, wherein the alkyl and cycloalkyl are defined as above.

“Optional” or “optionally” means that the event or circumstance described subsequently can, but need not occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 6, more preferably 1 to 3 hydrogen atom(s), independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

As a person skilled in the art would understand, the compounds of formula (IM) or Pharmaceutically acceptable salts thereof disclosed herein may exist in prodrug or solvate forms, which are all encompassed by the present disclosure.

“Prodrug” refers to compounds that can be transformed in vivo to yield the active parent compound under physiological conditions, such as through hydrolysis in blood. Common examples include, but are not limited to, ester and amide forms of a compound having an active form bearing a carboxylic acid moiety. Amides and esters of the compounds of the present disclosure may be prepared according to conventional methods. In particular, in the present disclosure, a prodrug may also be formed by acylation of an amino group or a nitrogen atom in a heterocyclyl ring structure, which acyl group can be hydrolyzed in vivo. Such acyl group includes, but is not limited to, a C1-C6 acyl, preferably C1-C4 acyl, and more preferably C1-C2 (formyl or acetyl) group, or benzoyl.

The term “solvate,” as used herein, means a physical association of a compound of this disclosure with one or more, preferably one to three, solvent molecules, whether organic or inorganic. This physical association includes hydrogen bonding. In certain instances, the solvate will be capable of isolation, for example, when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (D) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a RAF inhibitor (or, e.g., a metabolite thereof) at a particular time post-administration may be indicative of whether a therapeutically effective amount has been used.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

The term “subject” or “patient” refers to a mammalian animal.

The term “mammal” or “mammalian animal” includes, but is not limited to, humans, dogs, cats, horses, pigs, cows, monkeys, rabbits and mice. The preferred mammals are humans.

As used herein, the singular forms “a”, “an”, and “the” include plural reference, and vice versa, unless the context clearly dictates otherwise.

When the term “about” is applied to a parameter, such as pH, concentration, temperature, or the like, it indicates that the parameter can vary by ±10%, and sometimes more preferably within ±5%. As would be understood by a person skilled in the art, when a parameter is not critical, a number is often given only for illustration purpose, instead of being limiting.

### Synthesis Method of the Compounds of the Present Disclosure

In order to complete the purpose of the disclosure, the present disclosure applies, but is not limited to, the following technical solution:

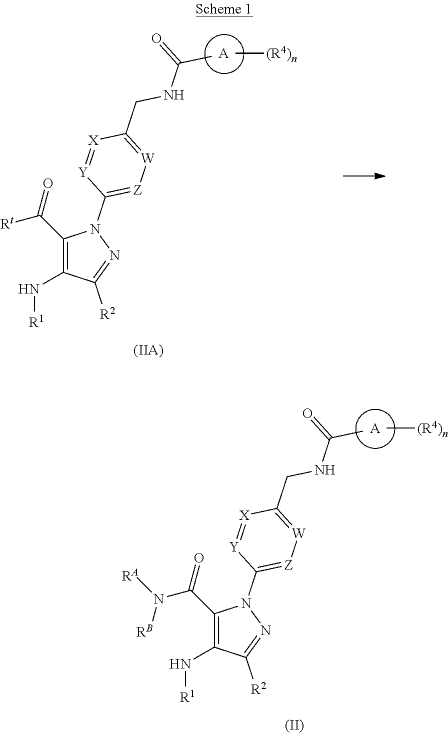

A process of preparing a compound of formula (II), or a pharmaceutically acceptable salt, solvate, or prodrug thereof, comprises a step of:

A compound of Formula (IIA) or a salt thereof was reacted with ammonia water or NHR^{A}R^{B}, or a salt thereof (preferably NH₄Cl) to obtain the compound of formula (II), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl; and

R^{A}, R^{B}, W, X, Y, Z, R¹, R², R⁴, ring A and n are as defined in formula (II).

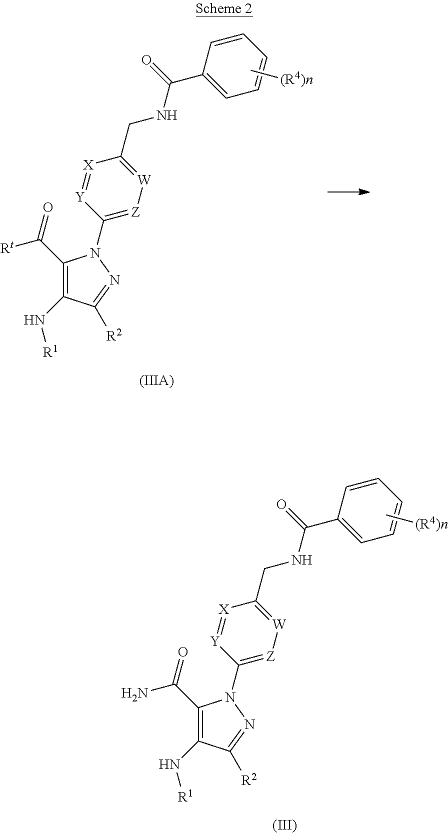

A process of preparing a compound of formula (III), or a pharmaceutically acceptable salt, solvate, or prodrug thereof, comprises a step of:

A compound of Formula (IIIA) or a salt thereof was reacted with ammonia water or NHR^{A}R^{B}, or a salt thereof (preferably NH₄Cl) to obtain the compound of formula (III), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl;

R^{A} and R^{B} are identical, and each is hydrogen; and

W, X, Y, Z, R¹, R², R⁴ and n are as defined in formula (III).

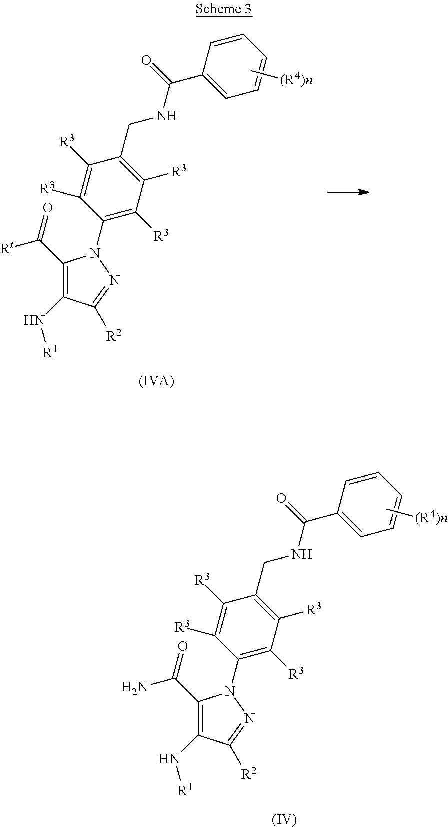

A process of preparing a compound of formula (IV), or a pharmaceutically acceptable salt, solvate, or prodrug thereof, comprises a step of:

A compound of Formula (IVA) or a salt thereof was reacted with ammonia water or NHR^{A}R^{B}, or a salt thereof (preferably NH₄Cl) to obtain the compound of formula (IV), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl;

R^{A} and R^{B} are identical, and each is hydrogen; and

R¹, R², R³, R⁴ and n are as defined in formula (IV).

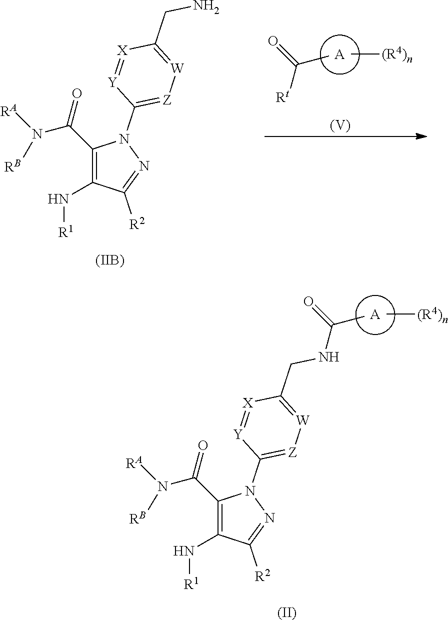

A process of preparing a compound of formula (II), or a pharmaceutically acceptable salt, solvate, or prodrug thereof, comprises a step of:

A compound of Formula (IIB) or a salt thereof was reacted with a compound of Formula (V) to obtain the compound of formula (II), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl; and

R^{A}, R^{B}, W, X, Y, Z, R¹, R², R⁴, ring A and n are as defined in formula (II).

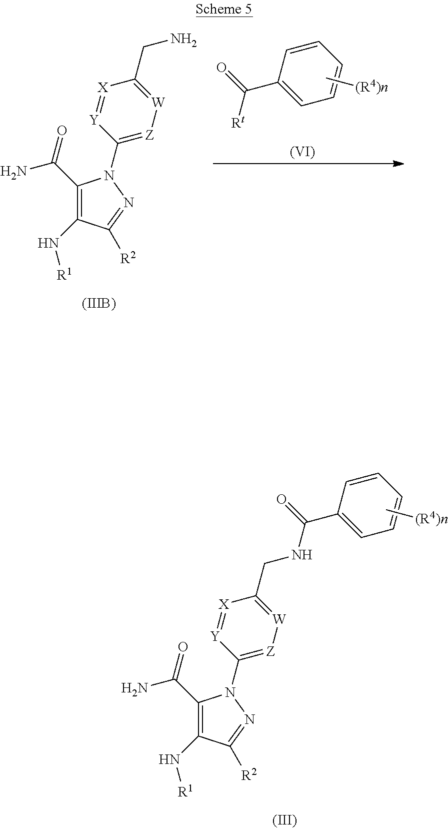

A process of preparing a compound of formula (III), or a pharmaceutically acceptable salt, solvate, or prodrug thereof, comprises a step of:

A compound of Formula (IIIB) or a salt thereof was reacted with a compound of Formula (VI) to obtain the compound of formula (III), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl; and

W, X, Y, Z, R¹, R², R⁴ and n are as defined in formula (III).

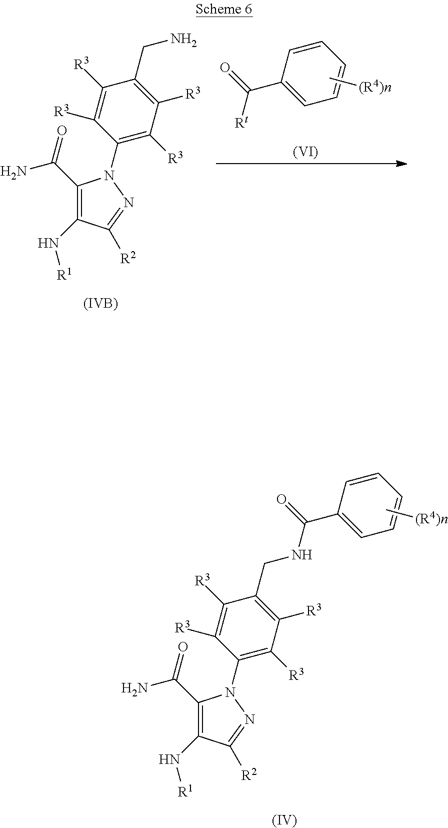

A process of preparing a compound of formula (IV), or a pharmaceutically acceptable salt, solvate, or prodrug thereof, comprises a step of:

A compound of Formula (IVB) or a salt thereof was reacted with a compound of Formula (VI) to obtain the compound of formula (IV), or a pharmaceutically acceptable salt, solvate, or prodrug thereof with coupling reagents under alkaline condition; wherein:

R^{t} is selected from the group consisting of halogen, hydroxyl and alkoxy; preferably R^{t} is hydroxyl; and

R¹, R², R³, R⁴ and n are as defined in formula (IV).

The coupling reagent is preferably HATU.

The agent which provides the alkaline condition includes organic bases and inorganic bases, wherein the organic base includes, but is not limited to, trimethylamine (TEA), N,N-diisopropylethylamine (DIPEA), n-butyllithium, lithium diisopropylamide, potassium acetate, sodium tertbutoxide and potassium tert-butoxide, preferably TEA or DIPEA, and wherein the inorganic base includes, but is not limited to, sodium hydride, potassium phosphate, sodium carbonate, potassium carbonate and cesium carbonate.

The reaction is preferably in solvent, wherein solvent used herein includes, but is not limited to, acetic acid, methanol, ethanol, toluene, acetone, tetrahydrofuran, dichloromethane, dichloroethane, dimethylsulfoxide, 1,4-dioxane, water, N,N-dimethylformamide, trimethylphosphate, methyl tert-butyl ether, pyridine and the mixture thereof.

### EXAMPLES

The following examples serve to illustrate the invention, but the examples should not be considered as limiting. If specific conditions for the experimental method are not specified in the examples of the present disclosure, they are generally in accordance with conventional conditions or recommended conditions of the raw materials and the product manufacturer. The reagents without a specific source indicated are commercially available, conventional reagents.

The structures of compounds were determined by mass spectrometry (MS) and/or nuclear magnetic resonance (NMR). NMR shift (δ) is given in units of 10⁻⁶ (ppm).

The mass spectrum (MS) was determined using a Shimadzu LCMS-2020 liquid chromatography-mass spectrometer.

The NMR measurement was performed on a Bruker AVANCE-400 and 500 Ultrashield nuclear magnetic resonance spectrometer. The solvents were deuterated dimethylsulfoxide (DMSO-d₆), deuterated chloroform (CDCl₃) and deuterated methanol (methanol-d₄) with internal tetramethylsilane (TMS) as reference.

HPLC was performed using a Shimadzu OPTION BOX-L high pressure liquid phase Chromatograph (Gemini 5 um NX-C18 100×21.2 mm column).

Thin-layer chromatography (TLC) silica gel plates used were Agela Technologies T-CSF10050-M silica gel plate with size of 50 mm,

Column chromatography was commonly done using CombiFlash Rf+ Automated Flash Chromatography System (TELEDYNE ISCO) with Agela Technologies Flash Column Silica—CS prepacked columns.

Known starting materials of the present disclosure may be synthesized according to methods known in the art or may be purchased from Acros Organics, Sigma-Aldrich Chemical Company, AstaTech and other companies. Unless otherwise specified in the examples, the reactions were carried out under an argon atmosphere or a nitrogen atmosphere. Argon or nitrogen atmosphere refers to the reaction flask connected to a volume of about 1 L argon or nitrogen balloon.

Hydrogen atmosphere refers to the reaction bottle connected to a volume of about 1 L hydrogen balloon. Hydrogenation reaction system was usually evacuated, refilled with hydrogen, repeatedly for 3 times before the reaction.

The microwave reaction was carried out using a CEM Discover-S 908860 microwave reactor.

Unless otherwise specified in the examples, the reaction temperature was room temperature between 20° C. to 30° C.

The progress of the reaction in the examples was monitored using thin layer chromatography (TLC) or LC-MS chromatography. The column chromatography eluent for the purification of compounds and developing system for thin-layer chromatography include: A: dichloromethane/methanol system, B: n-hexane/ethyl acetate system, C: dichloromethane/ethyl acetate system. The volume ratio of the solvents is adjusted according to the polarity of the compounds. A small amount of triethylamine, acetic acid, other alkaline or acidic reagents can be used to improve the separation.

HCl is in hydrochloric acid,

DMF.DMA is N,N-dimethylformamide dimethyl acetal,

Xantphos is 4,5-bis(diphenylphosphino)-9,9-dimethylxanthene,

Pd₂(dba)₃ is tris(dibenzylideneacetone)dipalladium,

TMEDA is N,N,N′,N′-tetramethylethylenediamine,

CDI is 1,1-carbonyldiimidazole,

(Boc)₂O is di-tert-butyl decarbonate,

NaIO₄ is sodium periodate,

RuCl₃ is ruthenium(III) chloride,

NBS is N-bromosuccinimide,

MeI is methyl iodide,

K₂CO₃ is potassium carbonate,

KOAc is potassium acetate,

Cs₂CO₃ is cesium carbonate,

K₃PO₄ is tripotassium phosphate,

NaOH is sodium hydroxide,

NaO^{t}Bu is sodium tert-butoxide,

KO^{t}Bu is potassium tert-butoxide,

NH₄OH is ammonium hydroxide,

H₂O₂ is hydrogen peroxide,

TFAA is trifluoroacetic anhydride,

DIPEA is N,N-diisopropylethylamine,

EtOAc is ethyl acetate,

MeOH is methanol,

DMSO is dimethyl sulfoxide,

THE is tetrahydrofuran,

DCM is dichloromethane

DMF is dimethylformamide,

^{t}BuOH is tert-butanol,

CCl₄ is carbon tetrachloride,

MgSO₄ is magnesium sulfate,

Na₂SO₄ is sodium sulfate,

NaNO₂ is sodium nitrite,

KOH is potassium hydroxide,

Na₂S₂O₃ is sodium thiosulfate, and

MS is mass spectroscopy with (+) referring to the positive mode which generally gives a

M+H absorption where M=the molecular mass.

### Intermediate 1 (Int-1)

### 4-amino-1-(4-(aminomethyl)phenyl)-3-isopropyl-1H-pyrazole-5-carboxamide

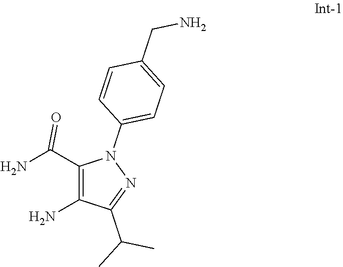 [SMILES: CC(C)c1nn(-c2ccc(CN)cc2)c(C(N)=O)c1N]

### Synthetic Route

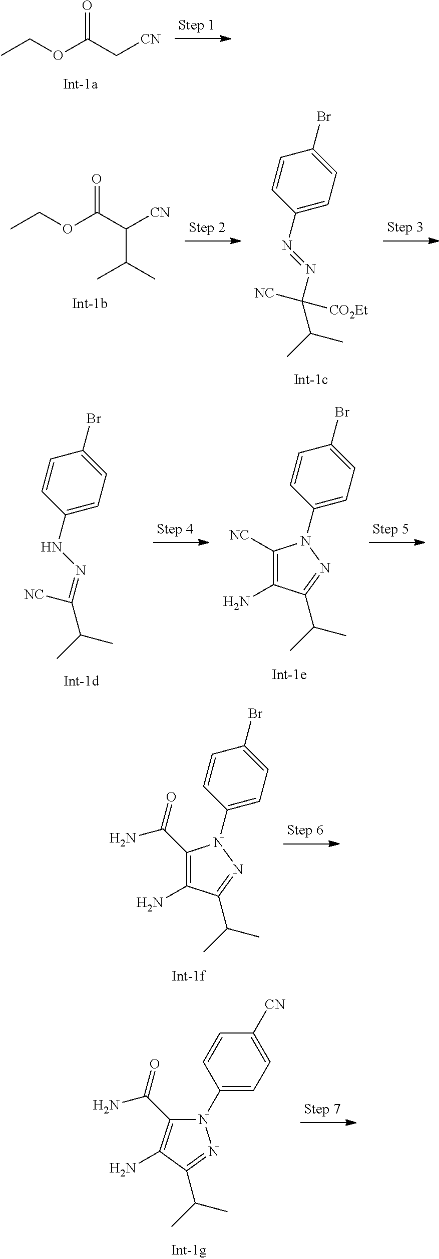

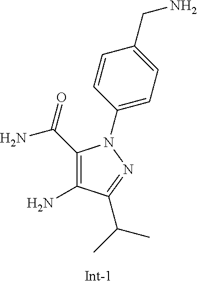 [SMILES: CC(C)c1nn(-c2ccc(CN)cc2)c(C(N)=O)c1N]

### Step 1: Ethyl 2-cyano-3-methylbutanoate Int-1b

To a solution of ethyl 2-cyanoacetate (Int-1a, 10 g, 88.41 mmol) in acetone (150 mL) at room temperature under nitrogen atmosphere was added K₂CO₃ (36.65 g, 265.22 mmol) and 2-iodopropane (22.54 g, 132.61 mmol). The resulting mixture was stirred at 70° C. for 48 hours. After cooling down, the reaction mixture was diluted with EtOAc (200 mL) and quenched with water, and then extracted with EtOAc (200 mL×3). The combined organic extracts were washed with brine (200 mL), dried over anhydrous Na₂SO₄, filtered and concentrated under reduced pressure. The residue was purified by silica gel column chromatography (EtOAc:petroleum ether=0:1˜1:5) to afford ethyl 2-cyano-3-methylbutanoate Int-1b (12 g, 87% yield). ¹H NMR (400 MHz, CDCl₃): δ 4.27 (q, J=7.2 Hz, 2H), 3.42 (d, J 5.2 Hz, 1H), 2.47-2.36 (m, 1H), 1.33 (t, J=7.2 Hz, 3H), 1.12 (dd, 13.6 Hz, 6.8 Hz, 6H) ppm.

### Step 2. Ethyl (E)-2-((4-bromophenyl)diazenyl)-2-cyano-3-methylbutanoate Int-1c

To a solution of 4-bromoaniline (22.17 g, 128.87 mmol) in HCl (11.75 g, 322.18 mmol) at room temperature under nitrogen atmosphere was added NaNO₂ (8.89 g, 128.87 mmol) in water dropwise. The resulting mixture was stirred for 1 h before added dropwise to an ice cooled solution of Int-1b (10 g, 64.44 mmol) in ethanol (20 mL) and water (280 mL). The pH of the reaction was maintained at 7 by adding KOAc portion-wise. The resulting mixture was slowly warmed up to room temperature overnight, and then quenched with saturated aqueous NH₄Cl (50 mL), followed by extraction with EtOAc (50 mL×3). The combined organic extracts were washed with brine (100 mL), dried over anhydrous Na₂SO₄, filtered, and concentrated under reduced pressure. The residue was purified by silica gel column chromatography (EtOAc:petroleum ether=0:1˜1:20) to afford ethyl (E)-2-((4-bromophenyl)diazenyl)-2-cyano-3-methylbutanoate Int-1c (15 g, 68% yield). ¹H NMR (400 MHz, DMSO-d₆): δ 7.85-7.81 (m, 2H), 7.75-7.71 (m, 2H), 4.35 (q, J=7.2 Hz, 2H), 3.13-3.02 (m, 1H), 1.26 (t, J=7.2 Hz, 3H), 1.15 (d, J=6.8 Hz, 3H), 0.98 (d, J=6.4 Hz, 3H) ppm.

### Step 3: (Z)—N-(4-bromophenyl)isobutyrohydrazonoyl cyanide Int-1d

To a solution of ethyl (E)-2-((4-bromophenyl)diazenyl)-2-cyano-3-methylbutanoate (Int-1c, 2 g, 5.91 mmol) in THE (20 mL) at 0° C. under nitrogen atmosphere was added NaOH (10 M in water, 11.83 mL). The resulting mixture was stirred for 3 h before quenched with saturated aqueous NH₄Cl (200 mL) followed by extraction with EtOAc (200 mL×3). The combined organic extracts were washed with brine (200 mL), dried over anhydrous Na₂SO₄, filtered and concentrated under reduced pressure. The residue was purified by silica gel column chromatography (EtOAc:petroleum ether=0:1˜1:15) to afford (Z)—N-(4-bromophenyl)isobutyrohydrazonoyl cyanide Int-1d (1.3 g, 82% yield). ¹H NMR (400 MHz, DMSO-d₆): δ 10.78 (s, 1H), 7.43 (d, J=8.8 Hz, 2H), 7.19 (d, J=8.8 Hz, 2H), 2.79-2.75 (m, 1H), 1.18 (d, J=7.2 Hz, 6H) ppm.

### Step 4: 4-amino-1-(4-bromophenyl)-3-isopropyl-1H-pyrazole-5-carbonitrile Int-1e

To a solution of Int-1d (1 g, 3.76 mmol) and 2-bromoacetonitrile (676.05 mg, 5.64 mmol) in BuOH (15 mL) at room temperature under nitrogen atmosphere was added NaO^{t}Bu (361.10 mg, 3.76 mmol). The resulting mixture was stirred for 5 h before quenched with NH₄Cl (200 mL) followed by extraction with EtOAc (200 mL×3). The combined organic extracts were washed with brine (200 mL), dried over anhydrous Na₂SO₄, filtered, and concentrated under reduced pressure. The residue was purified by silica gel column chromatography (EtOAc:petroleum ether=0:1˜1:3) to afford 4-amino-1-(4-bromophenyl)-3-isopropyl-1H-pyrazole-5-carbonitrile Int-1e (1 g, 87% yield). ¹H NMR (400 MHz, DMSO-d₆): δ 7.73-7.68 (m, 2H), 7.59-7.54 (m, 2H), 5.56 (s, 2H), 3.09-3.02 (m, 1H), 1.22 (d, J=6.8 Hz, 6H) ppm.

### Step 5: 4-amino-1-(4-bromophenyl)-3-isopropyl-1H-pyrazole-5-carboxamide Int-1f

To a solution of Int-1e (1 g, 3.28 mmol) in MeOH (10 mL) was added NaOH (1 M in water, 6.55 mL) and H₂O₂ (1.86 g, 16.38 mmol, 16.38 mL, 30% purity). The resulting mixture was stirred at ambient temperature for 16 h under nitrogen atmosphere. The reaction mixture was then quenched with saturated aqueous Na₂S₂O₃ (50 mL) and extracted with EtOAc (50 mL×3). The combined organic extracts were washed with brine (50 mL), dried over anhydrous Na₂SO₄, filtered, and concentrated under reduced pressure. The residue was purified by silica gel column chromatography (DCM:MeOH=1:0-15:1) to afford 4-amino-1-(4-bromophenyl)-3-isopropyl-1H-pyrazole-5-carboxamide Int-1f (1 g, 94% yield). ¹H NMR (400 MHz, MeOD): δ 7.61-7.57 (m, 2H), 7.34-7.29 (m, 2H), 3.10-2.99 (m, 1H), 1.29 (d, J=7.2 Hz, 6H) ppm. LCMS: MS m/z (ESI): 322.9 [M+H]⁺.

### Step 6: 4-amino-1-(4-cyanophenyl)-3-isopropyl-1H-pyrazole-5-carboxamide Int-1 g

To a solution of Int-1f (1 g, 3.09 mmol) in DMF (10 mL) at room temperature under nitrogen atmosphere was added Xantphos (179.03 mg, 309.42 μmol), Pd₂(dba)₃ (283.34 mg, 309.42 μmol), TMEDA (179.78 mg, 1.55 mmol) and zinc cyanide (726.67 mg, 6.19 mmol). The resulting mixture was stirred at 170° C. for 1 h. After cooling down, the reaction mixture was quenched with water (20 mL) and then extracted with EtOAc (20 mL×3). The combined organic extracts were washed with brine (50 mL), dried over anhydrous Na₂SO₄, filtered, and then concentrated under reduced pressure. The residue was purified by silica gel column chromatography (EtOAc:petroleum ether=0:1˜1:1) to afford 4-amino-1-(4-cyanophenyl)-3-isopropyl-1H-pyrazole-5-carboxamide Int-1 g (300 mg, 36% yield). ¹H NMR (400 MHz, MeOD): δ 7.76 (d, J=8.4 Hz, 2H), 7.57 (d, J=8.8 Hz, 2H), 3.10-2.99 (m, 1H), 1.30 (d, J=7.2 Hz, 6H) ppm; LCMS: MS m/z (ESI): 270.2 [M+H]⁺.

### Step 7: 4-amino-1-(4-(aminomethyl)phenyl)-3-isopropyl-1H-pyrazole-5-carboxamide Int-1

A mixture of Int-1 g (20 mg, 74.27 μmol) and Raney Nickel (21.79 mg, 371.33 μmol) in MeOH (2 mL) under hydrogen atmosphere (15 Psi) was stirred at ambient temperature for 16 h. The reaction mixture was filtered through Celite, and the filtrate was concentrated under vacuum to afford crude 4-amino-1-(4-(aminomethyl)phenyl)-3-isopropyl-1H-pyrazole-5-carboxamide Int-1 (20 mg, 98% yield). LCMS: MS m/z (ESI): 274.1 [M+H]⁺.

### Intermediate 2 (Int-2)

### 4-amino-1-(4-(aminomethyl)-2-ethoxyphenyl)-3-isopropyl-1H-pyrazole-5-carboxamide Int-2

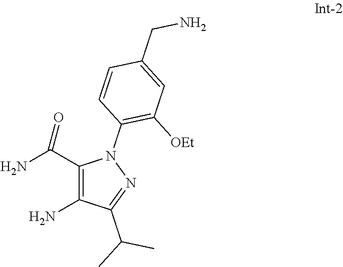 [SMILES: CCOc1cc(CN)ccc1-n1nc(C(C)C)c(N)c1C(N)=O]

### Synthetic Route

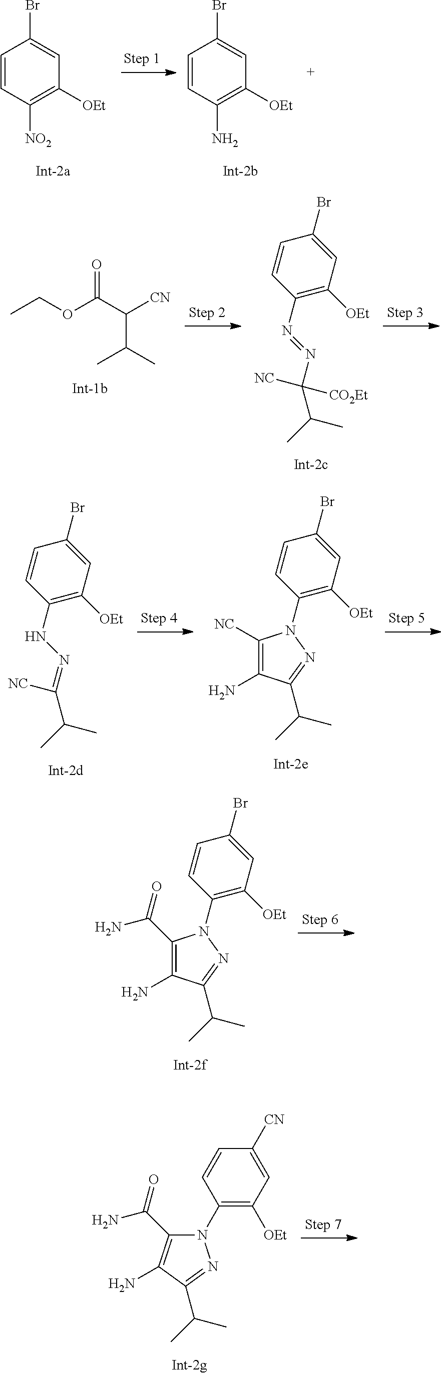

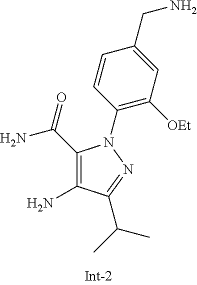 [SMILES: CCOc1cc(CN)ccc1-n1nc(C(C)C)c(N)c1C(N)=O]

### Step 1: 4-bromo-2-ethoxyaniline Int-2b

To a solution of 4-bromo-2-ethoxy-1-nitrobenzene Int-2a (15 g, 60.96 mmol) in EtOH (150 mL) and water (50 mL) at room temperature was added NH₄Cl (30.15 g, 609.61 mmol) and Fe (17.02 g, 304.81 mmol). The resulting solution was stirred at 80° C. for 1 hour. After cooling down, the reaction mixture was quenched with water (200 mL) and extracted with EtOAc (200 mL×3). The combined organic extracts were washed with brine (200 mL), dried over anhydrous Na₂SO₄, filtered, and concentrated under reduced pressure. The residue was purified by silica gel column chromatography (EtOA:petroleum ether-0:1 to 1:3) to afford title compound 4-bromo-2-ethoxyaniline Int-2b (12.5 g, 94.90% yield). ¹H NMR (400 MHz, DMSO-d₆): δ 6.89 (d, J=2.0 Hz, 1H), 6.81 (dd, J=8.4 Hz, 2.4 Hz, 1H), 6.57 (d, J=8.4 Hz, 1H), 4.83 (brs, 2H), 3.99 (q, J=7.2 Hz, 2H), 1.33 (t, J=7.2 Hz, 3H) ppm.

### Step 2: ethyl (E)-2-((4-bromo-2-ethoxyphenyl)diazenyl)-2-cyano-3-methylbutanoate Int-2c

To a solution of 4-bromo-2-ethoxyaniline Int-2b (13.87 g, 64.18 mmol) in HCl (1M in water, 267.41 mL) at room temperature under nitrogen atmosphere was added NaNO₂ (1M in water, 106.96 mL) dropwise. The resulting mixture was stirred for 1 h before added dropwise to an ice cooled solution of ethyl 2-cyano-3-methylbutanoate Int-1b (8.3 g, 53.48 mmol) in ethanol (10 mL) and water (140 mL). The pH of the reaction was maintained at 7 by adding KOAc portion-wise. The resulting mixture was slowly warmed up to room temperature overnight, and then quenched with saturated aqueous NH₄Cl (200 mL). After extraction with EtOAc (200 mL), the combined organic extracts were washed with brine (100 mL), dried over anhydrous Na₂SO₄, filtered, and concentrated under reduced pressure. The residue was purified by silica gel column chromatography (EtOAc:petroleum ether-0:1 to 1:5) to afford ethyl (E)-2-((4-bromo-2-ethoxyphenyl)diazenyl)-2-cyano-3-methylbutanoate Int-2c (14.5 g, 70.93% yield). ¹H NMR (400 MHz, DMSO-d₆): δ 7.51 (d, J=1.2 Hz, 1H), 7.28-7.22 (m, 2H), 4.33 (q, J=7.2 Hz, 2H), 4.24 (q, J=6.8 Hz, 2H), 3.04-2.96 (m, 1H), 1.34 (t, J=6.8 Hz, 3H), 1.25 (t, J=7.2 Hz, 3H), 1.12 (d, J=6.8 Hz, 3H), 1.00 (d, J=6.4 Hz, 3H) ppm.

### Step 3: (Z)—N-(4-bromo-2-ethoxyphenyl)isobutyrohydrazonoyl cyanide Int-2d

To a solution of ethyl (E)-2-((4-bromo-2-ethoxyphenyl)diazenyl)-2-cyano-3-methylbutanoate Int-2c (14.5 g, 37.93 mmol) in THE (145 mL) at 0° C. under nitrogen atmosphere was added NaOH (10 M in water, 75.87 mL). The resulting mixture was stirred at room temperature for 3 h, and then quenched with saturated aqueous NH₄Cl (200 mL). After extraction with EtOAc (200 mL), the combined organic extracts were washed with brine (200 mL), dried over anhydrous Na₂SO₄, filtered, and concentrated under reduced pressure. The residue was purified by silica gel column chromatography (EtOAc:petroleum ether-0:1 to 1:20) to afford title compound (Z)—N-(4-bromo-2-ethoxyphenyl)isobutyrohydrazonoyl cyanide Int-2d (7.4 g, 62.89% yield). ¹H NMR (400 MHz, DMSO-d₆): δ 8.98 (s, 1H), 7.23-7.19 (m, 2H), 7.11 (dd, J=8.4 Hz, 2.0 Hz, 1H), 4.15 (q, J=6.8 Hz, 2H), 2.84-2.76 (m, 1H), 1.36 (t, J=6.8 Hz, 3H), 1.19 (d, J=6.8 Hz, 6H) ppm.

### Step 4: 4-amino-2-(4-bromo-2-ethoxy-phenyl)-5-isopropyl-pyrazole-3-carbonitrile Int-2e

To a solution of (Z)—N-(4-bromo-2-ethoxyphenyl)isobutyrohydrazonoyl cyanide Int-2d (7.4 g, 23.86 mmol) and bromoacetonitrile (5.72 g, 47.71 mmol) in ^{t}BuOH (75 mL) at room temperature under nitrogen atmosphere was added NaO^{t}Bu (4.59 g, 47.71 mmol). The resulting mixture was stirred for 5 hours before quenched with NH₄Cl (200 mL). After extraction with EtOAc (200 mL×3), the combined organic extracts were washed with brine (200 mL), dried over anhydrous Na₂SO₄, filtered, and concentrated under reduced pressure. The residue was purified by silica gel column chromatography (EtOAc:petroleum ether-0:1 to 1:5) to afford title compound 4-amino-2-(4-bromo-2-ethoxy-phenyl)-5-isopropyl-pyrazole-3-carbonitrile Int-2e (3.8 g, 45.61% yield). ¹H NMR (400 MHz, DMSO-d₆): δ 7.42 (s, 1H), 7.25 (s, 2H), 5.26 (brs, 2H), 4.14 (q, J=6.8 Hz, 2H), 3.06-2.98 (m, 1H), 1.31 (t, J=7.2 Hz, 3H), 1.19 (d, J=6.8 Hz, 6H) ppm.

### Step 5: 4-amino-1-(4-bromo-2-ethoxyphenyl)-3-isopropyl-1H-pyrazole-5-carboxamide Int-2f

To a solution of 4-amino-2-(4-bromo-2-ethoxy-phenyl)-5-isopropyl-pyrazole-3-carbonitrile Int-2e (3.8 g, 10.88 mmol) in MeOH (40 mL) under nitrogen atmosphere was added NaOH (1 M in water, 23.94 mL) and H₂O₂ (6.17 g, 54.41 mmol, 30% purity in water). The resulting mixture was stirred at room temperature for 16 hours, and then quenched with saturated aqueous Na₂S₂O₃ (50 mL). After extraction with EtOAc (100 mL×3), the combined organic extracts were washed with brine (50 mL), dried over anhydrous Na₂SO₄, filtered, and concentrated under reduced pressure. The residue was purified by silica gel column chromatography to afford title compound 4-amino-1-(4-bromo-2-ethoxyphenyl)-3-isopropyl-1H-pyrazole-5-carboxamide Int-2f (3.2 g, 80.08% yield). ¹H NMR (400 MHz, DMSO-d₆): δ 7.25-7.22 (m, 2H), 7.18 (dd, J=8.4 Hz, 2.0 Hz, 1H), 4.37 (brs, 2H), 3.98 (q, J=6.8 Hz, 2H), 3.05-2.97 (m, 1H), 1.23-1.18 (m, 9H) ppm.

### Step 6: 4-amino-2-(4-cyano-2-ethoxy-phenyl)-5-isopropyl-pyrazole-3-carboxamide Int-2 g

To a solution of 4-amino-1-(4-bromo-2-ethoxyphenyl)-3-isopropyl-1H-pyrazole-5-carboxamide Int-2f (3.2 g, 8.71 mmol) in DMF (30 mL) at room temperature under nitrogen atmosphere was added zinc cyanide (1.53 g, 13.07 mmol), Xantphos (504.19 mg, 871.36 umol), Pd₂(dba)₃ (797.93 mg, 871.36 umol.) and TMEDA (101.26 mg, 871.36 umol). The resulting mixture was stirred at 140° C. for 16 h. After cooling down, the reaction mixture was quenched with water (100 mL) and then extracted with EtOAc (20 mL×3). The combined organic extracts were washed with brine (50 mL), dried over anhydrous Na₂SO₄, filtered, and then concentrated under reduced pressure. The residue was purified by silica gel column chromatography (DCM:MeOH-1:0 to 15:1) to afford title compound 4-amino-2-(4-cyano-2-ethoxy-phenyl)-5-isopropyl-pyrazole-3-carboxamide Int-2 g (2.08 g, 76.07% yield). ¹H NMR (400 MHz, DMSO-d₆): δ 7.53 (s, 1H), 7.50-7.43 (m, 2H), 4.40 (brs, 2H), 4.03 (q, J=6.8 Hz, 2H), 3.07-2.99 (m, 1H), 1.28-1.20 (m, 9H) ppm.

### Step 7: 4-amino-1-(4-(aminomethyl)-2-ethoxyphenyl)-3-isopropyl-1H-pyrazole-5-carboxamide Int-2

A mixture of 4-amino-2-(4-cyano-2-ethoxy-phenyl)-5-isopropyl-pyrazole-3-carboxamide Int-2 g (2.08 g, 6.64 mmol) and Raney Nickel (194.80 mg, 3.32 mmol) in MeOH (30 mL) under hydrogen atmosphere (15 Psi) was stirred at room temperature for 16 hours. The reaction mixture was filtered through Celite, and the filtrate was concentrated under vacuum to afford crude 4-amino-1-(4-(aminomethyl)-2-ethoxyphenyl)-3-isopropyl-1H-pyrazole-5-carboxamide Int-2 (2 g, 94.90% yield), which was used for next step without further purification. LCMS: MS m/z (ESI): 318.0[M+H]⁺.

### Intermediate 3 (Int-3)

### 4-amino-1-(4-(aminomethyl)-2-fluorophenyl)-3-isopropyl-1H-pyrazole-5-carboxamide Int-3

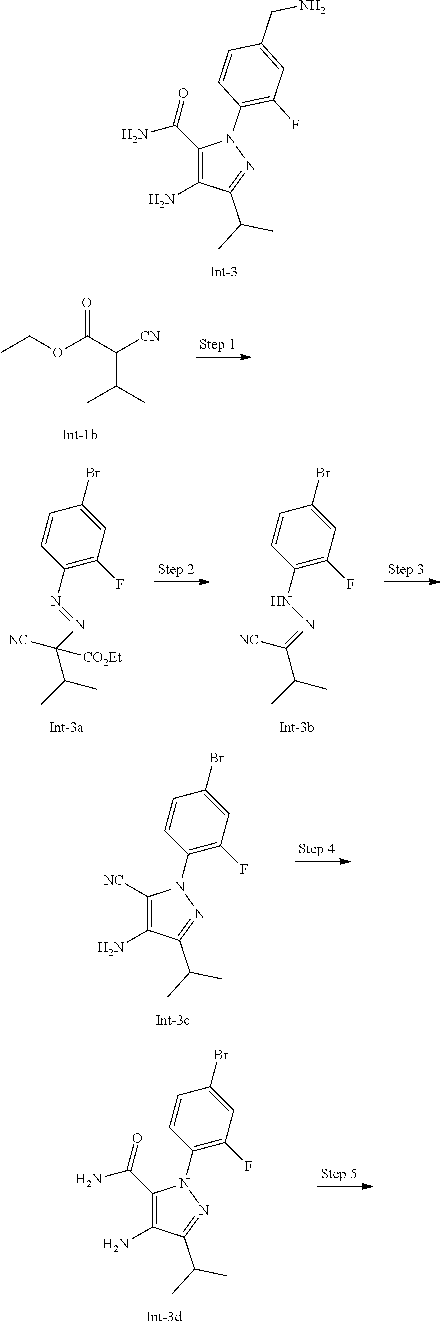 [SMILES: CC(C)c1nn(-c2ccc(CN)cc2F)c(C(N)=O)c1N ; CCOC(=O)C(C#N)C(C)C ; CCOC(=O)C(C#N)(/N=N/c1ccc(Br)cc1F)C(C)C ; CC(C)/C(C#N)=N/Nc1ccc(Br)cc1F ; CC(C)c1nn(-c2ccc(Br)cc2F)c(C#N)c1N ; CC(C)c1nn(-c2ccc(Br)cc2F)c(C(N)=O)c1N]

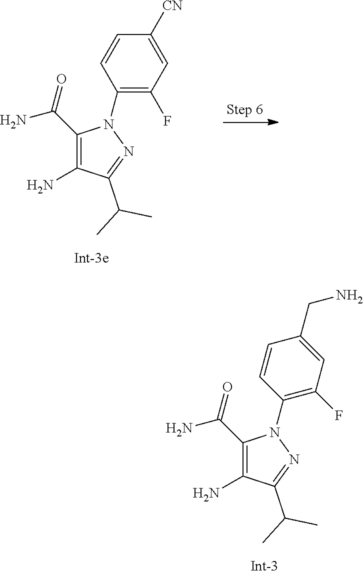 [SMILES: CC(C)c1nn(-c2ccc(C#N)cc2F)c(C(N)=O)c1N ; CC(C)c1nn(-c2ccc(CN)cc2F)c(C(N)=O)c1N]

Step 1: ethyl (E)-2-((4-bromo-2-fluorophenyl)diazenyl)-2-cyano-3-methylbutanoate Int-3a

To a solution of 4-bromo-2-fluoro-aniline (29.38 g, 154.65 mmol) in HCl (1M in water, 644.35 mL) at room temperature under nitrogen atmosphere at 0° C. was added NaNO₂ (1M in water, 257.74 mL) dropwise. The resulting mixture was slowly warmed up to room temperature and stirred for 1 h before added dropwise to an ice cooled solution of ethyl 2-cyano-3-methylbutanoate Int-1b (20 g, 128.87 mmol) in ethanol (10 mL) and water (140 mL). The pH of the reaction was maintained at 7 by adding KOAc portion-wise. The resulting mixture was slowly warmed up to room temperature overnight, and then quenched with saturated aqueous NH₄Cl (200 mL). After extraction with EtOAc (200 mL×3), the combined organic extracts were washed with brine (200 mL), dried over anhydrous Na₂SO₄, filtered, and concentrated under reduced pressure. The residue was purified by silica gel column chromatography (EtOAc:petroleum ether-0:1 to 1:5) to afford ethyl (E)-2-((4-bromo-2-fluorophenyl)diazenyl)-2-cyano-3-methylbutanoate Int-3a (14.6 g, 31.81% yield). ¹H NMR (400 MHz, DMSO-d₆): δ 7.95 (dd, 1H), 7.60 (dd, 1H), 7.50 (t, 1H), 4.34 (q, 2H), 3.11-3.03 (m, 1H), 1.25 (d, 3H), 1.14 (d, 3H), 0.98 (d, 3H) ppm.

### Step 2: (Z)—N-(4-bromo-2-fluorophenyl)isobutyrohydrazonoyl cyanide Int-3b

To a solution of ethyl (E)-2-((4-bromo-2-fluorophenyl)diazenyl)-2-cyano-3-methylbutanoate Int-3a (14 g, 39.30 mmol) in THE (150 mL) at 0° C. under nitrogen atmosphere was added NaOH (10 M in water, 78.61 mL). The resulting mixture was stirred at room temperature for 3 h, and then quenched with saturated aqueous NH₄Cl (200 mL). After extraction with EtOAc (200 mL×3), the combined organic extracts were washed with brine (200 mL), dried over anhydrous Na₂SO₄, filtered, and concentrated under reduced pressure. The residue was purified by silica gel column chromatography (EtOAc:petroleum ether-0:1 to 1:10) to afford (Z)—N-(4-bromo-2-fluorophenyl)isobutyrohydrazonoyl cyanide Int-3b (2.6 g, 23.28% yield). ¹H NMR (400 MHz, DMSO-d₆): δ 10.06 (s, 1H), 7.54 (dd, 1H), 7.35-7.34 (m, 2H), 2.84-2.77 (m, 1H), 1.19 (d, 6H) ppm.

### Step 3: 4-amino-1-(4-bromo-2-fluorophenyl)-3-isopropyl-1H-pyrazole-5-carbonitrile Int-3c

To a solution of (Z)—N-(4-bromo-2-fluorophenyl)isobutyrohydrazonoyl cyanide Int-3b (2.6 g, 9.15 mmol) and bromoacetonitrile (1.65 g, 13.73 mmol) in ^{t}BuOH (30 mL) at room temperature under nitrogen atmosphere was added NaO^{t}Bu (967.37 mg, 10.07 mmol). The resulting mixture was stirred for 5 hours before quenched with saturated aqueous NH₄Cl (200 mL). After extraction with EtOAc (200 mL×3), the combined organic extracts were washed with brine (200 mL), dried over anhydrous Na₂SO₄, filtered, and concentrated under reduced pressure. The residue was purified by silica gel column chromatography (EtOAc:petroleum ether-0:1 to 1:5) to afford 4-amino-1-(4-bromo-2-fluorophenyl)-3-isopropyl-1H-pyrazole-5-carbonitrile Int-3c (1.5 g, 50.72% yield). ¹H NMR (400 MHz, DMSO-d₆): δ 7.87 (dd, 1H), 7.59 (dd, 1H), 7.52 (t, 1H), 5.53 (s, 2H), 3.09-3.01 (m, 1H), 1.20 (d, 6H) ppm.

### Step 4: 4-amino-1-(4-bromo-2-fluorophenyl)-3-isopropyl-1H-pyrazole-5-carboxamide Int-3d

To a solution of 4-amino-1-(4-bromo-2-fluorophenyl)-3-isopropyl-1H-pyrazole-5-carbonitrile Int-3c (1.5 g, 4.64 mmol) in MeOH (20 mL) under nitrogen atmosphere was added NaOH (1 M in water, 10.21 mL) and H₂O₂ (2.63 g, 23.21 mmol, 30% purity in water). The resulting mixture was stirred at room temperature for 16 hours, and then quenched with saturated aqueous Na₂S₂O₃ (100 mL). After extraction with EtOAc (100 mL×3), the combined organic extracts were washed with brine (100 mL), dried over anhydrous Na₂SO₄, filtered, and concentrated under reduced pressure. The residue was purified by silica gel column chromatography (MeOH:DCM=0:100-1:15) to afford 4-amino-1-(4-bromo-2-fluorophenyl)-3-isopropyl-1H-pyrazole-5-carboxamide Int-3d (1.2 g, 75.78% yield). ¹H NMR (400 MHz, DMSO-d₆): δ 7.63 (dd, 1H), 7.47 (dd, 1H), 7.38 (t, 1H), 7.24 (brs, 2H), 4.51 (s, 2H), 3.09-3.01 (m, 1H), 1.20 (d, 6H) ppm; LCMS: MS m/z (ESI): 343.1 [M+H]⁺.

### Step 5: 4-amino-1-(4-cyano-2-fluorophenyl)-3-isopropyl-1H-pyrazole-5-carboxamide Int-3e

To a solution of 4-amino-1-(4-bromo-2-fluorophenyl)-3-isopropyl-1H-pyrazole-5-carboxamide Int-3d (1.2 g, 3.52 mmol) in DMF (15 mL) at room temperature under nitrogen atmosphere was added zinc cyanide (41.30 mg, 351.72 umol), Xantphos (203.51 mg, 351.72 umol), Pd₂(dba)₃ (321.83 mg, 351.72 umol) and TMEDA (40.87 mg, 351.72 umol). The resulting mixture was stirred at 140° C. for 16 h. After cooling down, the reaction mixture was quenched with water (100 mL) and then extracted with EtOAc (20 mL×3). The combined organic extracts were washed with brine (100 mL), dried over anhydrous Na₂SO₄, filtered, and then concentrated under reduced pressure. The residue was purified by silica gel column chromatography (DCM:MeOH-1:0 to 15:1) to afford 4-amino-1-(4-cyano-2-fluorophenyl)-3-isopropyl-1H-pyrazole-5-carboxamide Int-3e (200 mg, 19.79% yield). ¹H NMR (400 MHz, DMSO-d₆): δ 7.95 (d, 1H), 7.75 (d, 1H), 7.63 (t, 1H), 7.33 (brs, 2H), 4.65 (s, 2H), 3.11-3.03 (m, 1H), 1.22 (d, 6H) ppm.

### Step 6: 4-amino-1-(4-(aminomethyl)-2-fluorophenyl)-3-isopropyl-1H-pyrazole-5-carboxamide Int-3

A mixture of 4-amino-1-(4-cyano-2-fluorophenyl)-3-isopropyl-1H-pyrazole-5-carboxamide Int-3e (200 mg, 696.16 umol) and Raney Nickel (12.26 mg, 208.85 umol) in MeOH (5 mL) under hydrogen atmosphere (15 Psi) was stirred at room temperature for 16 hours. The reaction mixture was filtered through Celite, and the filtrate was concentrated under vacuum to afford crude Int-3 (100 mg, 49.31% yield), which was used for next step without further purification. ¹H NMR (400 MHz, MeOD): δ 7.54 (t, 1H), 7.37-7.34 (m, 2H), 4.17 (s, 2H), 3.11-3.05 (m, 1H), 1.30 (d, 6H) ppm; LCMS: MS m/z (ESI): 292.1 [M+H]⁺.

### Intermediate 4 (Int-4)

### 4-amino-1-(4-(aminomethyl)-2-ethoxy-6-fluorophenyl)-3-isopropyl-1H-pyrazole-5-carboxamide Int-4

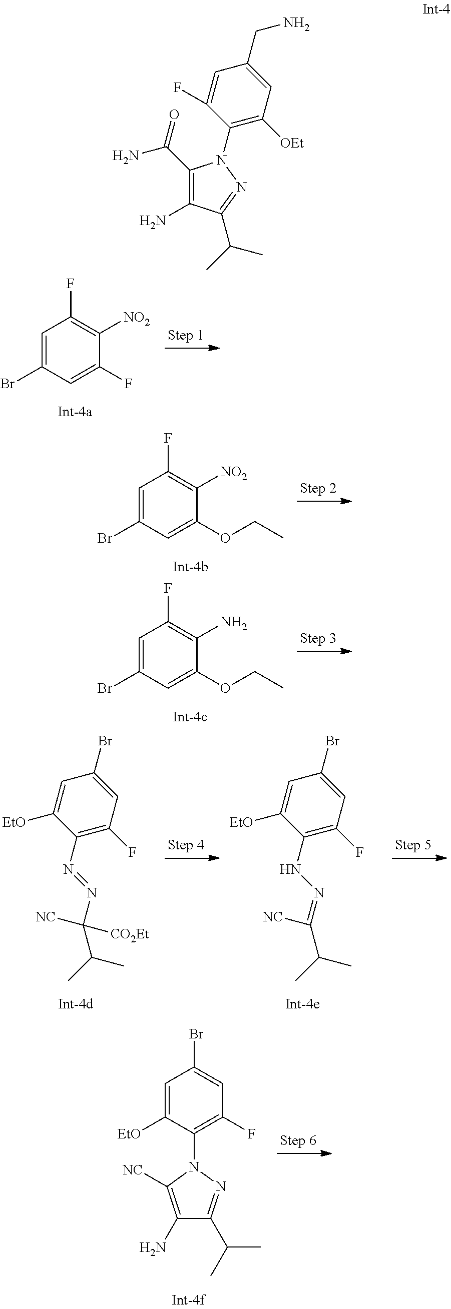

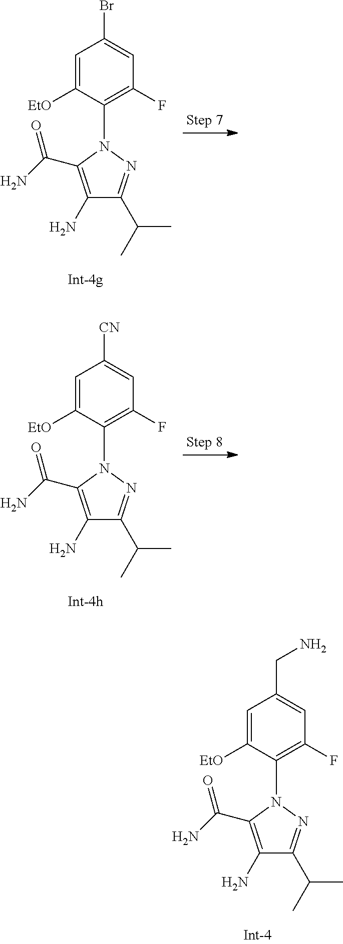 [SMILES: CCOc1cc(Br)cc(F)c1-n1nc(C(C)C)c(N)c1C(N)=O ; CCOc1cc(C#N)cc(F)c1-n1nc(C(C)C)c(N)c1C(N)=O ; CCOc1cc(CN)cc(F)c1-n1nc(C(C)C)c(N)c1C(N)=O]

### Step 1: 5-bromo-1-ethoxy-3-fluoro-2-nitrobenzene Int-4b

To a solution of 5-bromo-1,3-difluoro-2-nitrobenzene Int-4a (5.0 g, 21 mmol) in EtOH (80 mL) was added K₂CO₃ (8.71 g, 63 mmol). The resulting mixture was stirred overnight at room temperature, and then filtered through Celite. The filtrate was concentrated under vacuum, and the resulting residue was purified by silica gel column chromatography (PE/EtOAc=30/1) to afford 5-bromo-1-ethoxy-3-fluoro-2-nitrobenzene Int-4b (5.4 g, 96.25% yield). ¹H NMR (400 MHz, DMSO-d₆): δ 7.56 (dd, 1H), 7.51 (t, 1H), 4.27 (q, 2H), 1.30 (t, 3H) ppm.

### Step 2: 4-bromo-2-ethoxy-6-fluoroaniline Int-4c

To a solution of 5-bromo-1-ethoxy-3-fluoro-2-nitrobenzene Int-4b (4.2 g, 15.91 mmol,) in acetic acid (15 mL) was added Fe power (5.5 g, 79.53 mmol). The mixture was slowly warmed up and stirred at 70° C. for 40 min. After cooling down, the mixture was filtered through Celite. The filtrate was concentrated under vacuum, and the resulting residue was purified by silica gel column chromatography (PE/EtOAc=30/1) to afford 4-bromo-2-ethoxy-6-fluoroaniline Int-4c (3.4 g, 90.98% yield). 1H NMR (400 MHz, DMSO-d₆): δ 6.94 (dd, 1H), 6.84 (s, 1H), 4.77 (brs, 2H), 4.04 (q, 2H), 1.34 (t, 3H) ppm.

### Step 3: (E)-2-((4-bromo-2-ethoxy-6-fluorophenyl)diazenyl)-2-cyano-3-methylbutanoate Int-4d

To a solution of 4-bromo-2-ethoxy-6-fluoroaniline Int-4c (2.0 g, 8.54 mmol) in HCl (1 M in water, 50 mL) at room temperature under nitrogen atmosphere was added NaNO₂ (648.54 mg, 9.40 mmol) in water dropwise. The resulting mixture was stirred for 1 h before added dropwise to an ice cooled solution of ethyl 2-cyano-3-methylbutanoate Int-1b (1.39 g, 8.97 mmol) in ethanol (10 mL) and water (40 mL). The pH of the reaction was maintained at 7 by adding KOAc portion-wise. The resulting mixture was slowly warmed up to room temperature overnight, and then quenched with saturated aqueous NH₄Cl (50 mL), followed by extraction with EtOAc (60 mL×3). The combined organic extracts were washed with brine (50 mL), dried over anhydrous Na₂SO₄, filtered, and concentrated under reduced pressure. The residue was purified by silica gel column chromatography (EtOAc:petroleum ether=1:4) to afford ethyl (E)-2-((4-bromo-2-ethoxy-6-fluorophenyl)diazenyl)-2-cyano-3-methylbutanoate Int-4d (3.0 g, 87.72% yield). LCMS: MS m/z (ESI): 402.0 [M+H]⁺.

### Step 4: (Z)—N-(4-bromo-2-ethoxy-6-fluorophenyl)isobutyrohydrazonoyl cyanide Int-4e

To a solution of ethyl (E)-2-((4-bromo-2-ethoxy-6-fluorophenyl)diazenyl)-2-cyano-3-methylbutanoate Int-4d (300 mg, 749.55 umol) in THE (145 mL) at 0° C. under nitrogen atmosphere was added NaOH (149.91 mg, 3.75 mmol) in H₂O (2 mL). The resulting mixture was slowly warmed up to room temperature and stirred for 3 h before quenched with saturated aqueous NH₄Cl (200 mL) followed by extraction with EtOAc (200 mL×3). The combined organic extracts were washed with brine (200 mL), dried over anhydrous Na₂SO₄, filtered and concentrated under reduced pressure. The residue was purified by prep-HPLC, eluting with MeCN/H₂O/FA, to afford (Z)—N-(4-bromo-2-ethoxy-6-fluorophenyl)isobutyrohydrazonoyl cyanide Int-4e (100 mg, 40.65% yield). LCMS: MS m/z (ESI): 326.0 [M−H]⁻

### Step 5: 4-amino-1-(4-bromo-2-ethoxy-6-fluorophenyl)-3-isopropyl-1H-pyrazole-5-carbonitrile Int-4f

To a solution of (Z)—N-(4-bromo-2-ethoxy-6-fluorophenyl)isobutyrohydrazonoyl cyanide Int-4e (30 mg, 95.49 umol) and 2-bromoacetonitrile (57.27 mg, 477.47 umol) in BuOH (3 mL) at room temperature under nitrogen atmosphere was added NaO^{t}Bu (45.84 mg, 477.47 umol). The resulting mixture was stirred for 5 h before quenched with saturated aqueous NH₄Cl (50 mL) followed by extraction with EtOAc (50 mL×3). The combined organic extracts were washed with brine (200 mL), dried over anhydrous Na₂SO₄, filtered, and concentrated under reduced pressure. The residue was purified by silica gel column chromatography (EtOAc:petroleum ether=1:3) to afford 4-amino-1-(4-bromo-2-ethoxy-6-fluorophenyl)-3-isopropyl-1H-pyrazole-5-carbonitrile Int-4f (20 mg, 59.30% yield). LCMS: MS m/z (ESI): 368.9 [M+H]⁺.

### Step 6: 4-amino-1-(4-bromo-2-ethoxy-6-fluorophenyl)-3-isopropyl-1H-pyrazole-5-carboxamide Int-4 g

To a solution of 4-amino-1-(4-bromo-2-ethoxy-6-fluorophenyl)-3-isopropyl-1H-pyrazole-5-carbonitrile Int-4f (30 mg, 84.94 umol) in MeOH (3 mL) was added NaOH (10.19 mg, 254.82 umol) and H₂O₂ (8.66 mg, 254.82 umol, 30% purity). The resulting mixture was stirred at ambient temperature for 1 hour under nitrogen atmosphere. The reaction mixture was then quenched with saturated aqueous Na₂S₂O₃ (100 mL) and extracted with EtOAc (100 mL×3). The combined organic extracts were washed with brine (50 mL), dried over anhydrous Na₂SO₄, filtered, and concentrated under reduced pressure to afford crude 4-amino-1-(4-bromo-2-ethoxy-6-fluorophenyl)-3-isopropyl-1H-pyrazole-5-carboxamide Int-4 g (30 mg, 95.15% yield), which was used for next step without further purification. LCMS: MS m/z (ESI): 385.0 [M+H]⁺

### Step 7: 4-amino-1-(4-cyano-2-ethoxy-6-fluorophenyl)-3-isopropyl-1H-pyrazole-5-carboxamide Int-4 h

To a solution of 4-amino-1-(4-bromo-2-ethoxy-6-fluorophenyl)-3-isopropyl-1H-pyrazole-5-carboxamide Int-4 g (20 mg, 51.92 umol) in DMF (3 mL) at room temperature under nitrogen atmosphere was added Xantphos (30.01 mg, 51.92 umol), Pd₂(dba)₃ (47.50 mg, 51.92 umol), TMEDA (6.03 mg, 51.92 umol) and zinc cyanide (6.07 mg, 51.92 umol). The resulting mixture was stirred at 140° C. for 1 h. After cooling down, the reaction mixture was quenched with water (20 mL) and then extracted with EtOAc (20 mL×3). The combined organic extracts were washed with brine (50 mL), dried over anhydrous Na₂SO₄, filtered, and then concentrated under reduced pressure to afford crude 4-amino-1-(4-cyano-2-ethoxy-6-fluorophenyl)-3-isopropyl-1H-pyrazole-5-carboxamide Int-4 h (15 mg, 87.20% yield), which was used for next step without further purification. LCMS: MS m/z (ESI): 332.1 [M+H]⁺.

### Step 8: 4-amino-1-(4-(aminomethyl)-2-ethoxy-6-fluorophenyl)-3-isopropyl-1H-pyrazole-5-carboxamide Int-4

A mixture of 4-amino-1-(4-cyano-2-ethoxy-6-fluorophenyl)-3-isopropyl-1H-pyrazole-5-carboxamide Int-4 h (10 mg, 0.045 mmol), Pd/C (10 mg), and 3 drops of HCl in EtOH under hydrogen balloon was stirred for 1.5 hours at room temperature. The reaction mixture was filtered through Celite, and the filtrate was concentrated under vacuum to afford crude 4-amino-1-(4-(aminomethyl)-2-ethoxy-6-fluorophenyl)-3-isopropyl-1H-pyrazole-5-carboxamide Int-4, which was used for next step without further purification.

### Example A1

### 4-amino-1-(4-((5-fluoro-2-methoxybenzamido)methyl)phenyl)-3-isopropyl-1H-pyrazole-5-carboxamide A1

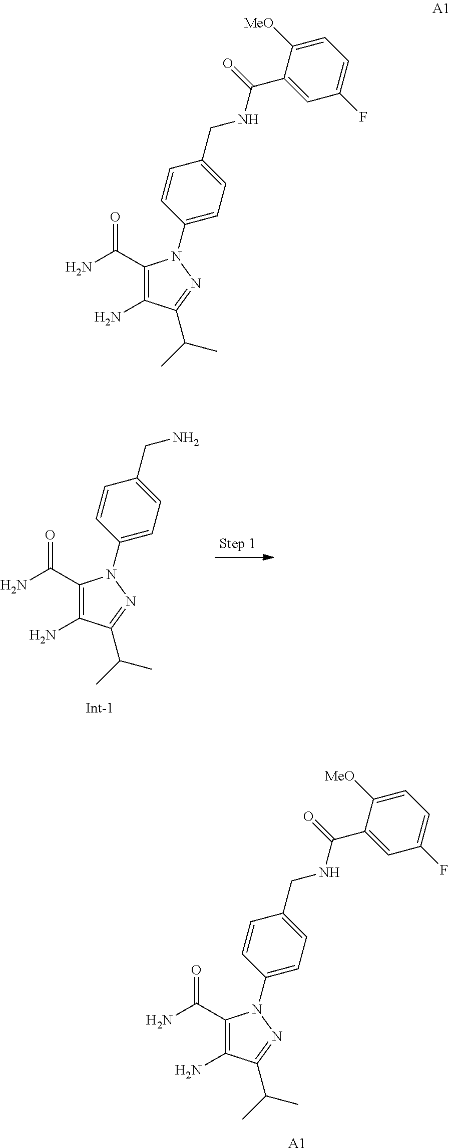

### Step 1: 4-amino-1-(4-((5-fluoro-2-methoxybenzamido)methyl)phenyl)-3-isopropyl-1H-pyrazole-5-carboxamide A1

To a solution of 5-fluoro-2-methoxybenzoic acid (87.14 mg, 512.20 μmol) in DMF (5 mL) under nitrogen atmosphere at room temperature was added HATU (556.44 mg, 1.46 mmol) The mixture was stirred at room temperature for 0.5 h, and then Int-1 (200 mg, 731.71 μmol) and DIPEA (283.70 mg, 2.20 mmol) were added to the reaction mixture. The resulting mixture was stirred for 3 h before quenched with water (100 mL), and then extracted with EtOAc (20 mL×3). The combined organic extracts were washed with brine (20 mL), dried over anhydrous Na₂SO₄, filtered and concentrated under reduced pressure. The residue was purified by prep-HPLC (NH₄OH in MeCN/H₂O) to afford 4-amino-1-(4-((5-fluoro-2-methoxybenzamido)methyl)phenyl)-3-isopropyl-1H-pyrazole-5-carboxamide (A1 (103.31 mg, 33% yield). ¹H NMR (400 MHz, MeOD): δ 7.61 (dd, J=9.2 Hz, 3.2 Hz, 1H), 7.47 (d, J=8.4 Hz, 2H), 7.41-7.34 (m, 2H), 7.27-7.22 (m, 1H), 7.15 (dd, J=9.2 Hz, 4.0 Hz, 1H), 4.65 (s, 2H), 3.95 (s, 3H), 3.09-3.01 (m, 1H), 1.30 (d, J=6.8 Hz, 6H) ppm; LCMS: MS m/z (ESI): 426.3 [M+H]⁺.

### Example A2

### 4-amino-1-(4-((2-cyclopropoxy-5-fluorobenzamido)methyl)phenyl)-3-isopropyl-1H-pyrazole-5-carboxamide A2

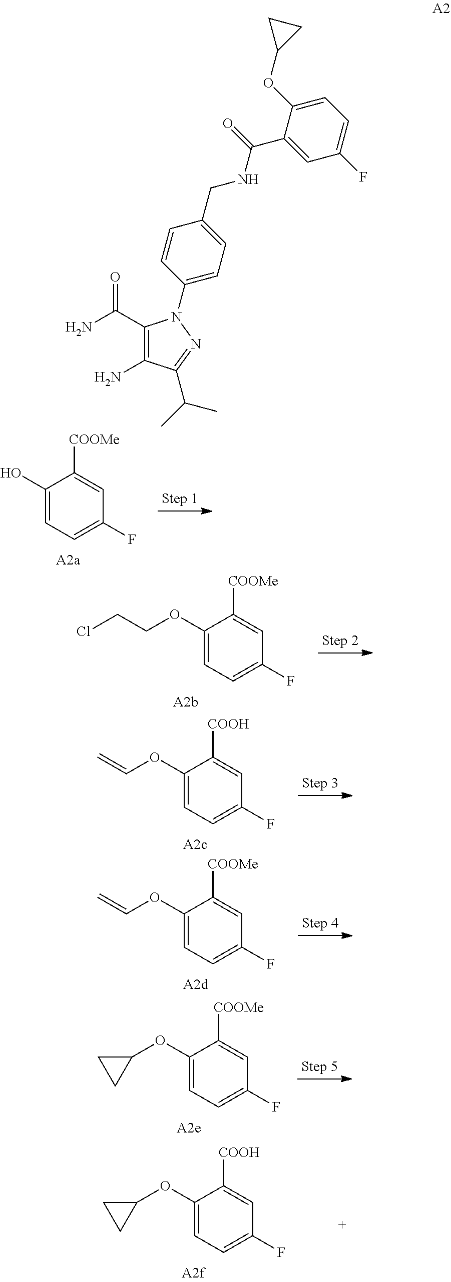

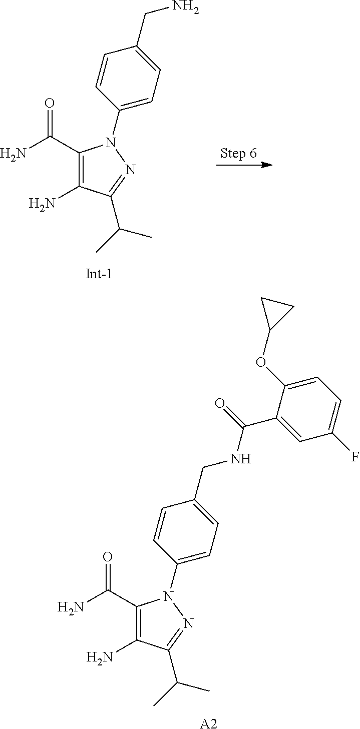

### Step 1: Methyl 2-(2-chloroethoxy)-5-fluorobenzoate A2b

A mixture of methyl 5-fluoro-2-hydroxybenzoate A2a (10 g, 58.78 mmol), 2-chloroethyl 4-methylbenzenesulfonate (14.48 g, 61.71 mmol) and K₂CO₃ (16.25 g, 117.56 mmol) in DMF (30 mL) under nitrogen atmosphere was stirred at 70° C. for 18 hrs. After cooling down, the mixture was diluted with EtOAc (25 mL) and extracted with H₂O (25 mL). The layers were separated, and the organic layer was dried over anhydrous Na₂SO₄, filtered, and concentrated under reduced pressure. The residue was purified by silica gel column chromatography (SiO₂, 75% hexanes in EtOAc) afforded the title compound A2b (11.27 g, 82% yield). ¹H NMR (400 MHz, CDCl₃): δ 7.52 (dd, J=8.8 Hz, 3.2 Hz, 1H), 7.20-7.14 (m, 1H), 6.97 (dd, J=9.2 Hz, 4.4 Hz, 1H), 4.27 (t, J=6.0 Hz, 2H), 3.90 (s, 3H), 3.84 (t, J=6.0 Hz, 2H) ppm.

### Step 2: 5-fluoro-2-(vinyloxy)benzoic acid A2c

To the solution of A2b (5 g, 21.49 mmol) in THE (50 mL) under nitrogen atmosphere at 0° C. was added KO^{t}Bu (7.24 g, 64.48 mmol) in portions with the internal temperature being maintained bellowed 5° C. After the addition completed, the mixture was heated to 75° C., and stirred for 4 hrs. After cooling down, the mixture was diluted with H₂O (35 mL), extracted with EtOAc (50 mL) and the layers were separated. The aqueous layer was acidified with 1N aqueous HCl (Sinopharm) to pH=3 and extracted with EtOAc (15 mL×3). The organic extracts were combined and dried over anhydrous Na₂SO₄, filtered, and concentrated under reduced pressure to afford the title compound 5-fluoro-2-(vinyloxy)benzoic acid A2c (3.61 g, 92% yield). LCMS: MS m/z (ESI): 183.3 [M+H]⁺.

### Step 3: Methyl 5-fluoro-2-(vinyloxy)benzoate A2d

To a solution of A2c (3.61 g, 19.82 mmol) in DMF (40 mL) was added K₂CO₃ (8.22 g, 59.46 mmol) and CH₃I (5.63 g, 39.64 mmol). After the addition completed, the reaction was stirred at 35° C. for 2 hrs. After cooling down, the mixture was diluted with EtOAc (25 mL) and extracted with H₂O (25 mL). The layers were separated, and the organic layer was dried over anhydrous Na₂SO₄, filtered, and concentrated under reduced pressure. The residue was purification by silica gel column chromatography (PE/EtOAc=10/1) to afford the title compound Methyl 5-fluoro-2-(vinyloxy)benzoate A2d (2.94 g, 75.62% yield). ¹H NMR (400 MHz, CDCl₃): δ 7.56 (dd, J=8.4 Hz, 3.2 Hz, 1H), 7.22-7.17 (m, 1H), 7.06 (dd, J=8.8 Hz, 4.4 Hz, 1H), 6.57 (dd, J=13.6 Hz, 6.0 Hz, 1H), 4.62 (dd, J=14.0 Hz, 2.0 Hz, 1H), 4.43 (dd, J=6.0 Hz, 2.0 Hz, 1H), 3.91 (s, 3H) ppm; LCMS: MS m/z (ESI): 197.1 [M+H]⁺

### Step 4: Methyl 2-cyclopropoxy-5-fluorobenzoate A2e

To a solution of A2d (1.5 g, 7.65 mmol) in DCM (30 mL) at −5° C. was added chloro-iodomethane (1.35 g, 7.65 mmol). A solution of diethylzinc (1 M, 7.65 mL) was added dropwise over 20 min. After the addition completed, the mixture was allowed to warm to ambient temperature and was stirred for 2 hours before cooling to 0° C. again. The reaction mixture was quenched with saturated aqueous NH₄Cl (5 mL) and concentrated NH₄OH (1 mL). The mixture was then extracted with EtOAc (2 mL) and the organic layers were dried over anhydrous Na₂SO₄, filtered, and concentrated under reduce pressure. The residue was purification by silica gel column chromatography (PE/EtOAc=10/1) afforded the title compound methyl 2-cyclopropoxy-5-fluorobenzoate A2e (1.58 g, 98% yield).

### Step 5: 2-cyclopropoxy-5-fluorobenzoic acid A2f

To a solution of A2e (1.58 g, 7.52 mmol) in EtOH (10 mL) at ambient temperature was added KOH (5.27 g, 37.58 mmol, 40% in water). The mixture was stirred at ambient temperature for 2 hours and partially concentrated to remove the ethanol. The aqueous residue was extracted with DCM (20 mL×3). The aqueous layer was acidified with 10% aqueous HCl to pH=1 and then extracted with DCM (20 mL×3). The combined organic extracts were dried over anhydrous Na₂SO₄, filtered, and concentrated under reduced pressure to afford the title compound 2-cyclopropoxy-5-fluorobenzoic acid A2f (1.53 g, 92% yield). ¹H NMR (400 MHz, CDCl₃): δ 7.84 (dd, J=8.8 Hz, 3.2 Hz, 1H), 7.41 (dd, J=9.2 Hz, 4.4 Hz, 1H), 7.31-7.25 (m, 1H), 4.02-3.95 (m, 1H), 0.98-0.95 (m, 4H) ppm; LCMS: MS m/z (ESI): 197.1 [M+H]⁺.

### Step 6: 4-amino-1-(4-((2-cyclopropoxy-5-fluorobenzamido)methyl)phenyl)-3-isopropyl-1H-pyrazole-5-carboxamide A2

The mixture of A2f (50.24 mg, 256.10 μmol) and HATU (208.66 mg, 548.78 μmol), DIPEA (141.59 mg, 1.10 mmol) in DMF (5 mL) was stirred at room temperature for 15 min before addition of Int-1 (100 mg, 365.85 μmol). The resulting mixture was stirred at room temperature for 30 min before diluted with EtOAc and further extracted with brine. The organic phase was concentrated, and the residue was purified by prep-TLC (DCM:MeOH=20:1) to afford 4-amino-1-(4-((2-cyclopropoxy-5-fluorobenzamido)methyl)phenyl)-3-isopropyl-1H-pyrazole-5-carboxamide A2 (111.4 mg, 67% yield). ¹H NMR (400 MHz, DMSO-d₆): δ 8.57-8.51 (m, 1H), 7.46-7.40 (m, 2H), 7.37-7.34 (m, 3H), 7.28 (d, J=9.2 Hz, 2H), 4.52 (brs, 2H), 4.49 (d, J=6.0 Hz, 2H), 4.02-3.92 (m, 1H), 3.06-2.94 (m, 1H), 1.21 (d, J=6.8 Hz, 6H), 0.83-0.73 (m, 4H) ppm; LCMS: MS m/z (ESI): 452.1 [M+H]⁺.

### Example A3

### 4-amino-1-(4-((5-fluoro-2-methoxybenzamido)methyl)phenyl)-3-(1,1,1-trifluoropropan-2-yl)-1H-pyrazole-5-carboxamide A3

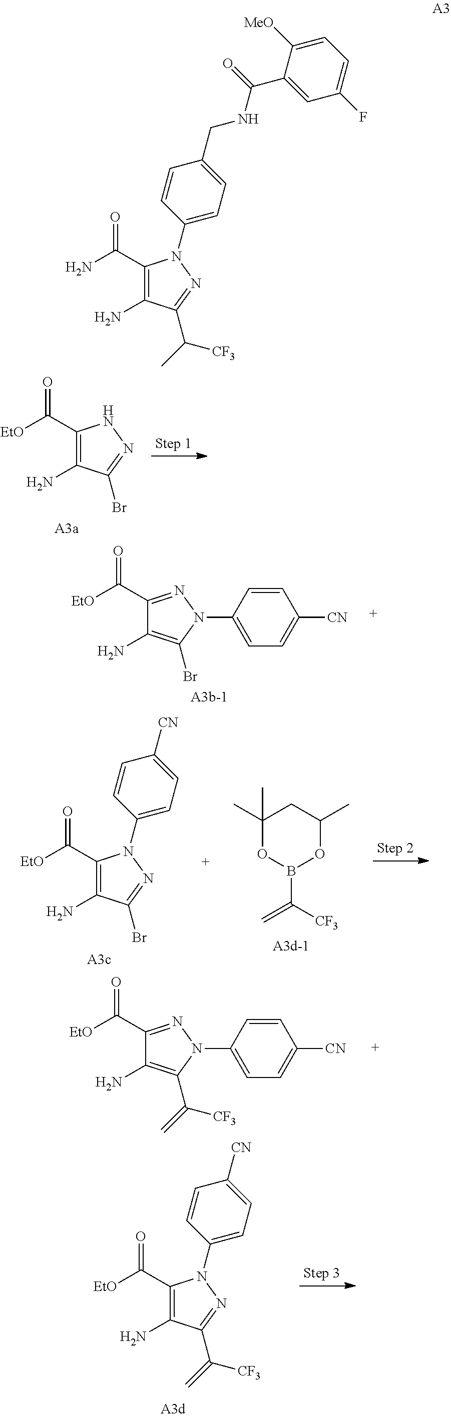

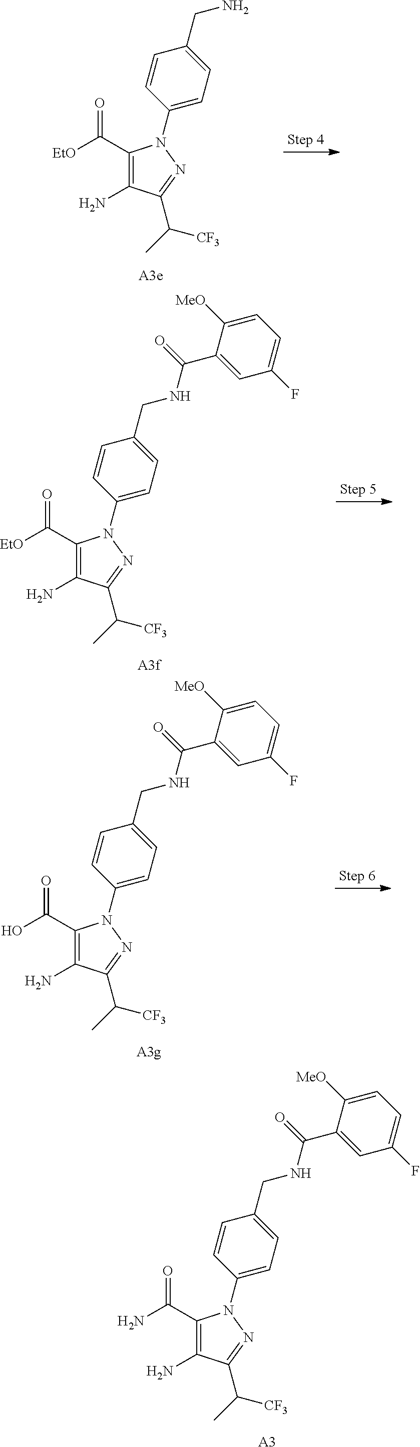

### Step 1: ethyl 4-amino-3-bromo-1-(4-cyanophenyl)-1H-pyrazole-5-carboxylate A3b and ethyl 4-amino-5-bromo-1-(4-cyanophenyl)-1H-pyrazole-3-carboxylate A3b-1

A mixture of ethyl 4-amino-3-bromo-1H-pyrazole-5-carboxylate A3a (950 mg, 4.06 mmol), 4-fluorobenzonitrile (1.48 g, 12.18 mmol) and K₂CO₃ (1.68 g, 12.18 mmol) in DMF (50 mL) under nitrogen atmosphere was stirred overnight at 80° C. After cooling down, the mixture was concentrated under vacuum. The residue was purified by silica gel column chromatography (DCM/EtOAc) to afford ethyl 4-amino-3-bromo-1-(4-cyanophenyl)-1H-pyrazole-5-carboxylate A3b and ethyl 4-amino-5-bromo-1-(4-cyanophenyl)-1H-pyrazole-3-carboxylate A3b-1 as a mixture, which was used for next step without further purification. LCMS: MS m/z (ESI): 337.0 [M+H]⁺.

### Step 2: ethyl 4-amino-1-(4-cyanophenyl)-3-(3,3,3-trifluoroprop-1-en-2-yl)-1H-pyrazole-5-carboxylate A3d and ethyl 4-amino-1-(4-cyanophenyl)-5-(3,3,3-trifluoroprop-1-en-2-yl)-1H-pyrazole-3-carboxylate A3d-1

A mixture of ethyl 4-amino-3-bromo-1-(4-cyanophenyl)-1H-pyrazole-5-carboxylate A3b and ethyl 4-amino-5-bromo-1-(4-cyanophenyl)-1H-pyrazole-3-carboxylate A3b-1 (4.08 mmol), 1-(trifluoromethyl)vinylboronic acid hexylene glycol ester A3c (1 g, 4.8 mmol), Pd(dppf)Cl₂ (300 mg, 0.408 mmol) and Cs₂CO₃ (4 g, 12.24 mmol) in dioxane:H₂O (10:1, 30 mL) under nitrogen atmosphere was stirred at 90° C. for 1 hour. After cooling down, the mixture was filtered through a silica pad, concentrated under vacuum. The resulting residue was purified by prep-HPLC, eluting with MeCN/H₂O/TFA, to afford the title compound ethyl 4-amino-1-(4-cyanophenyl)-3-(3,3,3-trifluoroprop-1-en-2-yl)-1H-pyrazole-5-carboxylate A3d (less polar, 120 mg, 8% yield two steps), and its regioisomer ethyl 4-amino-1-(4-cyanophenyl)-5-(3,3,3-trifluoroprop-1-en-2-yl)-1H-pyrazole-3-carboxylate A3d-1 (more polar). ¹H NMR (400 MHz, MeOD) A3d, less polar regioisomer, δ 7.84 (d, J=8.36 Hz, 2H), 7.63 (d, J=8.28 Hz, 2H), 6.29 (s, 1H), 6.15 (s, 1H), 4.28 (q, J=7.08 Hz, 2H), 1.21 (t, J=7.12 Hz, 3H) ppm; ¹H NMR (400 MHz, MeOD) A3d-1, more polar regioisomer, δ 7.89 (d, J=8.48 Hz, 2H), 7.43 (d, J=8.44 Hz, 2H), 6.53 (s, 1H), 6.15 (s, 1H), 4.45 (q, J=7.12 Hz, 2H), 1.43 (t, J=7.12 Hz, 3H) ppm; LCMS: MS m/z (ESI): 351.0 [M+H]⁺.

### Step 3: ethyl 4-amino-1-(4-(aminomethyl)phenyl)-3-(1,1,1-trifluoropropan-2-yl)-1H-pyrazole-5-carboxylate A3e

A mixture of ethyl 4-amino-1-(4-cyanophenyl)-3-(3,3,3-trifluoroprop-1-en-2-yl)-1H-pyrazole-5-carboxylate A3d (25 mg, 0.07 mmol), Pd/C (20 mg), and 10 drops of HCl in EtOH under hydrogen balloon was stirred for 4 hours at room temperature. The reaction mixture was filtered through Celite, and the filtrate was concentrated under vacuum to afford crude ethyl 4-amino-1-(4-(aminomethyl)phenyl)-3-(1,1,1-trifluoropropan-2-yl)-1H-pyrazole-5-carboxylate A3e, which was for next step without further purification. ¹H NMR (400 MHz, MeOD) δ 7.53 (d, J=8.12 Hz, 2H), 7.46 (d, J=8.16 Hz, 2H), 4.27-3.78 (m, 4H), 3.9-378 (m, 1H), 1.55 (d, J=7.20 Hz, 3H), 1.21 (t, J=7.04 Hz, 3H) ppm; LCMS: MS m/z (ESI): 358.0 [M+H]⁺.

### Step 4: ethyl 4-amino-1-(4-((5-fluoro-2-methoxybenzamido)methyl)phenyl)-3-(1,1,1-trifluoropropan-2-yl)-1H-pyrazole-5-carboxylate A3f

To a solution of 5-fluoro-2-methoxybenzoic acid (5 mg, 0.03 mmol) in DMF (3 mL) under nitrogen atmosphere at room temperature was added HATU (20 mg, 0.05 mmol) The mixture was stirred at room temperature for 0.5 h, and then ethyl 4-amino-1-(4-(aminomethyl)phenyl)-3-(1,1,1-trifluoropropan-2-yl)-1H-pyrazole-5-carboxylate A3e (0.07 mmol) and TEA (0.05 mL) were added to the reaction mixture. The resulting mixture was stirred at room temperature for 30 min. The resulting solution was purified by prep-HPLC, eluting with MeCN/H₂O/TFA, to afford ethyl 4-amino-1-(4-((5-fluoro-2-methoxybenzamido)methyl)phenyl)-3-(1,1,1-trifluoropropan-2-yl)-1H-pyrazole-5-carboxylate A3f (12 mg, 33% yield). LCMS: MS m/z (ESI): 509.0 [M+H]⁺.

### Step 5: 4-amino-1-(4-((5-fluoro-2-methoxybenzamido)methyl)phenyl)-3-(1,1,1-trifluoropropan-2-yl)-1H-pyrazole-5-carboxylic acid A3 g

To a solution of ethyl 4-amino-1-(4-((5-fluoro-2-methoxybenzamido)methyl)phenyl)-3-(1,1,1-trifluoropropan-2-yl)-1H-pyrazole-5-carboxylate A3f (12 mg, 0.023 mmol) in MeOH (4 mL) was added NaOH (10% in water, 5 mL). The resulting mixture was stirred at room temperature for 3 hours. After concentrated under vacuum (dry loading), The residue was purified by silica gel column chromatography (DCM:EtOAc with 10% NH₃ in MeOH) to afford 4-amino-1-(4-((5-fluoro-2-methoxybenzamido)methyl)phenyl)-3-(1,1,1-trifluoropropan-2-yl)-1H-pyrazole-5-carboxylic acid A3 g (10 mg, 90% yield). ¹H NMR (400 MHz, MeOD) δ 7.44-7.37 (m, 1H), 7.29-7.16 (m, 6H), 4.66 (s, 2H), 3.97 (s, 3H), 3.89-3.76 (m, 1H), 1.55 (d, J=7.2 Hz, 3H) ppm; LCMS: MS m/z (ESI): 481.0 [M+H]⁺.

### Step 6: 4-amino-1-(4-((5-fluoro-2-methoxybenzamido)methyl)phenyl)-3-(1,1,1-trifluoropropan-2-yl)-1H-pyrazole-5-carboxamide A3

A solution of 2-(4-((5-fluoro-2-methoxybenzamido)methyl)phenyl)-7-methyl-4,5,6,7-tetrahydro-2H-pyrazolo[4,3-b]pyridine-3-carboxylic acid A3 g (10 mg, 0.021 mmol), HATU (15 mg), NH₄Cl (200 mg), and TEA (0.05 mL) in DMF (2 mL) was stirred overnight at room temperature. The residue was directly purified by prep-HPLC, eluting MeCN/H₂O/TFA, to afford 4-amino-1-(4-((5-fluoro-2-methoxybenzamido)methyl)phenyl)-3-(1,1,1-trifluoropropan-2-yl)-1H-pyrazole-5-carboxamide A3 (5.2 mg, 50% yield). ¹H NMR (400 MHz, MeOD) δ 7.58-7.46 (m, 1H), 7.39 (d, J=8.32 Hz, 2H), 7.32 (d, J=8.44 Hz, 2H), 7.17-7.04 (m, 2H), 4.57 (s, 2H), 3.80 (s, 3H), 3.78-3.67 (m, 1H), 1.44 (d, J=7.2 Hz, 3H) ppm; LCMS: MS m/z (ESI): 480.0 [M+H]⁺.

### Example A4

### 2-(4-((5-fluoro-2-methoxybenzamido)methyl)phenyl)-7-methyl-4,5,6,7-tetrahydro-2H-pyrazolo[4,3-b]pyridine-3-carboxamide A4

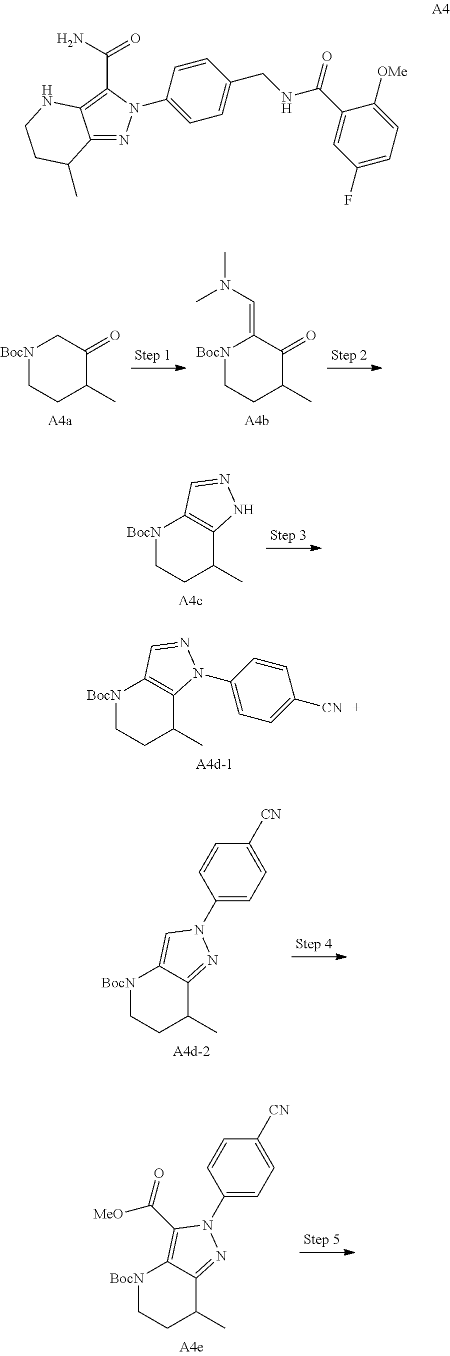

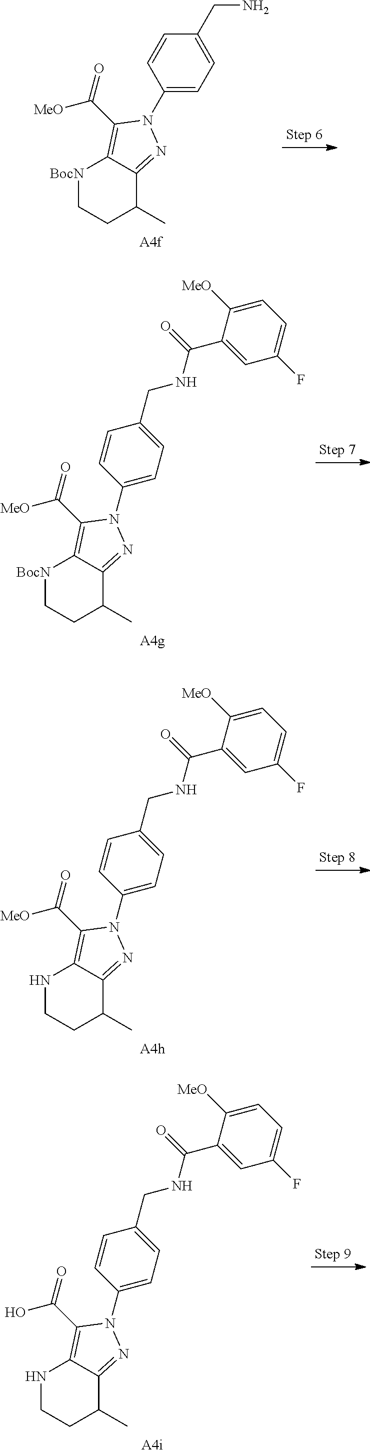 [SMILES: COC(=O)c1c2c(nn1-c1ccc(CN)cc1)C(C)CCN2C(=O)OC(C)(C)C ; COC(=O)c1c2c(nn1-c1ccc(CNC(=O)c3cc(F)ccc3OC)cc1)C(C)CCN2C(=O)OC(C)(C)C ; COC(=O)c1c2c(nn1-c1ccc(CNC(=O)c3cc(F)ccc3OC)cc1)C(C)CCN2 ; COc1ccc(F)cc1C(=O)NCc1ccc(-n2nc3c(c2C(=O)O)NCCC3C)cc1]

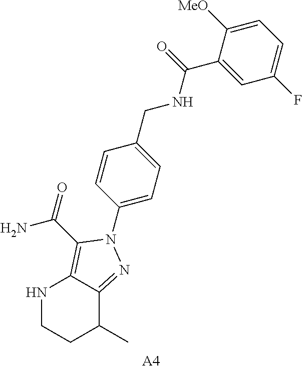

### Step 1: tert-butyl (Z)-2-((dimethylamino)methylene)-4-methyl-3-oxopiperidine-1-carboxylate A4b

To a solution of tert-butyl 4-methyl-3-oxopiperidine-1-carboxylate (A4a, 4 g, 18.75 mmol) in DMF (50 mL) was added DMF.DMA (3.8 mL, 28.13 mmol) under nitrogen atmosphere at room temperature. The resulting mixture was stirred overnight at 90° C. After cooling down, the organic phase was concentrated, and resulting residue was used for next step without further purification. LCMS: MS m/z (ESI): 269 [M+H]⁺.

### Step 2: tert-butyl 7-methyl-1,5,6,7-tetrahydro-4H-pyrazolo[4,3-b]pyridine-4-carboxylate A4c

To a solution of tert-butyl (Z)-2-((dimethylamino)methylene)-4-methyl-3-oxopiperidine-1-carboxylate A4b (crude, 18.75 mmoL) in EtOH (60 mL) in sealed tube was added hydrazine (8 mL). The resulting mixture was stirred overnight at 60° C. After cooling down, the mixture was concentrated under vacuum, the residue was purified by silica gel column chromatography (DCM:EtOAc) to afford title compound tert-butyl 7-methyl-1,5,6,7-tetrahydro-4H-pyrazolo[4,3-b]pyridine-4-carboxylate A4c (3.8 g, 85% yield two steps). ¹H NMR (400 MHz, CDCl₃) δ 8.11 (s, 0.5H), 7.63 (s, 0.5H), 4.01-3.86 (m, 1H), 3.46-3.42 (m, 1H), 3.00-2.96 (m, 1H), 2.06-2.04 (m, 1H), 1.64-1.31 (m, 14H) ppm; LCMS: MS m/z (ESI): 238 [M+H]⁺.

### Step 3: tert-butyl 2-(4-cyanophenyl)-7-methyl-2,5,6,7-tetrahydro-4H-pyrazolo[4,3-b]pyridine-4-carboxylate A4d-2

A mixture of tert-butyl 7-methyl-1,5,6,7-tetrahydro-4H-pyrazolo[4,3-b]pyridine-4-carboxylate A4c (1 g, 4.22 mmol), 4-fluorobenzonitrile (1.02 g, 8.44 mmol) and K₂CO₃ (2.33 g, 16.88 mmol) in DMF (30 mL) under nitrogen atmosphere was stirred overnight at 110° C. After cooling down, the mixture was concentrated under vacuum, diluted with DCM (150 mL), and then extracted with H₂O (150 mL). The organic layers were dried over anhydrous Na₂SO₄, filtered, and concentrated under reduce pressure. The residue was purified by silica gel column chromatography (DCM/EtOAc) to afford the title compound tert-butyl 2-(4-cyanophenyl)-7-methyl-2,5,6,7-tetrahydro-4H-pyrazolo[4,3-b]pyridine-4-carboxylate A4d-2 (less polar, 698 mg, 49% yield), and its regioisomer A4d-1 (more polar, 120 mg, 8.4% yield). ¹H NMR (400 MHz, CDCl₃) A4d-2, less polar regioisomer, δ 8.39 (s, 1H), 7.79 (d, J=8.6 Hz, 2H), 7.68 (d, J=8.6 Hz, 2H), 4.04-3.93 (m, 1H), 3.56-3.51 (m, 1H), 3.06-2.94 (m, 1H), 2.14-2.09 (m, 1H), 1.74-1.64 (m, 1H), 1.64 (s, 9H), 1.41 (d, J=6.8 Hz, 3H); ¹H NMR (400 MHz, CDCl₃) A4d-1, more polar regioisomer, δ 7.75-7.59 (m, 5H), 3.79-3.73 (m, 1H), 3.57-3.52 (m, 1H), 3.31-3.23 (m, 1H), 2.11-2.03 (m, 1H), 1.71-1.68 (m, 1H), 1.67-1.63 (m, 9H), 0.94 (d, J=6.8 Hz, 3H); LCMS: MS m/z (ESI): 339 [M+H]⁺.

### Step 4: 4-(tert-butyl) 3-methyl 2-(4-cyanophenyl)-7-methyl-2,5,6,7-tetrahydro-4H-pyrazolo[4,3-b]pyridine-3,4-dicarboxylate A4e

To a solution of tert-butyl 2-(4-cyanophenyl)-7-methyl-2,5,6,7-tetrahydro-4H-pyrazolo[4,3-b]pyridine-4-carboxylate A4d (340 mg, 1.01 mmol) in THE (20 mL) at −78° C. under nitrogen atmosphere was added n-butyllithium dropwise (1.6 M in hexanes, 1.57 mL, 2.5 mmol). The resulting mixture was stirred at −78° C. for 30 min before slow addition of methyl chloroformate (0.31 mL, 4.04 mmol). The resulting mixture was stirred at −78° C., and then additional 0.25 mL methyl chloroformate was added to the reaction. The resulting mixture was slowly warmed up to room temperature and then concentrated under reduced pressure. The residue was purified by prep-HPLC (TFA in MeCN/H₂O) to afford title compound 4-(tert-butyl) 3-methyl 2-(4-cyanophenyl)-7-methyl-2,5,6,7-tetrahydro-4H-pyrazolo[4,3-b]pyridine-3,4-dicarboxylate A4e (100 mg, 25% yield). ¹H NMR (400 MHz, CDCl₃) δ 7.72 (d, J=6.9 Hz, 2H), 7.60 (d, J=8.6 Hz, 2H), 4.02-3.97 (m, 1H), 3.89 (s, 3H), 3.63-3.57 (m, 1H), 3.08-2.95 (m, 1H), 2.1-2.06 (m, 1H), 1.73-1.68 (m, 1H), 1.66 (s, 9H), 1.63 (d, J=6.8 Hz, 3H) ppm; LCMS: MS m/z (ESI): 397 [M+H]⁺.

### Step 5: 4-(tert-butyl) 3-methyl 2-(4-(aminomethyl)phenyl)-7-methyl-2,5,6,7-tetrahydro-4H-pyrazolo[4,3-b]pyridine-3,4-dicarboxylate A4f

A mixture of 4-(tert-butyl) 3-methyl 2-(4-cyanophenyl)-7-methyl-2,5,6,7-tetrahydro-4H-pyrazolo[4,3-b]pyridine-3,4-dicarboxylate A4e (20 mg, 0.05 mmol), Pd/C (10 mg), and 2 drops of HCl in EtOH under hydrogen balloon was stirred for hours at room temperature. The reaction mixture was filtered through Celite, and the filtrate was concentrated under vacuum to afford crude 4-(tert-butyl) 3-methyl 2-(4-(aminomethyl)phenyl)-7-methyl-2,5,6,7-tetrahydro-4H-pyrazolo[4,3-b]pyridine-3,4-dicarboxylate A4f, which is further dried under lyophilization for 2 hours, and then is used for next step without further purification. LCMS: MS m/z (ESI): 401 [M+H]⁺.

### Step 6: 4-(tert-butyl) 3-methyl 2-(4-((5-fluoro-2-methoxybenzamido)methyl)phenyl)-7-methyl-2,5,6,7-tetrahydro-4H-pyrazolo[4,3-b]pyridine-3,4-dicarboxylate A4 g

To a solution of 4-(tert-butyl) 3-methyl 2-(4-(aminomethyl)phenyl)-7-methyl-2,5,6,7-tetrahydro-4H-pyrazolo[4,3-b]pyridine-3,4-dicarboxylate A4f (crude, 0.05 mmol) in THE (5 mL) at 0° C. under nitrogen atmosphere was added TEA (0.1 mL). The resulting mixture was stirred for 5 min before slow addition of 5-fluoro-2-methoxybenzoyl chloride (12 mg, 0.064 mol) in THE (2 mL). The resulting mixture was slowly warmed up to room temperature and concentrated under vacuum to afford crude 4-(tert-butyl) 3-methyl 2-(4-((5-fluoro-2-methoxybenzamido)methyl) phenyl)-7-methyl-2,5,6,7-tetrahydro-4H-pyrazolo[4,3-b]pyridine-3,4-dicarboxylate A4f, which was used for next step without further purification. LCMS: MS m/z (ESI): 553 [M+H]⁺.

### Step 7: methyl 2-(4-((5-fluoro-2-methoxybenzamido)methyl)phenyl)-7-methyl-4,5,6,7-tetrahydro-2H-pyrazolo[4,3-b]pyridine-3-carboxylate A4 h

To a solution of 4-(tert-butyl) 3-methyl 2-(4-((5-fluoro-2-methoxybenzamido)methyl)phenyl)-7-methyl-2,5,6,7-tetrahydro-4H-pyrazolo[4,3-b]pyridine-3,4-dicarboxylate A4 g (crude, 0.05 mmol) in DCM (1 mL) was added TFA (1 mL). The resulting mixture was stirred at room temperature for 1 hour. After concentrated under vacuum, the resulting crude methyl 2-(4-((5-fluoro-2-methoxybenzamido)methyl)phenyl)-7-methyl-4,5,6,7-tetrahydro-2H-pyrazolo[4,3-b]pyridine-3-carboxylate A4 h was used for next step without further purification. LCMS: MS m/z (ESI): 453 [M+H]⁺.

### Step 8: 2-(4-((5-fluoro-2-methoxybenzamido)methyl)phenyl)-7-methyl-4,5,6,7-tetrahydro-2H-pyrazolo[4,3-b]pyridine-3-carboxylic acid A4i

To a solution of methyl 2-(4-((5-fluoro-2-methoxybenzamido)methyl)phenyl)-7-methyl-4,5,6,7-tetrahydro-2H-pyrazolo[4,3-b]pyridine-3-carboxylate A4 h (crude, 0.05 mmol) in MeOH (4 mL) was added NaOH (10% in water, 5 mL). The resulting mixture was stirred overnight at room temperature. After concentrated under vacuum (dry loading), The residue was purified by silica gel column chromatography (DCM:EtOAc with 10% NH₃ in MeOH) to afford title compound 2-(4-((5-fluoro-2-methoxybenzamido)methyl)phenyl)-7-methyl-4,5,6,7-tetrahydro-2H-pyrazolo[4,3-b]pyridine-3-carboxylic acid A4i (12 mg, 55% yield four steps). ¹H NMR (400 MHz, MeOD) δ 7.51 (dd, J=9.2, 3.24 Hz, 1H), 7.35 (d, J=8.3 Hz, 2H), 7.27 (d, J=8.4 Hz, 2H), 7.18-7.05 (m, 2H), 4.57 (s, 2H), 3.86 (s, 3H), 3.35-3.22 (m, 2H), 2.94-2.89 (m, 1H), 2.08-2.02 (m, 1H), 1.65-1.56 (m, 1H), 1.25 (d, J=6.8 Hz, 3H) ppm; LCMS: MS m/z (ESI): 439 [M+H]⁺.

### Step 9: 2-(4-((5-fluoro-2-methoxybenzamido)methyl)phenyl)-7-methyl-4,5,6,7-tetrahydro-2H-pyrazolo[4,3-b]pyridine-3-carboxamide A4

A solution of 2-(4-((5-fluoro-2-methoxybenzamido)methyl)phenyl)-7-methyl-4,5,6,7-tetrahydro-2H-pyrazolo[4,3-b]pyridine-3-carboxylic acid A4i (12 mg, 0.027 mmol), HATU (15 mg), NH₄Cl (200 mg), and TEA (0.05 mL) in DMF (1 mL) was stirred for 6 hours at room temperature. The residue was directly purified by prep-HPLC (TFA in MeCN/H₂O) to afford 2-(4-((5-fluoro-2-methoxybenzamido)methyl)phenyl)-7-methyl-4,5,6,7-tetrahydro-2H-pyrazolo[4,3-b]pyridine-3-carboxamide A4 (4.8 mg, 40% yield). ¹H NMR (400 MHz, MeOD) δ 7.51 (dd, J=9.2, 3.24 Hz, 1H), 7.40 (d, J=8.3 Hz, 2H), 7.32 (d, J=8.4 Hz, 2H), 7.15-7.05 (m, 2H), 4.57 (s, 2H), 3.82 (s, 3H), 3.38-3.32 (m, 2H), 2.96-2.87 (m, 1H), 2.09-2.04 (m, 1H), 1.64-1.58 (m, 1H), 1.25 (d, J=6.8 Hz, 3H) ppm; LCMS: MS m/z (ESI): 438 [M+H]⁺.

### Example A5

### 4-amino-1-(2-ethoxy-4-((5-fluoro-2-methoxybenzamido)methyl)phenyl)-3-isopropyl-1H-pyrazole-5-carboxamide A5

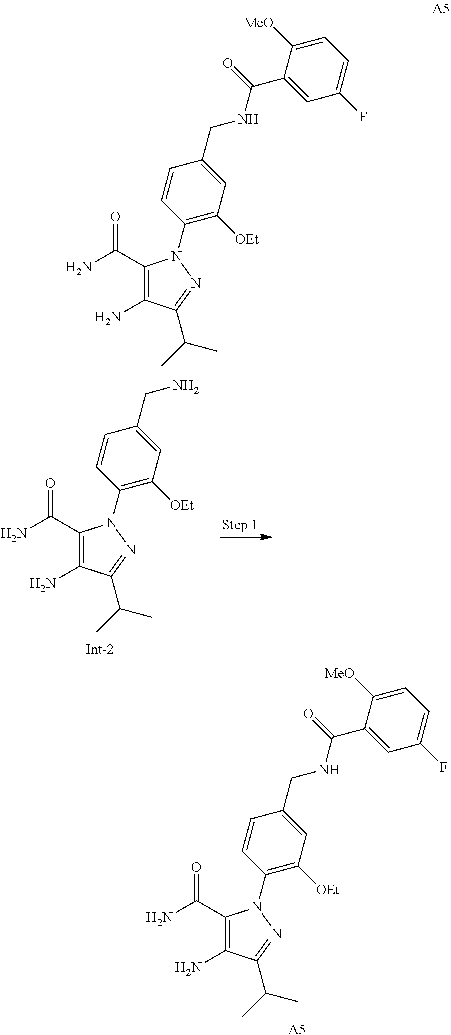

### Step 1: 4-amino-1-(2-ethoxy-4-((5-fluoro-2-methoxybenzamido)methyl)phenyl)-3-isopropyl-1H-pyrazole-5-carboxamide A5

To a solution of 5-fluoro-2-methoxybenzoic acid (96.49 mg, 567.13 umol) in DMF (5 mL) under nitrogen atmosphere at room temperature was added HATU (539.10 mg, 1.42 mmol). The mixture was stirred at room temperature for 20 min followed by addition of Int-2 (300 mg, 945.22 umol) and DIPEA (365.80 mg, 2.84 mmol). The resulting mixture was stirred at room temperature for 20 min, and then diluted with EtOAc and washed with water. The combined organic extracts were washed with brine (20 mL), dried over anhydrous Na₂SO₄, filtered, and concentrated under reduced pressure. The resulting residue was first purified by flash silica gel column chromatography (DCM:MeOH=15:1), and then prep-HPLC, eluting with MeCN/H₂O/NH₄OH, to afford title compound 4-amino-1-(2-ethoxy-4-((5-fluoro-2-methoxybenzamido)methyl)phenyl)-3-isopropyl-1H-pyrazole-5-carboxamide A5 (42.3 mg, 9.53% yield). ¹H NMR (400 MHz, DMSO-d₆): δ 8.81 (t, J=6.0 Hz, 1H), 7.49 (dd, J=9.2 Hz, 3.2 Hz, 1H), 7.36-7.30 (m, 1H), 7.26 (d, J=8.0 Hz, 1H), 7.18 (dd, J=9.2 Hz, 4.4 Hz, 1H), 7.06 (s, 1H), 6.97 (d, J=8.0 Hz, 1H), 4.52 (d, J=6.0 Hz, 2H), 4.42 (brs, 2H), 3.96 (q, J=7.2 Hz, 2H), 3.89 (s, 3H), 3.05-2.93 (m, 1H), 1.22-1.12 (m, 9H) ppm; LCMS: MS m/z (ESI): 470.3 [M+H]⁺.

### Example A6

### 4-amino-1-(4-((2-cyclopropoxy-5-fluorobenzamido)methyl)-2-ethoxyphenyl)-3-isopropyl-1H-pyrazole-5-carboxamide A6

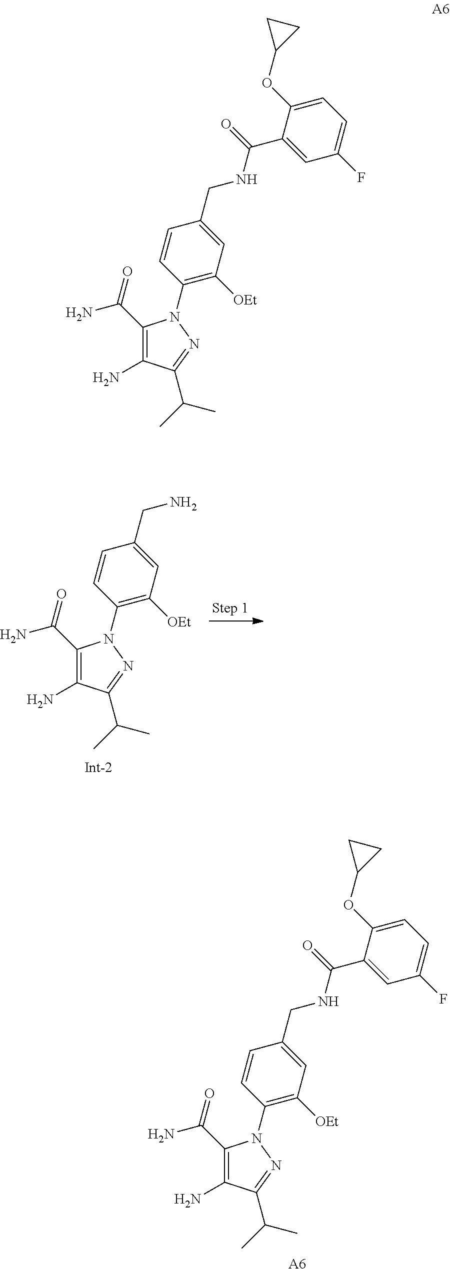

### Step 1: 4-amino-1-(4-((2-cyclopropoxy-5-fluorobenzamido)methyl)-2-ethoxyphenyl)-3-isopropyl-1H-pyrazole-5-carboxamide A6

To a solution of 2-cyclopropoxy-5-fluorobenzoic acid A2f (49.76 mg, 253.64 umol) in DMF (4 mL) under nitrogen atmosphere at room temperature was added HATU (206.66 mg, 543.50 umol). The resulting mixture was stirred at room temperature for 20 min followed by addition of Int-2 (115 mg, 362.34 umol) and DIPEA (140.22 mg, 1.09 mmol). The resulting mixture was stirred at room temperature for 30 min, and then diluted with EtOAc and washed with water. The combined organic extracts were washed with brine (20 mL), dried over anhydrous Na₂SO₄, filtered, and concentrated under reduced pressure. The resulting residue was purified by prep-HPLC, eluting with MeCN/H₂O/NH₄OH, to afford title compound 4-amino-1-(4-((2-cyclopropoxy-5-fluorobenzamido)methyl)-2-ethoxyphenyl)-3-isopropyl-1H-pyrazole-5-carboxamide A6 (11.3 mg, 6.46% yield). ¹H NMR (400 MHz, DMSO-d₆) δ 8.54 (t, J=6.0 Hz, 1H), 7.49-7.38 (m, 2H), 7.38-7.30 (m, 1H), 7.26 (d, J=8.0 Hz, 1H), 7.05 (s, 1H), 6.97 (d, J=8.0 Hz, 1H), 4.50 (d, J=6.0 Hz, 2H), 4.43 (brs, 2H), 4.02-3.92 (m, 3H), 3.05-2.96 (m, 1H), 1.23-1.16 (m, 9H), 0.82-0.71 (m, 4H) ppm; LCMS: MS m/z (ESI): 496.1 [M+H]⁺.

### Example A7

### 4-amino-1-(2-ethoxy-4-((5-fluoro-2-methoxybenzamido)methyl)phenyl)-3-(1,1,1-trifluoropropan-2-yl)-1H-pyrazole-5-carboxamide A7

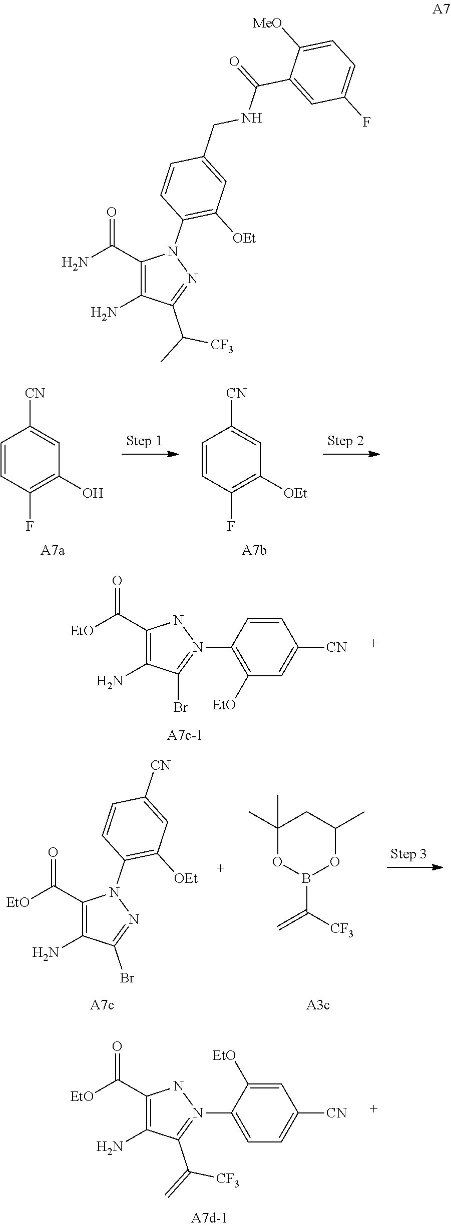

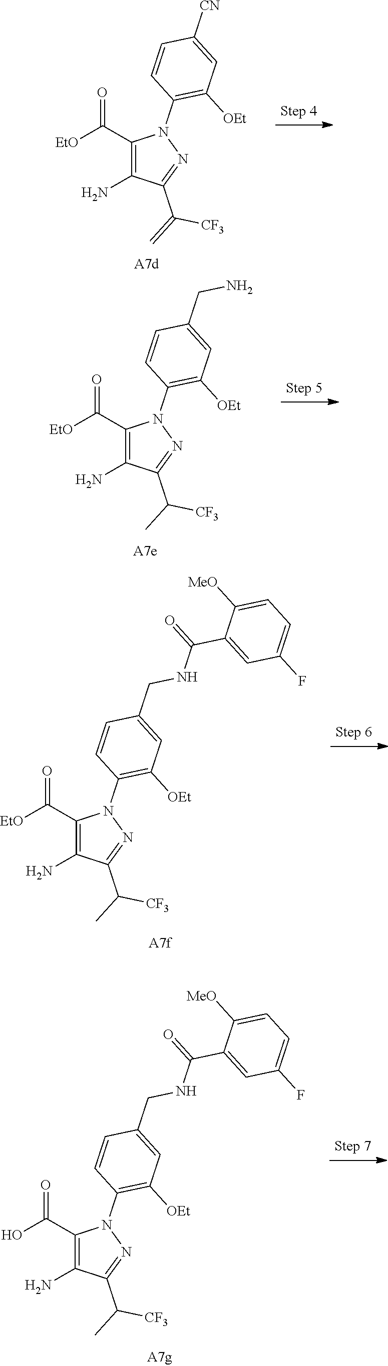 [SMILES: C=C(c1nn(-c2ccc(C#N)cc2OCC)c(C(=O)OCC)c1N)C(F)(F)F ; CCOC(=O)c1c(N)c(C(C)C(F)(F)F)nn1-c1ccc(CN)cc1OCC ; CCOC(=O)c1c(N)c(C(C)C(F)(F)F)nn1-c1ccc(CNC(=O)c2cc(F)ccc2OC)cc1OCC ; CCOc1cc(CNC(=O)c2cc(F)ccc2OC)ccc1-n1nc(C(C)C(F)(F)F)c(N)c1C(=O)O]

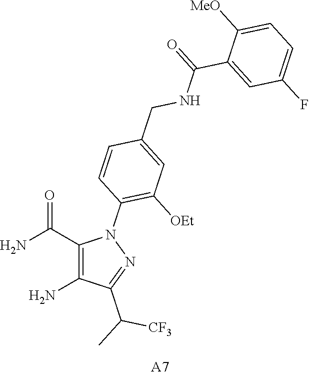

### Step 1: 3-ethoxy-4-fluorobenzonitrile A7b

To a solution of 4-fluoro-3-hydroxybenzonitrile A7a (2 g, 14.59 mmol) in DMF (30 mL) was added K₂CO₃ (6.1 g, 43.77 mmol) and iodoethane (4.6 g, 29.17 mmol). The resulting mixture was stirred at 35° C. for 4 hours. After cooling down, the mixture was diluted with EtOAc (25 mL) and washed with water (25 mL). The layers were separated, and the organic layer was dried over anhydrous Na₂SO₄, filtered and concentrated under reduced pressure. The resulting residue was purified by silica gel column chromatography (petroleum ether/EA=10/1) to afford the title compound 3-ethoxy-4-fluorobenzonitrile A7b (2.13 g, 88.39% yield). ¹H NMR (400 MHz, CDCl₃): δ 7.28-7.13 (m, 3H), 4.13 (q, J 7.2 Hz, 2H), 1.48 (t, J 7.2 Hz, 3H) ppm.

### Step 2: ethyl 4-amino-3-bromo-1-(4-cyano-2-ethoxyphenyl)-1H-pyrazole-5-carboxylate A7c and ethyl 4-amino-5-bromo-1-(4-cyano-2-ethoxyphenyl)-1H-pyrazole-3-carboxylate A7c-1

Step 2 of Examples A7 was prepared with the similar procedures as Step 1 in Example A3 by using 3-ethoxy-4-fluorobenzonitrile A7b.

### Step 3: ethyl 4-amino-1-(4-cyano-2-ethoxyphenyl)-3-(3,3,3-trifluoroprop-1-en-2-yl)-1H-pyrazole-5-carboxylate A7d and ethyl 4-amino-1-(4-cyano-2-ethoxyphenyl)-5-(3,3,3-trifluoroprop-1-en-2-yl)-1H-pyrazole-3-carboxylate A7d-1

Step 3 of Examples A7 was prepared with the similar procedures as Step 2 in Example A3 by using mixture of ethyl 4-amino-3-bromo-1-(4-cyano-2-ethoxyphenyl)-1H-pyrazole-5-carboxylate A7c and ethyl 4-amino-5-bromo-1-(4-cyano-2-ethoxyphenyl)-1H-pyrazole-3-carboxylate A7c-1

### Step 4: ethyl 4-amino-1-(4-(aminomethyl)-2-ethoxyphenyl)-3-(1,1,1-trifluoropropan-2-yl)-1H-pyrazole-5-carboxylate A7e

Step 4 of Examples A7 was prepared with the similar procedures as Step 3 in Example A3 by using less polar regioisomer ethyl 4-amino-1-(4-cyano-2-ethoxyphenyl)-3-(3,3,3-trifluoroprop-1-en-2-yl)-1H-pyrazole-5-carboxylate A7d

### Step 5: ethyl 4-amino-1-(2-ethoxy-4-((5-fluoro-2-methoxybenzamido)methyl)phenyl)-3-(1,1,1-trifluoropropan-2-yl)-1H-pyrazole-5-carboxylate A7f

Step 5 of Examples A7 was prepared with the similar procedures as Step 4 in Example A3 by using ethyl 4-amino-1-(4-(aminomethyl)-2-ethoxyphenyl)-3-(1,1,1-trifluoropropan-2-yl)-1H-pyrazole-5-carboxylate A7e

### Step 6: 4-amino-1-(2-ethoxy-4-((5-fluoro-2-methoxybenzamido)methyl)phenyl)-3-(1,1,1-trifluoropropan-2-yl)-1H-pyrazole-5-carboxylic acid A7 g

Step 6 of Examples A7 was prepared with the similar procedures as Step 5 in Example A3 by using ethyl 4-amino-1-(2-ethoxy-4-((5-fluoro-2-methoxybenzamido)methyl)phenyl)-3-(1,1,1-trifluoropropan-2-yl)-1H-pyrazole-5-carboxylate A7f

### Step 7: 4-amino-1-(2-ethoxy-4-((5-fluoro-2-methoxybenzamido)methyl)phenyl)-3-(1,1,1-trifluoropropan-2-yl)-1H-pyrazole-5-carboxamide A7

Step 7 of Examples A7 was prepared with the similar procedures as Step 6 in Example A3 by using 4-amino-1-(2-ethoxy-4-((5-fluoro-2-methoxybenzamido)methyl)phenyl)-3-(1,1,1-trifluoropropan-2-yl)-1H-pyrazole-5-carboxylic acid A7 g. The crude product was purified by prep-HPLC, eluting with MeCN/H₂O/TFA, to afford 4-amino-1-(4-((5-fluoro-2-methoxybenzamido)methyl)phenyl)-3-(1,1,1-trifluoropropan-2-yl)-1H-pyrazole-5-carboxamide A8. ¹H NMR (400 MHz, MeOD): δ: 7.50 (dd, J=9.44 Hz, 2.88 Hz, 1H), 7.29 (d, J=7.92 Hz, 1H), 7.18-7.13 (m, 1H), 7.10-7.05 (m, 2H), 6.99 (d, 1H), 4.55 (s, 2H), 3.94 (q, J=4.96, 2H), 3.86 (s, 3H), 3.76-3.67 (m, 1H), 1.43 (d, J=7.28 Hz, 3H), 1.16 (t, J=7 Hz, 3H) ppm; LCMS: MS m/z (ESI): 524.0 [M+H]⁺.

### Example A8

### 2-(2-ethoxy-4-((5-fluoro-2-methoxybenzamido)methyl)phenyl)-4,5,6,7-tetrahydro-2H-pyrazolo[4,3-b]pyridine-3-carboxamide A8

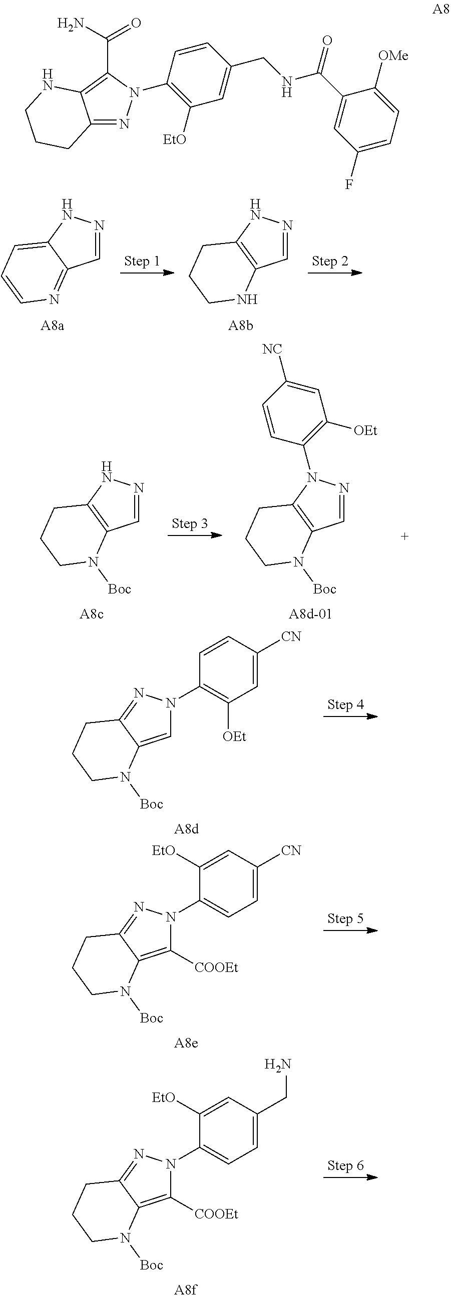

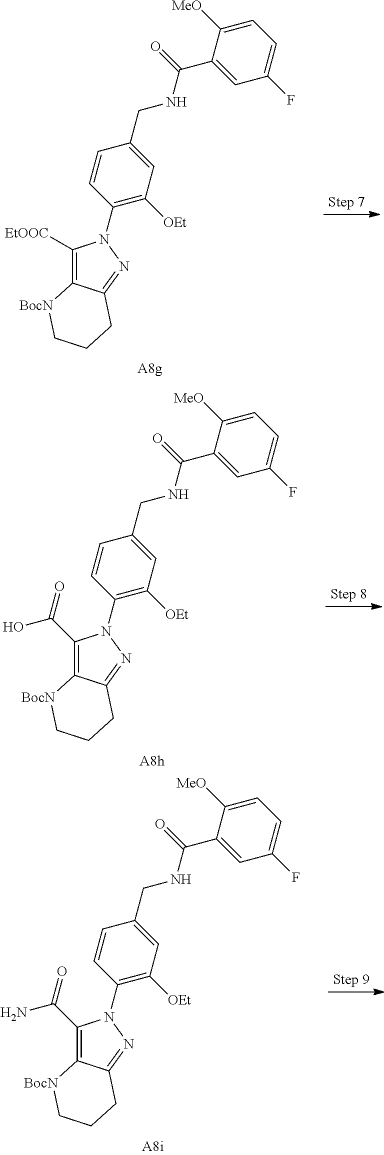 [SMILES: CCOC(=O)c1c2c(nn1-c1ccc(CNC(=O)c3cc(F)ccc3OC)cc1OCC)CCCN2C(=O)OC(C)(C)C ; CCOc1cc(CNC(=O)c2cc(F)ccc2OC)ccc1-n1nc2c(c1C(=O)O)N(C(=O)OC(C)(C)C)CCC2 ; CCOc1cc(CNC(=O)c2cc(F)ccc2OC)ccc1-n1nc2c(c1C(N)=O)N(C(=O)OC(C)(C)C)CCC2]

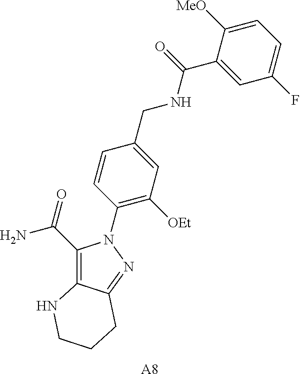 [SMILES: CCOc1cc(CNC(=O)c2cc(F)ccc2OC)ccc1-n1nc2c(c1C(N)=O)NCCC2 ; "A8"]

### Step 1: 4,5,6,7-tetrahydro-1H-pyrazolo[4,3-b]pyridine A8b

A mixture of 1H-pyrazolo[4,3-b]pyridine A8a (2 g, 16.8 mmol) and PtO₂ (380 mg, 1.7 mmol) in TFA (20 mL) was stirred under H₂ atmosphere (3.0 bar) at room temperature for 16 hours. The mixture was filtered through Celite, and the filtrate was concentrated in vacuum. The resulting residue was diluted with DCM and adjusted pH>7 with the addition of NH₄OH. The mixture was then concentrated to give tert-butyl 4,5,6,7-tetrahydro-1H-pyrazolo[4,3-b]pyridine A8b (2.5 g, overweight), which was used for next step without further purification. LCMS: MS m/z (ESI): 124.1 [M+H]⁺.

### Step 2: tert-butyl 1,5,6,7-tetrahydro-4H-pyrazolo[4,3-b]pyridine-4-carboxylate A8c

To a solution of 4,5,6,7-tetrahydro-1H-pyrazolo[4,3-b]pyridine A8b (2.5 g, 16.8 mmol) in DCM (100 mL) was added (Boc)₂O (5.5 g, 25.2 mmol). The resulting mixture was stirred at room temperature for 2 hours, and then washed with water (30 mL). The organic layers were dried over anhydrous Na₂SO₄, filtered, and concentrated under reduce pressure to give crude tert-butyl 1,5,6,7-tetrahydro-4H-pyrazolo[4,3-b]pyridine-4-carboxylate A8c, which was used directly for next step without further purification. LCMS: MS m/z (ESI): 224.1 [M+H]⁺.

### Step 3: tert-butyl 2-(4-cyano-2-ethoxyphenyl)-2,5,6,7-tetrahydro-4H-pyrazolo[4,3-b]pyridine-4-carboxylate A8d and tert-butyl 1-(4-cyano-2-ethoxyphenyl)-1,5,6,7-tetrahydro-4H-pyrazolo[4,3-b]pyridine-4-carboxylate A8d-1

To a solution of tert-butyl 1,5,6,7-tetrahydro-4H-pyrazolo[4,3-b]pyridine-4-carboxylate A8c (1.14 g, 5.38 mmol) in DMF (30 mL) under nitrogen atmosphere was added 3-ethoxy-4-fluorobenzonitrile A7b (1 g, 4.48 mmol) and K₂CO₃ (1.86 g, 13.44 mmoL). The resulting mixture was stirred at 130° C. for 12 hours. After cooling down, the mixture was diluted with EtOAc and washed with water (100 mL). The organic layers were dried over anhydrous Na₂SO₄, filtered, and concentrated under reduced pressure. The residue was purified by silica gel column chromatography (PE/EA=10/1˜5/1) to afford the title compound tert-butyl 2-(4-cyano-2-ethoxyphenyl)-2,5,6,7-tetrahydro-4H-pyrazolo[4,3-b]pyridine-4-carboxylate A8d (508 mg, 30.80% yield) and tert-butyl 1-(4-cyano-2-ethoxyphenyl)-1,5,6,7-tetrahydro-4H-pyrazolo[4,3-b]pyridine-4-carboxylate A8d-1 (150 mg, 0.41 mmol, 9.1% yield).

¹H NMR (400 MHz, CDCl₃) A8d, less polar regioisomer, δ 8.64 (brs, 0.5H), 8.13 (brs, 0.5H), 7.88 (d, J=8.4 Hz, 1H), 7.38-7.17 (m, 2H), 4.19 (br, 2H), 3.73 (br, 2H), 2.84 (t, 6.4 Hz, 2H), 2.05-2.01 (m, 2H), 1.60-1.53 (m, 12H) ppm;

¹H NMR (400 MHz, CDCl₃) A8d-1, more polar regioisomer, δ 8.25 (brs, 0.4H), 7.86 (brs, 0.6H), 7.49 (d, 8.0 Hz, 1H), 7.36 (d, J=8.0 Hz, 1H), 7.27 (s, 1H), 4.13-4.08 (m, 2H), 3.74 (br, 2H), 2.58 (t, J=6.4 Hz, 2H), 1.95-1.90 (m, 2H), 1.61 (s, 9H), 1.39 (t, J=6.8 Hz, 3H).

LCMS: MS m/z (ESI): 369.2 [M+H]⁺.

### Step 4: 4-(tert-butyl) 3-ethyl 2-(4-cyano-2-ethoxyphenyl)-2,5,6,7-tetrahydro-4H-pyrazolo[4,3-b]pyridine-3,4-dicarboxylate A8e

To a solution of tert-butyl 2-(4-cyano-2-ethoxyphenyl)-2,5,6,7-tetrahydro-4H-pyrazolo[4,3-b]pyridine-4-carboxylate A8d (130 mg, 0.4 mmol) in THE (10 mL) under nitrogen atmosphere at −78° C. was added n-BuLi (0.3 mL, 0.8 mmol, 2.5M) dropwise. The mixture was stirred at this temperature for 1 hour before dropwise addition of ethyl carbonochloridate (108 mg, 1.0 mmol). The resulting mixture was stirred at −78° C. for 1 h, and then slowly warmed up to room temperature. The reaction was quenched with saturated aqueous NH₄Cl (2 mL) and H₂O (2 mL). The resulting solution was extracted with EtOAc, and the combined organic layers were dried over anhydrous Na₂SO₄, filtered, and concentrated under reduced pressure. The residue was purified by silica gel column chromatography (petroleum ether/EA=10/1˜5/1) to afford the title compound 4-(tert-butyl) 3-ethyl 2-(4-cyano-2-ethoxyphenyl)-2,5,6,7-tetrahydro-4H-pyrazolo[4,3-b]pyridine-3,4-dicarboxylate A8e (36 mg, 20% yield). LCMS: MS m/z (ESI): 441.6 [M+H]⁺.

### Step 5: 4-(tert-butyl) 3-ethyl 2-(4-(aminomethyl)-2-ethoxyphenyl)-2,5,6,7-tetrahydro-4H-pyrazolo[4,3-b]pyridine-3,4-dicarboxylate A8f

A mixture of 4-(tert-butyl) 3-ethyl 2-(4-cyano-2-ethoxyphenyl)-2,5,6,7-tetrahydro-4H-pyrazolo[4,3-b]pyridine-3,4-dicarboxylate A8e (36 mg, 0.08 mmol) and Pd/C (30 mg) in EtOH (10 mL) was stirred under hydrogen atmosphere (15 psi) at room temperature for 3 hours. Then, the mixture was filtered through Celite, and the filtrate was concentrated under reduced pressure to afford the title compound 4-(tert-butyl) 3-ethyl 2-(4-(aminomethyl)-2-ethoxyphenyl)-2,5,6,7-tetrahydro-4H-pyrazolo[4,3-b]pyridine-3,4-dicarboxylate A8f, which was used for next step without further purification. LCMS: MS m/z (ESI): 445.2 [M+H]⁺.

### Step 6: 4-(tert-butyl) 3-ethyl 2-(2-ethoxy-4-((5-fluoro-2-methoxybenzamido)methyl)phenyl)-2,5,6,7-tetrahydro-4H-pyrazolo[4,3-b]pyridine-3,4-dicarboxylate A8 g

To a solution of 4-(tert-butyl) 3-ethyl 2-(4-(aminomethyl)-2-ethoxyphenyl)-2,5,6,7-tetrahydro-4H-pyrazolo[4,3-b]pyridine-3,4-dicarboxylate A8f (70 mg, 15.75 mmol) in anhydrous THE (10 mL) at room temperature was added DIPEA (0.1 mL, 0.41 mmol) followed by dropwise addition of 5-fluoro-2-methoxybenzoyl chloride (52 mg, 0.27 mmol) in THE (3 mL). The resulting mixture was stirred for 1 hour, and then quenched by saturated aqueous NH₄Cl solution (2 mL). After extraction with EtOAc, the combined organic layers were dried over anhydrous Na₂SO₄, filtered, and concentrated under reduced pressure. The residue was purified by silica gel column chromatography (petroleum ether/EA=5/1˜2/1) to afford the title compound 4-(tert-butyl) 3-ethyl 2-(2-ethoxy-4-((5-fluoro-2-methoxybenzamido)methyl)phenyl)-2,5,6,7-tetrahydro-4H-pyrazolo[4,3-b]pyridine-3,4-dicarboxylate A8 g, which was used for next step without further purification. LCMS: MS m/z (ESI): 597.3 [M+H]⁺.

### Step 7: 4-(tert-butoxycarbonyl)-2-(2-ethoxy-4-((5-fluoro-2-methoxybenzamido)methyl)phenyl)-4,5,6,7-tetrahydro-2H-pyrazolo[4,3-b]pyridine-3-carboxylic acid A8 h

To a solution of 4-(tert-butyl) 3-ethyl 2-(2-ethoxy-4-((5-fluoro-2-methoxybenzamido)methyl)phenyl)-2,5,6,7-tetrahydro-4H-pyrazolo[4,3-b]pyridine-3,4-dicarboxylate A8 g (62 mg, 0.10 mmol) in EtOH (3 mL) and H₂O (2 mL) was added LiOH.H₂O (5 mg, 0.10 mmol). The resulting mixture was stirred at 35° C. for 18 hours. After cooling down, the mixture was concentrated under reduced pressure to afford the crude title compound 4-(tert-butoxycarbonyl)-2-(2-ethoxy-4-((5-fluoro-2-methoxybenzamido)methyl)phenyl)-4,5,6,7-tetrahydro-2H-pyrazolo[4,3-b]pyridine-3-carboxylic acid A8 h, which used for next step without further purification. LCMS: MS m/z (ESI): 569.2 [M+H]⁺.

### Step 8: tert-butyl 3-carbamoyl-2-(2-ethoxy-4-((5-fluoro-2-methoxybenzamido)methyl)phenyl)-2,5,6,7-tetrahydro-4H-pyrazolo[4,3-b]pyridine-4-carboxylate A8i

To a solution of 4-(tert-butoxycarbonyl)-2-(2-ethoxy-4-((5-fluoro-2-methoxybenzamido)methyl)phenyl)-4,5,6,7-tetrahydro-2H-pyrazolo[4,3-b]pyridine-3-carboxylic acid A8 h (25 mg, 0.044 mmol) and CDI (14 mg, 0.088 mmol) in DMF (2 mL) at 0° C. was added NH₄OH (0.5 mL, 6.697 mmol). The resulting mixture was slowly warmed up to room temperature and stirred for additional 5 min. The reaction was quenched with saturated aqueous NH₄Cl (10 mL) and extracted with EtOAc (20 mL). The organic layer was dried over anhydrous Na₂SO₄, filtered, and concentrated under reduced pressure to afford the crude title compound tert-butyl 3-carbamoyl-2-(2-ethoxy-4-((5-fluoro-2-methoxybenzamido)methyl)phenyl)-2,5,6,7-tetrahydro-4H-pyrazolo[4,3-b]pyridine-4-carboxylate A8i, which used for next step without further purification. LCMS: MS m/z (ESI): 568.2 [M+H]⁺.

### Step 9: 2-(2-ethoxy-4-((5-fluoro-2-methoxybenzamido)methyl)phenyl)-4,5,6,7-tetrahydro-2H-pyrazolo[4,3-b]pyridine-3-carboxamide A8

A solution of tert-butyl 3-carbamoyl-2-(2-ethoxy-4-((5-fluoro-2-methoxybenzamido)methyl)phenyl)-2,5,6,7-tetrahydro-4H-pyrazolo[4,3-b]pyridine-4-carboxylate A8i (20 mg, 0.035 mmol) in HCl (4N in dioxane, 3 mL) was stirred at room temperature for 1 hour. After concentrated under reduced pressure, the resulting residue was purified by prep-HPLC, eluting with MeCN/H₂O/formic acid, to afford title compound 2-(2-ethoxy-4-((5-fluoro-2-methoxybenzamido)methyl)phenyl)-4,5,6,7-tetrahydro-2H-pyrazolo[4,3-b]pyridine-3-carboxamide A8 (2.66 mg, 16.27% yield). ¹H NMR (400 MHz, DMSO-d₆): δ 8.88-8.86 (m, 1H), 8.34 (brs, 1H), 7.47 (dd, J=9.2 Hz, 3.2 Hz, 1H), 7.36-7.31 (m, 1H), 7.22-7.17 (m, 3H), 7.06 (s, 1H), 7.00 (s, 1H), 4.55-4.50 (m, 2H), 4.04 (q, J 6.8 Hz, 2H), 3.89 (s, 3H), 3.72-3.70 (m, 1H), 3.37-3.35 (m, 1H), 3.03-3.00 (m, 1H), 2.60 (t, J 6.4 Hz, 2H), 1.81 (br, 2H), 1.22 (t, J 6.8 Hz, 3H) ppm; LCMS: MS m/z (ESI): 468.2 [M+H]⁺.

### Example A9

### 4-amino-3-isopropyl-1-(4-((2-methoxybenzamido)methyl)phenyl)-1H-pyrazole-5-carboxamide A9

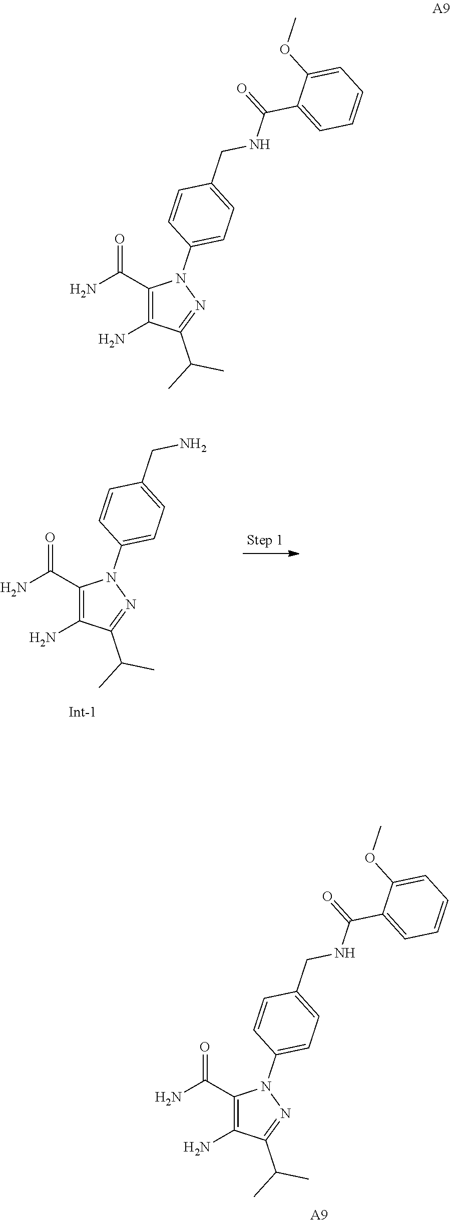

### Step 1: 4-amino-3-isopropyl-1-(4-((2-methoxybenzamido)methyl)phenyl)-1H-pyrazole-5-carboxamide A9

To a solution of 2-methoxybenzoic acid (50.65 mg, 332.93 umol) in DMF (5 mL) under nitrogen atmosphere at room temperature was added HATU (271.26 mg, 713.42 umol). The resulting mixture was stirred at room temperature for 30 min followed by addition of Int-1 (130 mg, 475.61 umol) and DIPEA (184.06 mg, 1.43 mmol). The resulting mixture was stirred at room temperature for 1 h, and then diluted with EtOAc and washed with water. The combined organic extracts were washed with brine (20 mL), dried over anhydrous Na₂SO₄, filtered, and concentrated under reduced pressure. The resulting residue was purified by silica gel column chromatography (DCM:MeOH=18:1) to afford the title compound 4-amino-3-isopropyl-1-(4-((2-methoxybenzamido)methyl)phenyl)-1H-pyrazole-5-carboxamide A9 (26.54 mg, 13.69% yield). ¹H NMR (400 MHz, DMSO-d₆) δ: 8.72 (t, 1H), 7.75 (dd, 1H), 7.52-7.44 (m, 1H), 7.36 (d, 2H), 7.27 (d, 2H), 7.15 (d, 1H), 7.03 (t, 1H), 4.53-4.50 (m, 4H), 3.90 (s, 3H), 3.05-2.98 (m, 1H), 1.21 (d, 6H) ppm; LCMS: MS m/z (ESI): 408.2 [M+H]⁺.

### Example A10

### 4-amino-1-(4-((2,3-dihydrobenzofuran-7-carboxamido)methyl)phenyl)-3-isopropyl-1H-pyrazole-5-carboxamide A10

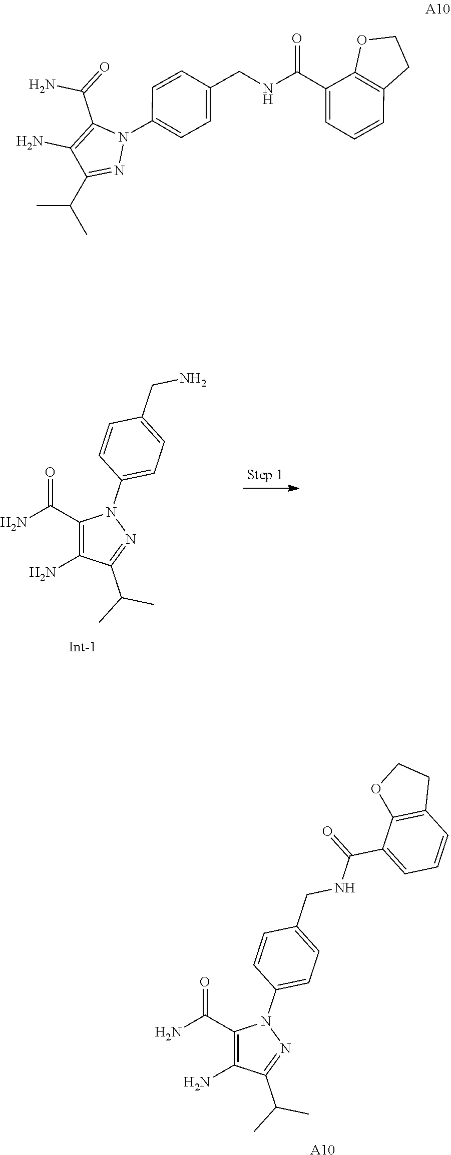

### Step 1: 4-amino-1-(4-((2,3-dihydrobenzofuran-7-carboxamido)methyl)phenyl)-3-isopropyl-1H-pyrazole-5-carboxamide A10

To a solution of 2,3-dihydrobenzofuran-7-carboxylic acid (42.04 mg, 256.10 umol) in DMF (3 mL) under nitrogen atmosphere at room temperature was added HATU (166.93 mg, 439.03 umol). The resulting mixture was stirred at room temperature for 30 min followed by addition of Int-1 (100 mg, 365.85 umol) and DIPEA (141.85 mg, 1.10 mmol). The resulting mixture was stirred at room temperature for 3 hours, and then diluted with EtOAc and washed with water. The combined organic extracts were washed with brine (20 mL), dried over anhydrous Na₂SO₄, filtered, and concentrated under reduced pressure. The residue was purified by prep-HPLC (NH₄OH in MeCN/H₂O) to afford 4-amino-1-(4-((2,3-dihydrobenzofuran-7-carboxamido)methyl)phenyl)-3-isopropyl-1H-pyrazole-5-carboxamide A10 (27.77 mg, 18.10% yield). ¹H NMR (400 MHz, CDCl₃): δ 8.02 (t, 1H), 7.95 (d, 1H), 7.47 (d, 2H), 7.42 (d, 2H), 7.33 (dd, 1H), 6.98 (t, 1H), 4.73-4.69 (m, 4H), 3.27 (t, 2H), 3.13-3.07 (m, 1H), 1.34 (d, 6H) ppm; LCMS: MS m/z (ESI): 420.5 [M+H]⁺.

### Example A11

### 4-amino-1-(2-fluoro-4-((5-fluoro-2-methoxybenzamido)methyl)phenyl)-3-isopropyl-1H-pyrazole-5-carboxamide A11

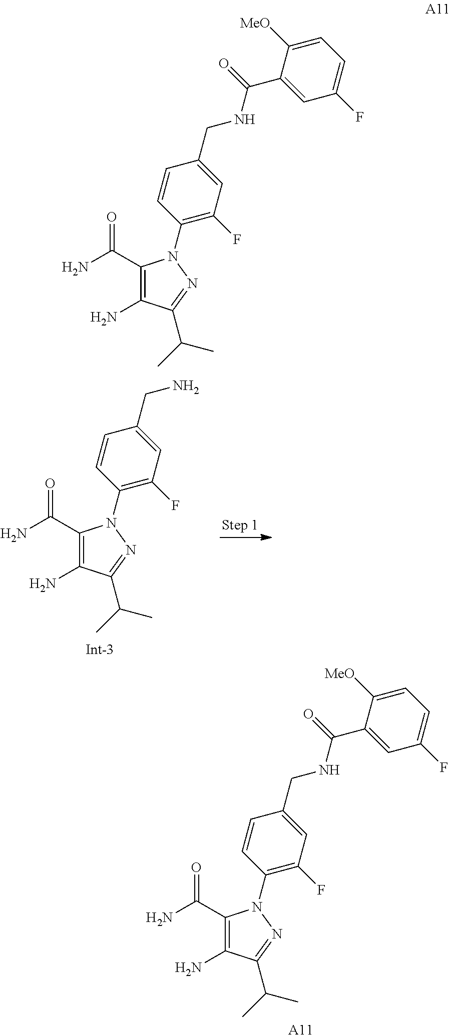

### Step 1: 4-amino-1-(2-fluoro-4-((5-fluoro-2-methoxybenzamido)methyl)phenyl)-3-isopropyl-1H-pyrazole-5-carboxamide A11

To a solution of 5-fluoro-2-methoxy-benzoic acid (14.60 mg, 85.82 umol) in DMF (2 mL) under nitrogen atmosphere at room temperature was added HATU (78.31 mg, 205.96 umol). The resulting mixture was stirred at room temperature for 30 min followed by addition of Int-3 (50 mg, 171.63 umol) and DIPEA (66.55 mg, 514.89 umol). The resulting mixture was stirred at room temperature for 3 hours, and then diluted with EtOAc and washed with water. The combined organic extracts were washed with brine (20 mL×3), dried over anhydrous Na₂SO₄, filtered, and concentrated under reduced pressure. The residue was purified by prep-HPLC (NH₄OH in MeCN/H₂O) to afford 4-amino-1-(2-fluoro-4-((5-fluoro-2-methoxybenzamido)methyl)phenyl)-3-isopropyl-1H-pyrazole-5-carboxamide A11 (9.26 mg, 12.17% yield). ¹H NMR (400 MHz, DMSO-d₆): δ 8.89 (t, 1H), 7.51 (dd, 1H), 7.42-7.32 (m, 2H), 7.23-7.15 (m, 5H), 4.53 (d, 2H), 4.46 (brs, 2H), 3.91 (s, 3H), 3.09-3.01 (m, 1H), 1.21 (d, 6H) ppm; LCMS: MS m/z (ESI): 444.1 [M+H]⁺.

### Example A12

### 4-amino-1-(4-((5-fluoro-2-methoxybenzamido)methyl)phenyl)-3-(tetrahydro-2H-pyran-4-yl)-1H-pyrazole-5-carboxamide A12

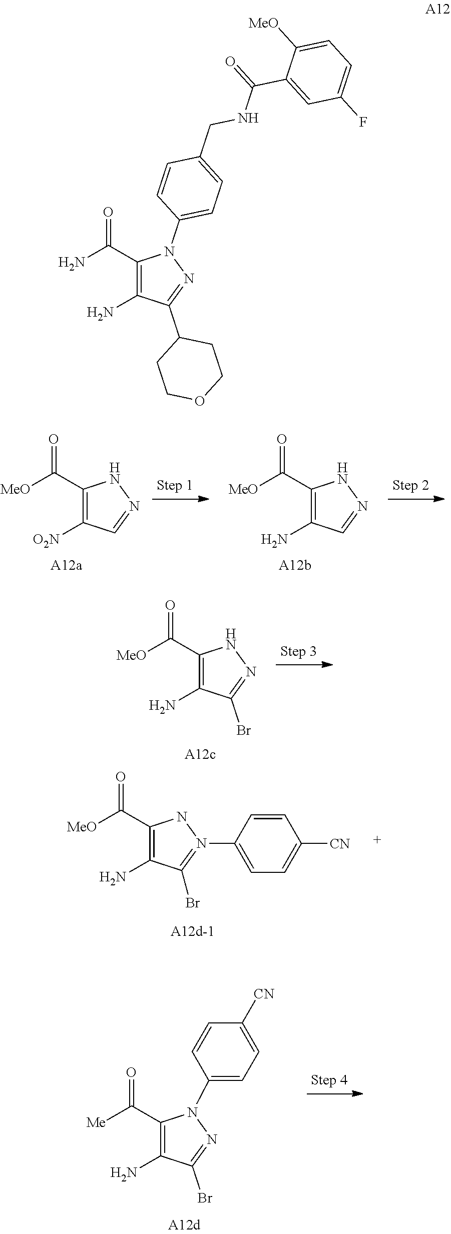

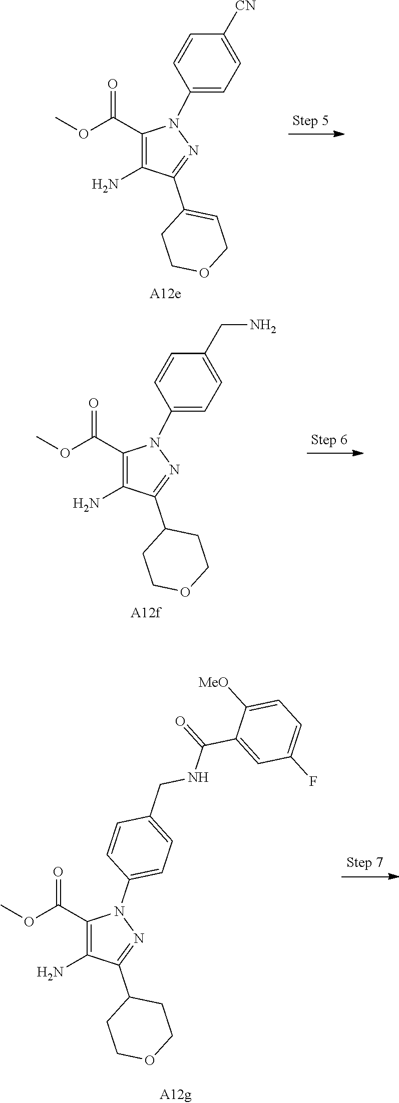 [SMILES: COC(=O)c1c(N)c(C2=CCOCC2)nn1-c1ccc(C#N)cc1 ; COC(=O)c1c(N)c(C2CCOCC2)nn1-c1ccc(CN)cc1 ; COC(=O)c1c(N)c(C2CCOCC2)nn1-c1ccc(CNC(=O)c2cc(F)ccc2OC)cc1]

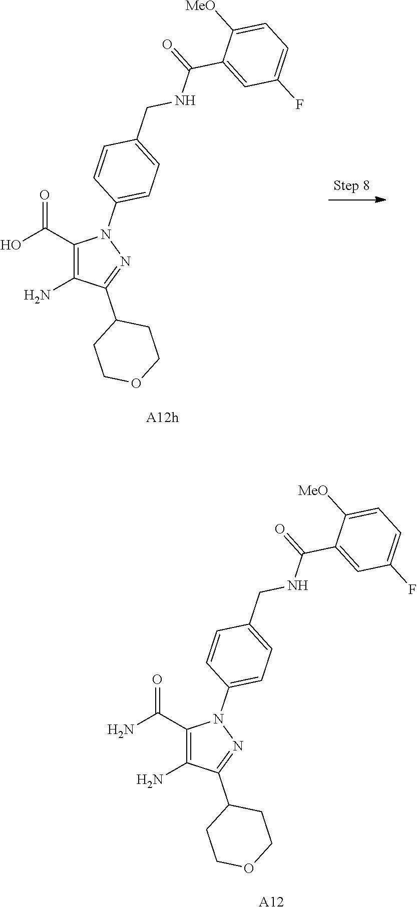

### Step 1: methyl 4-amino-1H-pyrazole-5-carboxylate A12b

A mixture of methyl 4-nitro-1H-pyrazole-5-carboxylate A12a (10 g, 58.44 mmol) and Pd/C (6.22 g, 5.84 mmol) in MeOH (100 mL) under hydrogen balloon was stirred for 16 hours at room temperature. The reaction mixture was filtered through Celite, and the filtrate was concentrated under vacuum to afford crude methyl 4-amino-1H-pyrazole-5-carboxylate A12b (8 g, 97.00% yield), which was for next step without further purification.

### Step 2: methyl 4-amino-3-bromo-1H-pyrazole-5-carboxylate A12c

To a solution of methyl 4-amino-1H-pyrazole-5-carboxylate A12b (24 g, 170.06 mmol) in MeCN (300 mL) at room temperature was slowly added NBS (33.29 g, 187.06 mmol). The resulting reaction mixture was stirred at room temperature for 3 hours before quenched with H₂O (200 mL). The resulting solution was extracted with EtOAc (200 mL×3), and the combined organic layers were dried over anhydrous Na₂SO₄, filtered, and concentrated under reduced pressure. The residue was purified by silica gel column chromatography (petroleum ether/EtOAc=4/1) to afford methyl 4-amino-3-bromo-1H-pyrazole-5-carboxylate A12c (27 g, 72.16% yield). ¹H NMR (400 MHz, DMSO-d₆): δ 13.36 (s, 1H), 4.82 (s, 2H), 3.82 (s, 3H) ppm; LCMS: MS m/z (ESI): 222.0[M+H]⁺.

### Step 3: methyl 4-amino-3-bromo-1-(4-cyanophenyl)-1H-pyrazole-5-carboxylate A12d and methyl 4-amino-5-bromo-1-(4-cyanophenyl)-1H-pyrazole-3-carboxylate A12d-1

A mixture of methyl 4-amino-3-bromo-1H-pyrazole-5-carboxylate A12c (3 g, 13.63 mmol), 4-fluorobenzonitrile (2.48 g, 20.45 mmol) and K₂CO₃ (1.88 g, 13.63 mmol) in DMF (30 mL) under nitrogen atmosphere was stirred overnight at 90° C. After cooling down, the mixture was quenched by H₂O (30 mL). The resulting solution was extracted with EtOAc (40 mL×3), and the combined organic layers were dried over anhydrous Na₂SO₄, filtered, and concentrated under reduced pressure. The residue was purified by silica gel column chromatography (petroleum ether/EtOAc=4/1) to afford methyl 4-amino-3-bromo-1-(4-cyanophenyl)-1H-pyrazole-5-carboxylate A12d (less polar, 50 mg, 1.14% yield), and its regioisomer methyl 4-amino-5-bromo-1-(4-cyanophenyl)-1H-pyrazole-3-carboxylate A12d-1 (more polar, 800 mg, 18.24% yield). ¹H NMR (400 MHz, DMSO-d₆): A12d, less polar regioisomer, δ 8.06 (d, 2H), 7.84 (d, 2H), 4.97 (s, 2H), 3.85 (s, 3H) ppm. ¹H NMR (400 MHz, DMSO-d₆): A12d-1, more polar regioisomer, δ 8.11 (d, 2H), 7.90 (d, 2H), 5.03 (s, 2H), 3.91 (s, 3H) ppm.

### Step 4: methyl 4-amino-1-(4-cyanophenyl)-3-(3,6-dihydro-2H-pyran-4-yl)-1H-pyrazole-5-carboxylate A12e

A mixture of methyl 4-amino-3-bromo-1-(4-cyanophenyl)-1H-pyrazole-5-carboxylate A12d (0.1 g, 311.40 umol), 2-(3,6-dihydro-2H-pyran-4-yl)-4,4,5,5-tetramethyl-1,3,2-dioxaborolane (98.13 mg, 467.10 umol), Pd(dppf)C12 (10.27 mg, 15.57 umol) and K₃PO₄ (198.30 mg, 934.20 umol) in dioxane:H₂O (4:1, 5 mL) under nitrogen atmosphere was stirred at 90° C. for 1 hour. After cooling down, the mixture was diluted with EtOAc (30 mL×3) and washed with water (20 mL). The combined organic extracts were washed with brine (20 mL×3), dried over anhydrous Na₂SO₄, filtered, and concentrated under reduced pressure. The resulting residue was first purified by prep-TLC (100% EtOAc) to afford methyl 4-amino-1-(4-cyanophenyl)-3-(3,6-dihydro-2H-pyran-4-yl)-1H-pyrazole-5-carboxylate A12e (35 mg, 34.65% yield). ¹H NMR (400 MHz, DMSO-d₆): δ 7.89 (d, 2H), 7.61 (d, 2H), 6.34 (s, 1H), 5.43 (s, 2H), 4.26 (d, 2H), 3.81 (t, 2H), 3.73 (s, 3H), 2.52-2.49 (m, 2H) ppm; LCMS: MS m/z (ESI): 325.1 [M+H]⁺.

### Step 5: methyl 4-amino-1-(4-(aminomethyl)phenyl)-3-(tetrahydro-2H-pyran-4-yl)-1H-pyrazole-5-carboxylate A12f

A mixture of methyl 4-amino-1-(4-cyanophenyl)-3-(3,6-dihydro-2H-pyran-4-yl)-1H-pyrazole-5-carboxylate A12e (50 mg, 154.16 umol), Pd/C (16.41 mg, 15.42 umol), and HCl (1 M, 154.16 uL) in MeOH under hydrogen balloon was stirred for 30 min at room temperature. The reaction mixture was filtered through Celite, and the filtrate was concentrated under vacuum to afford crude methyl 4-amino-1-(4-(aminomethyl)phenyl)-3-(tetrahydro-2H-pyran-4-yl)-1H-pyrazole-5-carboxylate A12f (40 mg, 70.73% yield), which was for next step without further purification.

### Step 6: methyl 4-amino-1-(4-((5-fluoro-2-methoxybenzamido)methyl)phenyl)-3-(tetrahydro-2H-pyran-4-yl)-1H-pyrazole-5-carboxylate A12 g

A mixture of 5-fluoro-2-methoxybenzoic acid (20.60 mg, 121.07 umol), methyl 4-amino-1-(4-(aminomethyl)phenyl)-3-(tetrahydro-2H-pyran-4-yl)-1H-pyrazole-5-carboxylate A12f (40 mg, 121.07 umol), HATU (4.60 mg, 12.11 umol) and TEA (61.26 mg, 605.36 umol) under nitrogen atmosphere was stirred at room temperature for 16 hours. The reaction was quenched with H₂O (10 mL) and extracted with EtOAc (15 mL×3). The combined organic layers were dried over anhydrous Na₂SO₄, filtered, and concentrated under reduced pressure. The residue was purified by silica gel column chromatography (petroleum ether/EA=1/5) to afford methyl 4-amino-1-(4-((5-fluoro-2-methoxybenzamido)methyl)phenyl)-3-(tetrahydro-2H-pyran-4-yl)-1H-pyrazole-5-carboxylate A12 g (40 mg, 68.47% yield). LCMS: MS m/z (ESI): 483.0[M+H]⁺.

### Step 7: 4-amino-1-(4-((5-fluoro-2-methoxybenzamido)methyl)phenyl)-3-(tetrahydro-2H-pyran-4-yl)-1H-pyrazole-5-carboxylic acid A12 h

To a solution of methyl 4-amino-1-(4-((5-fluoro-2-methoxybenzamido)methyl)phenyl)-3-(tetrahydro-2H-pyran-4-yl)-1H-pyrazole-5-carboxylate A12 g (40 mg, 82.90 umol) in MeOH (1.5 mL) was added NaOH (3 M, 2.76 uL) at room temperature. The resulting mixture was then slowly warm up to 50° C. and stirred for 2 hours. After cooling down, the pH of the mixture was adjusted to 5-6 by slow addition of 1M HCl solution. The resulting mixture was then extracted with EtOAc (20 mL×3). The combined organic layers were dried over anhydrous Na₂SO₄, filtered, and concentrated under reduced pressure to afford crude 4-amino-1-(4-((5-fluoro-2-methoxybenzamido)methyl)phenyl)-3-(tetrahydro-2H-pyran-4-yl)-1H-pyrazole-5-carboxylic acid A12 h (30 mg, 77.25% yield), which was used for next step without further purification.

### Step 8: 4-amino-1-(4-((5-fluoro-2-methoxybenzamido)methyl)phenyl)-3-(tetrahydro-2H-pyran-4-yl)-1H-pyrazole-5-carboxamide A12

A solution of 4-amino-1-(4-((5-fluoro-2-methoxybenzamido)methyl)phenyl)-3-(tetrahydro-2H-pyran-4-yl)-1H-pyrazole-5-carboxylic acid A12 h (30 mg, 64.04 umol), HATU (36.52 mg, 96.06 umol), NH₄Cl (10.28 mg, 192.11 umol), and TEA (32.40 mg, 320.19 umol) in THE (2 mL) was stirred overnight at room temperature. The resulting mixture was then quenched by water (20 mL), extracted with EtOAc (30 mL×3). The combined organic layers were dried over anhydrous Na₂SO₄, filtered, and concentrated under reduced pressure. The residue was then purified by prep-HPLC, eluting MeCN/H₂O/FA, to afford 4-amino-1-(4-((5-fluoro-2-methoxybenzamido)methyl)phenyl)-3-(tetrahydro-2H-pyran-4-yl)-1H-pyrazole-5-carboxamide A12 (8.15 mg, 27.22% yield). ¹H NMR (400 MHz, DMSO-d₆): δ 8.83 (t, 1H), 8.46 (s, 1H), 7.51 (dd, 1H), 7.42-7.26 (m, 6H), 7.18 (dd, 1H), 4.58 (brs, 2H), 4.52 (d, 2H), 3.93-3.89 (m, 5H), 3.43-3.40 (m, 2H), 2.99-2.93 (m, 1H), 1.79-1.65 (m, 4H) ppm; LCMS: MS m/z (ESI): 468.4[M+H]⁺.

### Example 13

### 4-amino-1-(4-((5-fluoro-2-methoxybenzamido)methyl)phenyl)-3-(tetrahydro-2H-pyran-3-yl)-1H-pyrazole-5-carboxamide A13

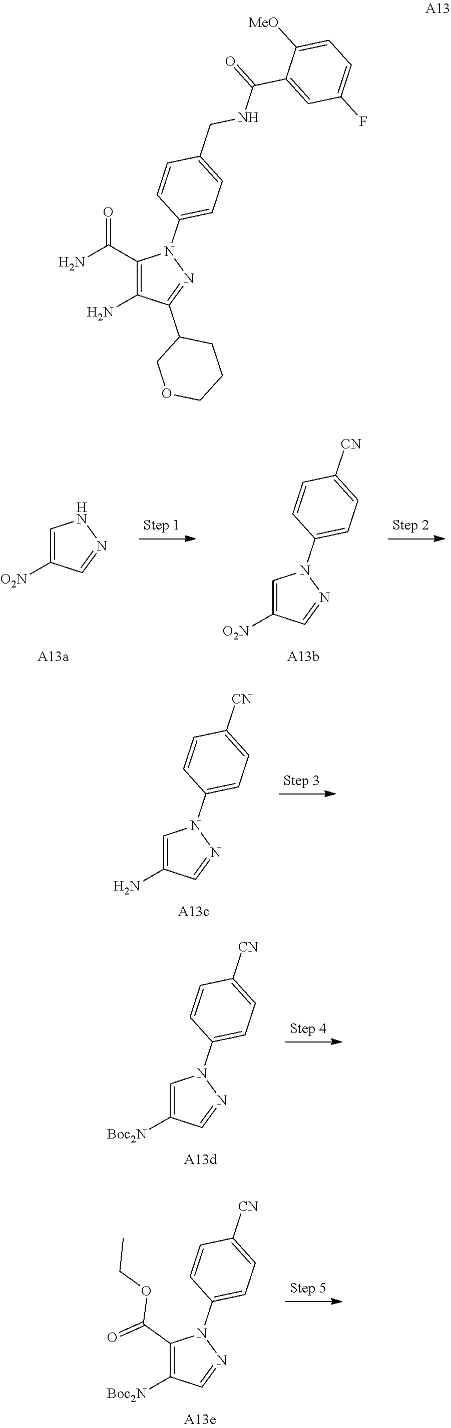

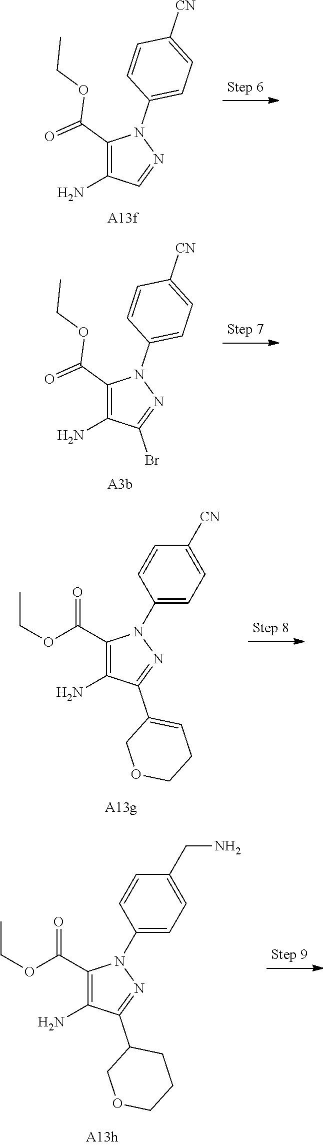 [SMILES: CCOC(=O)c1c(N)cnn1-c1ccc(C#N)cc1 ; CCOC(=O)c1c(N)c(Br)nn1-c1ccc(C#N)cc1 ; CCOC(=O)c1c(N)c(C2=CCCOC2)nn1-c1ccc(C#N)cc1 ; CCOC(=O)c1c(N)c(C2CCCOC2)nn1-c1ccc(CN)cc1]

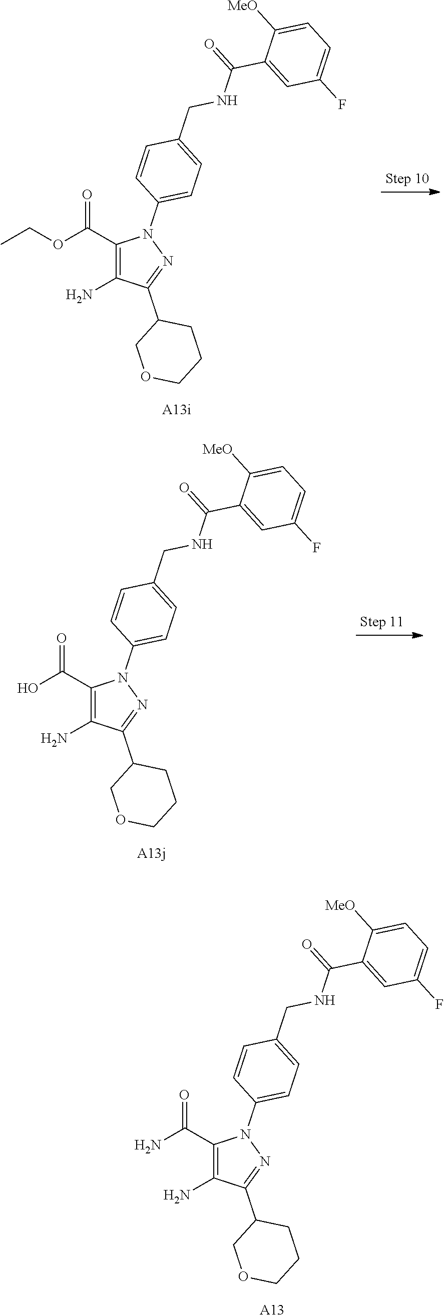

### Step 1: 4-(4-nitro-1H-pyrazol-1-yl)benzonitrile A13b

To a solution of 4-nitro-1H-pyrazole A13a (34 g, 300.69 mmol) and 4-fluorobenzonitrile (36.42 g, 300.69 mmol, Shaoyuan) in DMF (1000 mL) at room temperature under nitrogen atmosphere was added K₂CO₃ (81.79 g, 601.37 mmol). The resulting mixture was slowly warmed up to 100° C. and stirred for 2 hours. After cooling down, the reaction was quenched by water (1500 mL). The precipitate was filtered, washed with water, and dried to afford crude 4-(4-nitro-1H-pyrazol-1-yl)benzonitrile A13b (63 g, 97.82% yield), which was used for next step without further purification. ¹H NMR (400 MHz, CDCl₃): δ 8.72 (s, 1H), 8.32 (s, 1H), 7.91 (dd, 2H), 7.86 (dd, 2H) ppm.

### Step 2: 4-(4-amino-1H-pyrazol-1-yl)benzonitrile A13c

A mixture of 4-(4-nitro-1H-pyrazol-1-yl)benzonitrile A13b (70 g, 326.83 mmol) and 10% Pd/C (5 g) in MeOH (1000 mL) under hydrogen atmosphere was stirred at room atmosphere for 16 hours. The mixture was filtered over Celite, rinsed with MeOH, the filtrate was concentrated under vacuum to afford crude 4-(4-amino-1H-pyrazol-1-yl)benzonitrile A13c (55 g, 91.36% yield), which was used for next step without further purification.

### Step 3: tert-butyl (tert-butoxycarbonyl)(1-(4-cyanophenyl)-1H-pyrazol-4-yl)carbamate A13d

To a solution of 4-(4-amino-1H-pyrazol-1-yl)benzonitrile A13c (40 g, 217.39 mmol) in DCM (500 mL) was added (Boc)₂O (56.87 g, 260.87 mmol). The resulting reaction mixture was stirred at room atmosphere for 18 hours before concentrated under vacuum. The resulting residue was redissolved in THE (500 mL), and then (Boc)₂O (56.87 g, 260.87 mmol) and DMAP (2.65 g, 21.74 mmol) were added. The mixture was stirred at room atmosphere for additional 2 hours. The precipitate was filtered, washed with THF, and dried to afford crude tert-butyl (tert-butoxycarbonyl)(1-(4-cyanophenyl)-1H-pyrazol-4-yl)carbamate A13d (37.2 g, 96.77 mmol), which was used for next step without further purification. ¹H NMR (400 MHz, DMSO-d₆): δ 8.81 (s, 1H), 8.06 (d, 2H), 8.01 (d, 2H), 7.87 (s, 1H), 1.44 (s, 18H) ppm.

### Step 4: ethyl 4-(bis(tert-butoxycarbonyl)amino)-1-(4-cyanophenyl)-1H-pyrazole-5-carboxylate A13e

To a solution of tert-butyl (tert-butoxycarbonyl)(1-(4-cyanophenyl)-1H-pyrazol-4-yl)carbamate A13d (11.8 g, 30.69 mmol) in THE (200 mL) at −78° C. under nitrogen atmosphere was added LiHMDS (1M in THF, 92.07 mL, 92.07 mmol). The resulting mixture was stirred at −78° C. for 1 hour before slow addition of ethyl carbonochloridate (9.99 g, 92.07 mmol). The resulting mixture was stirred at −78° C. for 30 min, and then slowly warm up to room temperature. The reaction was quenched with saturated aqueous NH₄Cl. The resulting solution was extracted with EtOAc, and the combined organic layers were dried over anhydrous Na₂SO₄, filtered, and concentrated under reduced pressure. The residue was purified by silica gel column chromatography (PE/EA=8/1) to afford ethyl 4-(bis(tert-butoxycarbonyl)amino)-1-(4-cyanophenyl)-1H-pyrazole-5-carboxylate A13e (4.5 g, 32.12% yield). ¹H NMR (400 MHz, DMSO-d₆): δ 8.02-7.99 (m, 3H), 7.73 (d, 2H), 4.19 (q, 2H), 1.41 (s, 18H), 1.16 (t, 3H) ppm.

### Step 5: ethyl 4-amino-1-(4-cyanophenyl)-1H-pyrazole-5-carboxylate A13f

A solution of ethyl 4-(bis(tert-butoxycarbonyl)amino)-1-(4-cyanophenyl)-1H-pyrazole-5-carboxylate A13e (1.6 g, 3.50 mmol) in HCl (4M in dioxane, 10 mL) was stirred at 50° C. for 16 hours. After cooling down, the mixture was concentrated under vacuum, and the resulting was redissolved in H₂O (20 mL), and adjust pH to ˜5-6 with saturated NaHCO₃ solution. The resulting solution was extracted with EtOAc, and the combined organic layers were dried over anhydrous Na₂SO₄, filtered, and concentrated under reduced pressure to afford crude ethyl 4-amino-1-(4-cyanophenyl)-1H-pyrazole-5-carboxylate A13f (700 mg, 77.93% yield), which was used for next step without further purification. ¹H NMR (400 MHz, CDCl₃): δ7.71 (d, 2H), 7.53 (d, 2H), 7.50 (s, 1H), 4.41 (s, 2H), 4.28 (q, 2H), 1.26 (t, 3H).

### Step 6: ethyl 4-amino-3-bromo-1-(4-cyanophenyl)-1H-pyrazole-5-carboxylate A3b

To a solution of crude ethyl 4-amino-1-(4-cyanophenyl)-1H-pyrazole-5-carboxylate A13f (400 mg, 1.56 mmol) in MeCN (10 mL) at room temperature was dropwise added a solution of NBS (277.81 mg, 1.56 mmol) in MeCN (5 mL). The resulting mixture was then stirred at room temperature for 15 min. The precipitate was filtered, washed with MeCN, and dried to afford crude ethyl 4-amino-3-bromo-1-(4-cyanophenyl)-1H-pyrazole-5-carboxylate A3b (340 mg, 64.99% yield), which was used for next step without further purification. ¹H NMR (400 MHz, DMSO-d₆): δ7.93 (dd, 2H), 7.64 (dd, 2H), 5.39 (brs, 2H), 4.21 (q, 2H), 1.15 (t, 3H) ppm.

### Step 7: ethyl 4-amino-1-(4-cyanophenyl)-3-(5,6-dihydro-2H-pyran-3-yl)-1H-pyrazole-5-carboxylate A13 g

A mixture of ethyl 4-amino-3-bromo-1-(4-cyanophenyl)-1H-pyrazole-5-carboxylate A3b (0.2 g, 0.597 mmol), 2-(5,6-dihydro-2H-pyran-3-yl)-4,4,5,5-tetramethyl-1,3,2-dioxaborolane (151 mg, 716.4 umol), Pd(dppf)C12 (98 mg, 0.194 mmol) and K₃PO₄ (380 mg, 1.79 mmol) in dioxane (4 mL) and H₂O (0.8 mL) under nitrogen atmosphere was stirred at 90° C. for 1 hour. After cooling down, the mixture was quenched by water (20 mL), and extracted with EtOAc (20 mL×3). The organic layers were dried over anhydrous Na₂SO₄, filtered, and concentrated under reduce pressure. The residue was purified by prep-TLC (PE/EtOAc=1/1) to afford ethyl 4-amino-1-(4-cyanophenyl)-3-(5,6-dihydro-2H-pyran-3-yl)-1H-pyrazole-5-carboxylate A13 g (121 mg, 59.90% yield). LCMS: MS m/z (ESI): 339.0[M+H]⁺.

### Step 8: ethyl 4-amino-1-(4-(aminomethyl)phenyl)-3-(tetrahydro-2H-pyran-3-yl)-1H-pyrazole-5-carboxylate A13 h

A mixture of ethyl 4-amino-1-(4-cyanophenyl)-3-(5,6-dihydro-2H-pyran-3-yl)-1H-pyrazole-5-carboxylate A13 g (121 mg, 0.358 mmol), Pd/C (120 mg), and HCl (1 M, 154.16 uL) in MeOH under hydrogen balloon was stirred for 30 min at room temperature. The reaction mixture was filtered through Celite, and the filtrate was concentrated under vacuum to afford crude ethyl 4-amino-1-(4-(aminomethyl)phenyl)-3-(tetrahydro-2H-pyran-3-yl)-1H-pyrazole-5-carboxylate A13 h (142 mg), which was used for next step without further purification. LCMS: MS m/z (ESI): 328.1 [M+H−H₂O]⁺.

### Step 9: ethyl 4-amino-1-(4-((5-fluoro-2-methoxybenzamido)methyl)phenyl)-3-(tetrahydro-2H-pyran-3-yl)-1H-pyrazole-5-carboxylate A13i

A mixture of 5-fluoro-2-methoxybenzoic acid (100 mg, 0.597 mmol), ethyl 4-amino-1-(4-(aminomethyl)phenyl)-3-(tetrahydro-2H-pyran-3-yl)-1H-pyrazole-5-carboxylate A13 h (142 mg, 0.412 mmol), HATU (23 mg, 597 umol) and DIPEA (40 mg, 0.299 mmol) in THE (10 mL) under nitrogen atmosphere was stirred overnight at room temperature. The mixture was quenched by water (10 mL), and extracted with EtOAc (20 mL×3). The organic layers were dried over anhydrous Na₂SO₄, filtered, and concentrated under reduce pressure. The residue was purified by silica gel column chromatography (PE/EtOAc=1/2) to afford ethyl 4-amino-1-(4-((5-fluoro-2-methoxybenzamido)methyl)phenyl)-3-(tetrahydro-2H-pyran-3-yl)-1H-pyrazole-5-carboxylate A13i (35 mg, 11.81% yield). LCMS: MS m/z (ESI): 497.1[M+H]⁺.

### Step 10: 4-amino-1-(4-((5-fluoro-2-methoxybenzamido)methyl)phenyl)-3-(tetrahydro-2H-pyran-3-yl)-1H-pyrazole-5-carboxylic acid A13j

To a solution of ethyl 4-amino-1-(4-((5-fluoro-2-methoxybenzamido)methyl)phenyl)-3-(tetrahydro-2H-pyran-3-yl)-1H-pyrazole-5-carboxylate A13i (35 mg, 0.070 mmol) in MeOH (3 mL) and THE (1 mL) was added NaOH (3 M in water, 14.1 uL). The resulting mixture was slowly warmed up to 50° C. and stirred for 5 hours. After cooling down, the pH of the mixture was adjusted to 5-6 by slow addition of 1M HCl aqueous solution, and then extracted with EtOAc (20 mL×3). The organic layers were dried over anhydrous Na₂SO₄, filtered, and concentrated under reduce pressure to afford crude 4-amino-1-(4-((5-fluoro-2-methoxybenzamido)methyl)phenyl)-3-(tetrahydro-2H-pyran-3-yl)-1H-pyrazole-5-carboxylic acid A13j (48 mg), which was used for next step without further purification. LCMS: MS m/z (ESI): 469.1[M+H]⁺.

### Step 11: 4-amino-1-(4-((5-fluoro-2-methoxybenzamido)methyl)phenyl)-3-(tetrahydro-2H-pyran-3-yl)-1H-pyrazole-5-carboxamide A13

A solution of 4-amino-1-(4-((5-fluoro-2-methoxybenzamido)methyl)phenyl)-3-(tetrahydro-2H-pyran-3-yl)-1H-pyrazole-5-carboxylic acid A13j (48 mg, 0.102 mmol), HATU (340 mg, 0.895 mmol), NH₄Cl (96 mg, 1.791 mmol), and DIPEA (386 mg, 2.985 mmol) in THE (10 mL) was stirred overnight at room temperature. The reaction was quenched by water (20 mL), and then extracted with EtOAc (20 mL×3). The organic layers were dried over anhydrous Na₂SO₄, filtered, and concentrated under reduce pressure. The residue was directly purified by prep-HPLC, eluting with MeCN/H₂O/FA, to afford 4-amino-1-(4-((5-fluoro-2-methoxybenzamido)methyl)phenyl)-3-(tetrahydro-2H-pyran-3-yl)-1H-pyrazole-5-carboxamide A13 (23 mg, 0.049 mmol). ¹H NMR (400 MHz, DMSO-d₆): δ 8.85 (t, 1H), 7.51 (dd, 1H), 7.44-7.29 (m, 6H), 7.22-7.16 (m, 2H), 4.52 (d, 2H), 3.91-3.85 (m, 2H), 3.89 (s, 3H), 3.44-3.26 (m, 2H), 3.07-2.95 (m, 1H), 2.02-1.96 (m, 1H), 1.83-1.54 (m, 3H) ppm; LCMS: MS m/z (ESI): 468.1[M+H]⁺.

### Example A14

### 4-amino-1-(4-((5-fluoro-2-methoxybenzamido)methyl)phenyl)-3-(thiophen-2-yl)-1H-pyrazole-5-carboxamide A14

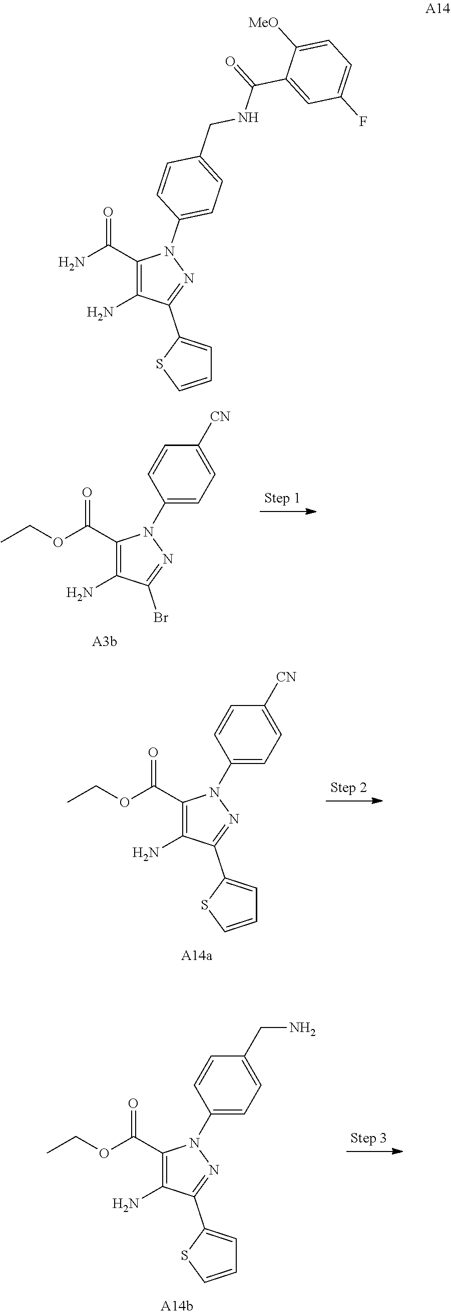

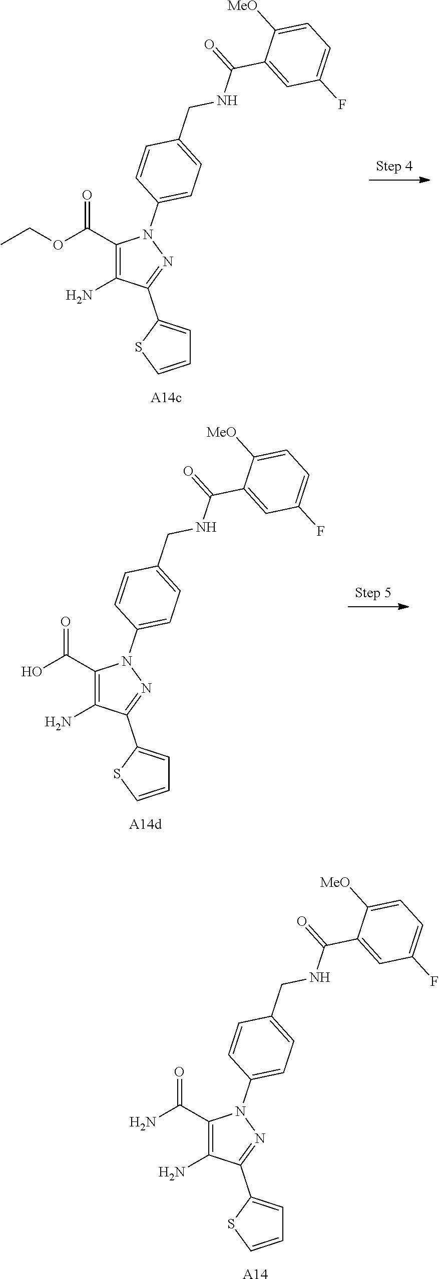

### Step 1: ethyl 4-amino-1-(4-cyanophenyl)-3-(thiophen-2-yl)-1H-pyrazole-5-carboxylate A14a

A mixture of ethyl 4-amino-3-bromo-1-(4-cyanophenyl)-1H-pyrazole-5-carboxylate A3b (0.1 g, 300 umol), thiophen-2-ylboronic acid (46 mg, 360 umol), Pd(dppf)C12 (49 mg, 60 umol) and K₃PO₄ (191 mg, 900 umol) in dioxane (5 mL) and H₂O (1 mL) under nitrogen atmosphere was stirred in microwave at 90° C. for 2 hours. After cooling down, the reaction was quenched by water (10 mL) and then extracted with EtOAc (20 mL×3). The combined organic extracts were washed with brine (20 mL×3), dried over anhydrous Na₂SO₄, filtered, and concentrated under reduced pressure. The resulting residue was purified by prep-TLC (PE/EtOAc=3/1) to afford ethyl 4-amino-1-(4-cyanophenyl)-3-(thiophen-2-yl)-1H-pyrazole-5-carboxylate A14a (50 mg, 49.11% yield). 1H NMR (400 MHz, DMSO-d₆): δ 7.94 (d, 2H), 7.68 (d, 2H), 7.60 (dd, 2H), 7.20 (dd, 1H), 5.52 (s, 2H), 4.22 (q, 2H), 1.15 (t, 3H) ppm; LCMS: MS m/z (ESI): 339.1 [M+H]⁺.

### Step 2: ethyl 4-amino-1-(4-(aminomethyl)phenyl)-3-(thiophen-2-yl)-1H-pyrazole-5-carboxylate A14b

A mixture of ethyl 4-amino-1-(4-cyanophenyl)-3-(thiophen-2-yl)-1H-pyrazole-5-carboxylate A14a (100 mg, 295.6 umol), Pd/C (720 mg, 676 umol), and HCl (12 M, 1 drop) in MeOH (40 mL) was stirred under hydrogen atmosphere (15 psi) at room temperature for 2 hours. Then, the mixture was filtered through Celite, and the filtrate was concentrated under reduced pressure to afford crude ethyl 4-amino-1-(4-(aminomethyl)phenyl)-3-(thiophen-2-yl)-1H-pyrazole-5-carboxylate A14b (58 mg, 57.43% yield), which was used for next step without further purification. LCMS: MS m/z (ESI): 326.0 [M+H−H₂O]⁺.

### Step 3: ethyl 4-amino-1-(4-((5-fluoro-2-methoxybenzamido)methyl)phenyl)-3-(thiophen-2-yl)-1H-pyrazole-5-carboxylate A14c

A mixture of 5-fluoro-2-methoxybenzoic acid (25 mg, 146 umol), ethyl 4-amino-1-(4-(aminomethyl)phenyl)-3-(thiophen-2-yl)-1H-pyrazole-5-carboxylate A14b (58 mg, 146 umol), HATU (83 mg, 219 umol), and DIPEA (90 mg, 700 umol) in anhydrous THF (20 mL) and DMF (2 mL) under nitrogen atmosphere was stirred overnight at room temperature. The reaction was quenched by water (10 mL) and then extracted with EtOAc (15 mL×3). The combined organic extracts were washed with brine, dried over anhydrous Na₂SO₄, filtered, and concentrated under reduced pressure to get crude ethyl 4-amino-1-(4-((5-fluoro-2-methoxybenzamido)methyl)phenyl)-3-(thiophen-2-yl)-1H-pyrazole-5-carboxylate A14c (25 mg, 34.72% yield), which was used for next step without further purification. ¹H NMR (400 MHz, DMSO-d₆): δ 8.85-8.83 (m, 1H), 7.56-7.15 (m, 8H), 6.95-6.85 (m, 1H), 5.34 (brs, 1H), 5.08 (brs, 1H), 4.15 (q, 2H), 3.90 (s, 3H), 1.14 (t, 3H) ppm; LCMS: MS m/z (ESI): 495.0 [M+H]⁺.

### Step 4: 4-amino-1-(4-((5-fluoro-2-methoxybenzamido)methyl)phenyl)-3-(thiophen-2-yl)-1H-pyrazole-5-carboxylic acid A14d

To a solution of ethyl 4-amino-1-(4-((5-fluoro-2-methoxybenzamido)methyl)phenyl)-3-(thiophen-2-yl)-1H-pyrazole-5-carboxylate A14c (25 mg, 51 umol) in MeOH (1.5 mL) and THE (0.5 mL) at room temperature was added NaOH (3 M, 2.76 uL). The resulting mixture was slowly warmed up and stirred at 50° C. for 2 hours. After cooling down, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-1-(4-((5-fluoro-2-methoxybenzamido)methyl)phenyl)-3-(thiophen-2-yl)-1H-pyrazole-5-carboxylic acid A14d (36 mg), which was used for next step without further purification. LCMS: MS m/z (ESI): 465.0 [M−H]⁻.

### Step 5: 4-amino-1-(4-((5-fluoro-2-methoxybenzamido)methyl)phenyl)-3-(thiophen-2-yl)-1H-pyrazole-5-carboxamide A14

A mixture of NH₄Cl (13 mg, 237 umol), HATU (45.06 mg, 118 umol), 4-amino-1-(4-((5-fluoro-2-methoxybenzamido)methyl)phenyl)-3-(thiophen-2-yl)-1H-pyrazole-5-carboxylic acid A14d (36 mg, 79 umol) and DIPEA (51.05 mg, 395 umol) under nitrogen atmosphere was stirred overnight at room temperature. The reaction mixture quenched by water (10 mL), and then extracted with EtOAc (15 mL×3). The combined organic extracts were washed with brine, dried over anhydrous Na₂SO₄, filtered, and concentrated under reduced pressure. The residue was purified by prep-HPLC, eluting with MeCN/H₂O/formic acid, to afford 4-amino-1-(4-((5-fluoro-2-methoxybenzamido)methyl)phenyl)-3-(thiophen-2-yl)-1H-pyrazole-5-carboxamide A14 (1.25 mg, 3.33% yield). 1H NMR (400 MHz, DMSO-d₆): δ 8.86 (t, J=6.0 Hz, 1H), 7.56-7.50 (m, 3H), 7.41 (q, 4H), 7.38-7.32 (m, 1H), 7.22-7.14 (m, 2H), 4.80 (s, 2H), 4.55 (d, 2H), 3.90 (s, 3H) ppm; LCMS: MS m/z (ESI): 466.0[M+H]⁺.

### Example A15

### 4-amino-3-cyclopentyl-1-(4-((5-fluoro-2-methoxybenzamido)methyl)phenyl)-1H-pyrazole-5-carboxamide A15

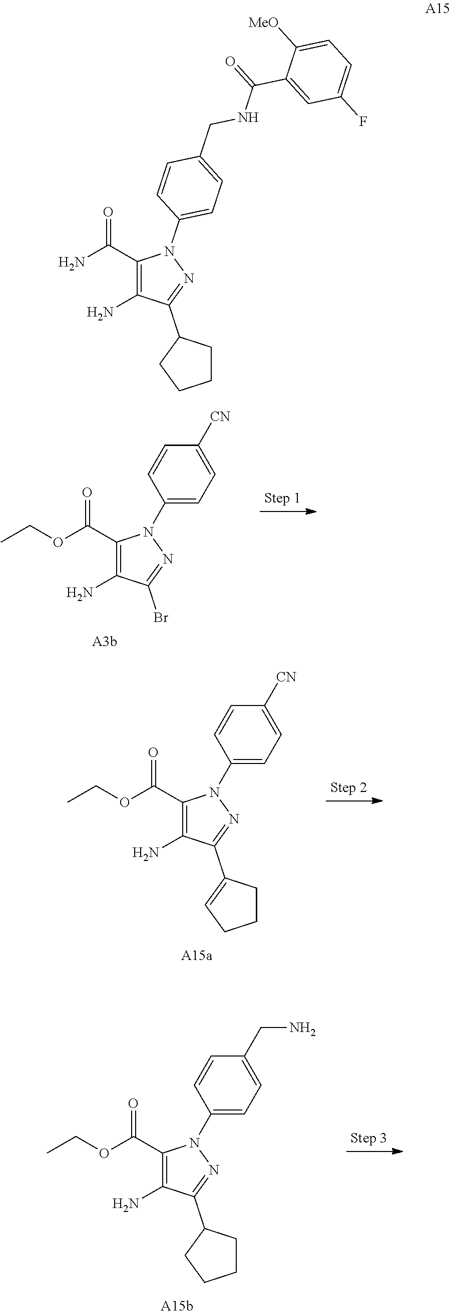

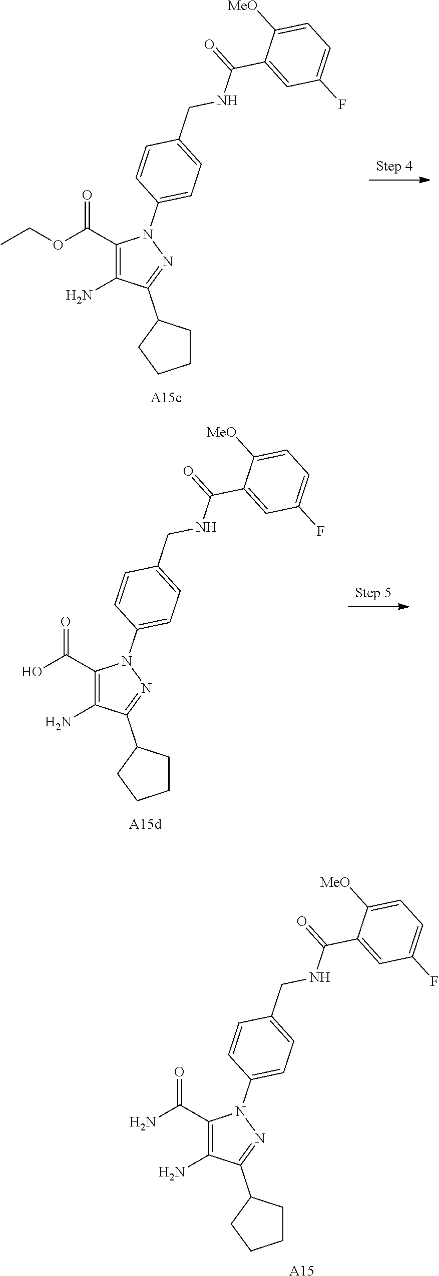

### Step 1: ethyl 4-amino-1-(4-cyanophenyl)-3-(cyclopent-1-en-1-yl)-1H-pyrazole-5-carboxylate A15a

A mixture of ethyl 4-amino-3-bromo-1-(4-cyanophenyl)-1H-pyrazole-5-carboxylate A3b (0.05 g, 0.149 mmol), Pd(dppf)C12 (10.98 mg, 0.015 mmol), K₃PO₄ ((98.77 mg, 0.465 mmol), and 2-(cyclopent-3-en-1-yl)-4,4,5,5-tetramethyl-1,3,2-dioxaborolane (45.15 mg, 0.233 mmol) in dioxane (4 mL) and H₂O (0.8 mL) under nitrogen atmosphere was stirred at 90° C. for 1 hour. After cooling down, the reaction was quenched by water (15 mL) and then extracted with EtOAc (20 mL×3). The combined organic extracts were washed with brine (20 mL×3), dried over anhydrous Na₂SO₄, filtered, and concentrated under reduced pressure. The resulting residue was purified by prep-TLC (PE/EtOAc=1/3) to afford ethyl 4-amino-1-(4-cyanophenyl)-3-(cyclopent-1-en-1-yl)-1H-pyrazole-5-carboxylate A15a (40 mg, 83.22% yield). LCMS: MS m/z (ESI): 323.0 [M+H]⁺.

### Step 2: ethyl 4-amino-1-(4-(aminomethyl)phenyl)-3-cyclopentyl-1H-pyrazole-5-carboxylate A15b

A mixture of ethyl 4-amino-1-(4-cyanophenyl)-3-(cyclopent-1-en-1-yl)-1H-pyrazole-5-carboxylate A15a (40 mg, 0.124 mmol), Pd/C (40 mg), and HCl (1 M in water, 77.08 uL) in MeOH (10 mL) was stirred under hydrogen atmosphere (15 psi) at room temperature for 30 min. Then, the mixture was filtered through Celite, and the filtrate was concentrated under reduced pressure to afford crude ethyl 4-amino-1-(4-(aminomethyl)phenyl)-3-cyclopentyl-1H-pyrazole-5-carboxylate A15b (58 mg), which was used for next step without further purification.

### Step 3: ethyl 4-amino-3-cyclopentyl-1-(4-((5-fluoro-2-methoxybenzamido)methyl)phenyl)-1H-pyrazole-5-carboxylate A15c

A mixture of 5-fluoro-2-methoxybenzoic acid (36.14 mg, 0.212 mmol), ethyl 4-amino-1-(4-(aminomethyl)phenyl)-3-cyclopentyl-1H-pyrazole-5-carboxylate A15b (58 mg, 0.177 mmol), HATU (101.14 mg, 0.266 mmol), and TEA (89.55 mg, 0.885 mmol) in anhydrous THE (8 mL) under nitrogen atmosphere was stirred overnight at room temperature. The reaction was quenched by water (10 mL) and then extracted with EtOAc (20 mL×3). The combined organic extracts were washed with brine, dried over anhydrous Na₂SO₄, filtered, and concentrated under reduced pressure. the residue was purified by prep-TLC (PE/EtOAc=1/2) to afford ethyl 4-amino-3-cyclopentyl-1-(4-((5-fluoro-2-methoxybenzamido)methyl)phenyl)-1H-pyrazole-5-carboxylate A15c (20 mg, 23.53% yield). LCMS: MS m/z (ESI): 481.0[M+H]⁺.

### Step 4: 4-amino-3-cyclopentyl-1-(4-((5-fluoro-2-methoxybenzamido)methyl)phenyl)-1H-pyrazole-5-carboxylic acid A15d

To a solution of ethyl 4-amino-3-cyclopentyl-1-(4-((5-fluoro-2-methoxybenzamido)methyl)phenyl)-1H-pyrazole-5-carboxylate A15c (20 mg, 0.043 mmol) in MeOH (1.5 mL) and THE (0.5 mL) at room temperature was added NaOH (3 M, 5.52 uL). The resulting mixture was slowly warmed up and stirred at 50° C. for 5 hours. After cooling down, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-3-cyclopentyl-1-(4-((5-fluoro-2-methoxybenzamido)methyl)phenyl)-1H-pyrazole-5-carboxylic acid A15d (13 mg, 67.02% yield), which was used for next step without further purification. LCMS: MS m/z (ESI): 452.1 [M+H]⁺.

### Step 5: 4-amino-3-cyclopentyl-1-(4-((5-fluoro-2-methoxybenzamido)methyl)phenyl)-1H-pyrazole-5-carboxamide A15

A mixture of NH₄Cl (4.61 mg, 0.086 mmol), HATU (16.39 mg, 0.043 mmol), 4-amino-3-cyclopentyl-1-(4-((5-fluoro-2-methoxybenzamido)methyl)phenyl)-1H-pyrazole-5-carboxylic acid A15d (13 mg, 0.029 mmol) and DIPEA (18.57 mg, 0.144 mmol) under nitrogen atmosphere was stirred at room temperature for 16 hours. The reaction mixture quenched by water (20 mL), and then extracted with EtOAc (30 mL×3). The combined organic extracts were washed with brine, dried over anhydrous Na₂SO₄, filtered, and concentrated under reduced pressure. The residue was purified by prep-HPLC, eluting with MeCN/H₂O/FA, to afford 4-amino-3-cyclopentyl-1-(4-((5-fluoro-2-methoxybenzamido)methyl)phenyl)-1H-pyrazole-5-carboxamide A15 (2.05 mg, 15.80% yield). ¹H NMR (400 MHz, DMSO-d₆): δ 8.82 (t, 1H), 7.51 (dd, 1H), 7.37-7.31 (m, 3H), 7.27 (d, 2H), 7.18 (dd, 1H), 4.50 (d, 4H), 3.89 (s, 3H), 3.15-3.10 (m, 1H), 1.98-1.92 (m, 2H), 1.79-1.52 (m, 6H) ppm; LCMS: MS m/z (ESI): 452.1[M+H]⁺.

### Example A16

### 4-amino-1-(4-((5-fluoro-2-methoxybenzamido)methyl)phenyl)-3-(tetrahydrofuran-3-yl)-1H-pyrazole-5-carboxamide A16

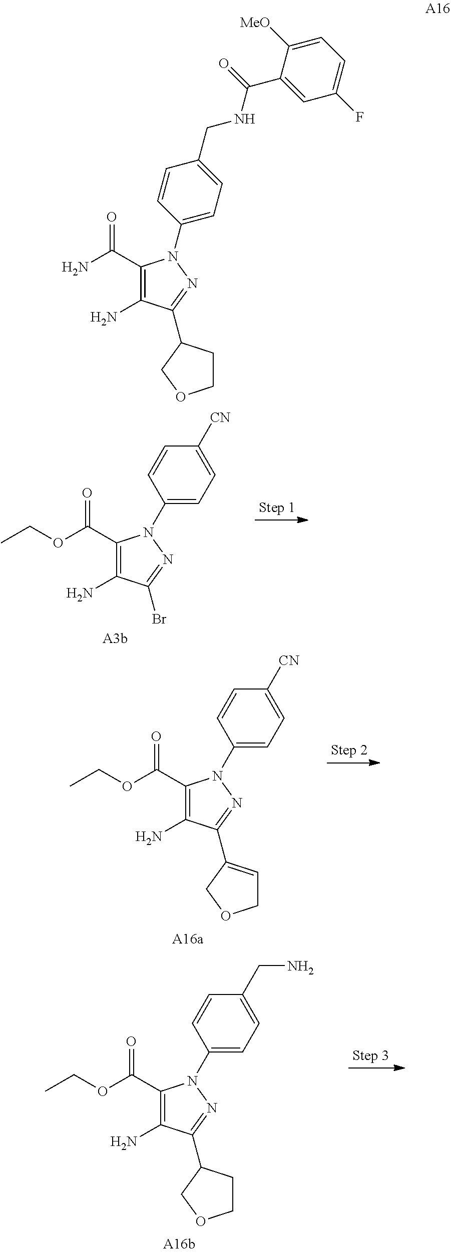

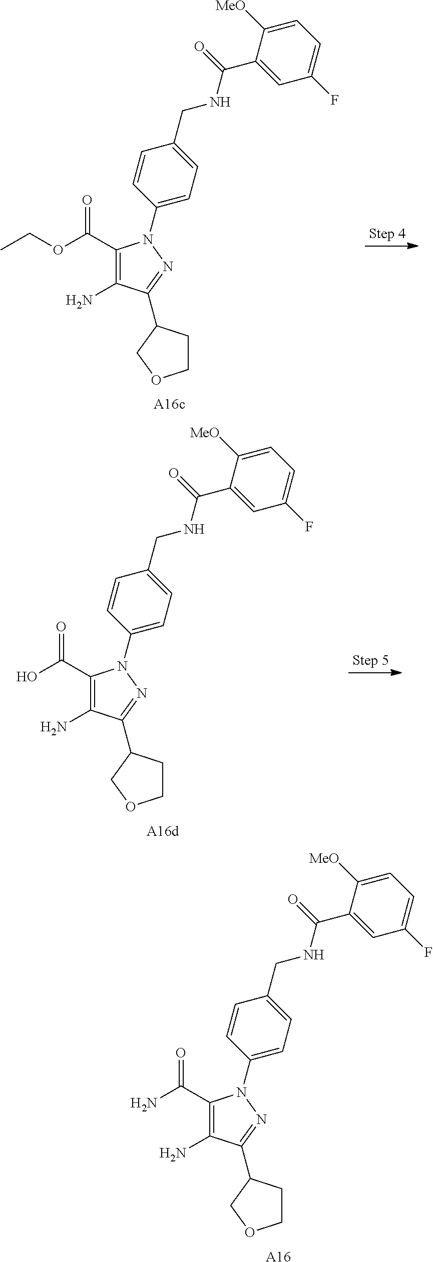

### Step 1: ethyl 4-amino-1-(4-cyanophenyl)-3-(2,5-dihydrofuran-3-yl)-1H-pyrazole-5-carboxylate A16a

A mixture of ethyl 4-amino-3-bromo-1-(4-cyanophenyl)-1H-pyrazole-5-carboxylate A3b (0.4 g, 1.11 mmol), Pd(dppf)C12 (182 mg, 0.221 mmol), K₃PO₄ (706 mg, 3.32 mmol), and 2-(2,5-dihydrofuran-3-yl)-4,4,5,5-tetramethyl-1,3,2-dioxaborolane (250 mg, 1.28 mmol) in dioxane (10 mL) and H₂O (2 mL) under nitrogen atmosphere was stirred in microwave at 90° C. for 2 hours. After cooling down, the reaction was quenched by water (20 mL) and then extracted with EtOAc (30 mL×3). The combined organic extracts were washed with brine (20 mL×3), dried over anhydrous Na₂SO₄, filtered, and concentrated under reduced pressure. The resulting residue was purified by prep-TLC (PE/EtOAc=1/2) to afford ethyl 4-amino-1-(4-cyanophenyl)-3-(2,5-dihydrofuran-3-yl)-1H-pyrazole-5-carboxylate A16a (126 mg, 34.95% yield). LCMS: MS m/z (ESI): 325.1[M+H]⁺.

### Step 2: ethyl 4-amino-1-(4-(aminomethyl)phenyl)-3-(tetrahydrofuran-3-yl)-1H-pyrazole-5-carboxylate A16b

A mixture of ethyl 4-amino-1-(4-cyanophenyl)-3-(2,5-dihydrofuran-3-yl)-1H-pyrazole-5-carboxylate A16a (126 mg, 0.388 mmol), Pd/C (126 mg), and HCl (1 M, 154.16 uL) in MeOH (15 mL) was stirred under hydrogen atmosphere (15 psi) at room temperature for 30 min. The mixture was filtered through Celite, and the filtrate was concentrated under reduced pressure to afford ethyl 4-amino-1-(4-(aminomethyl)phenyl)-3-(tetrahydrofuran-3-yl)-1H-pyrazole-5-carboxylate A16b (154 mg), which was used for next step without further purification.

### Step 3: ethyl 4-amino-1-(4-((5-fluoro-2-methoxybenzamido)methyl)phenyl)-3-(tetrahydrofuran-3-yl)-1H-pyrazole-5-carboxylate A16c

A mixture of 5-fluoro-2-methoxybenzoic acid (63 mg, 0.37 mmol), ethyl 4-amino-1-(4-(aminomethyl)phenyl)-3-(tetrahydrofuran-3-yl)-1H-pyrazole-5-carboxylate A16b (146 mg, 0.442 mmol), HATU (211 mg, 0.56 mmol), and DIPEA (240 mg, 0.56 mmol) in anhydrous THE (10 mL) under nitrogen atmosphere was stirred overnight at room temperature. The reaction was quenched by water (10 mL) and then extracted with EtOAc (15 mL×3). The combined organic extracts were washed with brine, dried over anhydrous Na₂SO₄, filtered, and concentrated under reduced pressure. The residue was purified by prep-TLC (PE/EtOAc=1/2) to afford ethyl 4-amino-1-(4-((5-fluoro-2-methoxybenzamido)methyl)phenyl)-3-(tetrahydrofuran-3-yl)-1H-pyrazole-5-carboxylate A16c (140 mg, 65.61% yield). LCMS: MS m/z (ESI): 483.2[M+H]⁺.

### Step 4: 4-amino-1-(4-((5-fluoro-2-methoxybenzamido)methyl)phenyl)-3-(tetrahydrofuran-3-yl)-1H-pyrazole-5-carboxylic acid A16d

To a solution of ethyl 4-amino-1-(4-((5-fluoro-2-methoxybenzamido)methyl)phenyl)-3-(tetrahydrofuran-3-yl)-1H-pyrazole-5-carboxylate A16c (130 mg, 0.270 mmol) in EtOH (3 mL) and THE (1 mL) at room temperature was added NaOH (3 M in water, 5.52 uL). The resulting mixture was slowly warmed up and stirred at 50° C. for 5 hours. After cooling down, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-1-(4-((5-fluoro-2-methoxybenzamido)methyl)phenyl)-3-(tetrahydrofuran-3-yl)-1H-pyrazole-5-carboxylic acid A16d (125 mg), which was used for next step without further purification. LCMS: MS m/z (ESI): 455.1[M+H]⁺.

### Step 5: 4-amino-1-(4-((5-fluoro-2-methoxybenzamido)methyl)phenyl)-3-(tetrahydrofuran-3-yl)-1H-pyrazole-5-carboxamide A16

A mixture of NH₄Cl (60 mg, 1.11 mmol), HATU (211 mg, 0.56 mmol), 4-amino-1-(4-((5-fluoro-2-methoxybenzamido)methyl)phenyl)-3-(tetrahydrofuran-3-yl)-1H-pyrazole-5-carboxylic acid A16d (125 mg, 0.276 mmol) and DIPEA (240 mg, 1.85 mmol) under nitrogen atmosphere was stirred overnight at room temperature. The reaction mixture quenched by water (20 mL), and then extracted with EtOAc (30 mL×3). The combined organic extracts were washed with brine, dried over anhydrous Na₂SO₄, filtered, and concentrated under reduced pressure. The residue was purified by prep-HPLC, eluting with MeCN/H₂O/TFA, to afford 4-amino-1-(4-((5-fluoro-2-methoxybenzamido)methyl)phenyl)-3-(tetrahydrofuran-3-yl)-1H-pyrazole-5-carboxamide A16 (83 mg, 66.32% yield). ¹H NMR (400 MHz, DMSO-d₆): δ 8.86 (t, 1H), 7.70 (br, 1H), 7.51 (dd, 1H), 7.41 (d, 2H), 7.37-7.30 (m, 4H), 7.18 (dd, 1H), 4.53 (d, 2H), 4.02 (t, 1H), 3.89 (s, 3H), 3.89-3.86 (m, 1H), 3.78 (q, 1H), 3.69 (t, 1H), 3.57-3.48 (m, 1H), 2.30-2.20 (m, 1H), 2.16-2.05 (m, 1H) ppm; LCMS: MS m/z (ESI): 454.1[M+H]⁺.

### Example A17

### 4-amino-1-(4-((5-fluoro-2-methoxybenzamido)methyl)phenyl)-3-morpholino-1H-pyrazole-5-carboxamide A17

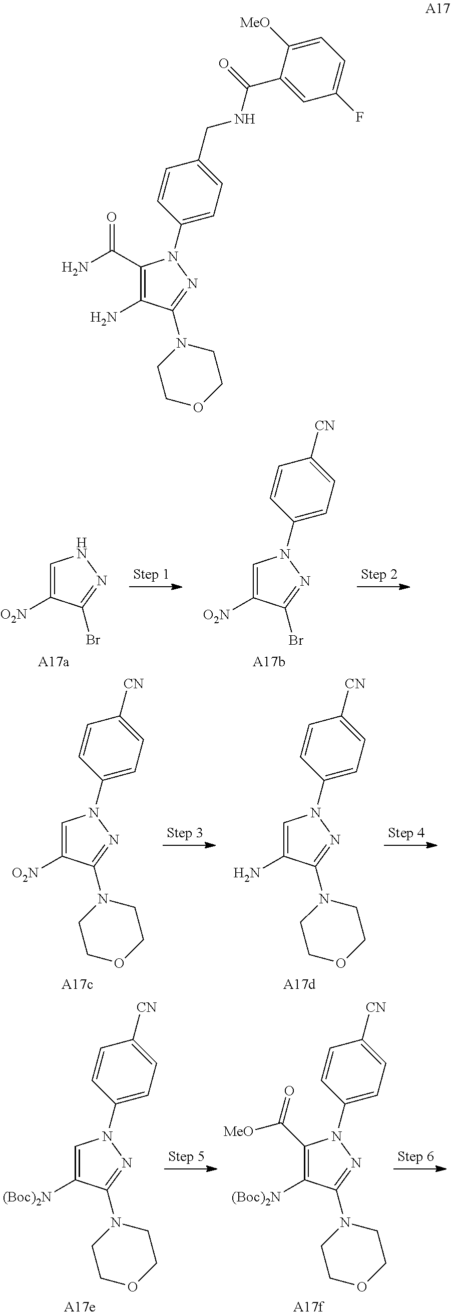

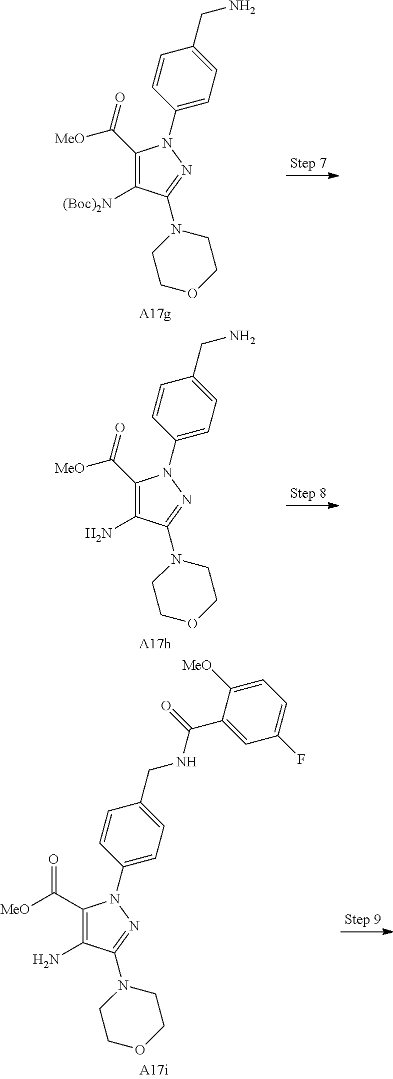

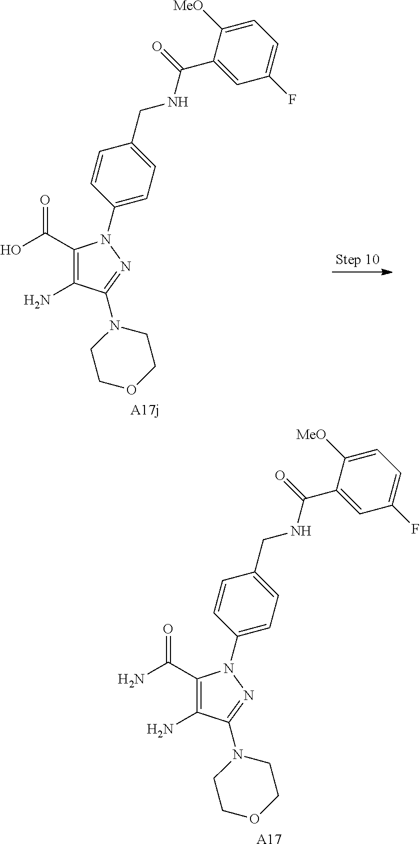

### Step 1: 4-(3-bromo-4-nitro-1H-pyrazol-1-yl)benzonitrile A17b

A mixture of 3-bromo-4-nitro-1H-pyrazole A17a (1.8 g, 9.4 mmol), 4-fluorobenzonitrile (2.3 g, 18.7 mmol) and K₂CO₃ (2.58 g, 18.7 mmol) in DMF (40 mL) under nitrogen atmosphere was stirred overnight at 100° C. After cooling down, the mixture was concentrated under vacuum. The residue was purified by short silica gel column chromatography (DCM/EtOAc) to afford crude 4-(3-bromo-4-nitro-1H-pyrazol-1-yl)benzonitrile A17b, which was used for next step without further purification. ¹H NMR (400 MHz, CDCl₃): 8.65 (s, 1H), 7.81-7.76 (m, 4H) ppm.

### Step 2: 4-(3-morpholino-4-nitro-1H-pyrazol-1-yl)benzonitrile A17c

To a solution of crude 4-(3-bromo-4-nitro-1H-pyrazol-1-yl)benzonitrile A17b (9.4 mmol) in DMF (30 mL) under nitrogen atmosphere in sealed tube was added morpholine (3 mL). The resulting mixture was slowly heat to 130° C. and stirred overnight at this temperature. After cooling down, the mixture was concentrated under vacuum. The residue was purified by silica gel column chromatography (hexane/EtOAc) to afford 4-(3-morpholino-4-nitro-1H-pyrazol-1-yl)benzonitrile A17c (980 mg, 35% two steps). ¹H NMR (400 MHz, CDCl₃): 862 (s, 1H), 7.76-7.70 (m, 4H), 3.81 (t, J=4.28 Hz, 4H), 3.34 (t, J=4.64 Hz, 4H) ppm; LCMS: MS m/z (ESI): 300 [M+H]⁺.

### Step 3: 4-(4-amino-3-morpholino-1H-pyrazol-1-yl)benzonitrile A17d

A mixture of 4-(3-morpholino-4-nitro-1H-pyrazol-1-yl)benzonitrile A17c (980 mg, 3.3 mmol) and Pd/C (150 mg) in EtOAc under hydrogen balloon was stirred overnight at room temperature. The reaction mixture was filtered through Celite, and the filtrate was concentrated under vacuum to afford crude 4-(4-amino-3-morpholino-1H-pyrazol-1-yl)benzonitrile A17d, which was for next step without further purification. LCMS: MS m/z (ESI): 270 [M+H]⁺.

### Step 4: tert-butyl (tert-butoxycarbonyl)(1-(4-cyanophenyl)-3-morpholino-1H-pyrazol-4-yl)carbamate A17e

To a solution of 4-(4-amino-3-morpholino-1H-pyrazol-1-yl)benzonitrile A17d (crude, 3.3 mmol) in THE (100 mL) was added DMAP (100 mg), TEA (2 mL) and (Boc)₂O (3 mL). The resulting mixture was stirred at 50° C. for 2 hours. After cooling down, the mixture was concentrated under vacuum. The residue was purified by silica gel column chromatography (DCM/EtOAc) to afford impure tert-butyl (tert-butoxycarbonyl)(1-(4-cyanophenyl)-3-morpholino-1H-pyrazol-4-yl)carbamate A17e. LCMS: MS m/z (ESI): 470 [M+H]⁺.

### Step 5: methyl 4-(bis(tert-butoxycarbonyl)amino)-1-(4-cyanophenyl)-3-morpholino-1H-pyrazole-5-carboxylate A17f

To a solution of tert-butyl (tert-butoxycarbonyl)(1-(4-cyanophenyl)-3-morpholino-1H-pyrazol-4-yl)carbamate A17e (230 mg, 0.49 mmol) in THE (20 mL) at −78° C. under nitrogen atmosphere was added LiHMDS dropwise (1 M in THF, 1.96 mL, 1.96 mmol). The resulting mixture was stirred at −78° C. for 30 min before slow addition of methyl chloroformate (0.76 mL, 9.8 mmol). The resulting mixture was slowly warmed up to room temperature and then concentrated under reduced pressure. The residue was purified silica gel column chromatography (DCM/EtOAc) to afford methyl 4-(bis(tert-butoxycarbonyl)amino)-1-(4-cyanophenyl)-3-morpholino-1H-pyrazole-5-carboxylate A17f (180 mg, 69% yield). ¹H NMR (400 MHz, MeOD): δ7.73 (d, J=8.12 Hz, 2H), 7.50 (d, J=8 Hz, 2H), 3.68-3.67 (m, 7H), 3.13 (t, J=4.52 Hz, 4H), 1.36 (s, 18H) ppm; LCMS: MS m/z (ESI): 528 [M+H]⁺.

### Step 6: methyl 1-(4-(aminomethyl)phenyl)-4-(bis(tert-butoxycarbonyl)amino)-3-morpholino-1H-pyrazole-5-carboxylate A17 g

A mixture of methyl 4-(bis(tert-butoxycarbonyl)amino)-1-(4-cyanophenyl)-3-morpholino-1H-pyrazole-5-carboxylate A17f (180 mg, 0.34 mmol), Pd/C (100 mg), HCl (1 mL) in EtOH (5 mL) under hydrogen balloon was stirred at room temperature for 1 hour. The reaction mixture was filtered through Celite, and the filtrate was concentrated under vacuum to afford crude methyl 1-(4-(aminomethyl)phenyl)-4-(bis(tert-butoxycarbonyl)amino)-3-morpholino-1H-pyrazole-5-carboxylate A17 g, which was for next step without further purification.

### Step 7: methyl 4-amino-1-(4-(aminomethyl)phenyl)-3-morpholino-1H-pyrazole-5-carboxylate A17 h

To a solution of crude methyl 1-(4-(aminomethyl)phenyl)-4-(bis(tert-butoxycarbonyl)amino)-3-morpholino-1H-pyrazole-5-carboxylate A17 g (crude, 0.34 mmol) in MeOH (10 mL) was added HCl (1 mL). The resulting mixture was stirred at room temperature for 1 hour, and then concentrated under vacuum to afford crude methyl 4-amino-1-(4-(aminomethyl)phenyl)-3-morpholino-1H-pyrazole-5-carboxylate A17 h, which was for next step without further purification. LCMS: MS m/z (ESI): 315 [M+H−H₂O]⁺.

### Step 8: methyl 4-amino-1-(4-((5-fluoro-2-methoxybenzamido)methyl)phenyl)-3-morpholino-1H-pyrazole-5-carboxylate A17i

To a solution of 2-cyclopropoxy-5-fluorobenzoic acid (100 mg, 0.59 mmol) in DCM (10 mL) and DMF (0.5 mL) under nitrogen atmosphere at room temperature was added HATU (224 mg, 0.59 mmol). The resulting mixture was stirred at room temperature for 20 min followed by addition of methyl 4-amino-1-(4-(aminomethyl)phenyl)-3-morpholino-1H-pyrazole-5-carboxylate A17 h (crude, 0.34 mmol) and TEA (1.36 mL, 9.8 mmol). The resulting mixture was stirred at room temperature for 30 min, and then concentrated under vacuum. The resulting residue was purified by silica gel column chromatography (DCM/EtOAc) to afford methyl 4-amino-1-(4-((5-fluoro-2-methoxybenzamido)methyl)phenyl)-3-morpholino-1H-pyrazole-5-carboxylate A17i (68 mg, 42% yield over three steps). LCMS: MS m/z (ESI): 484 [M+H]⁺.

### Step 9: 4-amino-1-(4-((5-fluoro-2-methoxybenzamido)methyl)phenyl)-3-morpholino-1H-pyrazole-5-carboxylic acid A17j

To a solution of methyl 4-amino-1-(4-((5-fluoro-2-methoxybenzamido)methyl)phenyl)-3-morpholino-1H-pyrazole-5-carboxylate A17i (12 mg, 0.025 mmol) in MeOH (10 mL) was added NaOH (10% in water, 4 mL). The resulting mixture was stirred at room temperature for 1 hour. After concentrated under vacuum (dry loading), The residue was purified by silica gel column chromatography (DCM:EtOAc with 10% NH₃ in MeOH) to afford crude 4-amino-1-(4-((5-fluoro-2-methoxybenzamido)methyl)phenyl)-3-morpholino-1H-pyrazole-5-carboxylic acid A17j, which was used for next step without further purification. ¹H NMR (400 MHz, CD₃OD): δ 7.51 (dd, J=9.1, 3.1 Hz, 1H), 7.32 (d, J=7.7 Hz, 2H), 7.21 (d, J=8.1 Hz, 2H), 7.17-7.12 (m, 1H), 7.06 (dd, J=9.08, 4.16 Hz, 1H), 4.54 (s, 2H), 3.85 (s, 3H), 3.73 (t, J=4.28 Hz, 4H), 3.02 (t, J=4.08 Hz, 4H) ppm; LCMS: MS m/z (ESI): 470 [M+H]⁺.

### Step 10: 4-amino-1-(4-((5-fluoro-2-methoxybenzamido)methyl)phenyl)-3-morpholino-1H-pyrazole-5-carboxamide A17

To a solution of 4-amino-1-(4-((5-fluoro-2-methoxybenzamido)methyl)phenyl)-3-morpholino-1H-pyrazole-5-carboxylic acid A17j (crude, 0.025 mmol), HATU (10 mg) and NH₄Cl (200 mg) in DMF (2 mL) was added TEA (0.1 mL). The resulting solution was stirred at room temperature for 1 hour. A After concentrated under vacuum, the residue was purified by prep-HPLC, eluting MeCN/H₂O/TFA, to afford 4-amino-1-(4-((5-fluoro-2-methoxybenzamido)methyl)phenyl)-3-morpholino-1H-pyrazole-5-carboxamide A17 (4.8 mg, 40% yield). LCMS: MS m/z (ESI): 469 [M+H]⁺.

### Example A18

### 4-amino-1-(4-((5-fluoro-2-methoxybenzamido)methyl)phenyl)-3-(trifluoromethyl)-1H-pyrazole-5-carboxamide A18

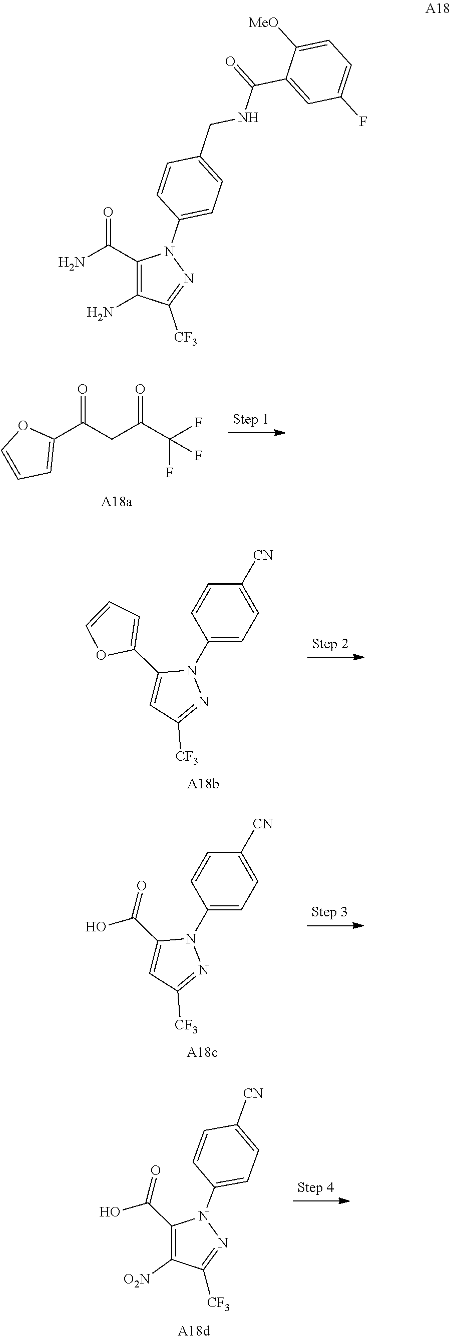 [SMILES: COc1ccc(F)cc1C(=O)NCc1ccc(-n2nc(C(F)(F)F)c(N)c2C(N)=O)cc1 ; O=C(CC(=O)C(F)(F)F)c1ccco1 ; N#Cc1ccc(-n2nc(C(F)(F)F)cc2-c2ccco2)cc1 ; N#Cc1ccc(-n2nc(C(F)(F)F)cc2C(=O)O)cc1 ; N#Cc1ccc(-n2nc(C(F)(F)F)c([N+](=O)[O-])c2C(=O)O)cc1]

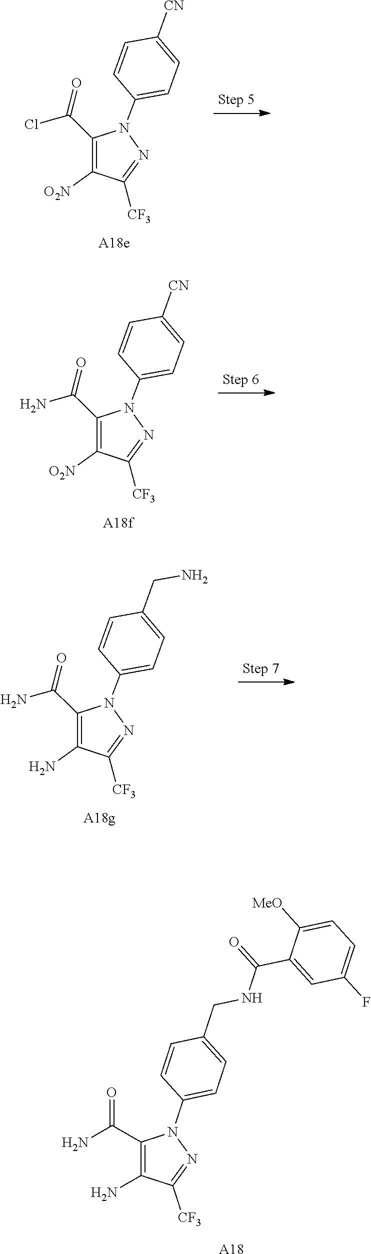 [SMILES: N#Cc1ccc(-n2nc(C(F)(F)F)c([N+](=O)[O-])c2C(=O)Cl)cc1 ; N#Cc1ccc(-n2nc(C(F)(F)F)c([N+](=O)[O-])c2C(N)=O)cc1 ; NCc1ccc(-n2nc(C(F)(F)F)c(N)c2C(N)=O)cc1 ; COc1ccc(F)cc1C(=O)NCc1ccc(-n2nc(C(F)(F)F)c(N)c2C(N)=O)cc1 ; "A18"]

### Step 1: 4-(5-(furan-2-yl)-3-(trifluoromethyl)-1H-pyrazol-1-yl)benzonitrile A18b

To a solution of 4,4,4-trifluoro-1-(furan-2-yl)butane-1,3-dione A18a (30 g, 145.55 mmol) in EtOH (100 mL) under nitrogen atmosphere was added 4-hydrazinobenzonitrile (19.38 g, 145.55 mmol). The resulting mixture was at 90° C. for 7 hours. After cooling down, the reaction was quenched by water (200 mL) and extracted with EtOAc (200 mL×3). The combined organic extracts were washed with brine (200 mL), dried over anhydrous Na₂SO₄, filtered and concentrated under reduced pressure. The residue was purified by silica gel column chromatography (EtOAc:PE=0:1 to 1:3) to afford 4-(5-(furan-2-yl)-3-(trifluoromethyl)-1H-pyrazol-1-yl)benzonitrile A18b (40 g, 90.63% yield). ¹H NMR (400 MHz, DMSO-d₆): δ 8.09-8.00 (m, 2H), 7.78 (s, 1H), 7.75-7.69 (m, 2H), 7.36 (s, 1H), 6.61 (dd, 1H), 6.57 (d, 1H) ppm.

### Step 2: 1-(4-cyanophenyl)-3-(trifluoromethyl)-1H-pyrazole-5-carboxylic acid A18c

To a solution of 4-(5-(furan-2-yl)-3-(trifluoromethyl)-1H-pyrazol-1-yl)benzonitrile A18b (20 g, 65.95 mmol) in CCl₄ (200 mL), MeCN (200 mL) and water (300 mL) under nitrogen atmosphere was added NaIO₄ (84.64 g, 395.73 mmol) and RuCl₃ (1.37 g, 6.60 mmol). The resulting mixture was stirred at room temperature for 16 hours, and then the pH of the solution was adjusted to 3 with HCl. The reaction mixture was diluted with EtOAc (200 mL) and extracted with water (200 mL). The combined organic extracts were washed with brine (200 mL), dried over anhydrous Na₂SO₄, filtered and concentrated under reduced pressure to afford crude 1-(4-cyanophenyl)-3-(trifluoromethyl)-1H-pyrazole-5-carboxylic acid A18c (16 g, 86.27% yield), which was used for next step without further purification. ¹H NMR (400 MHz, DMSO-d₆): δ 8.03 (br, 2H), 7.83 (br, 2H), 7.56 (br, 1H). LCMS: MS m/z (ESI): 561 [2M−H]⁻.

### Step 3: 1-(4-cyanophenyl)-4-nitro-3-(trifluoromethyl)-1H-pyrazole-5-carboxylic acid A18d

To a solution of 1-(4-cyanophenyl)-3-(trifluoromethyl)-1H-pyrazole-5-carboxylic acid A18c (500 mg, 1.78 mmol) in TFAA (8 mL) and TFA (8 mL) under nitrogen atmosphere was added fuming nitric acid (8 mL). The resulting mixture was slowly warmed up and stirred at 80° C. for 1 hour. After cooling down, the mixture was concentrated under vacuum (dry loading), the residue was purified by silica gel column chromatography (DCM:EtOAc with 10% NH₃ in MeOH) to afford 1-(4-cyanophenyl)-4-nitro-3-(trifluoromethyl)-1H-pyrazole-5-carboxylic acid A18d (280 mg, 48% yield). ¹H NMR (400 MHz, MeOD): 7.88 (d, J=8.5 Hz, 2H), 7.79 (d, J=8.5 Hz, 2H) ppm; LCMS: MS m/z (ESI): 651 [2M−H]⁻.

### Step 4: 1-(4-cyanophenyl)-4-nitro-3-(trifluoromethyl)-1H-pyrazole-5-carbonyl chloride A18e

To a solution of 1-(4-cyanophenyl)-4-nitro-3-(trifluoromethyl)-1H-pyrazole-5-carboxylic acid A18d (100 mg, 0.31 mmol) in DMF (4 mL) under nitrogen atmosphere was added SO₂Cl₂ (0.4 mL). The resulting solution was stirred at 80° C. for 1 hour. After cooling down, the mixture was concentrated under vacuum to afford crude 1-(4-cyanophenyl)-4-nitro-3-(trifluoromethyl)-1H-pyrazole-5-carbonyl chloride A18e, which was used for next step without further purification.

### Step 5: 1-(4-cyanophenyl)-4-nitro-3-(trifluoromethyl)-1H-pyrazole-5-carboxamide A18f

To a solution of crude 1-(4-cyanophenyl)-4-nitro-3-(trifluoromethyl)-1H-pyrazole-5-carbonyl chloride A18e (0.31 mmol) in DMF was added NH₃ in dioxane (0.4 M. 1 mL). The resulting mixture was stirred at room temperature for 30 min before concentrated under vacuum. The residue was directly purified by prep-HPLC, eluting MeCN/H₂O/TFA, to afford 1-(4-cyanophenyl)-4-nitro-3-(trifluoromethyl)-1H-pyrazole-5-carboxamide A18f (21 mg, 21% yield over two steps). ¹H NMR (400 MHz, MeOD): 7.89 (d, J=8.5 Hz, 2H), 7.79 (d, J=8.5 Hz, 2H) ppm; LCMS: MS m/z (ESI): 650 [2M−H].

### Step 6: 4-amino-1-(4-(aminomethyl)phenyl)-3-(trifluoromethyl)-1H-pyrazole-5-carboxamide A18 g

A mixture of 1-(4-cyanophenyl)-4-nitro-3-(trifluoromethyl)-1H-pyrazole-5-carboxamide A18f (21 mg, 0.065 mmol), Pd/C (25 mg), and 10 drops of HCl in EtOH under hydrogen balloon was stirred for 1 hour at room temperature. The reaction mixture was filtered through Celite, and the filtrate was concentrated under vacuum to afford crude 4-amino-1-(4-(aminomethyl)phenyl)-3-(trifluoromethyl)-1H-pyrazole-5-carboxamide A18 g, which was used for next step without further purification. LCMS: MS m/z (ESI): 300 [M+H]⁺.

### Step 7: 4-amino-1-(4-((5-fluoro-2-methoxybenzamido)methyl)phenyl)-3-(trifluoromethyl)-1H-pyrazole-5-carboxamide A18

A mixture of 5-fluoro-2-methoxybenzoic acid (15 mg) and HATU (45 mg) in DMF (3 mL) was stirred at room temperature for 15 min. 0.3 mL of this reaction mixture was then added to a solution of 4-amino-1-(4-(aminomethyl)phenyl)-3-(trifluoromethyl)-1H-pyrazole-5-carboxamide A18 g (8 mg, 0.027 mmol) in DMF (1 mL) followed by TEA (0.05 mL). The resulting mixture was stirred at room temperature for 30 min before concentrated under vacuum. The resulting solution was purified by prep-HPLC, eluting with MeCN/H₂O/TFA, to afford 4-amino-1-(4-((5-fluoro-2-methoxybenzamido)methyl)phenyl)-3-(trifluoromethyl)-1H-pyrazole-5-carboxamide A18 (4.1 mg, 30% yield). ¹H NMR (400 MHz, MeOD): 7.51 (dd, J=9.24, 3.12 Hz, 1H), 7.43 (d, J=8.28 Hz, 2H), 7.36 (d, J=8.48 Hz, 2H), 7.15-7.13 (m, 1H), 7.07 (dd, J=9.12, 4.16 Hz, 1H), 4.58 (s, 2H), 3.87 (s, 3H) ppm; LCMS: MS m/z (ESI): 452 [M+H]⁺.

### Example A19

### 4-amino-1-(2-ethoxy-6-fluoro-4-((5-fluoro-2-methoxybenzamido)methyl)phenyl)-3-isopropyl-1H-pyrazole-5-carboxamide A19

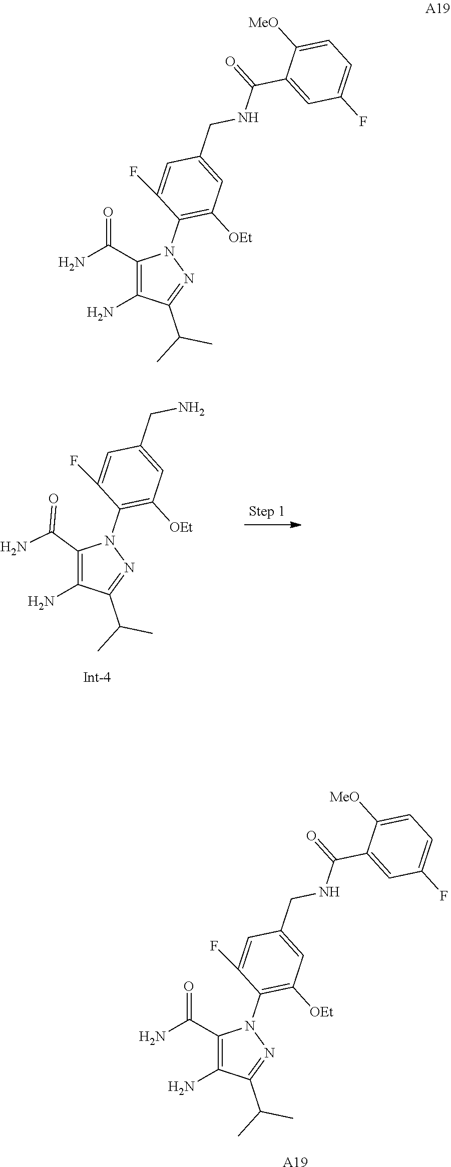

### Step 1: 4-amino-1-(2-ethoxy-6-fluoro-4-((5-fluoro-2-methoxybenzamido)methyl)phenyl)-3-isopropyl-1H-pyrazole-5-carboxamide A19

To a solution of 5-fluoro-2-methoxy-benzoic acid (15 mg) in DMF (3 mL) under nitrogen atmosphere at room temperature was added HATU (45 mg). The resulting mixture was stirred at room temperature for 30 min. 0.3 mL of this reaction mixture was added to a solution of 4-amino-1-(4-(aminomethyl)-2-ethoxy-6-fluorophenyl)-3-isopropyl-1H-pyrazole-5-carboxamide Int-4 (crude, 0.045 mmol) in DMF (1 mL) followed by addition of TEA (0.3 mL). The resulting solution was stirred at room temperature for 30 min and then concentrated under reduced pressure. The resulting residue was purified by prep-HPLC, eluting with MeCN/H₂O/TFA, to afford 4-amino-1-(2-ethoxy-6-fluoro-4-((5-fluoro-2-methoxybenzamido)methyl)phenyl)-3-isopropyl-1H-pyrazole-5-carboxamide A19 (2.4 mg, 11% yield). ¹H NMR (400 MHz, MeOD): δ7.50 (dd, J=9.12, 3.16 Hz, 1H), 7.20-7.15 (m, 1H), 7.10-7.05 (m, 1H), 6.90 (s, 1H), 6.84 (d, J=10.04 Hz, 1H), 4.56 (s, 2H), 3.99 (q, J=6.92 Hz, 2H), 3.88 (s, 3H), 3.03-2.97 (m, 1H), 1.22 (d, J=6.92 Hz, 6H), 1.15 (t, J=6.96 Hz, 3H) ppm; LCMS: MS m/z (ESI): 488 [M+H]⁺.

### Example A20

### 4-amino-1-(2,6-difluoro-4-((5-fluoro-2-methoxybenzamido)methyl)phenyl)-3-(1,1,1-trifluoropropan-2-yl)-1H-pyrazole-5-carboxamide A20

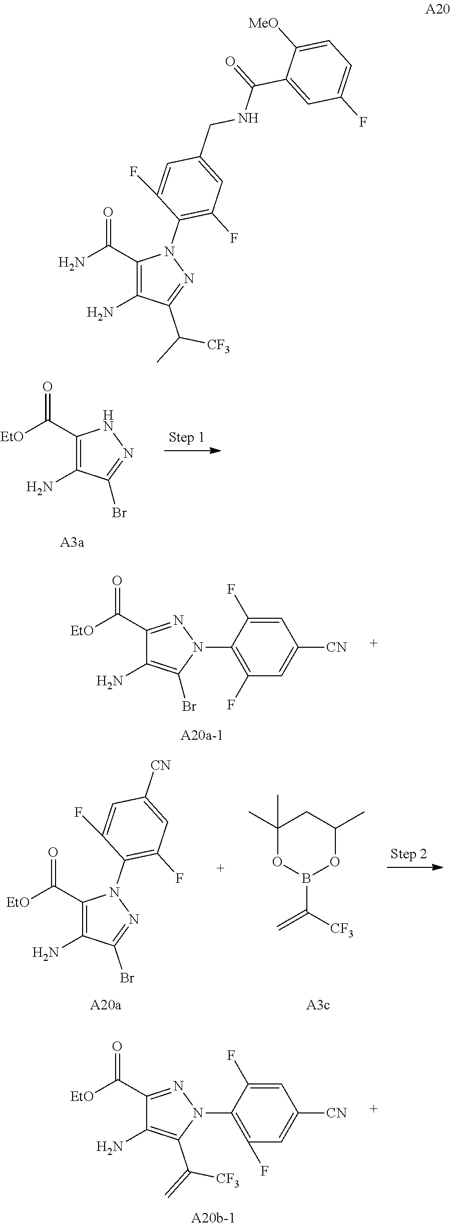

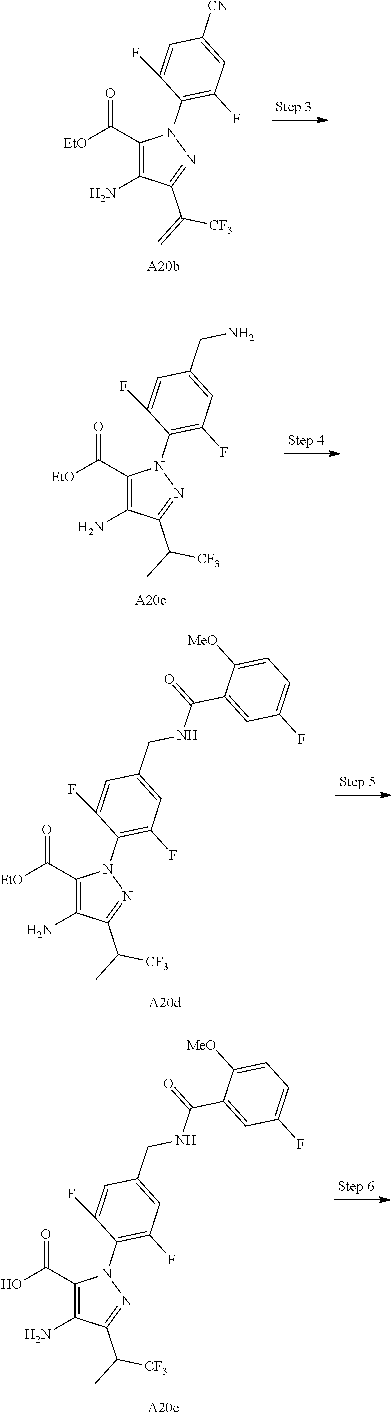 [SMILES: C=C(c1nn(-c2c(F)cc(C#N)cc2F)c(C(=O)OCC)c1N)C(F)(F)F ; CCOC(=O)c1c(N)c(C(C)C(F)(F)F)nn1-c1c(F)cc(CN)cc1F ; CCOC(=O)c1c(N)c(C(C)C(F)(F)F)nn1-c1c(F)cc(CNC(=O)c2cc(F)ccc2OC)cc1F ; COc1ccc(F)cc1C(=O)NCc1cc(F)c(-n2nc(C(C)C(F)(F)F)c(N)c2C(=O)O)c(F)c1]

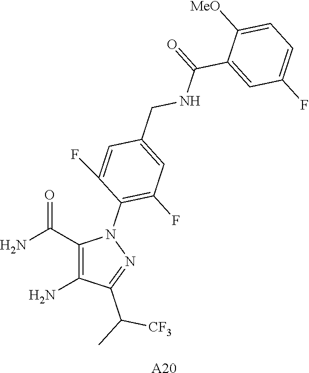

### Step 1: ethyl 4-amino-3-bromo-1-(4-cyano-2,6-difluorophenyl)-1H-pyrazole-5-carboxylate A20a and ethyl 4-amino-5-bromo-1-(4-cyano-2,6-difluorophenyl)-1H-pyrazole-3-carboxylate A20a-1

A mixture of ethyl 4-amino-3-bromo-1H-pyrazole-5-carboxylate A3a (914 mg, 3.90 mmol), 3,4,5-trifluorobenzonitrile (737 mg, 4.69 mmol) and K₂CO₃ (1.61 g, 11.7 mmol) in DMF (30 mL) under nitrogen atmosphere was stirred overnight at room temperature before concentrated under vacuum. The residue was purified by silica gel column chromatography (DCM/EtOAc) to afford ethyl 4-amino-3-bromo-1-(4-cyano-2,6-difluorophenyl)-1H-pyrazole-5-carboxylate A20a and ethyl 4-amino-5-bromo-1-(4-cyano-2,6-difluorophenyl)-1H-pyrazole-3-carboxylate A20a-1 as a mixture, which was used for next step without further purification. LCMS: MS m/z (ESI): 371.0 [M+H]⁺.

### Step 2: ethyl 4-amino-1-(4-cyano-2,6-difluorophenyl)-3-(3,3,3-trifluoroprop-1-en-2-yl)-1H-pyrazole-5-carboxylate A20b and ethyl 4-amino-1-(4-cyano-2,6-difluorophenyl)-5-(3,3,3-trifluoroprop-1-en-2-yl)-1H-pyrazole-3-carboxylate A20b-1

A mixture of ethyl 4-amino-3-bromo-1-(4-cyano-2,6-difluorophenyl)-1H-pyrazole-5-carboxylate A20a and ethyl 4-amino-5-bromo-1-(4-cyano-2,6-difluorophenyl)-1H-pyrazole-3-carboxylate A20a-1 (1.55 mmol), 1-(trifluoromethyl)vinylboronic acid hexylene glycol ester A3c (1 688 mg, 3.1 mmol), Pd(dppf)Cl₂ (113 mg, 0.155 mmol) and Cs₂CO₃ (1.5 g, 4.65 mmol) in dioxane:H₂O (10:1, 10 mL) under nitrogen atmosphere was stirred overnight at 90° C. After cooling down, the mixture was filtered through a silica pad, concentrated under vacuum. The resulting residue was purified by prep-HPLC, eluting with MeCN/H₂O/TFA, to afford ethyl 4-amino-1-(4-cyano-2,6-difluorophenyl)-3-(3,3,3-trifluoroprop-1-en-2-yl)-1H-pyrazole-5-carboxylate A20b (less polar, 106 mg). LCMS: MS m/z (ESI): 387.0 [M+H]⁺.

### Step 3: ethyl 4-amino-1-(4-(aminomethyl)-2,6-difluorophenyl)-3-(1,1,1-trifluoropropan-2-yl)-1H-pyrazole-5-carboxylate A20c

A mixture of ethyl 4-amino-1-(4-cyano-2,6-difluorophenyl)-3-(3,3,3-trifluoroprop-1-en-2-yl)-1H-pyrazole-5-carboxylate A20b (106 mg, 0.275 mmol), Pd/C (80 mg), and 10 drops of HCl in EtOH under hydrogen balloon was stirred for 4 hours at room temperature. The reaction mixture was filtered through Celite, and the filtrate was concentrated under vacuum to afford crude ethyl 4-amino-1-(4-(aminomethyl)-2,6-difluorophenyl)-3-(1,1,1-trifluoropropan-2-yl)-1H-pyrazole-5-carboxylate A20c, which was for next step without further purification. LCMS: MS m/z (ESI): 393.0 [M+H]⁺.

### Step 4: ethyl 4-amino-1-(2,6-difluoro-4-((5-fluoro-2-methoxybenzamido)methyl)phenyl)-3-(1,1,1-trifluoropropan-2-yl)-1H-pyrazole-5-carboxylate A20d

To a solution of 5-fluoro-2-methoxybenzoic acid (42 mg, 0.275 mmol) in DMF (3 mL) under nitrogen atmosphere at room temperature was added HATU (105 mg, 0.275 mmol). The mixture was stirred at room temperature for 0.5 h, and then ethyl 4-amino-1-(4-(aminomethyl)-2,6-difluorophenyl)-3-(1,1,1-trifluoropropan-2-yl)-1H-pyrazole-5-carboxylate A20c (0.275 mmol) and DIPEA (0.05 mL) were added to the reaction mixture. The resulting mixture was stirred at room temperature for 1.5 hours. The resulting solution was purified by prep-HPLC, eluting with MeCN/H₂O/TFA, to afford ethyl 4-amino-1-(2,6-difluoro-4-((5-fluoro-2-methoxybenzamido)methyl)phenyl)-3-(1,1,1-trifluoropropan-2-yl)-1H-pyrazole-5-carboxylate A20d (51 mg, 34% yield two steps). LCMS: MS m/z (ESI): 545.0 [M+H]⁺.

### Step 5: 4-amino-1-(2,6-difluoro-4-((5-fluoro-2-methoxybenzamido)methyl)phenyl)-3-(1,1,1-trifluoropropan-2-yl)-1H-pyrazole-5-carboxylic acid A20e

To a solution of ethyl 4-amino-1-(2,6-difluoro-4-((5-fluoro-2-methoxybenzamido)methyl)phenyl)-3-(1,1,1-trifluoropropan-2-yl)-1H-pyrazole-5-carboxylate A20d (51 mg, 0.093 mmol) in MeOH (4 mL) was added NaOH (10% in water, 5 mL). The resulting mixture was stirred at room temperature for 3 hours. After concentrated under vacuum (dry loading), The residue was purified by silica gel column chromatography (DCM:EtOAc with 10% NH₃ in MeOH) to afford 4-amino-1-(2,6-difluoro-4-((5-fluoro-2-methoxybenzamido)methyl)phenyl)-3-(1,1,1-trifluoropropan-2-yl)-1H-pyrazole-5-carboxylic acid A20e (32 mg, 63% yield). LCMS: MS m/z (ESI): 517.0 [M+H]⁺.

### Step 6: 4-amino-1-(2,6-difluoro-4-((5-fluoro-2-methoxybenzamido)methyl)phenyl)-3-(1,1,1-trifluoropropan-2-yl)-1H-pyrazole-5-carboxamide A20

A solution of 4-amino-1-(2,6-difluoro-4-((5-fluoro-2-methoxybenzamido)methyl)phenyl)-3-(1,1,1-trifluoropropan-2-yl)-1H-pyrazole-5-carboxylic acid A20e (32 mg, 0.062 mmol), HATU (45 mg), NH₄Cl (600 mg), and TEA (0.15 mL) in DMF (2 mL) was stirred overnight at room temperature. The residue was directly purified by prep-HPLC, eluting MeCN/H₂O/TFA, to afford 4-amino-1-(2,6-difluoro-4-((5-fluoro-2-methoxybenzamido)methyl)phenyl)-3-(1,1,1-trifluoropropan-2-yl)-1H-pyrazole-5-carboxamide A20 (7 mg, 22% yield). ¹H NMR (400 MHz, MeOD) δ 7.58-7.46 (m, 1H), 7.25-7.15 (m, 1H), 7.15-7.00 (m, 3H), 4.60 (s, 2H), 3.90 (s, 3H), 3.78-3.67 (m, 1H), 1.44 (d, J=7.2 Hz, 3H) ppm; LCMS: MS m/z (ESI): 516.0 [M+H]⁺.

### Example A21

### 4-amino-1-(4-((5-fluoro-2-methoxybenzamido)methyl)phenyl)-3-(4-hydroxycyclohexyl)-1H-pyrazole-5-carboxamide A21

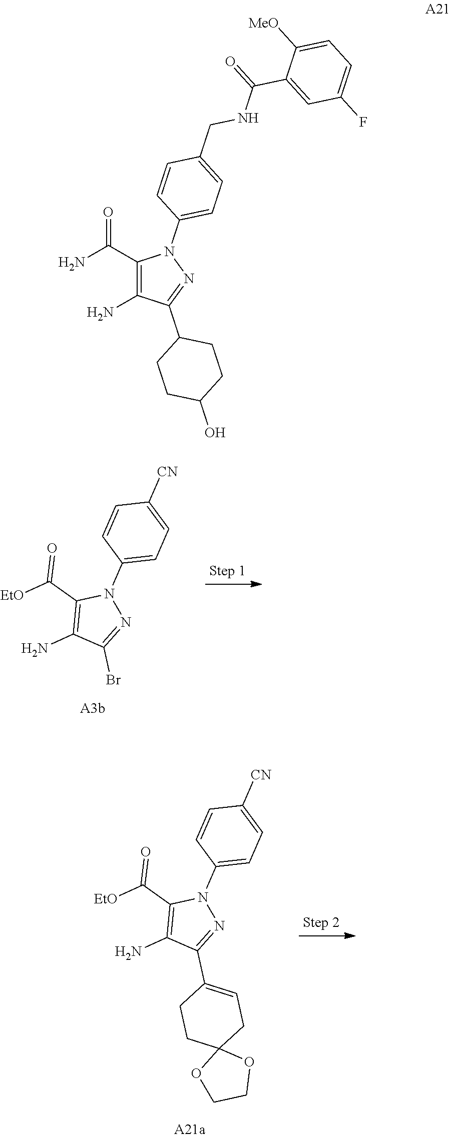 [SMILES: COc1ccc(F)cc1C(=O)NCc1ccc(-n2nc(C3CCC(O)CC3)c(N)c2C(N)=O)cc1 ; CCOC(=O)c1c(N)c(Br)nn1-c1ccc(C#N)cc1 ; CCOC(=O)c1c(N)c(C2=CCC3(CC2)OCCO3)nn1-c1ccc(C#N)cc1]

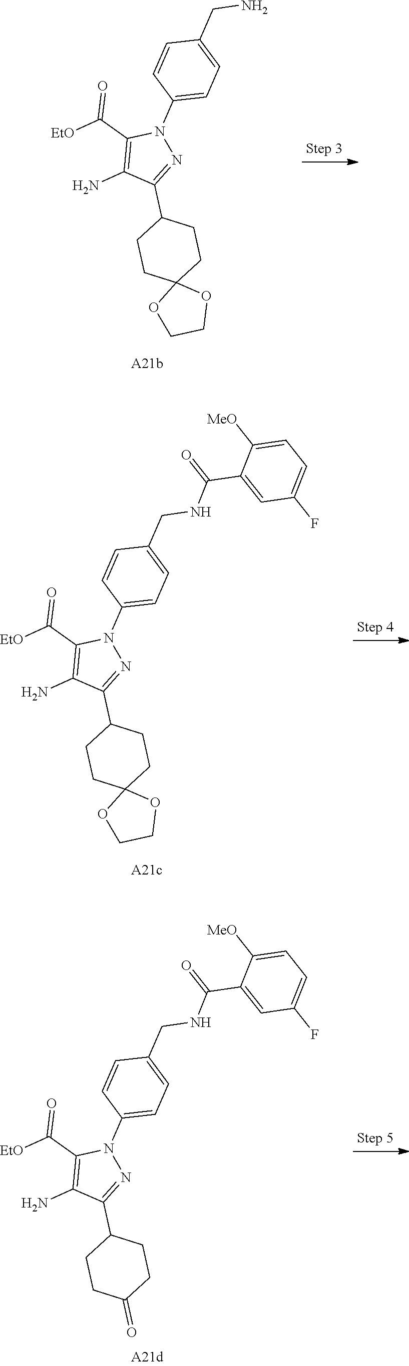 [SMILES: CCOC(=O)c1c(N)c(C2CCC3(CC2)OCCO3)nn1-c1ccc(CN)cc1 ; CCOC(=O)c1c(N)c(C2CCC3(CC2)OCCO3)nn1-c1ccc(CNC(=O)c2cc(F)ccc2OC)cc1 ; CCOC(=O)c1c(N)c(C2CCC(=O)CC2)nn1-c1ccc(CNC(=O)c2cc(F)ccc2OC)cc1]

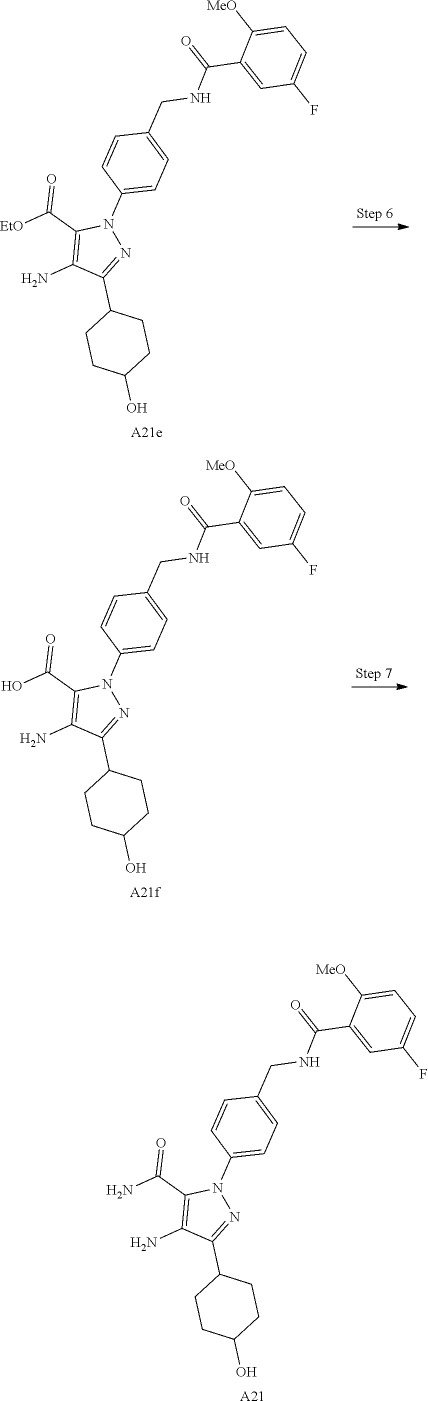

### Step 1: ethyl 4-amino-1-(4-cyanophenyl)-3-(1,4-dioxaspiro[4.5]dec-7-en-8-yl)-1H-pyrazole-5-carboxylate A21a

A mixture of ethyl 4-amino-3-bromo-1-(4-cyanophenyl)-1H-pyrazole-5-carboxylate A3b (1.5 g, 4.5 mmol), 4,4,5,5-tetramethyl-2-(1,4-dioxaspiro[4.5]dec-7-en-8-yl)-1,3,2-dioxaborolane (2.4 g, 9.0 mmol), Pd(dppf)C12 (330 mg, 0.45 mmol) and Cs₂CO₃ (14.4 g, 13.5 mmol) in dioxane:H ₂O (10:1, 22 mL) under nitrogen atmosphere was stirred overnight at 90° C. After cooling down, the mixture was filtered through a silica pad, concentrated under vacuum. The resulting residue was purified by prep-HPLC, eluting with MeCN/H₂O/TFA, to afford ethyl 4-amino-1-(4-cyanophenyl)-3-(1,4-dioxaspiro[4.5]dec-7-en-8-yl)-1H-pyrazole-5-carboxylate A21a (1.06 g, 60% yield). LCMS: MS m/z (ESI): 395.0 [M+H]⁺.

### Step 2: ethyl 4-amino-1-(4-(aminomethyl)phenyl)-3-(1,4-dioxaspiro[4.5]decan-8-yl)-1H-pyrazole-5-carboxylate A21b

A mixture of ethyl 4-amino-1-(4-cyanophenyl)-3-(1,4-dioxaspiro[4.5]dec-7-en-8-yl)-1H-pyrazole-5-carboxylate A21a (480 mg, 1.2 mmol), Pd/C (240 mg), and 10 drops of HCl in EtOH under hydrogen balloon was stirred for 4 hours at room temperature. The reaction mixture was filtered through Celite, and the filtrate was concentrated under vacuum to afford crude ethyl 4-amino-1-(4-(aminomethyl)phenyl)-3-(1,4-dioxaspiro[4.5]decan-8-yl)-1H-pyrazole-5-carboxylate A21b, which was for next step without further purification. LCMS: MS m/z (ESI): 401.0 [M+H]⁺.

### Step 3: ethyl 4-amino-1-(4-((5-fluoro-2-methoxybenzamido)methyl)phenyl)-3-(1,4-dioxaspiro[4.5]decan-8-yl)-1H-pyrazole-5-carboxylate A21c

To a solution of 5-fluoro-2-methoxybenzoic acid (187 mg, 1.1 mmol) in DMF (3 mL) under nitrogen atmosphere at room temperature was added HATU (627 mg, 1.65 mmol). The mixture was stirred at room temperature for 0.5 h, and then ethyl 4-amino-1-(4-(aminomethyl)phenyl)-3-(1,4-dioxaspiro[4.5]decan-8-yl)-1H-pyrazole-5-carboxylate A21b (1.1 mmol) and DIPEA (1.9 mL) were added to the reaction mixture. The resulting mixture was stirred at room temperature for 1.5 hours. The resulting solution was purified by prep-HPLC, eluting with MeCN/H₂O/TFA, to afford ethyl 4-amino-1-(4-((5-fluoro-2-methoxybenzamido)methyl)phenyl)-3-(1,4-dioxaspiro[4.5]decan-8-yl)-1H-pyrazole-5-carboxylate A21c (78 mg, 17% yield two steps). LCMS: MS m/z (ESI): 553.0 [M+H]⁺.

### Step 4: ethyl 4-amino-1-(4-((5-fluoro-2-methoxybenzamido)methyl)phenyl)-3-(4-oxocyclohexyl)-1H-pyrazole-5-carboxylate A21d

To a solution of ethyl 4-amino-1-(4-((5-fluoro-2-methoxybenzamido)methyl)phenyl)-3-(1,4-dioxaspiro[4.5]decan-8-yl)-1H-pyrazole-5-carboxylate A21c (78 mg, 0.141 mmol) in MeOH (4 mL) was added 4N HCl (4 mL). The resulting mixture was stirred at room temperature for 3 hours. After concentrated under vacuum, the resulting solution was purified by prep-HPLC, eluting with MeCN/H₂O/TFA, to afford ethyl 4-amino-1-(4-((5-fluoro-2-methoxybenzamido)methyl)phenyl)-3-(4-oxocyclohexyl)-1H-pyrazole-5-carboxylate A21d (13 mg, 18% yield). LCMS: MS m/z (ESI): 509.0 [M+H]⁺.

### Step 5: ethyl 4-amino-1-(4-((5-fluoro-2-methoxybenzamido)methyl)phenyl)-3-(4-hydroxycyclohexyl)-1H-pyrazole-5-carboxylate A21e

To a solution of ethyl 4-amino-1-(4-((5-fluoro-2-methoxybenzamido)methyl)phenyl)-3-(4-oxocyclohexyl)-1H-pyrazole-5-carboxylate A21d (13 mg, 0.026 mmol) in MeOH (3 mL) at 0° C. was added NaBH₄ (3 mg). The resulting solution was slowly warmed up to room temperature and stirred for 2 hours. After concentrated under vacuum, the resulting residue was purified by prep-HPLC, eluting with MeCN/H₂O/TFA, to afford ethyl 4-amino-1-(4-((5-fluoro-2-methoxybenzamido)methyl)phenyl)-3-(4-hydroxycyclohexyl)-1H-pyrazole-5-carboxylate A21e (7 mg, 52% yield). LCMS: MS m/z (ESI): 511.0 [M+H]⁺.

### Step 6: 4-amino-1-(4-((5-fluoro-2-methoxybenzamido)methyl)phenyl)-3-(4-hydroxycyclohexyl)-1H-pyrazole-5-carboxylic acid A21f

To a solution of ethyl 4-amino-1-(4-((5-fluoro-2-methoxybenzamido)methyl)phenyl)-3-(4-hydroxycyclohexyl)-1H-pyrazole-5-carboxylate A21e (7 mg) in MeOH (4 mL) was added NaOH (10% in water, 5 mL). The resulting mixture was stirred at room temperature for 3 hours. After concentrated under vacuum (dry loading), The residue was purified by prep-HPLC, eluting with MeCN/H₂O/TFA, to afford 4-amino-1-(4-((5-fluoro-2-methoxybenzamido)methyl)phenyl)-3-(4-hydroxycyclohexyl)-1H-pyrazole-5-carboxylic acid A21f (3 mg, 43% yield). LCMS: MS m/z (ESI): 483.0 [M+H]⁺.

### Step 7: 4-amino-1-(4-((5-fluoro-2-methoxybenzamido)methyl)phenyl)-3-(4-hydroxycyclohexyl)-1H-pyrazole-5-carboxamide A21

A solution of 4-amino-1-(4-((5-fluoro-2-methoxybenzamido)methyl)phenyl)-3-(4-hydroxycyclohexyl)-1H-pyrazole-5-carboxylic acid A21f (3 mg), HATU (5 mg), NH₄Cl (50 mg), and TEA (0.05 mL) in DMF (2 mL) was stirred overnight at room temperature. The residue was directly purified by prep-HPLC, eluting MeCN/H₂O/TFA, to afford 4-amino-1-(4-((5-fluoro-2-methoxybenzamido)methyl)phenyl)-3-(4-hydroxycyclohexyl)-1H-pyrazole-5-carboxamide A21 (0.8 mg, 26% yield). LCMS: MS m/z (ESI): 482.0 [M+H]⁺.

### Example A22

### 4-amino-3-(4,4-difluorocyclohexyl)-1-(4-((5-fluoro-2-methoxybenzamido)methyl)phenyl)-1H-pyrazole-5-carboxamide A22

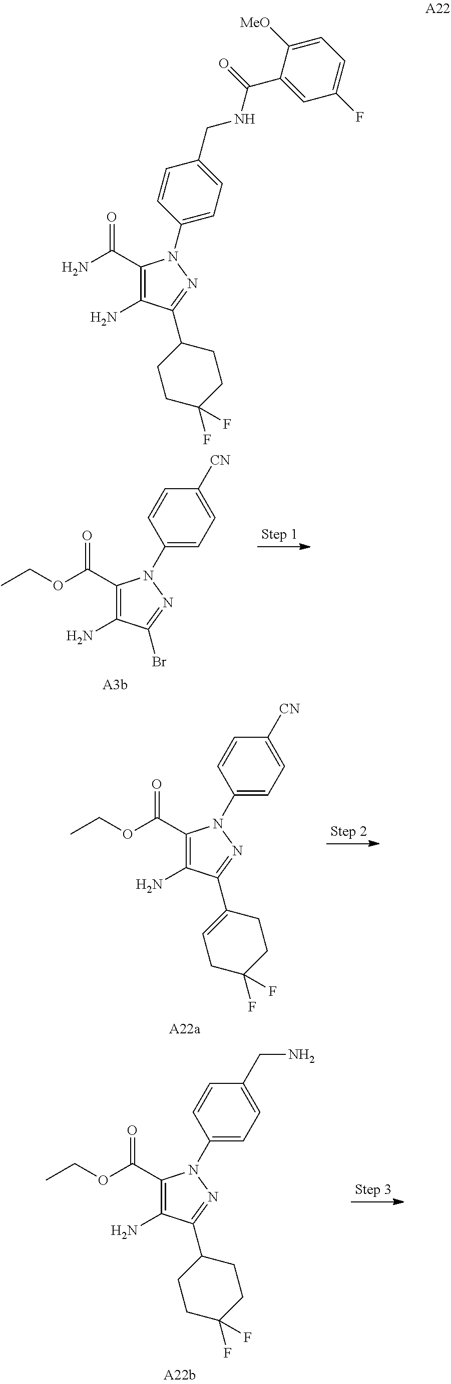

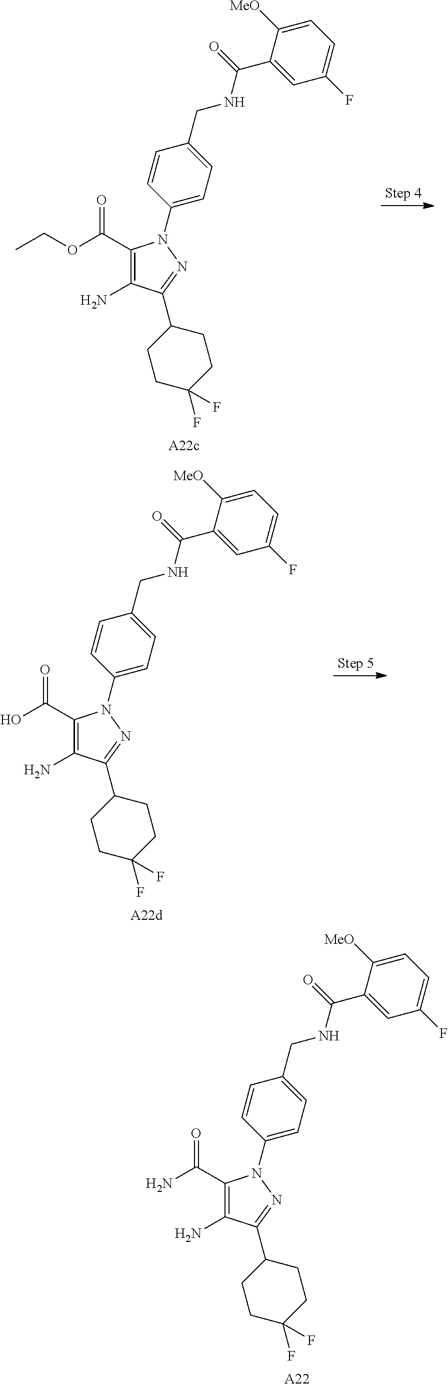

### Step 1: ethyl 4-amino-1-(4-cyanophenyl)-3-(4,4-difluorocyclohex-1-en-1-yl)-1H-pyrazole-5-carboxylate A22a

A mixture of ethyl 4-amino-3-bromo-1-(4-cyanophenyl)-1H-pyrazole-5-carboxylate A3b (450 mg, 1.34 mmol), Pd(dppf)C12 (55.89 mg, 67.13 umol), 2-(4,4-difluorocyclohex-1-en-1-yl)-4,4,5,5-tetramethyl-1,3,2-dioxaborolane (660.86 mg, 2.69 mmol), and K₂CO₃ (370.57 mg, 2.69 mmol) in dioxane (15 mL) and H₂O (3 mL) under nitrogen atmosphere was stirred overnight at 100° C. After cooling down and concentrated under reduced pressure, the resulting residue was purified by silica gel column chromatography (PE/EtOAc=3/1) to afford ethyl 4-amino-1-(4-cyanophenyl)-3-(4,4-difluorocyclohexyl)-1H-pyrazole-5-carboxylate A22a (396 mg, 78.78% yield). LCMS: MS m/z (ESI): 373.1 [M+H]⁺.

### Step 2: ethyl 4-amino-1-(4-(aminomethyl)phenyl)-3-(4,4-difluorocyclohexyl)-1H-pyrazole-5-carboxylate A22b

A mixture of ethyl 4-amino-1-(4-cyanophenyl)-3-(4,4-difluorocyclohex-1-en-1-yl)-1H-pyrazole-5-carboxylate A22a (0.2 g, 534.21 umol), Pd/C (64.88 mg, 534.21 umol), and HCl (12 M, 0.4 mL) in MeOH (4 mL) was stirred under hydrogen atmosphere (15 psi) at room temperature for 16 hours. Then, the mixture was filtered through Celite, and the filtrate was concentrated under reduced pressure to afford crude ethyl 4-amino-1-(4-(aminomethyl)phenyl)-3-(4,4-difluorocyclohexyl)-1H-pyrazole-5-carboxylate A22b (150 mg, 74.20% yield), which was used for next step without further purification. LCMS: MS m/z (ESI): 362.1 [M+1−H₂O]⁻.

### Step 3: ethyl 4-amino-3-(4,4-difluorocyclohexyl)-1-(4-((5-fluoro-2-methoxybenzamido)methyl)phenyl)-1H-pyrazole-5-carboxylate A22c

A mixture of 5-fluoro-2-methoxybenzoic acid (67.44 mg, 396.39 umol), ethyl 4-amino-1-(4-(aminomethyl)phenyl)-3-(4,4-difluorocyclohexyl)-1H-pyrazole-5-carboxylate A22b (150 mg, 396.39 umol), HATU (226.08 mg, 594.59 umol), and DIPEA (129 g, 1.98 mmol) in anhydrous DMF (3 mL) under nitrogen atmosphere was stirred overnight at room temperature. The reaction was quenched by water (10 mL) and then extracted with EtOAc (15 mL×3). The combined organic extracts were washed with brine, dried over anhydrous Na₂SO₄, filtered, and concentrated under reduced pressure. The residue was purification by silica gel column chromatography (DCM/MeOH=50/1) to afford ethyl 4-amino-3-(4,4-difluorocyclohexyl)-1-(4-((5-fluoro-2-methoxybenzamido)methyl)phenyl)-1H-pyrazole-5-carboxylate A22c (100 mg, 47.55% yield). LCMS: MS m/z (ESI): 531.0 [M+H]⁺.

### Step 4: 4-amino-3-(4,4-difluorocyclohexyl)-1-(4-((5-fluoro-2-methoxybenzamido)methyl)phenyl)-1H-pyrazole-5-carboxylic acid A22d

To a solution of ethyl 4-amino-3-(4,4-difluorocyclohexyl)-1-(4-((5-fluoro-2-methoxybenzamido)methyl)phenyl)-1H-pyrazole-5-carboxylate A22c (0.1 g, 188.49 umol) in MeOH (4 mL) at room temperature was added NaOH (3M in water, 4 mL). The resulting mixture was slowly warmed up and stirred overnight at 60° C. After cooling down, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-3-(4,4-difluorocyclohexyl)-1-(4-((5-fluoro-2-methoxybenzamido)methyl)phenyl)-1H-pyrazole-5-carboxylic acid A22d (80 mg, 84.47% yield), which was used for next step without further purification.

### Step 5: 4-amino-3-(4,4-difluorocyclohexyl)-1-(4-((5-fluoro-2-methoxybenzamido)methyl)phenyl)-1H-pyrazole-5-carboxamide A22

A mixture of NH₄Cl (17.03 mg, 318.42 umol), HATU (90.80 mg, 238.81 umol), 4-amino-3-(4,4-difluorocyclohexyl)-1-(4-((5-fluoro-2-methoxybenzamido)methyl)phenyl)-1H-pyrazole-5-carboxylic acid A22d (80 mg, 159.21 umol) and DIPEA (102.69 mg, 796.04 umol) under nitrogen atmosphere was stirred overnight at room temperature. The reaction mixture quenched by water (10 mL), and then extracted with EtOAc (15 mL×3). The combined organic extracts were washed with brine, dried over anhydrous Na₂SO₄, filtered, and concentrated under reduced pressure. The residue was purified by silica gel column chromatography (DCM/MeOH=50/1) to afford 4-amino-3-(4,4-difluorocyclohexyl)-1-(4-((5-fluoro-2-methoxybenzamido)methyl)phenyl)-1H-pyrazole-5-carboxamide A22 (64 mg, 80.16% yield). ¹H NMR (400 MHz, DMSO-d₆): δ 8.84 (t, 1H), 7.52 (dd, 1H), 7.37-7.27 (m, 5H), 7.18 (dd, 1H), 4.59 (s, 2H), 4.52 (d, 2H), 3.89 (s, 3H), 2.89-2.85 (m, 1H), 2.13-2.08 (m, 2H), 1.98-1.87 (m, 4H), 1.72-1.68 (m, 2H) ppm; LCMS: MS m/z (ESI): 502.0 [M+H]⁺.

### Example A23A and A23B

### 1-(4-((5-fluoro-2-methoxybenzamido)methyl)phenyl)-3-isopropyl-4-(methylamino)-1H-pyrazole-5-carboxamide A23A and

### 4-(dimethylamino)-1-(4-((5-fluoro-2-methoxybenzamido)methyl)phenyl)-3-isopropyl-1H-pyrazole-5-carboxamide A23B

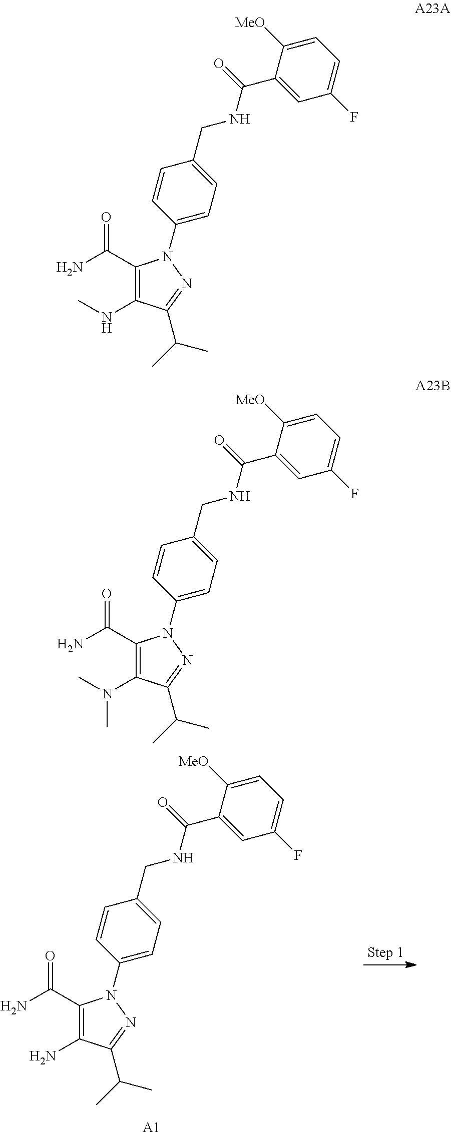

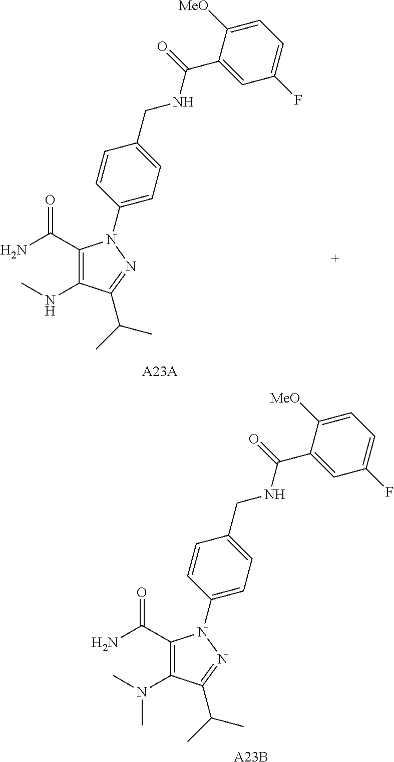

### Step 1: 1-(4-((5-fluoro-2-methoxybenzamido)methyl)phenyl)-3-isopropyl-4-(methylamino)-1H-pyrazole-5-carboxamide A23A and 4-(dimethylamino)-1-(4-((5-fluoro-2-methoxybenzamido)methyl)phenyl)-3-isopropyl-1H-pyrazole-5-carboxamide A23B

To a solution of 4-amino-1-(4-((5-fluoro-2-methoxybenzamido)methyl)phenyl)-3-isopropyl-1H-pyrazole-5-carboxamide A1 (8.5 mg, 0.02 mmol) and TEA (0.06 mg, 0.06 mmol) in DMF (2 mL) was added MeI (8.46 mg, 0.06 mmol). The reaction mixture was stirred at room temperature for 1 hour. The resulting mixture was directly purified by prep-HPLC, eluting with MeCN/H₂O/TFA, to provide 1-(4-((5-fluoro-2-methoxybenzamido)methyl)phenyl)-3-isopropyl-4-(methylamino)-1H-pyrazole-5-carboxamide A23A (2.1 mg, 24% yield) and 4-(dimethylamino)-1-(4-((5-fluoro-2-methoxybenzamido)methyl)phenyl)-3-isopropyl-1H-pyrazole-5-carboxamide A23B (5.6 mg, 62% yield).

1-(4-((5-fluoro-2-methoxybenzamido)methyl)phenyl)-3-isopropyl-4-(methylamino)-1H-pyrazole-5-carboxamide A23A, ¹H NMR (400 MHz, CD₃OD): δ 7.50-7.52 (d, 1H), 7.40-7.42 (d, 2H), 7.33-7.35 (d, 2H), 7.13-7.17 (t, 1H), 7.05-7.08 (d, 1H), 4.57 (s, 2H), 3.86 (s, 3H), 3.00-3.07 (m, 1H), 2.85 (s, 3H), 1.23-1.25 (d, 6H) ppm; LCMS: MS m/z (ESI): 440 [M+H]⁺.

4-(dimethylamino)-1-(4-((5-fluoro-2-methoxybenzamido)methyl)phenyl)-3-isopropyl-1H-pyrazole-5-carboxamide A23B, ¹H NMR (400 MHz, CD₃OD): δ 7.50-7.53 (d, 1H), 7.38-7.39 (d, 2H), 7.30-7.32 (d, 2H), 7.12-7.17 (t, 1H), 7.05-7.08 (d, 1H), 4.57 (s, 2H), 3.86 (s, 3H), 3.10-3.15 (m, 1H), 2.97 (s, 6H), 1.24-1.26 (d, 6H) ppm; LCMS: MS m/z (ESI): 454 [M+H]⁺.

### Example A24

### 4-acetamido-1-(4-((5-fluoro-2-methoxybenzamido)methyl)phenyl)-3-isopropyl-1H-pyrazole-5-carboxamide A24

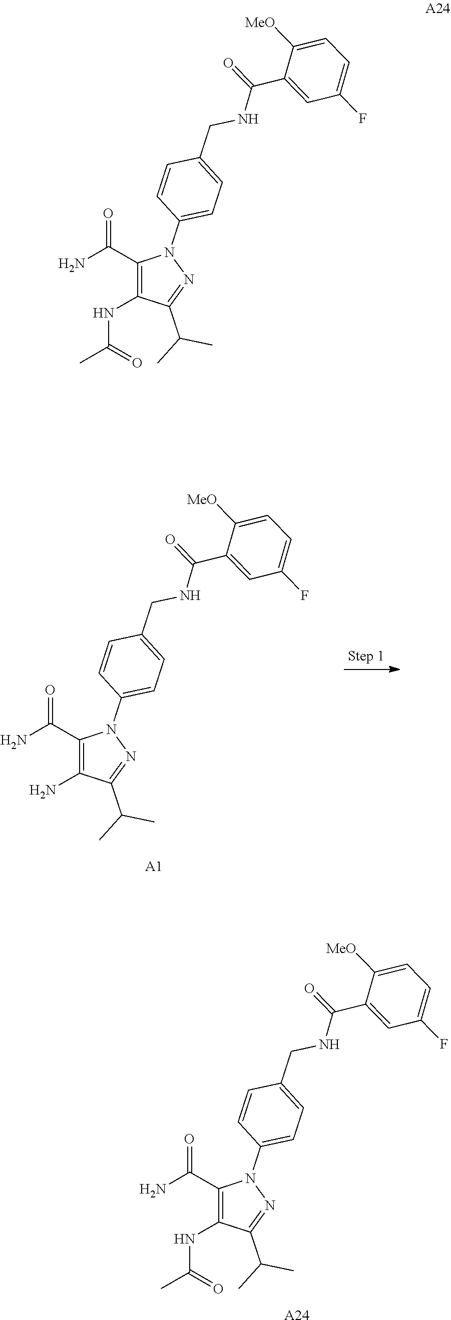

### Step 1: 4-acetamido-1-(4-((5-fluoro-2-methoxybenzamido)methyl)phenyl)-3-isopropyl-1H-pyrazole-5-carboxamide A24

To a solution of 4-amino-1-(4-((5-fluoro-2-methoxybenzamido)methyl)phenyl)-3-isopropyl-1H-pyrazole-5-carboxamide A1 (4.3 mg, 0.01 mmol) and TEA (3.03 mg, 0.03 mmol) in DMF (0.2 mL) was added acetic anhydride (1.5 mg, 0.015 mmol). The reaction mixture was stirred at room temperature for 6 hours. The resulting mixture was directly purified by prep-HPLC, eluting with MeCN/H₂O/TFA, to provide 4-acetamido-1-(4-((5-fluoro-2-methoxybenzamido)methyl)phenyl)-3-isopropyl-1H-pyrazole-5-carboxamide A24 (2 mg, 43% yield). ¹H NMR (400 MHz, CD₃OD): δ δ: 7.49-7.52 (d, 1H), 7.36 (s, 4H), 7.12-7.17 (t, 1H), 7.04-7.07 (d, 1H), 4.55 (s, 2H), 3.85 (s, 3H), 2.87-2.92 (m, 1H), 2.06 (s, 3H), 1.17-1.19 (d, 6H) ppm; LCMS: MS m/z (ESI): 468 [M+H]⁺.

### Example A25

### 4-amino-3-cyano-1-(4-((5-fluoro-2-methoxybenzamido)methyl)phenyl)-1H-pyrazole-5-carboxamide A25

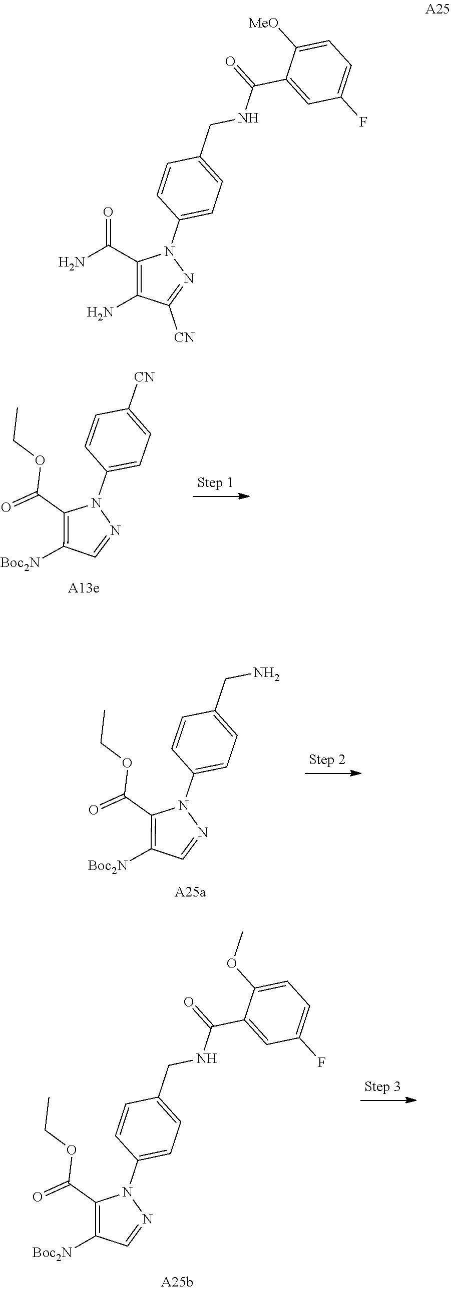

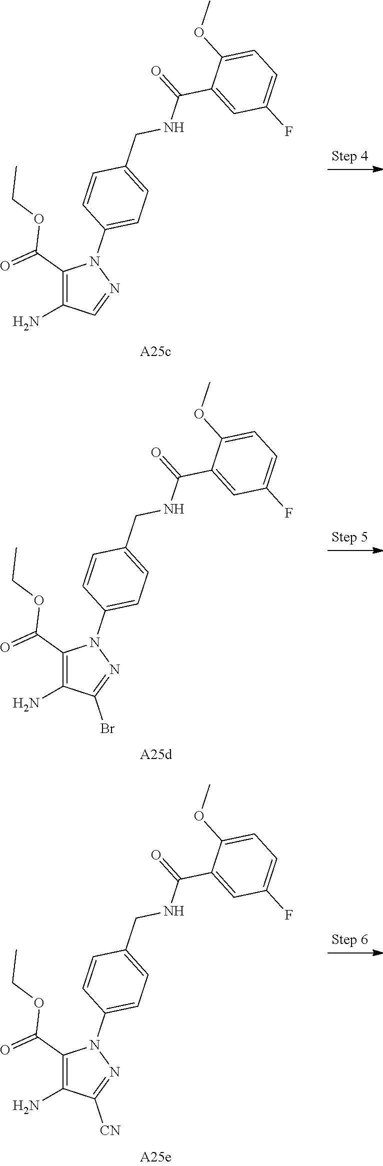 [SMILES: CCOC(=O)c1c(N)cnn1-c1ccc(CNC(=O)c2cc(F)ccc2OC)cc1 ; CCOC(=O)c1c(N)c(Br)nn1-c1ccc(CNC(=O)c2cc(F)ccc2OC)cc1 ; CCOC(=O)c1c(N)c(C#N)nn1-c1ccc(CNC(=O)c2cc(F)ccc2OC)cc1]

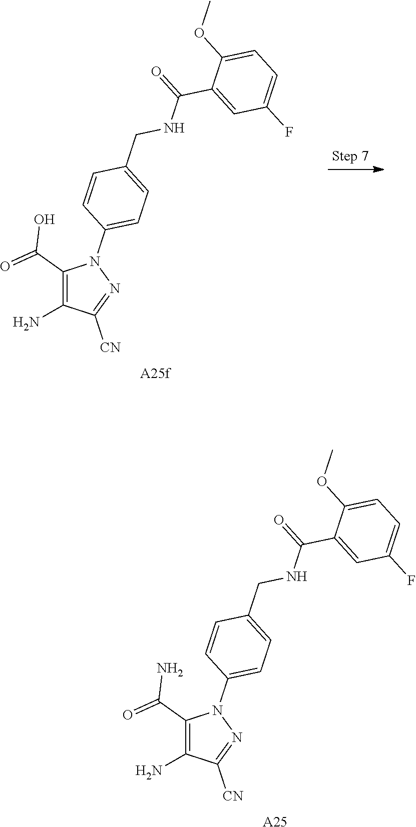 [SMILES: COc1ccc(F)cc1C(=O)NCc1ccc(-n2nc(C#N)c(N)c2C(=O)O)cc1 ; COc1ccc(F)cc1C(=O)NCc1ccc(-n2nc(C#N)c(N)c2C(N)=O)cc1 ; "A25"]

### Step 1: ethyl 1-(4-(aminomethyl)phenyl)-4-(bis(tert-butoxycarbonyl)amino)-1H-pyrazole-5-carboxylate A25a

A mixture of ethyl 4-(bis(tert-butoxycarbonyl)amino)-1-(4-cyanophenyl)-1H-pyrazole-5-carboxylate A13e (5 g, 10.95 mmol) and Raney nickel (1 g) in EtOH (100 mL) and NH₄OH (0.5 mL) under hydrogen atmosphere was stirred at room atmosphere for 16 hours. The mixture was filtered over Celite, rinsed with MeOH, the filtrate was concentrated under vacuum to afford crude ethyl 1-(4-(aminomethyl)phenyl)-4-(bis(tert-butoxycarbonyl)amino)-1H-pyrazole-5-carboxylate A25a (4 g, 79% yield), which was used for next step without further purification.

### Step 2: ethyl 4-(bis(tert-butoxycarbonyl)amino)-1-(4-((5-fluoro-2-methoxybenzamido)methyl)phenyl)-1H-pyrazole-5-carboxylate A25b

A mixture of 5-fluoro-2-methoxybenzoic acid (2.2 g, 12.95 mmol), ethyl 1-(4-(aminomethyl)phenyl)-4-(bis(tert-butoxycarbonyl)amino)-1H-pyrazole-5-carboxylate A25a (4 g, 8.68 mmol), HATU (4.95 g, 13.04 mmol) and DIPEA (5.60 g, 43.40 mmol) in DMF (100 mL) under nitrogen atmosphere was stirred overnight at room temperature. The mixture was quenched by water (10 mL), and extracted with EtOAc (20 mL×3). The organic layers were dried over anhydrous Na₂SO₄, filtered, and concentrated under reduce pressure. The residue was purified by silica gel column chromatography (DCM/MeOH=50/1) to afford ethyl 4-(bis(tert-butoxycarbonyl)amino)-1-(4-((5-fluoro-2-methoxybenzamido)methyl)phenyl)-1H-pyrazole-5-carboxylate A25b (4.8 g, 90% yield). LCMS: MS m/z (ESI): 635.1 [M+Na]⁺.

### Step 3: ethyl 4-amino-1-(4-((5-fluoro-2-methoxybenzamido)methyl)phenyl)-1H-pyrazole-5-carboxylate A25c

A solution of ethyl 4-(bis(tert-butoxycarbonyl)amino)-1-(4-((5-fluoro-2-methoxybenzamido)methyl)phenyl)-1H-pyrazole-5-carboxylate A25b (4.8 g, 9.79 mmol) in HCl (4M in dioxane, 100 mL) was stirred at room temperature for 2 hours before concentrated under vacuum. The resulting residue was redissolved in H₂O (20 mL), and adjust pH to ˜ 5-6 with saturated NaHCO₃ solution. The resulting solution was extracted with EtOAc, and the combined organic layers were dried over anhydrous Na₂SO₄, filtered, and concentrated under reduced pressure to afford crude ethyl 4-amino-1-(4-((5-fluoro-2-methoxybenzamido)methyl)phenyl)-1H-pyrazole-5-carboxylate A25c (3.3 g, 81.72% yield), which was used for next step without further purification. LCMS: MS m/z (ESI): 413.0 [M+H]⁺.

### Step 4: ethyl 4-amino-3-bromo-1-(4-((5-fluoro-2-methoxybenzamido)methyl)phenyl)-1H-pyrazole-5-carboxylate A25d

To a solution of rude ethyl 4-amino-1-(4-((5-fluoro-2-methoxybenzamido)methyl)phenyl)-1H-pyrazole-5-carboxylate A25c (5 g, 12.12 mmol) in MeCN (100 mL) at room temperature was dropwise added a solution of NBS (2.22 g, 12.48 mmol) in MeCN (10 mL). The resulting mixture was then stirred at room temperature for 2 hours. The mixture was filtered, and the filtrate was washed with saturated Na₂S₂O₃ and brine, dried over anhydrous Na₂SO₄, and concentrated under reduced pressure. The residue was purified by silica gel column chromatography (DCM/MeOH=80/1) to afford ethyl 4-amino-3-bromo-1-(4-((5-fluoro-2-methoxybenzamido)methyl)phenyl)-1H-pyrazole-5-carboxylate A25d (1.7 g, 28.55% yield). ¹H NMR (400 MHz, DMSO-d₆): δ 8.88 (t, 1H), 7.51 (dd, 1H), 7.40-7.32 (m, 5H), 7.19 (dd, 1H), 5.17 (brs, 2H), 4.55 (d, 2H), 4.16 (q, 2H), 3.90 (s, 3H), 1.11 (t, 3H) ppm.

### Step 5: ethyl 4-amino-3-cyano-1-(4-((5-fluoro-2-methoxybenzamido)methyl)phenyl)-1H-pyrazole-5-carboxylate A25e

A mixture of ethyl 4-amino-3-bromo-1-(4-((5-fluoro-2-methoxybenzamido)methyl)phenyl)-1H-pyrazole-5-carboxylate A25d (80 mg, 162.83 umol), Pd(PPh₃)₄ (18.80 mg, 16.28 umol), and Zn(CN)₂ (230.76 mg, 1.96 mmol) in DMF (4 mL) under nitrogen atmosphere was stirred at 100° C. for 16 hours. After cooling down, the reaction mixture was quenched with water (20 mL) and then extracted with EtOAc (20 mL×3). The combined organic extracts were dried over anhydrous Na₂SO₄, filtered, and then concentrated under reduced pressure to afford crude 4-amino-3-cyano-1-(4-((5-fluoro-2-methoxybenzamido)methyl)phenyl)-1H-pyrazole-5-carboxylate A25e (61 mg, 85.64% yield), which was used directly for the next step without further purification. LCMS: MS m/z (ESI): 438.3 [M+H]⁺.

### Step 6: 4-amino-3-cyano-1-(4-((5-fluoro-2-methoxybenzamido)methyl)phenyl)-1H-pyrazole-5-carboxylic acid A25f

To a solution of ethyl 4-amino-3-cyano-1-(4-((5-fluoro-2-methoxybenzamido)methyl)phenyl)-1H-pyrazole-5-carboxylate A25e (61 mg, 137.16 umol) in MeOH (2 mL) was added LiOH (2 M in water, 0.5 mL). The resulting mixture was stirred at room temperature for 30 min. After concentration, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-3-cyano-1-(4-((5-fluoro-2-methoxybenzamido)methyl)phenyl)-1H-pyrazole-5-carboxylic acid A25f, which was used for next step without further purification. LCMS: MS m/z (ESI): 410.2 [M+H]⁺.

### Step 7: 4-amino-3-cyano-1-(4-((5-fluoro-2-methoxybenzamido)methyl)phenyl)-1H-pyrazole-5-carboxamide A25

A mixture of NH₄Cl (12.95 mg, 244.28 umol), HATU (69.62 mg, 183.21 umol), 4-amino-3-cyano-1-(4-((5-fluoro-2-methoxybenzamido)methyl)phenyl)-1H-pyrazole-5-carboxylic acid A25f (50 mg, 122.14 umol) and DIPEA (78.78 mg, 610.70 umol) under nitrogen atmosphere was stirred overnight at room temperature. The reaction mixture quenched by water (20 mL), and then extracted with EtOAc (30 mL×2). The combined organic extracts were washed with brine, dried over anhydrous Na₂SO₄, filtered, and concentrated under reduced pressure. The residue was purification by silica gel column chromatography (DCM/MeOH=50/1) to afford 4-amino-3-cyano-1-(4-((5-fluoro-2-methoxybenzamido)methyl)phenyl)-1H-pyrazole-5-carboxamide A25 (20.86 mg, 41.82% yield). ¹H NMR (400 MHz, DMSO-d₆): δ 8.89 (t, 1H), 7.51 (dd, 1H), 7.45-7.36 (m, 4H), 7.33 (dd, 1H), 7.19 (dd, 1H), 5.41 (brs, 2H), 4.56 (d, 2H), 3.89 (s, 3H) ppm; LCMS: MS m/z (ESI): 409.2 [M+H]⁺.

### Example A26

### 4-amino-1-(4-((5-fluoro-2-methoxybenzamido)methyl)phenyl)-3-(piperidin-4-yl)-1H-pyrazole-5-carboxamide A26

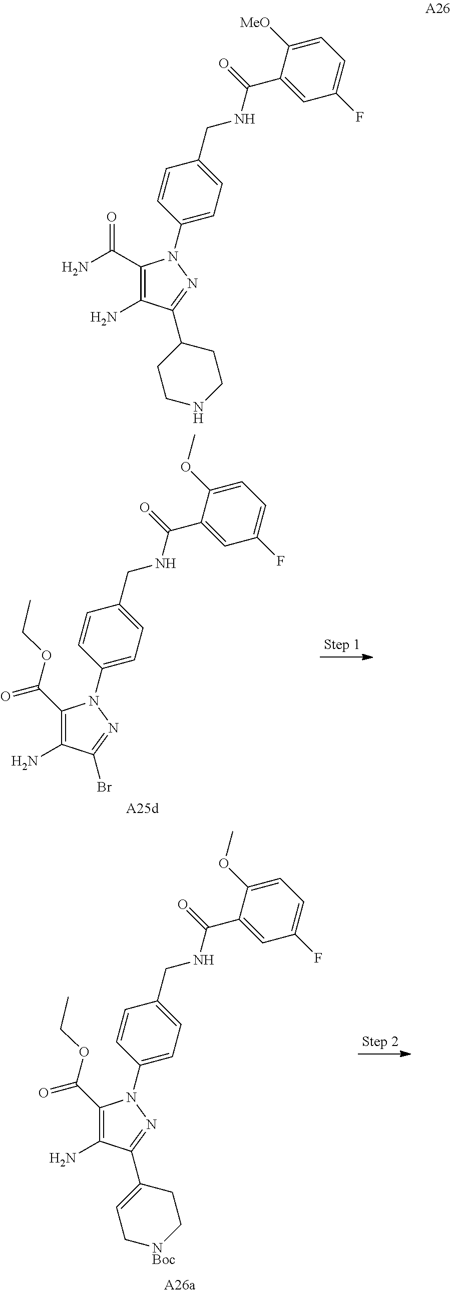

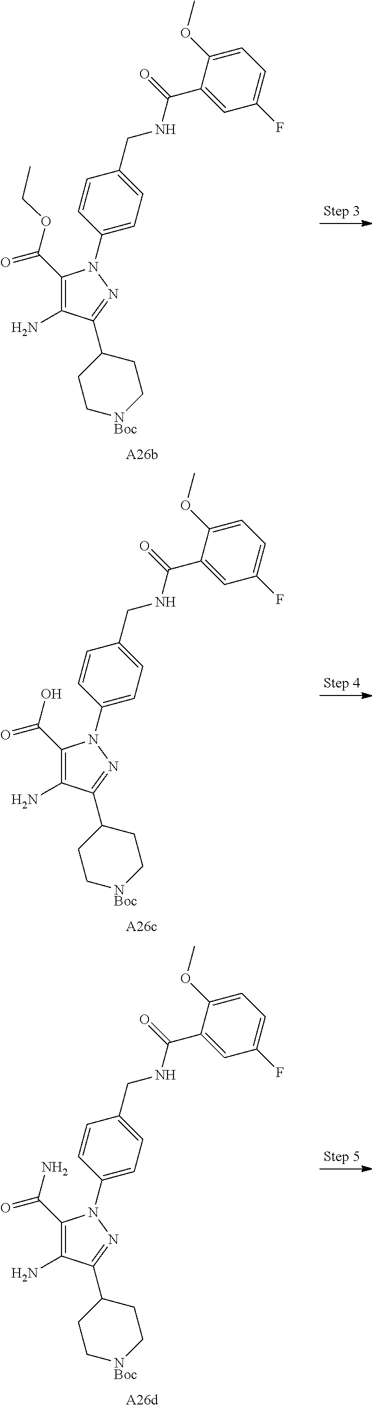 [SMILES: CCOC(=O)c1c(N)c(C2CCN(C(=O)OC(C)(C)C)CC2)nn1-c1ccc(CNC(=O)c2cc(F)ccc2OC)cc1 ; COc1ccc(F)cc1C(=O)NCc1ccc(-n2nc(C3CCN(C(=O)OC(C)(C)C)CC3)c(N)c2C(=O)O)cc1 ; COc1ccc(F)cc1C(=O)NCc1ccc(-n2nc(C3CCN(C(=O)OC(C)(C)C)CC3)c(N)c2C(N)=O)cc1]

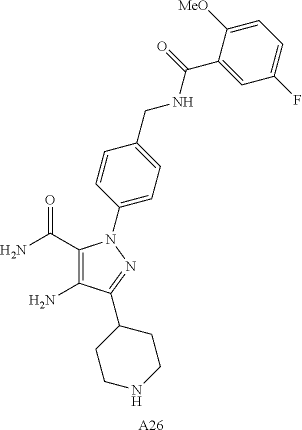

### Step 1: tert-butyl 4-(4-amino-5-(ethoxycarbonyl)-1-(4-((5-fluoro-2-methoxybenzamido)methyl)phenyl)-1H-pyrazol-3-yl)-3,6-dihydropyridine-1(2H)-carboxylate A26a

A mixture of ethyl 4-amino-3-bromo-1-(4-((5-fluoro-2-methoxybenzamido)methyl)phenyl)-1H-pyrazole-5-carboxylate A25d (100 mg, 203.53 umol), Pd(dppf)C12 (16.62 mg, 20.35 umol), tert-butyl 4-(4,4,5,5-tetramethyl-1,3,2-dioxaborolan-2-yl)-3,6-dihydropyridine-1(2H)-carboxylate (94.40 mg, 305.3 umol), and K₂CO₃ (56.17 mg, 407.06 umol) in dioxane (3 mL) and H₂O (0.3 mL) under nitrogen atmosphere was stirred overnight at 100° C. After cooling down and concentrated under reduced pressure, the resulting residue was purified by silica gel column chromatography (PE/EA=3/1) to afford tert-butyl 4-(4-amino-5-(ethoxycarbonyl)-1-(4-((5-fluoro-2-methoxybenzamido)methyl)phenyl)-1H-pyrazol-3-yl)-3,6-dihydropyridine-1(2H)-carboxylate A26a (100 mg, 82.76% yield). LCMS: MS m/z (ESI): 594.4 [M+H]⁺.

### Step 2: tert-butyl 4-(4-amino-5-(ethoxycarbonyl)-1-(4-((5-fluoro-2-methoxybenzamido)methyl)phenyl)-1H-pyrazol-3-yl)piperidine-1-carboxylate A26b

A mixture of tert-butyl 4-(4-amino-5-(ethoxycarbonyl)-1-(4-((5-fluoro-2-methoxybenzamido)methyl)phenyl)-1H-pyrazol-3-yl)-3,6-dihydropyridine-1(2H)-carboxylate A26a (100 mg, 168.45 umol) and Pd/C (0.1 g) in MeOH (4 mL) was stirred under hydrogen atmosphere (15 psi) at room temperature for 16 hours. Then, the mixture was filtered through Celite, and the filtrate was concentrated under reduced pressure to afford crude tert-butyl 4-(4-amino-5-(ethoxycarbonyl)-1-(4-((5-fluoro-2-methoxybenzamido)methyl)phenyl)-1H-pyrazol-3-yl)piperidine-1-carboxylate A26b (80 mg, 79.73% yield), which was used for next step without further purification. LCMS: MS m/z (ESI): 618.4 [M+Na]⁺

### Step 3: 4-amino-3-(1-(tert-butoxycarbonyl)piperidin-4-yl)-1-(4-((5-fluoro-2-methoxybenzamido)methyl)phenyl)-1H-pyrazole-5-carboxylic acid A26c

To a solution of tert-butyl 4-(4-amino-5-(ethoxycarbonyl)-1-(4-((5-fluoro-2-methoxybenzamido)methyl)phenyl)-1H-pyrazol-3-yl)piperidine-1-carboxylate A26b (80 mg, 134.30 umol) in MeOH (4 mL) at room temperature was added NaOH (3M in water, 2 mL). The resulting mixture was slowly warmed up and stirred overnight at 40° C. After cooling down, the pH of the mixture was adjusted to 5-6 by slow addition of 1M HCl aqueous solution. The reaction mixture was then extracted with EtOAc (20 mL×3). The combined organic extracts were washed with brine, dried over anhydrous Na₂SO₄, filtered, and concentrated under reduced pressure to get crude 4-amino-3-(1-(tert-butoxycarbonyl)piperidin-4-yl)-1-(4-((5-fluoro-2-methoxybenzamido)methyl)phenyl)-1H-pyrazole-5-carboxylic acid A26c (71 mg, 93.13% yield), which was used for next step without further purification. LCMS: MS m/z (ESI): 566.2 [M−H]⁻.

### Step 4: tert-butyl 4-(4-amino-5-carbamoyl-1-(4-((5-fluoro-2-methoxybenzamido)methyl)phenyl)-1H-pyrazol-3-yl)piperidine-1-carboxylate A26d

A mixture of NH₄Cl (13.38 mg, 250.16 umol), HATU (71.30 mg, 187.62 umol), 4-amino-3-(1-(tert-butoxycarbonyl)piperidin-4-yl)-1-(4-((5-fluoro-2-methoxybenzamido)methyl)phenyl)-1H-pyrazole-5-carboxylic acid A26c (71 mg, 125.08 umol) and DIPEA (80.68 mg, 625.40 umol) under nitrogen atmosphere was stirred overnight at room temperature. The reaction mixture quenched by water (10 mL), and then extracted with EtOAc (15 mL×3). The combined organic extracts were washed with brine, dried over anhydrous Na₂SO₄, filtered, and concentrated under reduced pressure. The residue was purified by silica gel column chromatography (DCM/MeOH=50/1) to afford crude tert-butyl 4-(4-amino-5-carbamoyl-1-(4-((5-fluoro-2-methoxybenzamido)methyl)phenyl)-1H-pyrazol-3-yl)piperidine-1-carboxylate A26d (65 mg, 91.71% yield), which was used for next step without further purification.

### Step 5: 4-amino-1-(4-((5-fluoro-2-methoxybenzamido)methyl)phenyl)-3-(piperidin-4-yl)-1H-pyrazole-5-carboxamide A26

A solution of tert-butyl 4-(4-amino-5-carbamoyl-1-(4-((5-fluoro-2-methoxybenzamido)methyl)phenyl)-1H-pyrazol-3-yl)piperidine-1-carboxylate A26d (65 mg, 114.71 umol) in HCl (4M in dioxane, 3 mL) was stirred at room temperature for 2 hours before concentrated under vacuum to afford 4-amino-1-(4-((5-fluoro-2-methoxybenzamido)methyl)phenyl)-3-(piperidin-4-yl)-1H-pyrazole-5-carboxamide A26 (26 mg, 45.13% yield). ¹H NMR (400 MHz, DMSO-d₆): δ 9.35-9.33 (m, 1H), 9.04-9.01 (m, 1H), 8.89 (t, 1H), 8.20-8.03 (m, 2H), 7.52 (dd, 1H), 7.45-7.37 (m, 4H), 7.36-7.32 (m, 1H), 7.19 (dd, 1H), 4.55 (d, 2H), 3.89 (s, 3H), 3.36-3.32 (m, 3H), 2.97-2.88 (m, 2H), 2.07-1.87 (m, 4H) ppm; LCMS: MS m/z (ESI): 467.3 [M+H]⁺.

### BIOLOGICAL ASSAYS

The present disclosure will be further described with reference to the following test examples, but the examples should not be considered as limiting the scope of the disclosure.

### Test Example 1. BTK-WT and BTK-C481S Kinase Activity Assays

These assays are to determine the degree of inhibition of the BTK-WT and BTK-C481S mutant kinase activity by measuring the amount of ADP produced during the enzymatic reactions. The full-length human BTK-WT (ABCAM205800) and the BTK-C481S (ABCAM 204166) kinase proteins were expressed and purified from Baculovirus infected Sf9 cells. The Km values of ATP and the substrate Poly (4:1 Glu, Tyr) (Signal Chem P61-58) in the assays were determined as 30 μM and 2 ng/μL respectively. Compounds, BTK enzymes, ATP and substrate were all prepared in ix kinase assay buffer by diluting 5× stock of kinase assay buffer III (Signal Chem K03-09), in H₂O, and adding DTT (Thermo ScientificA39255) for a final concentration of 50 μM. Compounds and BTK kinase proteins, with a total volume of 5 μL, were dispensed into a solid white flat bottom 384 well-plate (Corning 3824) and spun down at 1000 rpm for 2 minutes. Then the plate was kept shaking for 30 min at room temperature to allow the binding of the compounds to the proteins. After the preincubation, ATP and the substrate, a total of 5 μL was added into each well. The plate was then spun down at 1000 rpm for 2 minutes and kept shaking for 90 min at room temperature. ADP detection was performed following the guidance from the ADP-Glo Kinase Assay kits (Promega V9101). Briefly, 10 μL of ADP-Glo reagent was added into each well. The plate was then spun down at 1000 rpm for 2 minutes and kept shaking at room temperature for 60 min. Twenty μL of the kinase detection solution was added into each well. The plate was incubated at room temperature for 30 min in dark. The plates were read for luminescence signals in Tecan M1000 directly following the 30 min incubation. Relative inhibition of BTK kinase activities were analyzed by calculating the luminescence signal change:

$$Inhibition \% = ( 1 - \frac{RLU ( Sample ) - RLU ( Negative Control )}{RLU ( Positive Control ) - RLU ( Negative Control )} ) \times 100 \%$$

Data was input into GraphPad Prism, and the IC₅₀ values were calculated using function “log(inhibitor) vs. response—Variable slope (four parameters)”.

TABLE 1
BTK Kinase Inhibition

| Example No | BTK-WT IC₅₀ (nM) | BTK-C481S IC₅₀ (nM) |
|---|---|---|
| A1 | 5.6 | 3.6 |
| A3 | 90 | 43 |
| A5 | 11 | 11 |
| A9 | 14 | 14 |
| A10 | 62 | 60 |
| A11 | 35 | 27 |
| A12 | 26 | 5.8 |
| A13 | 4.4 | 2.7 |
| A14 | 7.9 | 3.1 |
| A15 | 3.7 | 4.2 |
| A16 | 15 | 11 |
| A17 | 87 | 93 |
| A19 | 63 | 68 |
| A22 | 8.1 | 8.0 |

The compounds of the present disclosure had significant inhibitory effects on both the wild type BTK and the mutant BTK-C481S kinase activities.

### Test Example 2. The BTK-Y223 Autophosphorylation in HEK293 Cells Carrying Stable Expression of BTK-WT and BTK-C481S

HEK293 cells (purchased from ATCC, CRL1573) stably expressing wild type BTK or mutant C481S BTK were generated by lentiviral transduction of constructs containing human BTK-WT or BTK-C481S mutant (Genecopoeia, Lv201 vector). Cells expressing BTK proteins were selected with puromycin treatment (1 μM). The protein expression was confirmed by western blotting of BTK and BTK-Y223 autophosphorylation. The cells were cultured in MEM medium (Sigma M2279), 10% heat-Inactivated FBS (Gibco 10100), penicillin streptomycin (Thermal Fisher 15140122), and puromycin (InvivoGen QLL-41-03). The BTK-Y223 phosphorylation was quantified using BTK phospho-Y223 kit (Cisbio 63ADK017PEG). HEK293/BTK stable cells were seeded in a 96-well plate at a density of 10 thousand cells/well in a total volume of 100 μL of media. Cells were incubated overnight in a humidified, 5% CO₂ cell culture incubator at 37° C. The next day, the cells were treated with diluted compounds in a humidified, 5% CO₂ cell culture incubator at 37° C. for 2 hours. The medium was removed from each well, and 50 μL of 1× lysis buffer from the assay kit was added into each well. The cells were incubated for 30 min at room temperature with shaking. Then 16 μL of lysate was transferred to a PROXIPLATE 384-plate (PerkinElmer 6008230), followed by addition of 4 μL of HTRF pre-mixed antibodies. After incubation overnight at room temperature, fluorescence signals in the plates were read on PHERAstar FSX instrument, using HTRF settings (665 nM/620 nM).

The relative cellular pBTK inhibition was calculated by HTRF signal change: The average of positive control well reading, and average of negative control reading were used as controls to calculate inhibition percentage.

$$Inhibition \% = [ 1 - \frac{Signal ( Sample ) - Signal ( Neg Ctrl )}{Signal ( Pos Ctrl ) - Signal ( Neg Ctrl )} ] \times 100 \%$$

The data was input into GraphPad Prism, using function “log(inhibitor) vs. response—Variable slope (four parameters)” to get IC₅₀ values.

TABLE 2
BTK-Y223 autophosphorylation in cells

| Example No | BTK-WT IC₅₀ (nM) | BTK-C481S IC₅₀ (nM) |
|---|---|---|
| A1 | 55 | 115 |
| A9 | 84 | 170 |
| A12 | 130 | 180 |
| A13 | 57 | 120 |
| A14 | 81 | 210 |
| A15 | 27 | 84 |
| A16 | 120 | 230 |
| A22 | 74 | 120 |

The compounds of the present disclosure had significant inhibitory effects on the cellular activities of both the wild type BTK and the mutant BTK-C481S kinases.

### Test Example 3. Effects of Compounds on the BTK-Dependent Cell Proliferation in Human TMD-8 Diffuse Large B-Cell Lymphoma Cells

The human TMD-8 DLBCL cancer cells were cultured in RPMI media with high glucose and glutamine (Genesee 25-506), 20% heat-inactivated FBS (Gibco 10100), penicillin streptomycin (Thermal Fisher 15140122) and 1 mM sodium pyruvate (Thermal Fisher 11360070). TMD-8 cells were centrifuged at 300 g for 5 minutes and the cells were resuspended into fresh cell culture media. The cells were counted and made to a cell stock of 1.3 million/mL. Then 75 μL of cells were seeded into each well of a white 96-well cell culture plate (Corning 353286). A serial dilution of compounds was made and 25 μL was added to each well. The plates were incubated for 3 days in a humidified, 5% CO₂ atmosphere at 37° C. Inhibition of cell growth by the compounds was determined by measuring the ATP level generated by the cells with the Cell Titer-Glo Luminescent Cell Viability Kit (Promega G7572). Assay procedures were conducted according to the protocols provided by Promega. In brief, the treated cell-culture plate and its contents were equilibrated at room temperature for approximately 30 minutes. 100 μL of CellTiter-Glo® Reagent was added into each cell-culture well, and contents were mixed for 2 minutes on an orbital shaker to induce cell lysis. The plate was incubated at room temperature for 10 minutes to stabilize luminescent signal. The resulting luminescence signal was immediately read using a TECAN plate reader. The relative cell grow inhibition was calculated by Cell Titer-Glo luminescent signal change.

The average readings of media-only wells and the readings of positive control wells (no compound treatment) were used to calculate response percentage.

$$Inhibition \% = [ 1 - \frac{Luminescence ( Sample ) - Luminescence ( Media only )}{Luminescence ( Positive CTRL ) - Luminescence ( Media only )} ] \times 100 \%$$

The data was input into GraphPad Prism, and the curve fitting was made using function “log(inhibitor) vs. response—Variable slope (four parameters)”. The interpolation function was used to calculate absolute IC₅₀ values.

TABLE 3
Effects of compounds on the BTK-dependent cell
proliferation in human TMD-8 cell

| Example No | IC₅₀ (nM) |
|---|---|
| A1 | 47 |
| A5 | 240 |
| A9 | 140 |
| A12 | 160 |
| A13 | 95 |
| A14 | 95 |
| A15 | 29 |
| A16 | 120 |
| A22 | 110 |

The compounds of the present disclosure had significant inhibitory effects on the TMD-8 DLBCL cell growth.

The foregoing embodiments and examples are provided for illustration only and are not intended to limit the scope of the invention. Various changes and modifications to the disclosed embodiments will be apparent to those skilled in the art based on the present disclosure, and such changes and modifications may be made without departure from the spirit and scope of the present invention. All patent or non-patent references cited are incorporated herein by reference in their entireties without admission of them as prior art.

What is claimed is:

1. A compound of formula (IM): 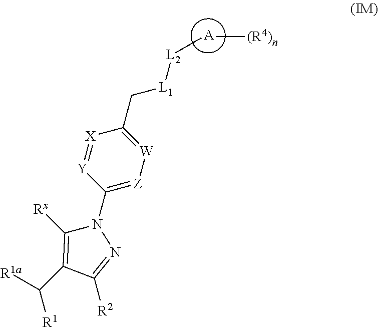
or a pharmaceutically acceptable salt, solvate, or prodrug thereof, wherein:
R^{x} is selected from the group consisting of C(O)NR^{A}R^{B}, alkyl, haloalkyl and hydroxyalkyl; wherein the alkyl is optionally substituted with one or more groups selected from the group consisting of alkoxy and haloalkoxy;
R¹ and R^{1a} are identical or different, and each is independently selected from the group consisting of hydrogen, alkyl, haloalkyl, —C(O)R⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;
R² is selected from the group consisting of alkyl, haloalkyl, hydroxyalkyl, —COR⁵, cyano, —OR⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;
or R¹ and R² together with the nitrogen and the carbon to which they are attached form a heterocycle; wherein the heterocycle is optionally substituted with one or more groups independently selected from the group consisting of halogen, alkyl, haloalkyl, alkoxy, haloalkoxy, cyano, amino, hydroxyl and hydroxyalkyl;
X, Y, Z and W are identical or different, and each is independently CR³ or N, provided that X, Y, Z and W are not all N at the same time;
R³ at each occurrence is identical or different, and each is selected from the group consisting of hydrogen, halogen, alkyl, haloalkyl, alkoxy, haloalkoxy, cyano, amino, hydroxyl and hydroxyalkyl;
L₁ and L₂ are different, and each is independently —NR^{7a} or C(O);
R^{7a} is selected from the group consisting of hydrogen, alkyl, haloalkyl and hydroxyalkyl;
ring A is aryl or heteroaryl;
R⁴ at each occurrence is identical or different, and each is selected from the group consisting of hydrogen, halogen, alkyl, haloalkyl, cyano, —OR^{6a}, —NR^{7b}R^{8b} and hydroxyalkyl;
R^{A}, R^{B}, R⁵, R⁶, R^{6a}, R⁷, R^{7b}, R⁸ and R^{8b} are identical or different, and each is independently selected from the group consisting of hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and
n is 0, 1, 2, 3, 4, 5 or 6.

2. The compound of claim 1, or a pharmaceutically acceptable salt, solvate, or prodrug thereof, wherein R^{1a} is hydrogen, being a compound of formula (I): 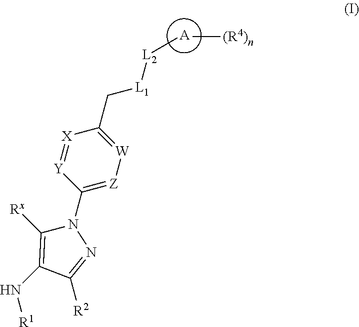
or a pharmaceutically acceptable salt, solvate, or prodrug thereof, wherein:
R^{x}, W, X, Y, Z, L₁, L₂, R¹, R², R⁴, ring A and n are as defined in claim 1.

3. The compound of claim 1, or a pharmaceutically acceptable salt, solvate, or prodrug thereof, wherein L₁ is NR^{7a}, L₂ is C(O), and R^{7a} is as defined in claim 1.

4. The compound of claim 1, or a pharmaceutically acceptable salt, solvate, or prodrug thereof, wherein R^{x} is C(O)NR^{A}R^{B}, and R^{A} and R^{B} are as defined in claim 1.

5. The compound of claim 3, or a pharmaceutically acceptable salt, solvate, or prodrug thereof, wherein R^{x} is C(O)NR^{A}R^{B}, R^{1a} is hydrogen and R^{7a} is hydrogen, being a compound of formula (II): 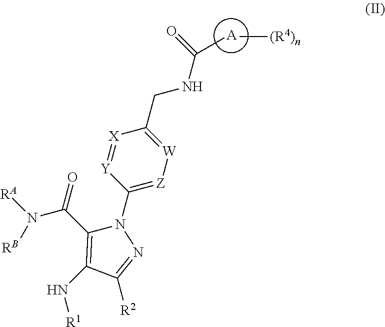
or a pharmaceutically acceptable salt, solvate, or prodrug thereof.

6. The compound of claim 5, or a pharmaceutically acceptable salt, solvate, or prodrug thereof, wherein ring A is selected from 6-10 membered aryl and 5-10 membered heteroaryl.

7. The compound of claim 5, or a pharmaceutically acceptable salt, solvate, or prodrug thereof, wherein ring A is phenyl or

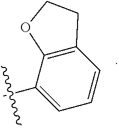

8. The compound of claim 5, or a pharmaceutically acceptable salt, solvate, or prodrug thereof, wherein R^{A} and R^{B} are identical, and each is hydrogen.

9. The compound of claim 8, or a pharmaceutically acceptable salt, solvate, or prodrug thereof, wherein ring A is phenyl, being a compound of formula (III): 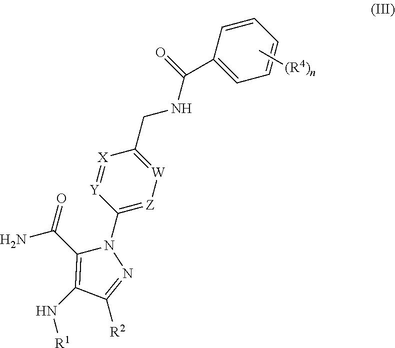
or a pharmaceutically acceptable salt, solvate, or prodrug thereof.

10. The compound of claim 9, or a pharmaceutically acceptable salt, solvate, or prodrug thereof, wherein W, X, Y, and Z are each CR³, being a compound of formula (IV): 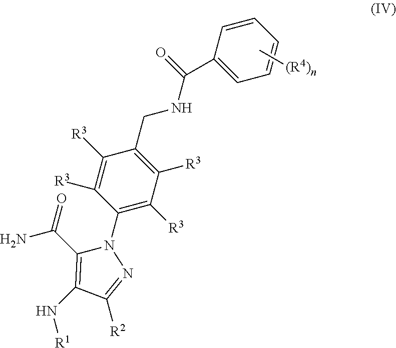
or a pharmaceutically acceptable salt, solvate, or prodrug thereof.

11. The compound of claim 1, or a pharmaceutically acceptable salt, solvate, or prodrug thereof, wherein R¹ is selected from the group consisting of hydrogen, C₁₋₆ alkyl and —C(O)R⁵, and R⁵ is C₁₋₆ alkyl.

12. The compound of claim 1, or a pharmaceutically acceptable salt, solvate, or prodrug thereof, wherein R² is selected from the group consisting of C₁₋₆ alkyl, C₁₋₆ haloalkyl, C₁₋₆ hydroxyalkyl, cyano, —OR⁵, 3-8 membered cycloalkyl, 3-12 membered heterocyclyl, 6-10 membered aryl and 5-10 membered heteroaryl; wherein the C₁₋₆ alkyl, 3-8 membered cycloalkyl, 3-12 membered heterocyclyl, 6-10 membered aryl and 5-10 membered heteroaryl are each optionally substituted with one or more groups independently selected from the group consisting of halogen, C₁₋₆ alkyl, C₁₋₆ haloalkyl, hydroxyl, C₁₋₆ alkoxy, C₁₋₆ haloalkoxy, oxo and C₁₋₆ hydroxyalkyl, and R⁵ is C₁₋₆ alkyl.

13. The compound of claim 1, or a pharmaceutically acceptable salt, solvate, or prodrug thereof, wherein R¹ and R² together with the nitrogen and the carbon to which they are attached form a 5-12 membered heterocycle; wherein the 5-12 membered heterocycle is optionally substituted with one or more groups independently selected from the group consisting of halogen, C₁₋₆ alkyl, C₁₋₆ haloalkyl, C₁₋₆ alkoxy, C₁₋₆ haloalkoxy, cyano, amino, hydroxyl and C₁₋₆ hydroxyalkyl.

14. The compound of claim 1, or a pharmaceutically acceptable salt, solvate, or prodrug thereof, wherein R⁴ at each occurrence is identical or different, and each is independently selected from the group consisting of hydrogen, halogen, C₁₋₆ alkyl, C₁₋₆ haloalkyl and —OR^{6a}; R^{6a} is selected from the group consisting of hydrogen, C₁₋₆ alkyl, C₁₋₆ haloalkyl, 3-8 membered cycloalkyl and 3-12 membered heterocyclyl.

15. The compound of claim 1, or a pharmaceutically acceptable salt, solvate, or prodrug thereof, wherein R³ is at each occurrence independently selected from the group consisting of hydrogen, halogen, C₁₋₆ alkyl, C₁₋₆ haloalkyl, C₁₋₆ alkoxy and C₁₋₆ haloalkoxy.

16. The compound of claim 1, or a pharmaceutically acceptable salt, solvate, or prodrug thereof, wherein the compound is selected from the group consisting of:

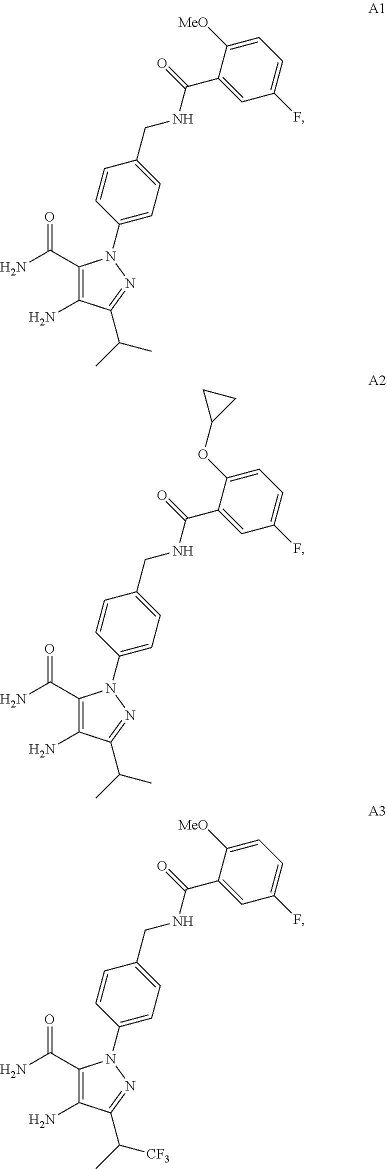 [SMILES: COc1ccc(F)cc1C(=O)NCc1ccc(-n2nc(C(C)C)c(N)c2C(N)=O)cc1 ; CC(C)c1nn(-c2ccc(CNC(=O)c3cc(F)ccc3OC3CC3)cc2)c(C(N)=O)c1N ; COc1ccc(F)cc1C(=O)NCc1ccc(-n2nc(C(C)C(F)(F)F)c(N)c2C(N)=O)cc1]

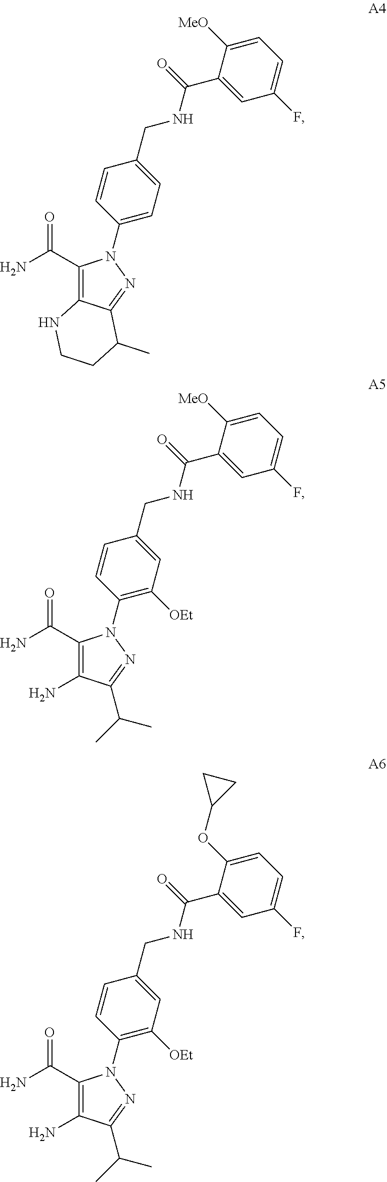 [SMILES: COc1ccc(F)cc1C(=O)NCc1ccc(-n2nc3c(c2C(N)=O)NCCC3C)cc1 ; CCOc1cc(CNC(=O)c2cc(F)ccc2OC)ccc1-n1nc(C(C)C)c(N)c1C(N)=O ; CCOc1cc(CNC(=O)c2cc(F)ccc2OC2CC2)ccc1-n1nc(C(C)C)c(N)c1C(N)=O]

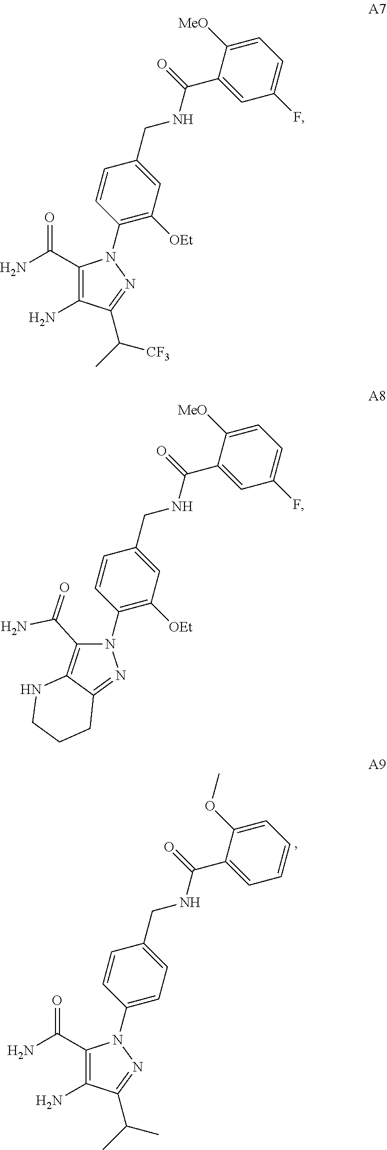 [SMILES: CCOc1cc(CNC(=O)c2cc(F)ccc2OC)ccc1-n1nc(C(C)C(F)(F)F)c(N)c1C(N)=O ; CCOc1cc(CNC(=O)c2cc(F)ccc2OC)ccc1-n1nc2c(c1C(N)=O)NCCC2 ; COc1ccccc1C(=O)NCc1ccc(-n2nc(C(C)C)c(N)c2C(N)=O)cc1]

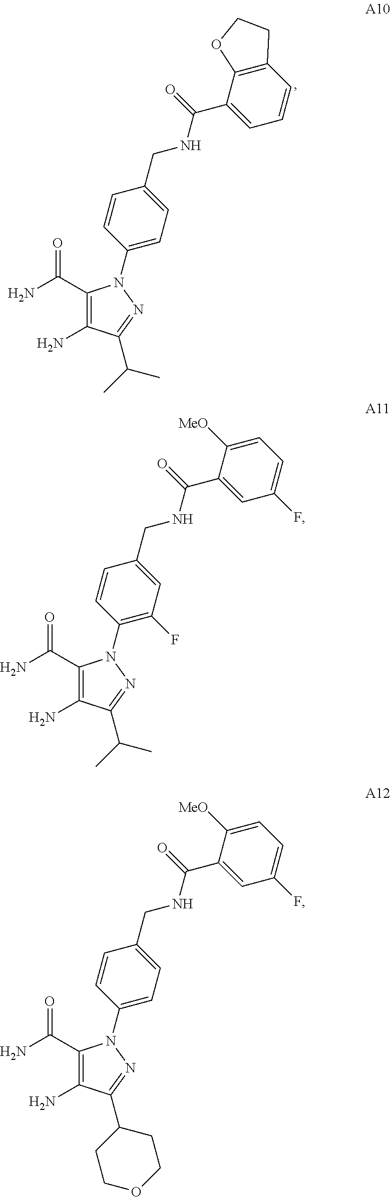 [SMILES: CC(C)c1nn(-c2ccc(CNC(=O)c3cccc4c3OCC4)cc2)c(C(N)=O)c1N ; COc1ccc(F)cc1C(=O)NCc1ccc(-n2nc(C(C)C)c(N)c2C(N)=O)c(F)c1 ; COc1ccc(F)cc1C(=O)NCc1ccc(-n2nc(C3CCOCC3)c(N)c2C(N)=O)cc1]

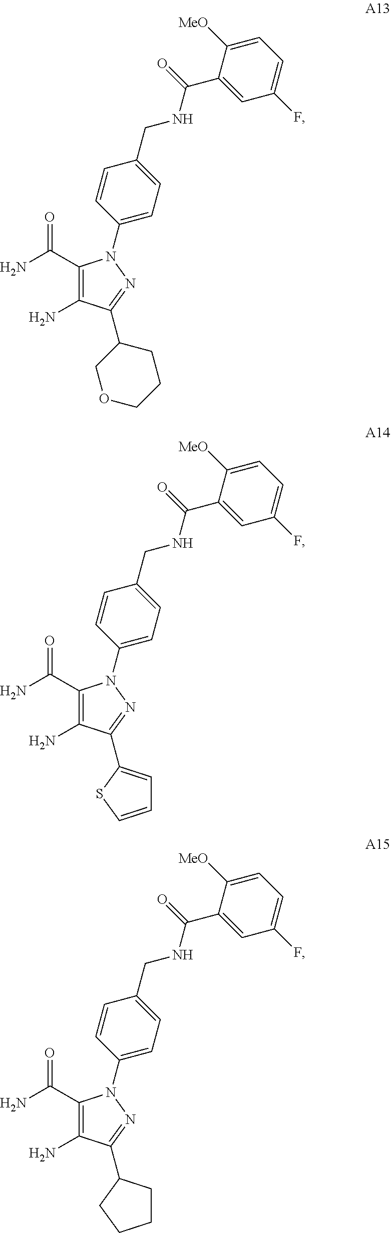 [SMILES: COc1ccc(F)cc1C(=O)NCc1ccc(-n2nc(C3CCCOC3)c(N)c2C(N)=O)cc1 ; COc1ccc(F)cc1C(=O)NCc1ccc(-n2nc(-c3cccs3)c(N)c2C(N)=O)cc1 ; COc1ccc(F)cc1C(=O)NCc1ccc(-n2nc(C3CCCC3)c(N)c2C(N)=O)cc1]

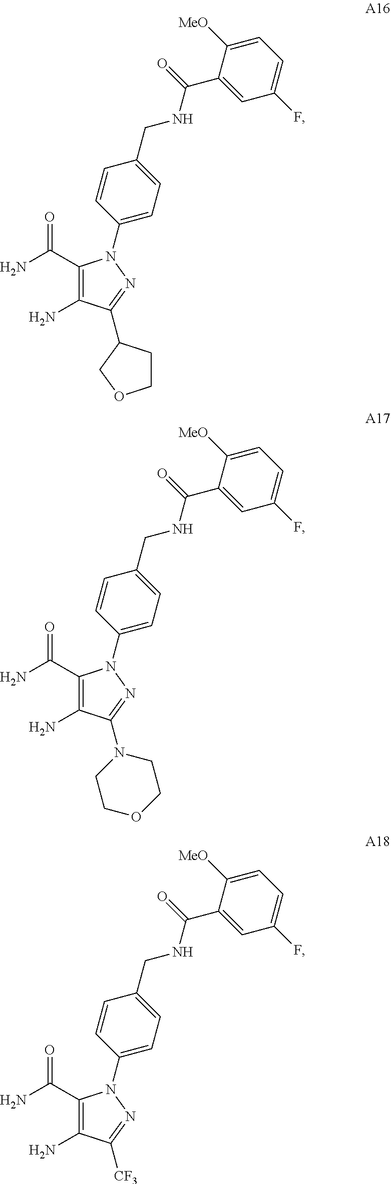 [SMILES: COc1ccc(F)cc1C(=O)NCc1ccc(-n2nc(C3CCOC3)c(N)c2C(N)=O)cc1 ; COc1ccc(F)cc1C(=O)NCc1ccc(-n2nc(N3CCOCC3)c(N)c2C(N)=O)cc1 ; COc1ccc(F)cc1C(=O)NCc1ccc(-n2nc(C(F)(F)F)c(N)c2C(N)=O)cc1]

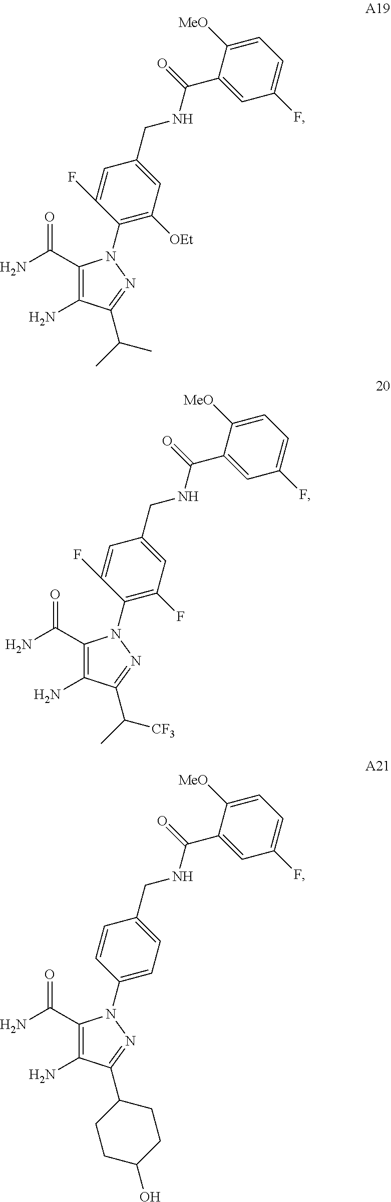 [SMILES: CCOc1cc(CNC(=O)c2cc(F)ccc2OC)cc(F)c1-n1nc(C(C)C)c(N)c1C(N)=O ; COc1ccc(F)cc1C(=O)NCc1cc(F)c(-n2nc(C(C)C(F)(F)F)c(N)c2C(N)=O)c(F)c1 ; COc1ccc(F)cc1C(=O)NCc1ccc(-n2nc(C3CCC(O)CC3)c(N)c2C(N)=O)cc1]

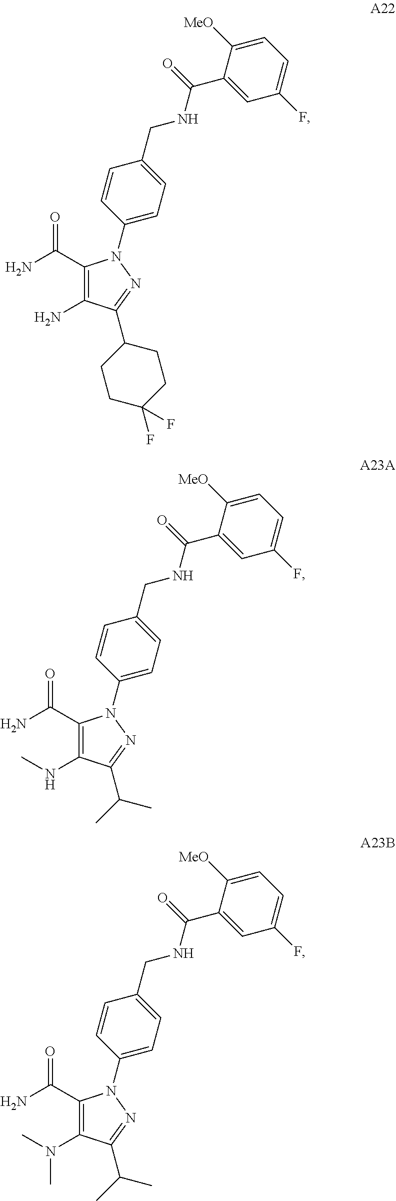 [SMILES: COc1ccc(F)cc1C(=O)NCc1ccc(-n2nc(C3CCC(F)(F)CC3)c(N)c2C(N)=O)cc1 ; CNc1c(C(C)C)nn(-c2ccc(CNC(=O)c3cc(F)ccc3OC)cc2)c1C(N)=O ; COc1ccc(F)cc1C(=O)NCc1ccc(-n2nc(C(C)C)c(N(C)C)c2C(N)=O)cc1]

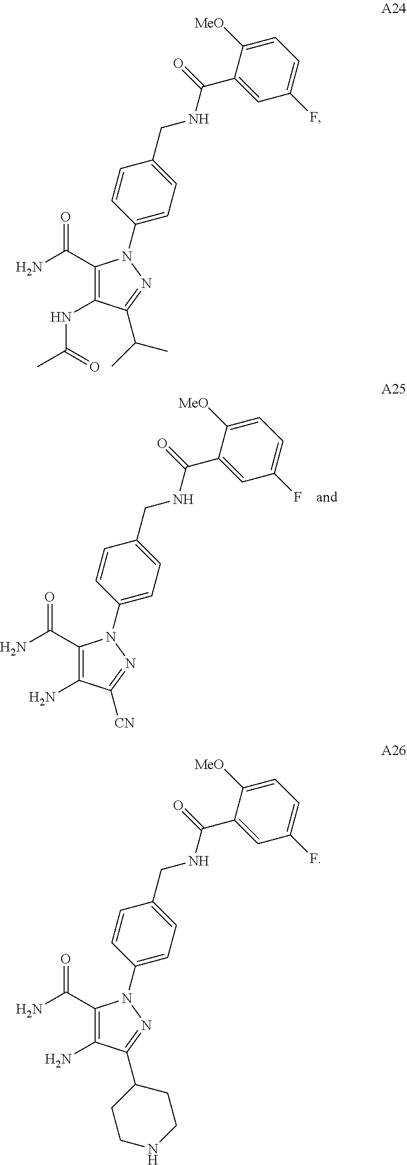 [SMILES: COc1ccc(F)cc1C(=O)NCc1ccc(-n2nc(C(C)C)c(NC(C)=O)c2C(N)=O)cc1 ; COc1ccc(F)cc1C(=O)NCc1ccc(-n2nc(C#N)c(N)c2C(N)=O)cc1 ; COc1ccc(F)cc1C(=O)NCc1ccc(-n2nc(C3CCNCC3)c(N)c2C(N)=O)cc1]

17. A compound of formula (IIA): 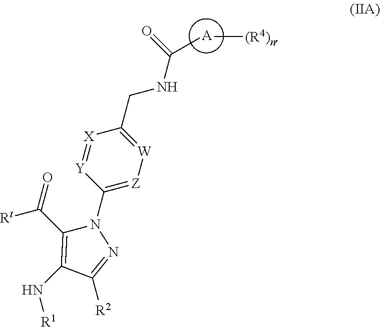
or a salt thereof, wherein:
R^{t} is selected from the group consisting of halogen, hydroxyl and alkoxy;
R¹ is selected from hydrogen, alkyl, haloalkyl, —C(O)R⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;
R² is selected from alkyl, haloalkyl, hydroxyalkyl, —COR⁵, cyano, —OR⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;
or R¹ and R² together with the nitrogen and the carbon to which they are attached form a heterocycle; wherein the heterocycle is optionally substituted with one or more groups independently selected from the group consisting of halogen, alkyl, haloalkyl, alkoxy, haloalkoxy, cyano, amino, hydroxyl and hydroxyalkyl;
X, Y, Z and W are identical or different, and each is independently CR³ or N, provided that X, Y, Z and W are not all N at the same time;
R³ at each occurrence is independently selected from hydrogen, halogen, alkyl, haloalkyl, alkoxy, haloalkoxy, cyano, amino, hydroxyl and hydroxyalkyl;
ring A is aryl or heteroaryl;
R⁴ at each occurrence is independently selected from hydrogen, halogen, alkyl, haloalkyl, cyano, —OR^{6a}, —NR^{7b}R^{8b} and hydroxyalkyl;
R^{7a} is selected from hydrogen, alkyl, haloalkyl and hydroxyalkyl;
R⁵, R⁶, R^{6a}, R⁷, R^{7b}, R⁸ and R^{8b} are each independently selected from hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and
n is 0, 1, 2, 3, 4, 5 or 6.

18. A compound of claim 17, or a pharmaceutically acceptable salt, solvate, or prodrug thereof, selected from:

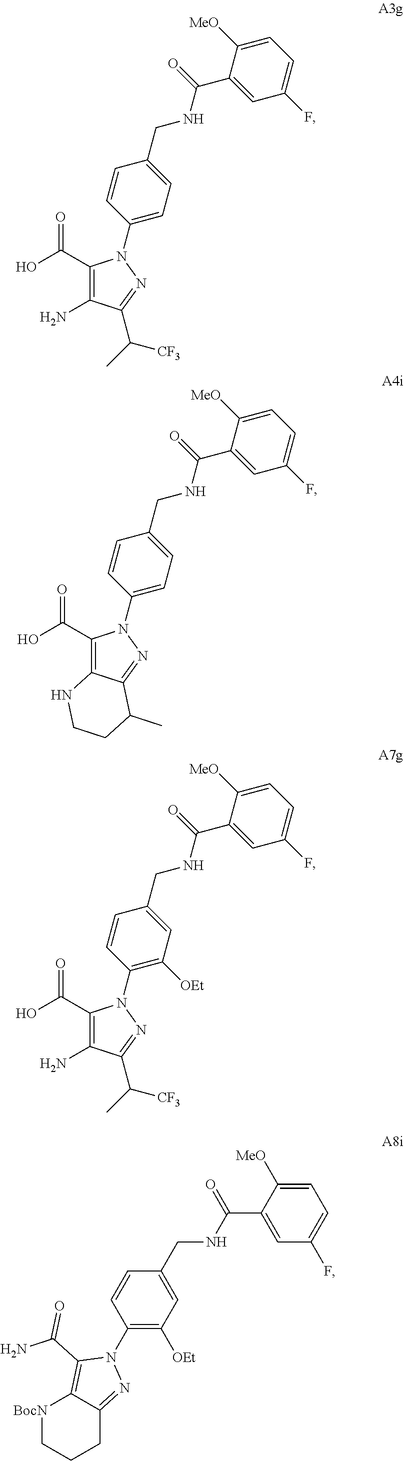 [SMILES: COc1ccc(F)cc1C(=O)NCc1ccc(-n2nc(C(C)C(F)(F)F)c(N)c2C(=O)O)cc1 ; COc1ccc(F)cc1C(=O)NCc1ccc(-n2nc3c(c2C(=O)O)NCCC3C)cc1 ; CCOc1cc(CNC(=O)c2cc(F)ccc2OC)ccc1-n1nc(C(C)C(F)(F)F)c(N)c1C(=O)O ; CCOc1cc(CNC(=O)c2cc(F)ccc2OC)ccc1-n1nc2c(c1C(N)=O)N(C(=O)OC(C)(C)C)CCC2]

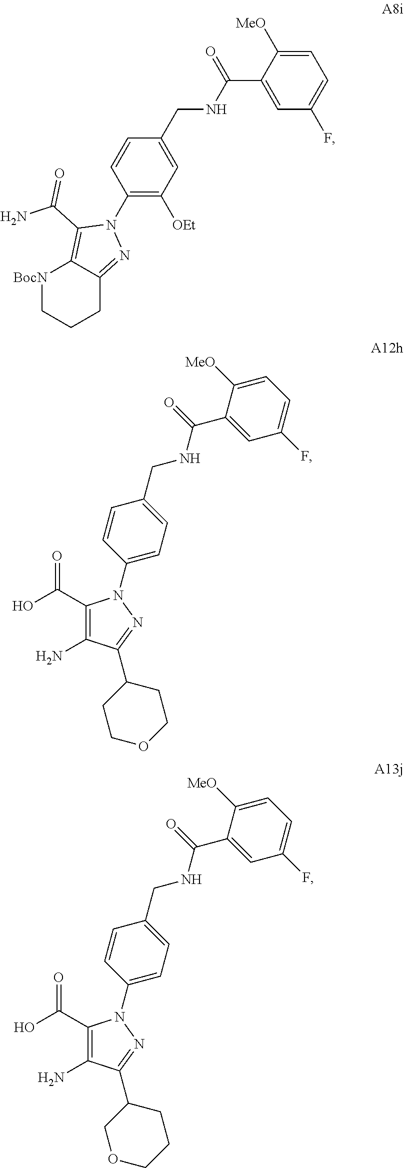 [SMILES: CCOc1cc(CNC(=O)c2cc(F)ccc2OC)ccc1-n1nc2c(c1C(N)=O)N(C(=O)OC(C)(C)C)CCC2 ; COc1ccc(F)cc1C(=O)NCc1ccc(-n2nc(C3CCOCC3)c(N)c2C(=O)O)cc1 ; COc1ccc(F)cc1C(=O)NCc1ccc(-n2nc(C3CCCOC3)c(N)c2C(=O)O)cc1]

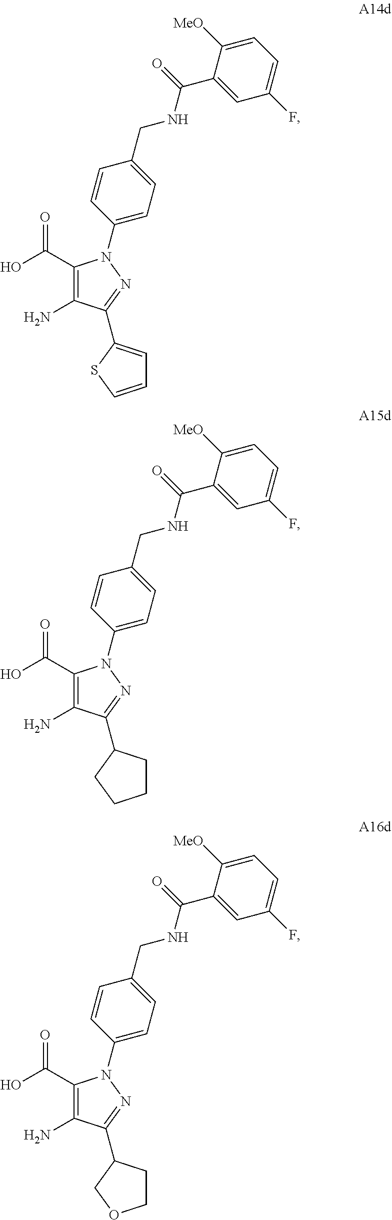 [SMILES: COc1ccc(F)cc1C(=O)NCc1ccc(-n2nc(-c3cccs3)c(N)c2C(=O)O)cc1 ; COc1ccc(F)cc1C(=O)NCc1ccc(-n2nc(C3CCCC3)c(N)c2C(=O)O)cc1 ; COc1ccc(F)cc1C(=O)NCc1ccc(-n2nc(C3CCOC3)c(N)c2C(=O)O)cc1]

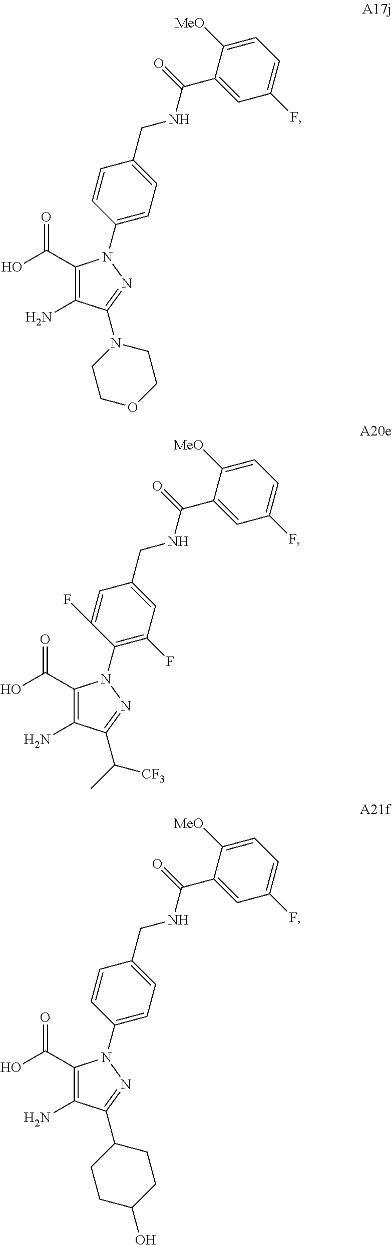 [SMILES: COc1ccc(F)cc1C(=O)NCc1ccc(-n2nc(N3CCOCC3)c(N)c2C(=O)O)cc1 ; COc1ccc(F)cc1C(=O)NCc1cc(F)c(-n2nc(C(C)C(F)(F)F)c(N)c2C(=O)O)c(F)c1 ; COc1ccc(F)cc1C(=O)NCc1ccc(-n2nc(C3CCC(O)CC3)c(N)c2C(=O)O)cc1]

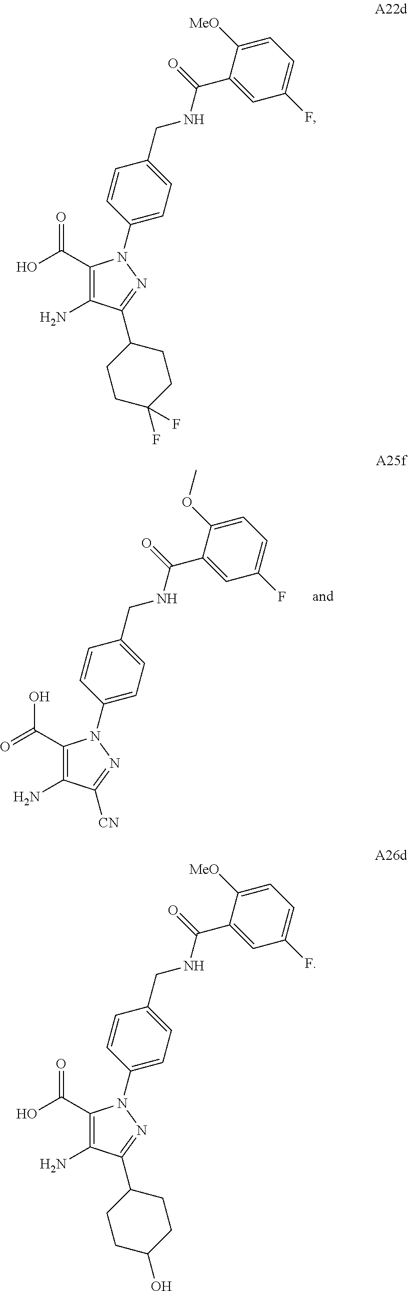 [SMILES: COc1ccc(F)cc1C(=O)NCc1ccc(-n2nc(C3CCC(F)(F)CC3)c(N)c2C(=O)O)cc1 ; COc1ccc(F)cc1C(=O)NCc1ccc(-n2nc(C#N)c(N)c2C(=O)O)cc1 ; COc1ccc(F)cc1C(=O)NCc1ccc(-n2nc(C3CCC(O)CC3)c(N)c2C(=O)O)cc1]

19. A compound of formula (IIB): 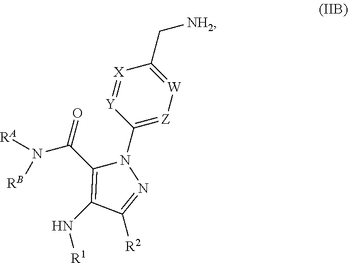
or a salt thereof, wherein:
R¹ is selected from hydrogen, alkyl, haloalkyl, —C(O)R⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, hydroxyalkyl, cycloalkyl, heterocyclyl, aryl and heteroaryl;
R² is selected from alkyl, haloalkyl, hydroxyalkyl, —COR⁵, cyano, —OR⁵, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, —OR⁶, —NR⁷R⁸, —OC(O)R⁶, —C(O)R⁶, —C(O)OR⁶, —NR^{7a}C(O)R⁶, —C(O)NR⁷R⁸, —NR^{7a}S(O)₂R⁶, —S(O)₂NR⁷R⁸, cyano, oxo, hydroxyalkyl, cycloalkyl, heterocyclyl, aryl and heteroaryl;
or R¹ and R² together with the nitrogen and the carbon to which they are attached form a heterocycle; wherein the heterocycle is optionally substituted with one or more groups independently selected from the group consisting of halogen, alkyl, haloalkyl, alkoxy, haloalkoxy, cyano, amino, hydroxyl and hydroxyalkyl;
X, Y, Z and W are identical or different, and each is independently CR³ or N, provided that X, Y, Z and W are not all N at the same time;
R³ at each occurrence is identical or different, and each is selected from the group consisting of hydrogen, halogen, alkyl, haloalkyl, alkoxy, haloalkoxy, cyano, amino, hydroxyl and hydroxyalkyl;
R^{7a} is selected from the group consisting of hydrogen, alkyl, haloalkyl and hydroxyalkyl;
R^{A}, R^{B}, R⁵, R⁶, R⁷, and R⁸ are each independently selected from hydrogen, alkyl, haloalkyl, hydroxyalkyl, cycloalkyl, heterocyclyl, aryl and heteroaryl; wherein the alkyl, cycloalkyl, heterocyclyl, aryl and heteroaryl are each optionally substituted with one or more groups independently selected from the group consisting of halogen, alkyl, haloalkyl, alkoxy, haloalkoxy, cyano, amino, hydroxyl, oxo, hydroxyalkyl, cycloalkyl, heterocyclyl, aryl and heteroaryl; and
n is 0, 1, 2, 3, 4, 5 or 6.

20. The compound according to claim 19, or a salt thereof, wherein the compound is selected from:

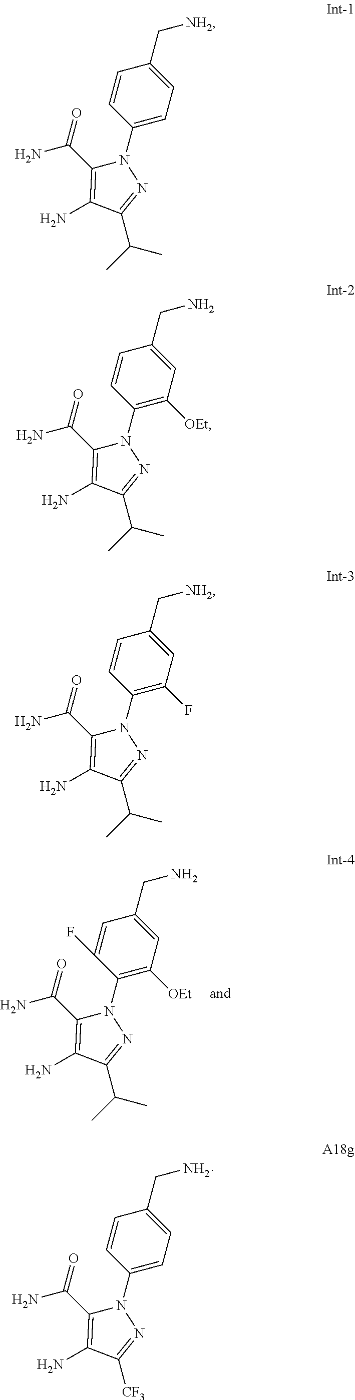 [SMILES: CC(C)c1nn(-c2ccc(CN)cc2)c(C(N)=O)c1N ; CCOc1cc(CN)ccc1-n1nc(C(C)C)c(N)c1C(N)=O ; CC(C)c1nn(-c2ccc(CN)cc2F)c(C(N)=O)c1N ; CCOc1cc(CN)cc(F)c1-n1nc(C(C)C)c(N)c1C(N)=O ; NCc1ccc(-n2nc(C(F)(F)F)c(N)c2C(N)=O)cc1]

21. A process of preparing the compound of formula (II) according to claim 5, or a pharmaceutically acceptable salt, solvate, or prodrug thereof, comprising a step of: 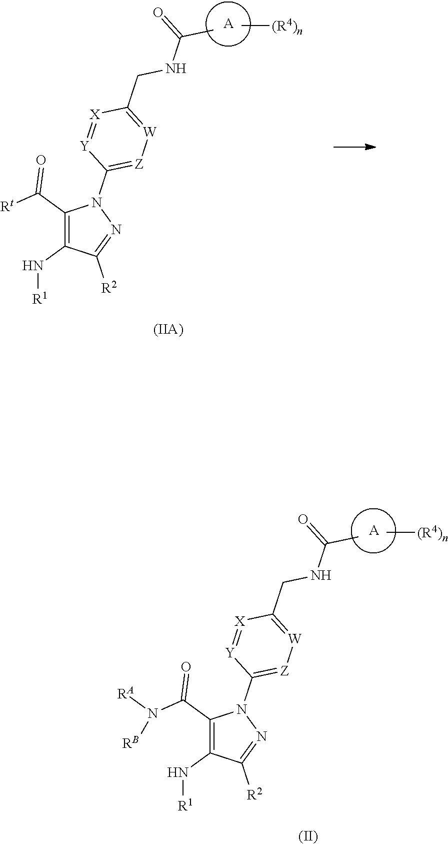
reacting a compound of Formula (IIA) or a salt thereof with NHR^{A}R^{B} to obtain the compound of formula(II) or a pharmaceutically acceptable salt, solvate, or prodrug thereof; wherein:
R^{t} is selected from the group consisting of halogen, hydroxyl and alkoxy; and
R^{A}, R^{B}, W, X, Y, Z, R¹, R², R⁴, ring A and n are as defined in claim 5.

22. A process of preparing the compound of formula (II) according to claim 5, or a pharmaceutically acceptable salt, solvate, or prodrug thereof, comprising a step of: 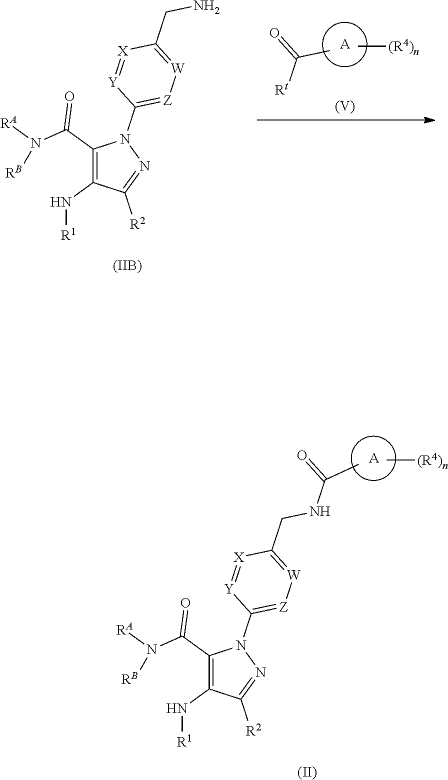
reacting a compound of Formula (IIB) or a salt thereof with a compound of Formula (V) to obtain the compound of formula(II) or a pharmaceutically acceptable salt, solvate, or prodrug thereof; wherein:
R^{t} is selected from the group consisting of halogen, hydroxyl and alkoxy; and
R^{A}, R^{B}, W, X, Y, Z, R¹, R², R⁴, ring A and n are as defined in claim 5.

23. A pharmaceutical composition, comprising a compound of claim 1, or a pharmaceutically acceptable salt, solvate, or prodrug thereof, and a pharmaceutically acceptable carrier.

24. A method of treating a disease or condition modulated by BTK, comprising administering to a subject in need thereof a therapeutically effective amount of a compound of claim 1, or a pharmaceutically acceptable salt, solvate, prodrug, or composition thereof.

25. The method according to claim 24, wherein the disease or condition modulated by BTK is selected from cancers, immune disorders, cardiovascular diseases, viral infections, inflammation, metabolism/endocrine function disorders, neurological disorders; preferably, the condition modulated by BTK is selected from the group consisting of B-cell malignancy, B-cell lymphoma, diffuse large B cell lymphoma, chronic lymphocyte leukemia, non-Hodgkin lymphoma (for example ABC-DLBCL), mantle cell lymphoma, follicular lymphoma, hairy cell leukemia, B-cell non-Hodgi lymphoma, Waldenstrom's macroglobulinemia, multiple myeloma, bone cancer, bone metastasis, arthritis, multiple sclerosis, osteoporosis, irritable bowel syndrome, inflammatory bowel disease, Crohn's disease, Sjogren's syndrome and lupus, chronic lymphocytic lymphoma, B-cell prolymphocyte leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, burkitt lymphoma/leukemia, lymphomatoid granulomatosis, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, vulvodynia, graft versus host disease, transplantation, transfusion, anaphylaxis, allergy, type I hypersensitivity, allergic conjunctivitis, allergic rhinitis, atopic dermatitis, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, vulvitis, pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, bronchiectasis, fatty liver disease, steatosis (e.g., nonalcoholic steatohepatitis (NASH), cholestatic liver disease (e.g., primary biliary cirrhosis (PBC), cirrhosis, alcohol-induced liver fibrosis, biliary duct injury, biliary fibrosis, cholestasis or cholangiopathies. In some embodiments, hepatic or liver fibrosis includes, but is not limited to, hepatic fibrosis associated with alcoholism, viral infection, e.g., hepatitis (e.g., hepatitis C, B or D), autoimmune hepatitis, nonalcoholic fatty liver disease (NAFLD), progressive massive fibrosis, exposure to toxins or irritants (e.g., alcohol, pharmaceutical drugs and environmental toxins), renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent, fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease, age-related macular degeneration, diabetic retinopathy, retinopathy of prematurity and neovascular glaucoma.
